(19) United States
(12) Patent Application Publication
(10) Pub. No.: US 2013/0029962 A1
(43) Pub. Date: Jan. 31, 2013

(54) Substituted Heteroaromatic Pyrazole-Containing Carboxamide and Urea Compounds as Vanilloid Receptor Ligands

(71) Applicants: Robert FRANK, Aachen (DE); Gregor BAHRENBERG, Monschau-Konzen (DE); Thomas CHRISTOPH, Aachen (DE); Bernhard LESCH, Aachen (DE); Jeewoo LEE, Seoul (KR)

(72) Inventors: Robert FRANK, Aachen (DE); Gregor BAHRENBERG, Monschau-Konzen (DE); Thomas CHRISTOPH, Aachen (DE); Bernhard LESCH, Aachen (DE); Jeewoo LEE, Seoul (KR)

(73) Assignee: Gruenenthal GmbH, Aachen (DE)

(21) Appl. No.: 13/557,941

(22) Filed: Jul. 25, 2012

Related U.S. Application Data
(60) Provisional application No. 61/511,752, filed on Jul. 26, 2011.

(30) Foreign Application Priority Data
Jul. 26, 2011 (EP) 11 006 114.0

(51) Int. Cl. A61K 31/4439; C07D 403/12; A61K 31/506; C07D 401/14; A61P 29/00; A61P 25/06; A61P 25/30; A61P 25/28; A61P 25/16; A61P 3/04; A61P 9/12; A61P 11/08; A61P 9/00; A61P 17/02; A61P 25/00; A61P 23/00; A61P 15/00; C07D 401/12

(52) U.S. Cl. 514/210.2; 546/275.4; 514/341; 544/333; 514/256

(57) ABSTRACT

Substituted heteroaromatic pyrazole-containing carboxamide and urea compounds as vanilloid receptor ligands, pharmaceutical compositions containing these compounds and also to a method of using these compounds for treating and/or inhibiting pain and further diseases and/or disorders.

### CROSS REFERENCE TO RELATED APPLICATIONS

This application claims priority from co-pending U.S. provisional patent application No. 61/511,752, filed Jul. 26, 2011, the entire disclosure of which is incorporated herein by reference. Priority is also claimed based on European patent application no. EP 11 006 114.0, filed Jul. 26, 2011, the entire disclosure of which is likewise incorporated herein by reference.

### BACKGROUND OF THE INVENTION

The invention relates to substituted heteroaromatic pyrazole-containing carboxamide and urea derivatives as vanilloid receptor ligands, to pharmaceutical compositions containing these compounds and also to these compounds for use in the treatment and/or inhibition of pain and further diseases and/or disorders.

The treatment of pain, in particular of neuropathic pain, is very important in medicine. There is a worldwide demand for effective pain therapies. The urgent need for action for a patient-focused and target-oriented treatment of chronic and non-chronic states of pain, this being understood to mean the successful and satisfactory treatment of pain for the patient, is also documented in the large number of scientific studies which have recently appeared in the field of applied analgesics or basic research on nociception.

The subtype 1 vanilloid receptor (VR1/TRPV1), which is often also referred to as the capsaicin receptor, is a suitable starting point for the treatment of pain, in particular of pain selected from the group consisting of acute pain, chronic pain, neuropathic pain and visceral pain. This receptor is stimulated inter alia by vanilloids such as capsaicin, heat and protons and plays a central role in the formation of pain. In addition, it is important for a large number of further physiological and pathophysiological processes and is a suitable target for the therapy of a large number of further disorders such as, for example, migraine, depression, neurodegenerative diseases, cognitive disorders, states of anxiety, epilepsy, coughs, diarrhoea, pruritus, inflammations, disorders of the cardiovascular system, eating disorders, medication dependency, misuse of medication and urinary incontinence.

There is a need for further compounds having comparable or better properties, not only with regard to affinity to vanilloid receptors 1 (VR1/TRPV1 receptors) per se (potency, efficacy).

Thus, it may be advantageous to improve the metabolic stability, the solubility in aqueous media or the permeability of the compounds. These factors can have a beneficial effect on oral bioavailability or can alter the PK/PD (pharmacokinetic/pharmacodynamic) profile; this can lead to a more beneficial period of effectiveness, for example.

### SUMMARY OF THE INVENTION

It was therefore an object of the invention to provide novel compounds, preferably having advantages over the prior-art compounds.

Another object of the invention was to provide new compounds with vanilloid receptor 1 activity which are suitable in particular as pharmacological active ingredients in pharmaceutical compositions.

It was a particular object of the invention to provide new compounds which are useful in pharmaceutical compositions for the treatment and/or inhibition of disorders or diseases which are at least partially mediated by vanilloid receptors 1 (VR1/TRPV1 receptors).

These and other objects have been achieved by the invention as described and claimed hereinafter.

It has surprisingly been found that the substituted compounds of general formula (I), as given below, display outstanding affinity to the subtype 1 vanilloid receptor (VR1/TRPV1 receptor) and are therefore particularly suitable for the inhibition and/or treatment of disorders or diseases which are at least partially mediated by vanilloid receptors 1 (VR1/TRPV1).

The present invention therefore relates to a substituted compound of general formula (I),

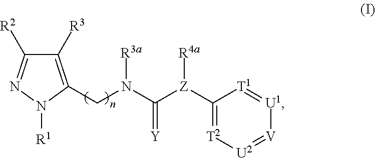

wherein
R⁰ represents a C₁₋₁₀ aliphatic residue, unsubstituted or mono- or polysubstituted; a C₃₋₁₀ cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R¹ represents H; R⁰; C(═O)—R⁰; C(═O)—OH; C(═O)—OR⁰; C(═O)—NHR⁰; C(═O)—N(R⁰)₂; OH; O—R⁰; SH; S—R⁰; S(═O)₂—R⁰; S(═O)₂—OR⁰; S(═O)₂—NHR⁰; S(═O)₂—N(R⁰)₂; NH₂; NHR⁰; N(R⁰)₂; NH—S(═O)₂—R⁰; N(R⁰)(S(═O)₂—R⁰; or SCl_{S};
R² represents H; R⁰; F; Cl; Br; I; CN; NO₂; OH; SH; CF₃; CF₂H; CFH₂; CF₂Cl; CFCl₂; CH₂CF₃; OCF₃; OCF₂H; OCFH₂; OCF₂Cl; OCFCl₂; SCF₃; SCF₂H; SCFH₂; SCF₂Cl; SCFCl₂; S(═O)₂—CF₃; S(═O)₂—CF₂H; S(═O)₂—CFH₂; or SF₅;
R³ represents H or a C₁₋₁₀ aliphatic residue, unsubstituted or mono- or polysubstituted;
R^{3a} represents H or a C₁₋₄ aliphatic residue, unsubstituted or mono- or polysubstituted;
n represents 0, 1, 2, 3 or 4, preferably represents 1, 2, 3 or 4, more preferably represents 1, 2 or 3, even more preferably represents 1 or 2, most preferably denotes 1;
R^{4a} represents H or a C₁₋₄ aliphatic residue, unsubstituted or mono- or polysubstituted, a C₃₋₆ cycloaliphatic residue, unsubstituted or mono- or polysubstituted, or an aryl, unsubstituted or mono- or polysubstituted;
Y represents O, S, or N—CN, preferably represents O;
Z represents N or C—R^{4b},
R^{4b} represents H or a C₁₋₄ aliphatic residue, unsubstituted or mono- or polysubstituted; or
R^{4a} and R^{4b} together with the carbon atom connecting them form a C₃₋₆ cycloaliphatic residue, unsubstituted or mono- or polysubstituted;
T¹ represents N or C—R⁵,
U¹ represents N or C—R⁶,
V represents N or C—R⁷,
U² represents N or C—R⁸,
T² represents N or C—R⁹,
   with the proviso that 1, 2 or 3 of variables T¹, U¹, V, U² and T² represent(s) a nitrogen atom,
   R⁵, R⁶, R⁷, R⁸ and R⁹ each independently of one another represent H; F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NH—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
   in which an “aliphatic group” and “aliphatic residue” can in each case, independently of one another, be branched or unbranched, saturated or unsaturated;
   in which a “cycloaliphatic residue” and a “heterocycloaliphatic residue” can in each case, independently of one another, be saturated or unsaturated;
   in which “mono- or polysubstituted” with respect to an “aliphatic group”, an “aliphatic residue”, a “cycloaliphatic residue” and a “heterocycloaliphatic residue” relates in each case independently of one another, with respect to the corresponding residues or groups, to the replacement of one or more hydrogen atoms each independently of one another by at least one substituent selected from the group consisting of F; Cl; Br; I; NO₂; CN; ═O; ═NH; ═N(OH); ═C(NH₂)₂; CF₃; CF₂H; CFH₂; CF₂Cl; CFCl₂; R⁰; C(═O)—H; C(═O)—R⁰; C(═O)—OH; C(═O)—OR⁰; CO—NH₂; C(═O)—NHR⁰; C(═O)—N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NH—R⁰; N(R⁰)₂; NH—C(═O)—R⁰; NH—C(═O)—O—R⁰; NH—C(═O)—NH₂; NH—C(═O)—NHR⁰; NH—C(═O)—N(R)₂; NR⁰—C(═O)—R⁰; NR⁰—C(═O)—O—R⁰; NR⁰—C(═O)—NH₂; NR⁰—C(═O)—NHR⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂—R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and)S(═O)₂—N(R⁰)₂;
   in which “mono- or polysubstituted” with respect to “aryl” and a “heteroaryl” relates, with respect to the corresponding residues, in each case independently of one another, to the replacement of one or more hydrogen atoms each independently of one another by at least one substituent selected from the group consisting of F; Cl; Br; I; NO₂; CN; CF₃; CF₂H; CFH₂; CF₂Cl; CFCl₂; R⁰; C(═O)—H; C(═O)—R⁰; C(═O)—OH; C(═O)—OR⁰; CO—NH₂; C(═O)—NHR⁰; C(═O)—N(R⁰)₂; OH;

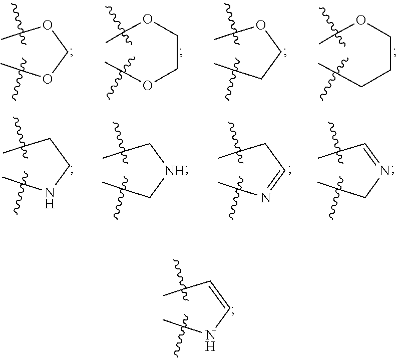

OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NHR⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and)S(═O)₂—N(R⁰)₂;
optionally in the form of a single stereoisomer or a mixture of stereoisomers, in the form of the free compound and/or a physiologically acceptable salt or a solvate, in particular hydrate, thereof.

### DETAILED DESCRIPTION

The term “single stereoisomer” comprises in the sense of this invention an individual enantiomer or diastereomer. The term “mixture of stereoisomers” comprises in the sense of this invention the racemate and mixtures of enantiomers and/or diastereomers in any mixing ratio.

The term “physiologically acceptable salt” comprises in the sense of this invention a salt of at least one compound according to the present invention and at least one physiologically acceptable acid or base.

The terms “C₁₋₁₀ aliphatic residue”, “C₁₋₈ aliphatic residue”, and “C₁₋₄ aliphatic residue” comprise in the sense of this invention acyclic saturated or unsaturated aliphatic hydrocarbon residues, which can be branched or unbranched and also unsubstituted or mono- or polysubstituted, which contain 1 to 10, or 1 to 8, or 1 to 4 carbon atoms respectively, i.e. C₁₋₁₀ alkanyls (C₁₋₁₀ alkyls), C₂₋₁₀ alkenyls and C₂₋₁₀ alkynyls as well as C₁₋₈ alkanyls (C₁₋₈ alkyls), C₂₋₈ alkenyls and C₂₋₈ alkynyls as well as C₁₋₄ alkanyls (C₁₋₄ alkyls), C₂₋₄ alkenyls and C₂₋₄ alkynyls, respectively. Alkenyls comprise at least one C—C double bond (a C═C-bond) and alkynyls comprise at least one C—C triple bond (a C≡C-bond). Preferably, aliphatic residues are selected from the group consisting of alkanyl (alkyl) and alkenyl residues, more preferably are alkanyl (alkyl) residues. Preferred C₁₋₁₀ alkanyl residues are selected from the group consisting of methyl, ethyl, n-propyl, 2-propyl, n-butyl, isobutyl, sec.-butyl, tert.-butyl, n-pentyl, isopentyl, neopentyl, n-hexyl, n-heptyl, n-octyl, n-nonyl and n-decyl. Preferred C₁₋₈ alkanyl residues are selected from the group consisting of methyl, ethyl, n-propyl, 2-propyl, n-butyl, isobutyl, sec.-butyl, tert.-butyl, n-pentyl, isopentyl, neopentyl, n-hexyl, n-heptyl and n-octyl. Preferred C₁₋₄ alkanyl residues are selected from the group consisting of methyl, ethyl, n-propyl, 2-propyl, n-butyl, isobutyl, sec.-butyl and tert.-butyl. Preferred C₂₋₁₀ alkenyl residues are selected from the group consisting of ethenyl (vinyl), propenyl (—CH₂CH═CH₂, —CH═CH—CH₃, —C(═CH₂)—CH₃), butenyl, pentenyl, hexenyl heptenyl, octenyl, nonenyl and decenyl. Preferred C₂₋₈ alkenyl residues are selected from the group consisting of ethenyl (vinyl), propenyl (—CH₂CH═CH₂, —CH═CH—CH₃, —C(═CH₂)—CH₃), butenyl, pentenyl, hexenyl heptenyl and octenyl. Preferred C₂₋₄ alkenyl residues are selected from the group consisting of ethenyl (vinyl), propenyl (—CH₂CH═CH₂, —CH═CH—CH₃, —C(═CH₂)—CH₃) and butenyl. Preferred C₂₋₁₀ alkynyl residues are selected from the group consisting of ethynyl, propynyl (—CH₂—C≡CH, —C≡C—CH₃), butynyl, pentynyl, hexynyl, heptynyl, octynyl, nonynyl and decynyl. Preferred C₂₋₈ alkynyl residues are selected from the group consisting of ethynyl, propynyl (—CH₂—C≡CH, —C≡C—CH₃), butynyl, pentynyl, hexynyl, heptynyl and octynyl. Preferred C₂₋₄ alkynyl residues are selected from the group consisting of ethynyl, propynyl (—CH₂—C≡CH, —C≡C—CH₃) and butynyl.

The terms “C₃₋₆ cycloaliphatic residue” and “C₃₋₁₀ cycloaliphatic residue” mean for the purposes of this invention cyclic aliphatic hydrocarbons containing 3, 4, 5 or 6 carbon atoms and 3, 4, 5, 6, 7, 8, 9 or 10 carbon atoms, respectively, wherein the hydrocarbons in each case can be saturated or unsaturated (but not aromatic), unsubstituted or mono- or polysubstituted. The cycloaliphatic residues can be bound to the respective superordinate general structure via any desired and possible ring member of the cycloaliphatic residue. The cycloaliphatic residues can also be condensed with further saturated, (partially) unsaturated, (hetero)cyclic, aromatic or heteroaromatic ring systems, i.e. with cycloaliphatic, heterocycloaliphatic, aryl or heteroaryl residues, which in each case can in turn be unsubstituted or mono- or polysubstituted. C₃₋₁₀ cycloaliphatic residue can furthermore be singly or multiply bridged such as, for example, in the case of adamantyl, bicyclo[2.2.1]heptyl or bicyclo[2.2.2]octyl. Preferred C₃₋₁₀ cycloaliphatic residues are selected from the group consisting of cyclopropyl, cyclobutyl, cyclopentyl, cyclohexyl, cycloheptyl, cyclooctyl, cyclononyl, cyclodecyl, adamantyl,

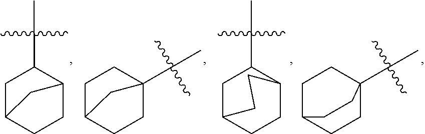

cyclopentenyl, cyclohexenyl, cycloheptenyl and cyclooctenyl. Preferred C₃₋₆ cycloaliphatic residues are selected from the group consisting of cyclopropyl, cyclobutyl, cyclopentyl, cyclohexyl, cyclopentenyl and cyclohexenyl. Particularly preferred C₃₋₁₀ cycloaliphatic and C₃₋₆ cycloaliphatic residues are C₆₋₆ cycloaliphatic residues such as cyclopentyl, cyclohexyl, cyclopentenyl and cyclohexenyl.

The terms “3-6-membered heterocycloaliphatic residue”, and “3-10-membered heterocycloaliphatic residue” mean for the purposes of this invention heterocycloaliphatic saturated or unsaturated (but not aromatic) residues having 3-6, i.e. 3, 4, 5 or 6 ring members, and 3-10, i.e. 3, 4, 5, 6, 7, 8, 9 or 10 ring members, respectively, in which in each case at least one, if appropriate also two or three carbon atoms are replaced by a heteroatom or a heteroatom group each selected independently of one another from the group consisting of O, S, S(═O)₂, N, NH and N(C₁₋₈ alkyl) such as N(CH₃), preferably are replaced by a heteroatom or a heteroatom group each selected independently of one another from the group consisting of O, S, N, NH and N(C₁₋₈ alkyl) such as N(CH₃), wherein the ring members can be unsubstituted or mono- or polysubstituted. The heterocycloaliphatic residue can be bound to the superordinate general structure via any desired and possible ring member of the heterocycloaliphatic residue if not indicated otherwise. The heterocycloaliphatic residues can also be condensed with further saturated, (partially) unsaturated (hetero)cycloaliphatic or aromatic or heteroaromatic ring systems, i.e. with cycloaliphatic, heterocycloaliphatic, aryl or heteroaryl residues, which can in turn be unsubstituted or mono- or polysubstituted. Preferred heterocycloaliphatic residues are selected from the group consisting of azetidinyl, aziridinyl, azepanyl, azocanyl, diazepanyl, dithiolanyl, dihydroquinolinyl, dihydropyrrolyl, dioxanyl, dioxolanyl, dioxepanyl, dihydroindenyl, dihydropyridinyl, dihydrofuranyl, dihydroisoquinolinyl, dihydroindolinyl, dihydroisoindolyl, imidazolidinyl, isoxazolidinyl, morpholinyl, oxiranyl, oxetanyl, oxazepanyl, pyrrolidinyl, piperazinyl, 4-methylpiperazinyl, piperidinyl, pyrazolidinyl, pyranyl, tetrahydropyrrolyl, tetrahydropyranyl, tetrahydro-2H-pyran-4-yl, tetrahydroquinolinyl, tetrahydroisoquinolinyl, tetrahydroindolinyl, tetrahydrofuranyl, tetrahydropyridinyl, tetrahydrothiophenyl, tetrahydropyridoindolyl, tetrahydronaphthyl, tetrahydrocarbolinyl, tetrahydroisoxazololyl, tetrahydropyridinyl, thiazolidinyl and thiomorpholinyl.

The term “aryl” means for the purpose of this invention aromatic hydrocarbons having 6 to 14, i.e. 6, 7, 8, 9, 10, 11, 12, 13 or 14 ring members, preferably having 6 to 10, i.e. 6, 7, 8, 9 or 10 ring members, including phenyls and naphthyls. Each aryl residue can be unsubstituted or mono- or polysubstituted, wherein the aryl substituents can be the same or different and in any desired and possible position of the aryl. The aryl can be bound to the superordinate general structure via any desired and possible ring member of the aryl residue. The aryl residues can also be condensed with further saturated, (partially) unsaturated, (hetero)cycloaliphatic, aromatic or heteroaromatic ring systems, i.e. with a cycloaliphatic, heterocycloaliphatic, aryl or heteroaryl residue, which can in turn be unsubstituted or mono- or polysubstituted. Examples of condensed aryl residues are benzodioxolanyl and benzodioxanyl. Preferably, aryl is selected from the group consisting of phenyl, 1-naphthyl, 2-naphthyl, fluorenyl and anthracenyl, each of which can be respectively unsubstituted or mono- or polysubstituted. A particularly preferred aryl is phenyl, unsubstituted or mono- or polysubstituted.

The term “heteroaryl” for the purpose of this invention represents a 5 or 6-membered cyclic aromatic residue containing at least 1, if appropriate also 2, 3, 4 or 5 heteroatoms, wherein the heteroatoms are each selected independently of one another from the group S, N and O and the heteroaryl residue can be unsubstituted or mono- or polysubstituted; in the case of substitution on the heteroaryl, the substituents can be the same or different and be in any desired and possible position of the heteroaryl. The binding to the superordinate general structure can be carried out via any desired and possible ring member of the heteroaryl residue if not indicated otherwise. The heteroaryl can also be part of a bi- or polycyclic system having up to 14 ring members, wherein the ring system can be formed with further saturated, (partially) unsaturated, (hetero)cycloaliphatic or aromatic or heteroaromatic rings, i.e. with a cycloaliphatic, heterocycloaliphatic, aryl or heteroaryl residue, which can in turn be unsubstituted or mono- or polysubstituted. It is preferable for the heteroaryl residue to be selected from the group consisting of benzofuranyl, benzoimidazolyl, benzothienyl, benzothiadiazolyl, benzothiazolyl, benzotriazolyl, benzooxazolyl, benzooxadiazolyl, quinazolinyl, quinoxalinyl, carbazolyl, quinolinyl, dibenzofuranyl, dibenzothienyl, furyl (furanyl), imidazolyl, imidazothiazolyl, indazolyl, indolizinyl, indolyl, isoquinolinyl, isoxazoyl, isothiazolyl, indolyl, naphthyridinyl, oxazolyl, oxadiazolyl, phenazinyl, phenothiazinyl, phthalazinyl, pyrazolyl, pyridyl (2-pyridyl, 3-pyridyl, 4-pyridyl), pyrrolyl, pyridazinyl, pyrimidinyl, pyrazinyl, purinyl, phenazinyl, thienyl (thiophenyl), triazolyl, tetrazolyl, thiazolyl, thiadiazolyl and triazinyl.

The term “bridged via a C₁₋₄ aliphatic group or via a C₁₋₈ aliphatic group” with respect to residues as aryl, heteroaryl, a heterocycloaliphatic residue and a cycloaliphatic residue mean for the purpose of the invention that these residues have the above-defined meanings and that each of these residues is bound to the respective superordinate general structure via a C₁₋₄ aliphatic group or via a C₁₋₈ aliphatic group, respectively. The C₁₋₄ aliphatic group and the C₁₋₈-aliphatic group can in all cases be branched or unbranched, unsubstituted or mono- or polysubstituted. The C₁₋₄ aliphatic group can in all cases be furthermore saturated or unsaturated, i.e. can be a C₁₋₄ alkylene group, a C₂₋₄ alkenylene group or a C₂₋₄ alkynylene group. The same applies to a C₁₋₈-aliphatic group, i.e. a C₁₋₈-aliphatic group can in all cases be furthermore saturated or unsaturated, i.e. can be a C₁₋₈ alkylene group, a C₂₋₈ alkenylene group or a C₂₋₈ alkynylene group. Preferably, the C₁₋₄-aliphatic group is a C₁₋₄ alkylene group or a C₂₋₄ alkenylene group, more preferably a C₁₋₄ alkylene group. Preferably, the C₁₋₈-aliphatic group is a C₁₋₈ alkylene group or a C₂₋₈ alkenylene group, more preferably a C₁₋₈ alkylene group. Preferred C₁₋₄ alkylene groups are selected from the group consisting of —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)— and —C(CH₃)(CH₂CH₃)—. Preferred C₂₋₄ alkenylene groups are selected from the group consisting of —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)— and —C(CH₂CH₃)═CH—. Preferred C₂₋₄ alkynylene groups are selected from the group consisting of —C═C—, —C≡C—CH₂—, —C≡C—CH₂—CH₂—, —C≡C—CH(CH₃)—, —CH₂—C≡C—CH₂— and —C≡C—C≡C—. Preferred C₁₋₈ alkylene groups are selected from the group consisting of —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH₂—(CH₂)₃—CH₂—, —CH(CH₃)—CH₂—CH₂—CH₂—, —CH₂—CH(CH₃)—CH₂—CH₂—, —CH(CH₃)—CH₂—CH(CH₃)—, —CH(CH₃)—CH(CH₃)—CH₂—, —C(CH₃)₂—CH₂—CH₂—, —CH₂—C(CH₃)₂—CH₂—, —CH(CH₂CH₃)—CH₂—CH₂—, —CH₂—CH(CH₂CH₃)—CH₂—, —C(CH₃)₂—CH(CH₃)—, —CH(CH₂CH₃)—CH(CH₃)—, —C(CH₃)(CH₂CH₃)—CH₂—, —CH(CH₂CH₂CH₃)—CH₂—, —C(CH₂CH₂CH₃)—CH₂—, —CH(CH₂CH₂CH₂CH₃)—, —C(CH₃)(CH₂CH₂CH₃)—, —C(CH₂CH₃)₂— and —CH₂—(CH₂)₄—CH₂—. Preferred C₂₋₈ alkenylene groups are selected from the group consisting of —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)—, —C(CH₂CH₃)═CH—, —CH═CH—CH₂—CH₂—CH₂—, —CH₂—CH═CH₂—CH₂—CH₂—, —CH═CH═CH—CH₂—CH₂— and —CH═CH₂—CH—CH═CH₂—. Preferred C₂₋₈ alkynylene groups are selected from the group consisting of —C≡C—, —C≡C—CH₂—, —C≡C—CH₂—CH₂—, —C≡C—CH(CH₃)—, —CH₂—C≡C—CH₂—, —C≡C—C≡C—, —C≡C—C(CH₃)₂—, —C≡C—CH₂—CH₂—CH₂—, —CH₂—C≡C—CH₂—CH₂—, —C≡C—C≡C—CH₂— and —C≡C—CH₂—C≡C.

In relation to the terms “aliphatic residue”, “aliphatic group”, “cycloaliphatic residue” and “heterocycloaliphatic residue”, the term “mono- or polysubstituted” refers in the sense of this invention, with respect to the corresponding residues or groups, to the single substitution or multiple substitution, e.g. disubstitution, trisubstitution, tetrasubstitution, or pentasubstitution, of one or more hydrogen atoms each independently of one another by at least one substituent selected from the group consisting of F; Cl; Br; I; NO₂; CN; ═O; ═NH; ═N(OH); ═C(NH₂)₂; CF₃; CF₂H; CFH₂; CF₂Cl; CFCl₂; R⁰; C(═O)—H; C(═O)—R⁰; C(═O)—OH; C(═O)—OR⁰; CO—NH₂; C(═O)—NHR⁰; C(═O)—N(R⁰)₂; OH; OCF₃; OCF₂H; OCFH₂; OCF₂Cl; OCFCl₂; OR⁰; O—C(═O)—W; O—C(═O)—O—W; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NH—R⁰; N(R⁰)₂; NH—C(═O)—R⁰; NH—C(═O)—O—R⁰; NH—C(═O)—NH₂; NH—C(═O)—NHR⁰; NH—C(═O)—N(R⁰)₂; NR⁰—C(═O)—R⁰; NR⁰—C(═O)—O—R⁰; NR⁰—C(═O)—NH₂; NR⁰—C(═O)—NHR⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂—R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and)S(═O)₂—N(R⁰)₂. The term “polysubstituted” with respect to polysubstituted residues and groups includes the polysubstitution of these residues and groups either on different or on the same atoms, for example trisubstituted on the same carbon atom, as in the case of CF₃, CH₂CF₃ or 1,1-difluorocyclohexyl, or at various points, as in the case of CH(OH)—CH═CH—CHCl₂ or 1-chloro-3-fluorocyclohexyl. A substituent can if appropriate for its part in turn be mono- or polysubstituted. The multiple substitution can be carried out using the same or using different substituents.

Preferred substituents of “aliphatic residue” and “aliphatic group” are selected from the group consisting of F; Cl; Br; I; NO₂; CF₃; CN; ═O; ═NH; R⁰; (C₁₋₈ alkylene)-OH; C(═O)(R⁰ or H); C(═O)O(R⁰ or H); C(═O)N(R⁰ or H)₂; OH; OR⁰; O—C(═O)—W; O—(C₁₋₈ alkylene)-OH; O—(C₁₋₈ alkylene)-O—C₁₋₈ alkyl; OCF₃; N(R⁰ or H)₂; N(R⁰ or H)—C(═O)—R⁰; N(R⁰ or H)—S(═O)₂—R⁰; N(R⁰ or H)—C(═O)—N(R⁰ or H)₂; SH; SCF₃; SW; S(═O)₂R⁰; S(═O)₂O(R⁰ or H) and S(═O)₂—N(R⁰ or H)₂.

Particularly preferred substituents of “aliphatic residue” and “aliphatic group” are selected from the group consisting of F; Cl; Br; I; NO₂; CF₃; CN; ═O; C₁₋₈ aliphatic residue; aryl; heteroaryl; C₃₋₆ cycloaliphatic residue; 3 to 6 membered heterocycloaliphatic residue; aryl, heteroaryl, C₃₋₆ cycloaliphatic residue or 3 to 6 membered heterocycloaliphatic bridged via a C₁ aliphatic group; CHO; C(═O)—C₁₋₈ aliphatic residue; C(═O)aryl; C(═O)heteroaryl; CO₂H; C(═O)O—C₁₋₈ aliphatic residue; C(═O)O-aryl; C(═O)O-heteroaryl; C(═O)—NH₂; C(═O)NH—C₁₋₈ aliphatic residue; C(═O)N(C₁₋₈ aliphatic residue)₂; C(═O)NH-aryl; C(═O)N(aryl)₂; C(═O)NH-heteroaryl; C(═O)N(heteroaryl)₂; C(═O)N(C₁₋₈ aliphatic residue)(aryl); C(═O)N(C₁₋₈ aliphatic residue)(heteroaryl); C(═O)N(heteroaryl)(aryl); OH; O—C₁₋₈ aliphatic residue; OCF₃; O—(C₁₋₈ aliphatic residue)-OH; O—(C₁₋₈ aliphatic group)-O—C₁₋₈ aliphatic residue; O-benzyl; O-aryl; O-heteroaryl; O—C(═O)—C₁₋₈ aliphatic residue; O—C(═O)aryl; O—C(═O)heteroaryl; NH₂ NH—C₁₋₈ aliphatic residue; NH—(C₁₋₈ aliphatic group)-OH; N(C₁₋₈ aliphatic residue)[(C₁₋₈ aliphatic group)-OH]; N(C₁₋₈ aliphatic residue)₂; NH—C(═O)—C₁₋₈ aliphatic residue; NH—S(═O)₂—C₁₋₈ aliphatic residue; N(C₁₋₈ aliphatic residue)[S(═O)₂—C₁₋₈ aliphatic residue]; NH—S(═O)₂—NH₂; NH—C(═O)-aryl; NH—C(═O)-heteroaryl; SH; S—C₁₋₈ aliphatic residue; SCF₃; S-benzyl; S-aryl; S-heteroaryl; S(═O)₂—C₁₋₈ aliphatic residue; S(═O)₂ aryl; S(═O)₂ heteroaryl; S(═O)₂OH; S(═O)₂O—C₁₋₈ aliphatic residue; S(═O)₂O-aryl; S(═O)₂O-heteroaryl; S(═O)₂—NH—C₁₋₈ aliphatic residue; S(═O)₂—NH-aryl; and S(═O)₂—NH-heteroaryl.

Most preferred substituents of “aliphatic residue” and “aliphatic group” are selected from the group consisting of F; Cl; Br; I; CF₃; C(═O)—NH₂; C(═O)NH—C₁₋₈ aliphatic residue; C(═O)N(C₁₋₈ aliphatic residue)₂; OH; O—C₁₋₈ aliphatic residue; O—(C₁₋₈ aliphatic residue)-OH; O—(C₁₋₈ aliphatic group)-O—C₁₋₈ aliphatic residue; NH₂; NH—C₁₋₈ aliphatic residue; N(C₁₋₈ aliphatic residue)₂; NH—(C₁₋₈ aliphatic group)-OH; N(C₁₋₈ aliphatic residue)[(C₁₋₈ aliphatic group)-OH]; NH—C(═O)—C₁₋₈ aliphatic residue; NH—S(═O)₂—C₁₋₈ aliphatic residue; N(C₁₋₈ aliphatic residue)[S(═O)₂—C₁₋₈ aliphatic residue]; NH—S(═O)₂—NH₂; SH; S—C₁₋₈ aliphatic residue; S(═O)₂—C₁₋₈ aliphatic residue; and S(═O)₂—NH—C₁₋₈ aliphatic residue.

Preferred substituents of “cycloaliphatic residue” and “heterocycloaliphatic residue” are selected from the group consisting of F; Cl; Br; I; NO₂; CF₃; CN; ═O; ═NH; R⁰; C(═O)(R⁰ or H); C(═O)O(R⁰ or H); C(═O)N(R⁰ or H)₂; OH; OR⁰; O—C(═O)—W; O—(C₁₋₈ alkyl)-OH; O—(C₁₋₈ alkyl)-O—C₁₋₈ alkyl; OCF₃; N(R⁰ or H)₂; N(R⁰ or H)—C(═O)—R⁰; N(R⁰ or H)—S(═O)₂—R⁰; N(R⁰ or H)—C(═O)—N(R⁰ or H)₂; SH; SCF₃; SW; S(═O)₂R⁰; S(═O)₂O(R⁰ or H) and S(═O)₂—N(R⁰ or H)₂.

Particularly preferred substituents of “cycloaliphatic residue” and “heterocycloaliphatic residue” are selected from the group consisting of F; Cl; Br; I; NO₂; CF₃; CN; ═O; C₁₋₈ aliphatic residue; aryl; heteroaryl; C₃₋₆ cycloaliphatic residue; 3 to 6 membered heterocycloaliphatic residue; aryl, heteroaryl, C₃₋₆ cycloaliphatic residue or 3 to 6 membered heterocycloaliphatic bridged via a C₁ aliphatic group; CHO; C(═O)—C₁₋₈ aliphatic residue; C(═O)aryl; C(═O)heteroaryl; CO₂H; C(═O)O—C₁₋₈ aliphatic residue; C(═O)O-aryl; C(═O)O-heteroaryl; CONH₂; C(═O)NH—C₁₋₈ aliphatic residue; C(═O)N(C₁₋₈ aliphatic residue)₂; C(═O)NH-aryl; C(═O)N(aryl)₂; C(═O)NH-heteroaryl; C(═O)N(heteroaryl)₂; C(═O)N(C₁₋₈ aliphatic residue)(aryl); C(═O)N(C₁₋₈ aliphatic residue)(heteroaryl); C(═O)N(heteroaryl)(aryl); OH; O—C₁₋₈ aliphatic residue; OCF₃; O—(C₁₋₈ aliphatic group)-OH; O—(C₁₋₈ aliphatic group)-O—C₁₋₈ aliphatic residue; O-benzyl; O-aryl; O-heteroaryl; O—C(═O)—C₁₋₈ aliphatic residue; O—C(═O)aryl; O—C(═O)heteroaryl; NH₂, NH—C₁₋₈ aliphatic residue; N(C₁₋₈ aliphatic residue)₂; NH—C(═O)—C₁₋₈ aliphatic residue; NH—C(═O)-aryl; NH—C(═O)-heteroaryl; SH; S—C₁₋₈ aliphatic residue; SCF₃; S-benzyl; S-aryl; S-heteroaryl; S(═O)₂—C₁₋₈ aliphatic residue; S(═O)₂ aryl; S(═O)₂ heteroaryl; S(═O)₂OH; S(═O)₂O—C₁₋₈ aliphatic residue; S(═O)₂O-aryl; S(═O)₂O-heteroaryl; S(═O)₂—NH—C₁₋₈ aliphatic residue; S(═O)₂—NH-aryl; and S(═O)₂—NH-heteroaryl.

In relation to the terms “aryl” and “heteroaryl”, the term “mono- or polysubstituted” refers in the sense of this invention, with respect to the corresponding residues or groups, to the single substitution or multiple substitution, e.g. disubstitution, trisubstitution, tetrasubstitution, or pentasubstitution, of one or more hydrogen atoms each independently of one another by at least one substituent selected from the group consisting of F; Cl; Br; I; NO₂; CN; CF₃; CF₂H; CFH₂; CF₂Cl; CFCl₂; R⁰; C(═O)—H; C(═O)—R⁰; C(═O)—OH; C(═O)—OR⁰; CO—NH₂; C(═O)—NHR⁰; C(═O)—N(R)₂;

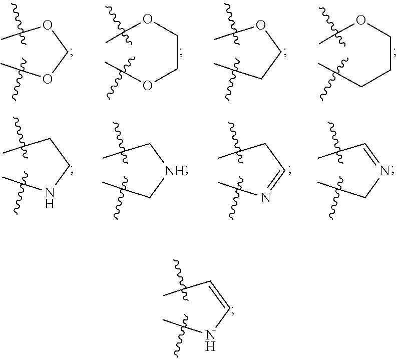

OH; OCF₃; OCF₂H; OCFH₂; OCF₂Cl; OCFCl₂; OR⁰; O—C(═O)—W; O—C(═O)—O—W; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NHR⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and)S(═O)₂—N(R⁰)₂;

Preferred substituents of “aryl” and “heteroaryl” are selected from the group consisting of F; Cl; Br; I; NO₂; CF₃; CN; R⁰; C(═O)(R⁰ or H); C(═O)O(R⁰ or H); C(═O)N(R⁰ or H)₂; OH; OR⁰;

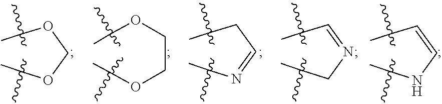

O—C(═O)—R⁰; O—(C₁₋₈ alkyl)-O—C₁₋₈ alkyl; OCF₃; N(R⁰) or H)₂; N(R⁰ or H)—C(═O)—R⁰; N(R⁰ or H)—S(═O)₂—R⁰; N(R⁰ or H)—C(═O)—N(R⁰ or H)₂; SH; SCF₃; SR⁰; S(═O)₂R⁰; S(═O)₂O(R⁰ or H) and S(═O)₂—N(R⁰ or H)₂.

Particularly preferred substituents of “aryl” and “heteroaryl” are selected from the group consisting of F; Cl; Br; I; NO₂; CF₃; CF₂H; CFH₂; CN; C₁₋₈ aliphatic residue; aryl; heteroaryl; C₃₋₆ cycloaliphatic residue; 3 to 6 membered heterocycloaliphatic residue; aryl, heteroaryl, C₃₋₆ cycloaliphatic residue or 3 to 6 membered heterocycloaliphatic bridged via a C₁₋₄ aliphatic group; (C₁₋₈ aliphatic group)-O—C₁₋₈ aliphatic residue; CHO; C(═O)—C₁₋₈ aliphatic residue; C(═O)aryl; C(═O)heteroaryl; CO₂H; C(═O)O—C₁₋₈ aliphatic residue; C(═O)O-aryl; C(═O)O-heteroaryl; CONH₂; C(═O)NH—C₁₋₈ aliphatic residue; C(═O)N(C₁₋₈ aliphatic residue)₂; C(═O)NH-aryl; C(═O)N(aryl)₂; C(═O)NH-heteroaryl; C(═O)N(heteroaryl)₂; C(═O)N(C₁₋₈ aliphatic residue)(aryl); C(═O)N(C₁₋₈ aliphatic residue)(heteroaryl); C(═O)N(heteroaryl)(aryl); OH;

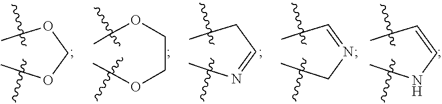

O—C₁₋₈ aliphatic residue; OCF₃; O—(C₁₋₈ aliphatic group)-OH; O—(C₁₋₈ aliphatic group)-O—C₁₋₈ aliphatic residue; O-benzyl; O-aryl; O-heteroaryl; O—C(═O)—C₁₋₈ aliphatic residue; O—C(═O)aryl; O—C(═O)heteroaryl; NH₂, NH—C₁₋₈ aliphatic residue; N(C₁₋₈ aliphatic residue)₂; NH—C(═O)—C₁₋₈ aliphatic residue; NH—C(═O)-aryl; NH—C(═O)-heteroaryl; SH; S—C₁₋₈ aliphatic residue; SCF₃; S-benzyl; S-aryl; S-heteroaryl; S(═O)₂—C₁₋₈ aliphatic residue; S(═O)₂ aryl; S(═O)₂ heteroaryl; S(═O)₂OH; S(═O)₂O—C₁₋₈ aliphatic residue; S(═O)₂O-aryl; S(═O)₂O-heteroaryl; S(═O)₂—NH—C₁₋₈ aliphatic residue; S(═O)₂—NH-aryl; and S(═O)₂—NH-heteroaryl.

The compounds according to the invention are defined by substituents, for example by R¹, R² and R³ (1^{st} generation substituents) which are for their part if appropriate themselves substituted (2^{nd} generation substituents). Depending on the definition, these substituents of the substituents can for their part be resubstituted (3^{rd} generation substituents). If, for example, R¹=a C₁₋₁₀ aliphatic residue (1^{st} generation substituent), then the C₁₋₁₀ aliphatic residue can for its part be substituted, for example with a NH—C₁₋₁₀ aliphatic residue (2^{nd} generation substituent). This produces the functional group R¹═(C₁₋₁₀ aliphatic residue-NH—C₁₋₁₀ aliphatic residue). The NH—C₁₋₁₀ aliphatic residue can then for its part be resubstituted, for example with Cl (3^{rd} generation substituent). Overall, this produces the functional group R¹═C₁₋₁₀ aliphatic residue-NH—C₁₋₁₀ aliphatic residue, wherein the C₁₋₁₀ aliphatic residue of the NH—C₁₋₁₀ aliphatic residue is substituted by Cl.

However, in a preferred embodiment, the 3^{rd} generation substituents may not be resubstituted, i.e. there are then no 4^{th} generation substituents.

In another preferred embodiment, the 2^{nd} generation substituents may not be resubstituted, i.e. there are then not even any 3^{rd} generation substituents. In other words, in this embodiment, in the case of general formula (I), for example, the functional groups for R¹ to R⁹ can each if appropriate be substituted; however, the respective substituents may then for their part not be resubstituted.

In some cases, the compounds according to the invention are defined by substituents which are or carry an aryl or heteroaryl residue, respectively unsubstituted or mono- or polysubstituted, or which form together with the carbon atom(s) or heteroatom(s) connecting them, as the ring member or as the ring members, a ring, for example an aryl or heteroaryl, in each case unsubstituted or mono- or polysubstituted. Both these aryl or heteroaryl residues and the (hetero)aromatic ring systems formed in this way can if appropriate be condensed with a cycloaliphatic, preferably a C₃₋₆ cycloaliphatic residue, or heterocycloaliphatic residue, preferably a 3 to 6 membered heterocycloaliphatic residue, or with aryl or heteroaryl, e.g. with a C₃₋₆ cycloaliphatic residue such as cyclopentyl, or a 3 to 6 membered heterocycloaliphatic residue such as morpholinyl, or an aryl such as phenyl, or a heteroaryl such as pyridyl, wherein the cycloaliphatic or heterocycloaliphatic residues, aryl or heteroaryl residues condensed in this way can for their part be respectively unsubstituted or mono- or polysubstituted.

In some cases, the compounds according to the invention are defined by substituents which are or carry a cycloaliphatic residue or a heterocycloaliphatic residue, respectively, in each case unsubstituted or mono- or polysubstituted, or which form together with the carbon atom(s) or heteroatom(s) connecting them, as the ring member or as the ring members, a ring, for example a cycloaliphatic or a heterocycloaliphatic ring system. Both these cycloaliphatic or heterocycloaliphatic ring systems and the (hetero)cycloaliphatic ring systems formed in this manner can if appropriate be condensed with aryl or heteroaryl, preferably selected from the group consisting of phenyl, pyridyl and thienyl, or with a cycloaliphatic residue, preferably a C₃₋₆ cycloaliphatic residue, or a heterocycloaliphatic residue, preferably a 3 to 6 membered heterocycloaliphatic residue, e.g. with an aryl such as phenyl, or a heteroaryl such as pyridyl, or a cycloaliphatic residue such as cyclohexyl, or a heterocycloaliphatic residue such as morpholinyl, wherein the aryl or heteroaryl residues or cycloaliphatic or heterocycloaliphatic residues condensed in this way can for their part be respectively unsubstituted or mono- or polysubstituted.

Within the scope of the present invention, the symbol

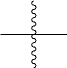

used in the formulas denotes a link of a corresponding residue to the respective superordinate general structure.

If a residue occurs multiply within a molecule, then this residue can have respectively different meanings for various substituents: if, for example, both R¹ and R² denote a 3 to 10 membered heterocycloaliphatic residue, then the 3 to 10 membered heterocycloaliphatic residue can e.g. represent morpholinyl for R¹ and can represent piperazinyl for R².

If a residue occurs multiply within a molecule, such as for example the residue R⁰, then this residue can have respectively different meanings for various substituents.

The term “(R⁰ or H)” within a residue means that R⁰ and H can occur within this residue in any possible combination. Thus, for example, the residue “N(R⁰ or H)₂” can represent “NH₂”, “NHR⁰” and)“N(R⁰)₂”. If, as in the case of)“N(R⁰)₂”, R⁰ occurs multiply within a residue, then R⁰ can respectively have the same or different meanings: in the present example of)“N(R⁰)₂”, R⁰ can for example represent aryl twice, thus producing the functional group “N(aryl)₂”, or R⁰ can represent once aryl and once a C₁₋₁₀ aliphatic residue, thus producing the functional group “N(aryl)(C₁₋₁₀ aliphatic residue)”.

The terms “salt formed with a physiologically compatible acid” or “salt of physiologically acceptable acids” refers in the sense of this invention to salts of the respective active ingredient with inorganic or organic acids which are physiologically compatible—in particular when used in human beings and/or other mammals. Examples of physiologically acceptable acids are: hydrochloric acid, hydrobromic acid, sulfuric acid, methanesulfonic acid, p-toluenesulfonic acid, carbonic acid, formic acid, acetic acid, oxalic acid, succinic acid, tartaric acid, mandelic acid, fumaric acid, maleic acid, lactic acid, citric acid, glutamic acid, saccharic acid, monomethylsebacic acid, 5-oxoproline, hexane-1-sulfonic acid, nicotinic acid, 2, 3 or 4-aminobenzoic acid, 2,4,6-trimethylbenzoic acid, α-lipoic acid, acetyl glycine, hippuric acid, phosphoric acid, aspartic acid. Citric acid and hydrochloric acid are particularly preferred.

The terms “salt formed with a physiologically compatible base” or “salt of physiologically acceptable bases” refers in the sense of this invention to salts of the respective compound according to the invention—as an anion, e.g. upon deprotonation of a suitable functional group—with at least one cation or base preferably with at least one inorganic cation which are physiologically acceptable in particular when used in human beings and/or other mammals. Particularly preferred are the salts of the alkali and alkaline earth metals, in particular (mono-) or (di)sodium, (mono-) or (di)potassium, magnesium or calcium salts, but also ammonium salts [NHₓR₄₋ₓ]⁺, in which x=0, 1, 2, 3 or 4 and R represents a branched or unbranched C₁₋₄ aliphatic residue.

The term “inhibition” in the sense of this invention means to retard or lessen.

Further preferred embodiments of the compound according to the invention of general formula (I) have general formulae (I-a), (I-b), (I-c) and/or (I-d):

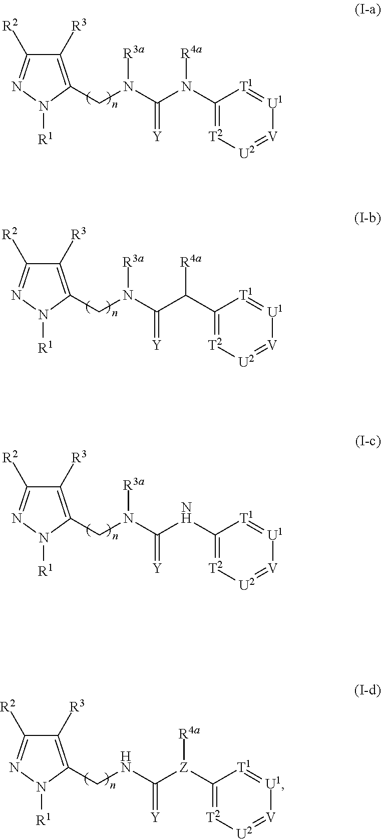

wherein the particular radicals, variables and indices have the meanings described herein in connection with the compounds according to the invention and preferred embodiments thereof.

Moreover, preferred embodiments of the compound according to the invention of general formula (I) have general formulae (I-e), (I-f), (I-g), (I-h), (I-i) and/or (I-j):

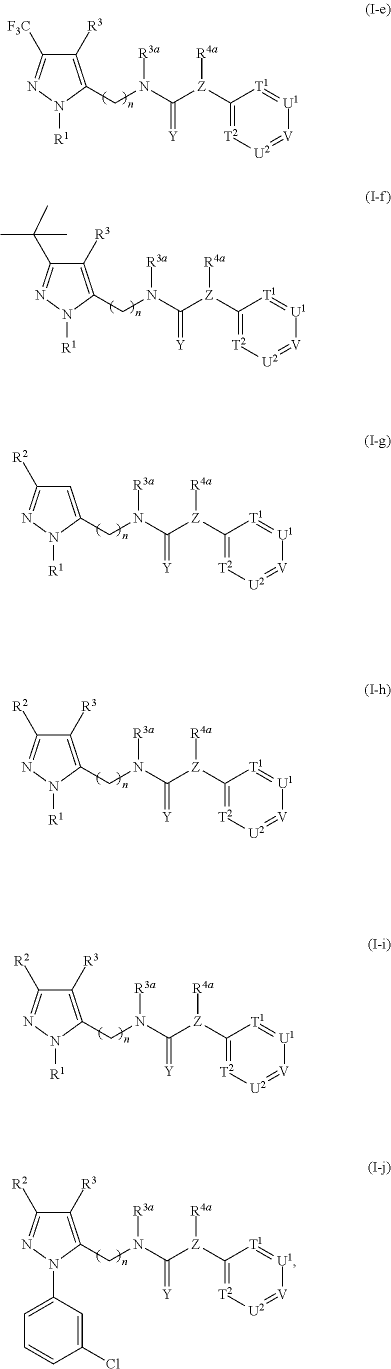

wherein the particular radicals, variables and indices have the meanings described herein in connection with the compounds according to the invention and preferred embodiments thereof.

In addition, preferred embodiments of the compound according to the invention of general formula (I) have general formulae (I-k), (I-l), (I-m) and/or (I-n):

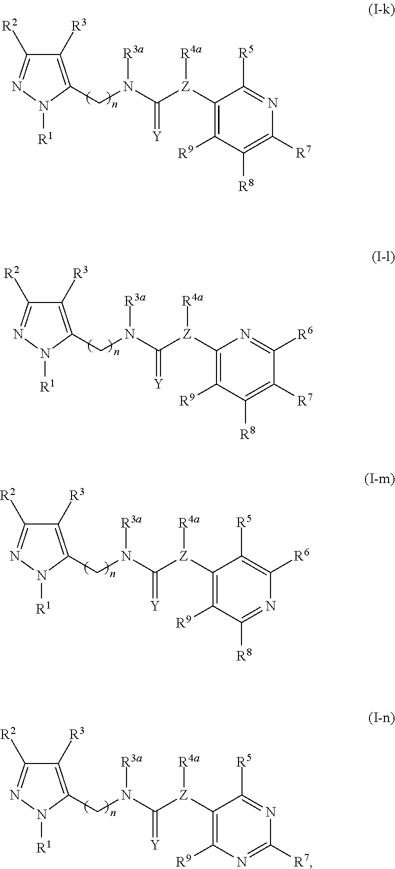

wherein the particular radicals, variables and indices have the meanings described herein in connection with the compounds according to the invention and preferred embodiments thereof.

A particular preferred embodiment of the compound according to the invention of general formula (I) has general formulae (I-k).

Particularly preferred embodiments of general formulae (I-k), (I-l), (I-m) and (I-n), respectively, have general formulae (I-k-1), (I-l-1), (I-m-1) and (I-n-1), respectively

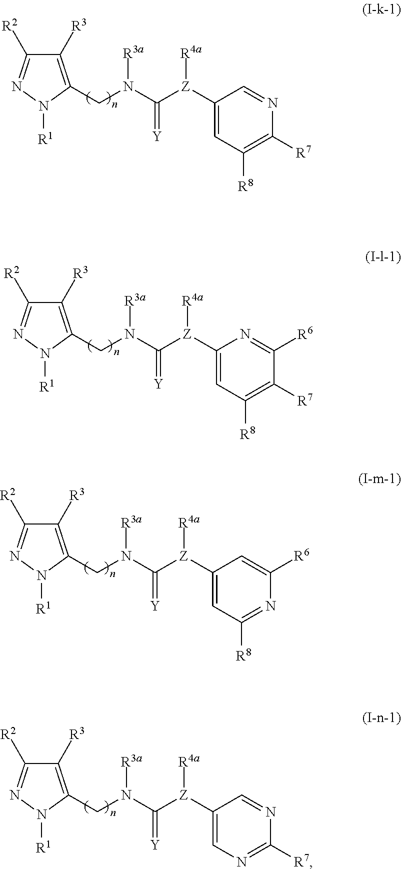

wherein the particular radicals, variables and indices have the meanings described herein in connection with the compounds according to the invention and preferred embodiments thereof.

Particularly preferred is also a compound according to the invention of general formula (I-k-1) which has general formula (I-k2a):

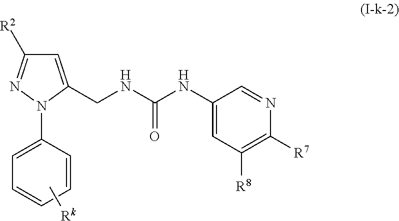

wherein
R^{k} represents phenyl, unsubstituted or mono- or polysubstituted with one or more substituents each selected independently of one another from the group consisting of F, Cl, Br, I, CN, OH, O—CH₃, CH₃, CH(CH₃)₂, N(CH₃)₂, CF₃, CHF₂ and tert.-butyl, preferably phenyl mono- or disubstituted with one or two substituents each selected independently of one another from the group consisting of F, Cl, Br, I, CN, OH, O—CH₃, CH₃, CH(CH₃)₂, N(CH₃)₂, CF₃, CHF₂ and tert.-butyl, more preferably phenyl mono-substituted in meta position with one substituent selected from the group consisting of F, Cl, Br, I, CN, OH, O—CH₃, CH₃, CH(CH₃)₂, N(CH₃)₂, CF₃, CHF₂ and tert.-butyl,
and R², R⁷ and R⁸ have the meanings described herein in connection with the compounds according to the invention and preferred embodiments thereof.

Particularly preferred is also a compound according to the invention of general formula (I-k-1) which has general formula (O-1) and/or (O-2):

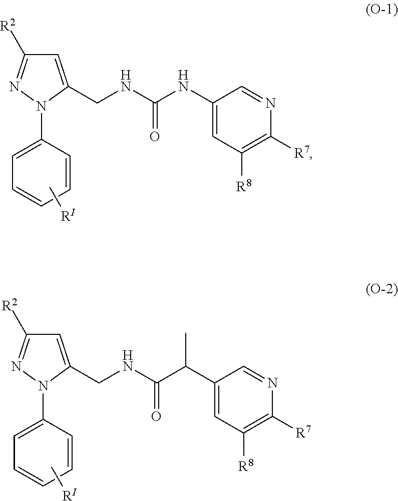

wherein
R^{I} in each case represents one or more such as one or two substituents, preferably one substituent, more preferably one substituent in meta-position of the phenyl ring, selected independently of one another from the group consisting of F, Cl, Br, I, CN, OH, O—CH₃, CH₃, CH(CH₃)₂, N(CH₃)₂, CF₃, CHF₂ and tert.-butyl, more preferably selected from the group consisting of F, Cl, Br, I, OH, O—CH₃, CH₃, CF₃, CHF₂ and tert.-butyl, even more preferably selected from the group consisting of F, Cl, Br, I, OH, O—CH₃, CH₃, CF₃, still more preferably selected from the group consisting of F, Cl, OH, and O—CH₃, most preferred selected from the group consisting of F and Cl,
and R², R⁷ and R⁸ have the meanings described herein in connection with the compounds according to the invention and preferred embodiments thereof,
preferably wherein R² denotes CF₃, cyclopropyl or tert.-butyl, more preferably CF₃ or tert.-butyl, even more preferably CF₃, preferably wherein R⁸ denotes F, Cl, CH₃ or H, more preferably wherein R⁸ denotes H, preferably wherein R⁷ is selected from the group consisting of CH₃, C₂H₅, CH₂—OH, C₂H₄—OH, CH(OH)—CH₂—OH, CH₂—O—CH₃, C₂H₄—O—CH₃, CH₂—O—CH₂—OH, CH₂—O—C₂H₄—OH, CH₂—O—CH₂—O—CH₃, CH₂—O—C₂H₄—O—CH₃, CH₂—S(═O)₂—CH₃, C₂H₄—S(═O)₂—CH₃, CH₂—NH—S(═O)₂—CH₃, CH₂—NH—S(═O)₂—NH₂, CH₂—NH—CH₂—OH, CH₂—NH—C₂H₄—OH, CH₂—NH—C₂H₄—O—CH₃, CH₂—N(CH₃)—C₂H₄—OH, CH₂—N(CH₃)—C₂H₄—O—CH₃, O—CH₃, O—C₂H₄—OH, O—C₂H₄—O—CH₃, NH—CH₃, N(CH₃)₂, NH—C₂H₄—OH, NH—C₂H₄—O—CH₃, N(CH₃)—[C₂H₄—OH], N(CH₃)—[C₂H₄—O—CH₃], NH—S(═O)₂—CH₃,
cyclopropyl, cyclobutyl, cyclopentyl, cyclohexyl, O-cyclopropyl, tetrahydropyranyl, preferably tetrahydro-2H-pyran-4-yl, azetidinyl, piperidinyl, morpholinyl or pyrrolidinyl, in each case independently of one another unsubstituted or monosubstituted with one substituent selected independently of one another from the group consisting of F, Cl, Br, I, OH, O—CH₃, NH₂, N(CH₃)₂, CH₃, C₂H₅ and tert.-butyl,
more preferably wherein R⁷ is selected from the group consisting of CH₂—OH, C₂H₄—OH, CH(OH)—CH₂OH, CH₂—O—C₂H₄—OH, CH₂—NH—S(═O)₂—CH₃, CH₂—NH—S(═O)₂—NH₂, CH₂—S(═O)₂—CH₃, C₂H₄—S(═O)₂—CH₃, O—CH₃, N(CH₃)₂, NH—C₂H₄—OH, NH—C₂H₄—O—CH₃, N(CH₃)—[C₂H₄—OH], NH—S(═O)₂—CH₃, azetidinyl, wherein azetidinyl can be unsubstituted or monosubstituted with OH.

Particularly preferred is a compound according to the invention of general formula (I-k-1) which has general formula (I-k-2) and/or general formula (I-k-3) and/or general formula (I-k-4) and/or general formula (I-k-5):

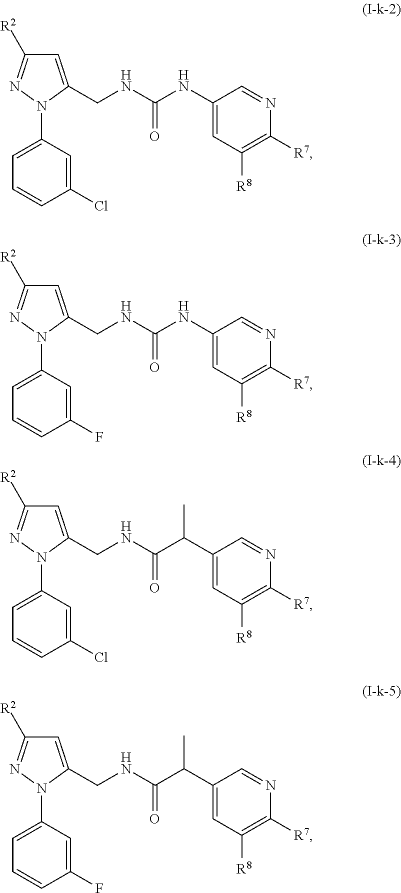

wherein R², R⁷ and R⁸ have the meanings described herein in connection with the compounds according to the invention and preferred embodiments thereof,
preferably wherein R² denotes CF₃, cyclopropyl or tert.-butyl, more preferably CF₃ or tert.-butyl, even more preferably CF₃,
preferably wherein R⁸ denotes F, Cl, CH₃ or H, more preferably wherein R⁸ denotes H,
preferably wherein R⁷ is selected from the group consisting of CH₃, C₂H₅, CH₂—OH, C₂H₄—OH, CH(OH)—CH₂—OH, CH₂—O—CH₃, C₂H₄—O—CH₃, CH₂—O—CH₂—OH, CH₂—O—C₂H₄—OH, CH₂—O—CH₂—O—CH₃, CH₂—O—C₂H₄—O—CH₃, CH₂—S(═O)₂—CH₃, C₂H₄—S(═O)₂—CH₃, CH₂—NH—S(═O)₂—CH₃, CH₂—NH—S(═O)₂—NH₂, CH₂—NH—CH₂—OH, CH₂—NH—C₂H₄—OH, CH₂—NH—C₂H₄—O—CH₃, CH₂—N(CH₃)—C₂H₄—OH, CH₂—N(CH₃)—C₂H₄—O—CH₃, O—CH₃, O—C₂H₄—OH, O—C₂H₄—O—CH₃, NH—CH₃, N(CH₃)₂, NH—C₂H₄—OH, NH—C₂H₄—O—CH₃, N(CH₃)—[C₂H₄—OH], N(CH₃)—[C₂H₄—O—CH₃], NH—S(═O)₂—CH₃,
cyclopropyl, cyclobutyl, cyclopentyl, cyclohexyl, O-cyclopropyl, tetrahydropyranyl, preferably tetrahydro-2H-pyran-4-yl, azetidinyl, piperidinyl, morpholinyl or pyrrolidinyl, in each case independently of one another unsubstituted or monosubstituted with one substituent selected independently of one another from the group consisting of F, Cl, Br, I, OH, O—CH₃, NH₂, N(CH₃)₂, CH₃, C₂H₅ and tert.-butyl,
more preferably wherein R⁷ is selected from the group consisting of CH₂—OH, C₂H₄—OH, CH(OH)—CH₂OH, CH₂—O—C₂H₄—OH, CH₂—NH—S(═O)₂—CH₃, CH₂—NH—S(═O)₂—NH₂, CH₂—S(═O)₂—CH₃, C₂H₄—S(═O)₂—CH₃, O—CH₃, N(CH₃)₂, NH—C₂H₄—OH, NH—C₂H₄—O—CH₃, N(CH₃)—[C₂H₄—OH], NH—S(═O)₂—CH₃, azetidinyl, wherein azetidinyl can be unsubstituted or monosubstituted with OH.

In a particular preferred embodiment of the present invention R¹ of general formula (I) is ≠H.

In another preferred embodiment of the compound of general formula (I) according to the present invention
R¹ represents H, a C₁₋₁₀ aliphatic residue, a O—C₁₋₁₀ aliphatic residue, a S—C₁₋₁₀ aliphatic residue, a NH—C₁₋₁₀ aliphatic residue, a N(C₁₋₁₀ aliphatic residue)₂, a C(═O)—C₁₋₁₀ aliphatic residue, a C(═O)—NH—C₁₋₁₀ aliphatic residue, a C(═O)—N(C₁₋₁₀ aliphatic residue)₂, a NH—C(═O)—C₁₋₁₀ aliphatic residue, a NH—S(═O)₂—C₁₋₁₀ aliphatic residue, a N(C₁₋₁₀ aliphatic residue)-S(═O)₂—C₁₋₁₀ aliphatic residue, a S(═O)₂—C₁₋₁₀ aliphatic residue, a S(═O)₂—NH—C₁₋₁₀ aliphatic residue, a S(═O)₂—N(C₁₋₁₀ aliphatic residue)₂,
   wherein each of the aforementioned residues can in each case be optionally bridged via a C₁₋₈ aliphatic group, which in turn may be unsubstituted or mono- or polysubstituted with one or more substituents each selected independently of one another from the group consisting of F, Cl, Br, I, NO₂, CN, OH, ═O, O—C₁₋₄ alkyl, O—C₁₋₄ alkylene-OH, OCF₃, CF₃, NH₂, NH(C₁₋₄ alkyl), N(C₁₋₄ alkyl)₂, SH, S—C₁₋₄ alkyl, and SCF₃,
   wherein in each case independently of one another the C₁₋₁₀ aliphatic residue can be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
   or represents a C₃₋₁₀ cycloaliphatic residue, a C(═O)—C₃₋₁₀ cycloaliphatic residue, a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—(C₁₋₈ aliphatic group)-C₃₋₁₀ cycloaliphatic residue, a N(C₁₋₁₀ aliphatic residue)(C₃₋₁₀ cycloaliphatic residue), a 3 to 10 membered heterocycloaliphatic residue, a C(═O)-(3 to 10 membered heterocycloaliphatic residue), a O-(3 to 10 membered heterocycloaliphatic residue), a O—(C₁₋₈ aliphatic group)-(3 to 10 membered heterocycloaliphatic residue), a S-(3 to 10 membered heterocycloaliphatic residue), a S—(C₁₋₈ aliphatic group)-(3 to 10 membered heterocycloaliphatic residue), a NH-(3 to 10 membered heterocycloaliphatic residue), NH—(C₁₋₈ aliphatic group)-(3 to 10 membered heterocycloaliphatic residue), a N(C₁₋₁₀ aliphatic residue)(3 to 10 membered heterocycloaliphatic residue),
      wherein each of the aforementioned residues can in each case be optionally bridged via a C₁₋₈ aliphatic group, which in turn may be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl, and SCF₃,
      wherein in each case independently of one another the C₁₋₁₀ aliphatic residue, the C₁₋₈ aliphatic group, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   or represents aryl, C(═O)-aryl, O-aryl, a O—(C₁₋₈ aliphatic group)-aryl, S-aryl, a S—(C₁₋₈ aliphatic group)-aryl, a NH-aryl, NH—C(═O)-aryl, NH—S(═O)₂-aryl a NH—(C₁₋₈ aliphatic group)-aryl, a N(C₁₋₁₀ aliphatic residue)(aryl), heteroaryl, C(═O)-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)-(heteroaryl), N(C₁₋₁₀ aliphatic residue)(heteroaryl),
      wherein each of the aforementioned residues can in each case be optionally bridged via a C₁₋₈ aliphatic group, which in turn may be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl, and SCF₃,
      wherein in each case independently of one another the C₁₋₁₀ aliphatic residue, the C₁₋₈ aliphatic group, aryl and heteroaryl of the aforementioned residues, respectively, can be unsubstituted or mono- or polysubstituted with one or more substituents each selected independently of one another from the group consisting of F, Cl, Br, I, NO₂, CN,

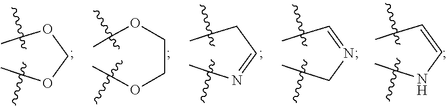

OH, O—C₁₋₄ alkyl, O—C₁₋₄ alkylene-O—C₁₋₄ alkyl, OCF₃, C₁₋₄ alkyl, C₁₋₄ alkylene-O—C₁₋₄-alkyl, CF₃, CF₂H, CHF₂, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH.

In another preferred embodiment of the compound according to the invention of general formula (I), the residue
R¹ represents substructure (T1)

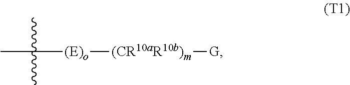

in which
E represents C(═O), O, S, S(═O)₂, NH—C(═O) or NR¹¹, preferably represents O, S or NR¹¹,
   wherein R¹¹ represents H or a C₁₋₄ aliphatic residue, unsubstituted or mono- or polysubstituted with one or more substituents each selected independently of one another from the group consisting of F, Cl, Br, I, OH, O—C₁₋₄ alkyl, OCF₃, NH₂, NH—C₁₋₄ alkyl and N(C₁₋₄ alkyl)₂;
o represents 0 or 1, preferably denotes 0;
R^{10a} and R^{10b} each independently of one another represent H; F; Cl; Br; I; or a C₁₋₄ aliphatic residue, unsubstituted or mono- or polysubstituted with one or more substituents each selected independently of one another from the group consisting of F, Cl, Br, I, OH, O—C₁₋₄ alkyl, OCF₃, NH₂, NH—C₁₋₄ alkyl and N(C₁₋₄ alkyl)₂;
m represents 0, 1, 2, 3 or 4, preferably 0, 1 or 2, more preferably 0 or 1;
G represents a C₁₋₈ aliphatic residue, unsubstituted or mono- or polysubstituted with one or more substituents each selected independently of one another from the group consisting of F, Cl, Br, I, NO₂, CN, OH, ═O, O—C₁₋₄ alkyl, O—C₁₋₄ alkylene-OH, O—C₁₋₄ alkylene-O—C₁₋₄ alkyl, OCF₃, CF₃, NH₂, NH(C₁₋₄ alkyl), N(C₁₋₄ alkyl)₂, SH, S—C₁₋₄ alkyl, and SCF₃,
   or represents a C₃₋₁₀ cycloaliphatic residue or a 3 to 10 membered heterocyclo-aliphatic residue, in each case unsubstituted or mono- or polysubstituted with one or more substituents each selected independently of one another from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
   or represents an aryl or heteroaryl, in each case unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl,

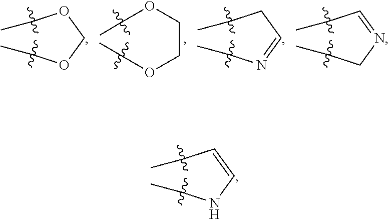

OCF₃, C₁₋₄ alkyl, C₁₋₄ alkylene-O—C₁₋₄-alkyl, CF₃, CF₂H, CFH₂, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

In a particularly preferred embodiment of the compound according to the invention of general formula (I), the residue

R¹ represents substructure (T1), wherein o denotes 0.

Preferably, the residue
R¹ represents substructure (T1) in which
E represents O, S or NR¹¹,
   wherein R¹¹ represents H or an unsubstituted C₁₋₄ aliphatic residue, preferably selected from the group consisting of methyl, ethyl, n-propyl, isopropyl, n-butyl, sec.-butyl, tert.-butyl;
o represents 0 or 1, preferably denotes 0;
R^{10a} and R^{10b} each independently of one another represent H, F, Cl, Br, I or an unsubstituted C₁₋₄ aliphatic residue, preferably selected from the group consisting of methyl, ethyl, n-propyl, isopropyl, n-butyl, sec.-butyl, tert.-butyl;
m represents 0, 1 or 2, preferably denotes 0 or 1;
G represents a C₁₋₈ aliphatic residue, unsubstituted or mono- or polysubstituted with one or more substituents each selected independently of one another from the group consisting of F, Cl, Br, I, OH, O—C₁₋₄ alkyl, O—C₁₋₄ alkylene-OH, O—C₁₋₄ alkylene-O—C₁₋₄ alkyl, OCF₃, CF₃, NH₂, NH(C₁₋₄ alkyl), N(C₁₋₄ alkyl)₂, SH, S—C₁₋₄ alkyl, and SCF₃;
   or represents a C₃₋₁₀ cycloaliphatic residue or a 3 to 10 membered heterocyclo-aliphatic residue, in each case unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, CF₃, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl, and SCF₃;
   or represents an aryl or heteroaryl, in each case unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl,

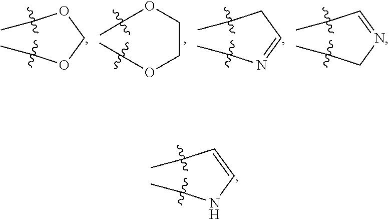

OCF₃, C₁₋₄ alkyl, C₁₋₄ alkylene-O—C₁₋₄-alkyl, CF₃, CF₂H, CFH₂, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl, and SCF₃.

More preferably, the residue
R¹ represents substructure (T1) in which
E represents O, S, or NR¹¹, preferably represents O or S,
   wherein R¹¹ represents H or is selected from the group consisting of methyl, ethyl, n-propyl, and isopropyl,
o represents 0 or 1, preferably 0;
R^{10a} and R^{10b} are independently of one another selected from the group consisting of H, methyl, ethyl, n-propyl, isopropyl, n-butyl, sec.-butyl, tert.-butyl;
m represents 0, 1 or 2, more preferably 0 or 1;
G represents a C₁₋₈ aliphatic residue, preferably represents methyl, ethyl, n-propyl, isopropyl, n-butyl, sec.-butyl, tert.-butyl, pentyl, hexyl, or

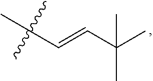 [SMILES: */C=C/C(C)(C)C]

in each case unsubstituted or mono- or polysubstituted with one or more substituents each selected independently of one another from the group consisting of F, Cl, Br, I, OH, O—C₁₋₄ alkyl, O—C₁₋₄ alkylene-OH, O—C₁₋₄ alkylene-O—C₁₋₄ alkyl, CF₃, NH₂, NH(C₁₋₄ alkyl), and N(C₁₋₄ alkyl)₂;
or represents a C₃₋₆ cycloaliphatic residue, preferably selected from the group consisting of cyclopropyl, cyclobutyl, cyclopentyl and cyclohexyl, or a 3 to 6 membered heterocyclo-aliphatic residue, pyrrolidinyl, piperazinyl, 4-methylpiperazinyl, piperidinyl, morpholinyl, tetrahydropyrrolyl, tetrahydroquinolinyl, tetrahydroiso-quinolinyl, dihydroquinolinyl, dihydropyrrolyl, dihydropyridinyl, dihydroisoquinolinyl, tetrahydropyranyl, preferably tetrahydro-2H-pyran-4-yl, tetrahydrofuranyl, tetrahydropyridinyl and thiomorpholinyl, in each case independently of one another unsubstituted or mono- or polysubstituted with one or more substituents each selected independently of one another from the group consisting of F, Cl, Br, I, CN, OH, O—C₁₋₄ alkyl, C₁₋₄ alkyl, CF₃, NH₂, NH(C₁₋₄ alkyl), N(C₁₋₄ alkyl)₂, and phenyl, wherein phenyl is unsubstituted or mono- or polysubstituted with one or more substituents each selected independently of one another from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, and SCF₃;
or represents an aryl, preferably phenyl, or heteroaryl, preferably pyridyl, furyl or thienyl, in each case unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl,

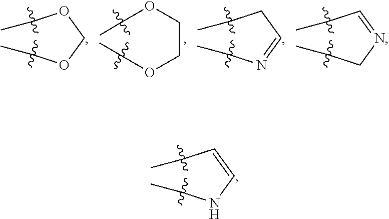

OCF₃, C₁₋₄ alkyl, C₁₋₄ alkylene-O—C₁₋₄-alkyl, CF₃, CF₂H, CFH₂, SCF₃, NH₂, NH(C₁₋₄ alkyl), N(C₁₋₄ alkyl)₂, and phenyl, wherein phenyl is unsubstituted or mono- or polysubstituted with one or more substituents each selected independently of one another from the group consisting of F, Cl, Br, I, CN, OH, O—C₁₋₄ alkyl, OCF₃, C₁₋₄ alkyl, CF₃, NH₂, NH(C₁₋₄ alkyl), N(C₁₋₄ alkyl)₂, and SCF₃.

Even more preferably, the residue
R¹ represents substructure (T1) in which
E represents O, S, or NR¹¹, preferably represents O or S,
   wherein R¹¹ represents H or is selected from the group consisting of methyl and ethyl,
o represents 0 or 1, preferably 0;
R^{10a} and R^{10b} are independently of one another selected from the group consisting of H, methyl and ethyl,
m represents 0, 1 or 2, more preferably 0 or 1;
G represents methyl, ethyl, n-propyl, isopropyl, n-butyl, sec.-butyl, tert.-butyl, pentyl, hexyl, in each case unsubstituted or mono- or polysubstituted with one or more substituents each selected independently of one another from the group consisting of F, Cl, Br, I, OH, O—C₁₋₄ alkyl, O—C₁₋₄ alkylene-OH, and O—C₁₋₄ alkylene-O—C₁₋₄ alkyl, or represents

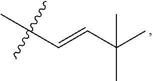 [SMILES: */C=C/C(C)(C)C]

or represents cyclopropyl, cyclobutyl, cyclopentyl and cyclohexyl, or is selected from the group consisting of pyrrolidinyl, piperazinyl, 4-methylpiperazinyl, piperidinyl, morpholinyl, tetrahydropyrrolyl, tetrahydroquinolinyl, tetrahydroisoquinolinyl, tetrahydropyranyl, preferably tetrahydro-2H-pyran-4-yl, tetrahydrofuranyl, tetrahydropyridinyl and thiomorpholinyl, in each case independently of one another unsubstituted or mono- or polysubstituted with one or more substituents each selected independently of one another from the group consisting of F, Cl, Br, I, OH, O—C₁₋₄ alkyl, C₁₋₄ alkyl, NH₂, NH(C₁₋₄ alkyl), and N(C₁₋₄ alkyl)₂, or represents phenyl, pyridyl, furyl or thienyl, in each case unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl

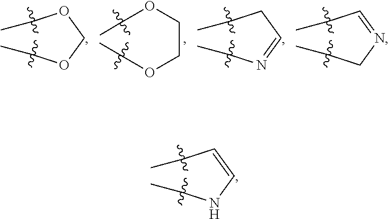

OCF₃, C₁₋₄ alkyl, C₁₋₄ CF₃, CF₂H, CFH₂, SCF₃, NH₂, NH(C₁₋₄ alkyl), and N(C₁₋₄ alkyl)₂.

Still more preferably, the residue
R¹ represents substructure (T1) in which
E represents O or S,
o represents 0 or 1, preferably represents 0,
R^{10a} and R^{10b} are independently of one another selected from the group consisting of H, methyl and ethyl, preferably each denote H;
m represents 0, 1 or 2, more preferably 0 or 1;
G represents methyl, ethyl, n-propyl, isopropyl, n-butyl, sec.-butyl, tert.-butyl, pentyl, hexyl, or represents

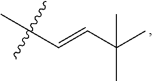 [SMILES: */C=C/C(C)(C)C]

or represents cyclopropyl, cyclobutyl, cyclopentyl and cyclohexyl, or is selected from the group consisting of piperidinyl, morpholinyl, tetrahydropyrrolyl, tetrahydroquinolinyl, tetrahydroisoquinolinyl, dihydroquinolinyl, dihydropyrrolyl, dihydropyridinyl, dihydroisoquinolinyl, tetrahydropyranyl, preferably tetrahydro-2H-pyran-4-yl, tetrahydrofuranyl and tetrahydropyridinyl, in each case independently of one another unsubstituted or mono- or polysubstituted with one or more substituents each selected independently of one another from the group consisting of F, Cl, Br, I, OH, O—C₁₋₄ alkyl, C₁₋₄ alkyl, NH₂, NH(C₁₋₄ alkyl), and N(C₁₋₄ alkyl)₂,
or represents furyl or thienyl, in each case unsubstituted, or denotes phenyl or pyridyl, in each case unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl,

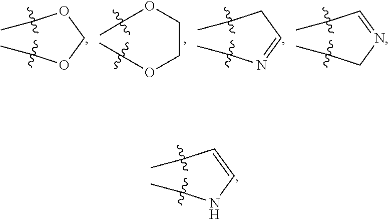

OCF₃, C₁₋₄ alkyl, C₁₋₄ alkylene-O—C₁₋₄-alkyl, CF₃, CF₂H, CFH₂, SCF₃, NH₂, NH(C₁₋₄ alkyl), and N(C₁₋₄ alkyl)₂.

Most preferred,

R¹ represents phenyl, unsubstituted or mono- or polysubstituted with one or more substituents each selected independently of one another from the group consisting of F, Cl, Br, I, CN, OH, O—CH₃, CH₃, CH(CH₃)₂, N(CH₃)₂, CF₃, CHF₂ and tert.-butyl, preferably phenyl mono- or disubstituted with one or two substituents each selected independently of one another from the group consisting of F, Cl, Br, I, CN, OH, O—CH₃, CH₃, CH(CH₃)₂, N(CH₃)₂, CF₃, CHF₂ and tert.-butyl, more preferably phenyl mono-substituted in meta position with one substituent selected from the group consisting of F, Cl, Br, I, CN, OH, O—CH₃, CH₃, CH(CH₃)₂, N(CH₃)₂, CF₃, CHF₂ and tert.-butyl.

In a particular preferred embodiment of the present invention R² of general formula (I) is ≠H.

In another preferred embodiment of the compound of general formula (I) according to the present invention
R² represents H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂;
   or a C₁₋₁₀ aliphatic residue, unsubstituted or mono- or polysubstituted with one or more substituents each selected independently of one another from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
   or a C₃₋₁₀ cycloaliphatic residue or a 3 to 10 membered heterocycloaliphatic residue, in each case unsubstituted or mono- or polysubstituted with one or more substituents selected independently of one another from the group consisting of F, Cl, Br, I, OH, ═O, C₁₋₄ alkyl, O—C₁₋₄ alkyl, OCF₃, C(═O)—OH and CF₃;
      wherein each of the aforementioned residues, i.e. the C₃₋₁₀ cycloaliphatic residue or the 3 to 10 membered heterocycloaliphatic residue, can in each case be optionally bridged via a C₁₋₈ aliphatic group, which in turn may be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl, and SCF₃,
   or aryl or heteroaryl, respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₈ alkyl, SCF₃, S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₈ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH;
      wherein each of the aforementioned residues, i.e. aryl and heteroaryl, can in each case be optionally bridged via a C₁₋₈ aliphatic group, which in turn may be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl, and SCF₃.

Preferably,
R² represents F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂;
   or represents a C₁₋₈ aliphatic residue, unsubstituted or mono- or polysubstituted with one or more substituents each selected independently of one another from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ S(═O)₂OH, or represents
   a C₃₋₆ cycloaliphatic residue or a 3 to 6 membered heterocycloaliphatic residue, in each case unsubstituted or mono- or polysubstituted with one or more substituents selected independently of one another from the group consisting of F, Cl, Br, I, OH, ═O, C₁₋₄ alkyl, O—C₁₋₄ alkyl, OCF₃, C(═O)—OH and CF₃;
   or represents phenyl or pyridyl, in each case unsubstituted or mono- or polysubstituted with one or more substituents each selected independently of one another from the group F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl) and N(C₁₋₄ alkyl)₂.

More preferably,
R² represents a C₁₋₈ aliphatic residue, unsubstituted or mono- or polysubstituted with one or more substituents each selected independently of one another from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁ alkyl, SCF₃ S(═O)₂OH, or represents
   a C₃₋₆ cycloaliphatic residue or a 3 to 6 membered heterocycloaliphatic residue, in each case unsubstituted or mono- or polysubstituted with one or more substituents selected independently of one another from the group consisting of F, Cl, Br, I, OH, ═O, C₁ alkyl, O—C₁₋₄ alkyl, OCF₃, C(═O)—OH and CF₃.

Even more preferably
R² represents a C₁ aliphatic residue, unsubstituted or mono- or polysubstituted with one or more substituents each selected independently of one another from the group consisting of F, Cl, Br, I, and OH, or represents
   a C₃₋₆ cycloaliphatic residue or a 3 to 6 membered heterocycloaliphatic residue, in each case unsubstituted or mono- or polysubstituted with one or more substituents selected independently of one another from the group consisting of F, Cl, Br, I, and OH.

Still more preferably
R² represents a C₁ aliphatic residue, unsubstituted or mono- or polysubstituted with one or more substituents each selected independently of one another from the group consisting of F, Cl, Br, I, or represents
   a C₃₋₆ cycloaliphatic residue or a 3 to 6 membered heterocycloaliphatic residue, preferably a C₃₋₆ cycloaliphatic residue, in each case unsubstituted.

Particularly preferably
R² is selected from the group consisting of CF₃, methyl, ethyl, n-propyl, isopropyl, n-butyl, sec.-butyl, and tert.-butyl, or
   is selected from the group consisting of cyclopropyl, cyclobutyl, cyclopentyl, and cyclohexyl.

Most preferred,

R² is selected from the group consisting of tert-Butyl, CF₃, cyclopropyl, cyclobutyl, cyclopentyl, and cyclohexyl, preferably from the group consisting of tert-Butyl, CF₃ and cyclopropyl, more preferably from the group consisting of tert-Butyl and CF₃.

In yet another preferred embodiment of the compound of general formula (I) according to the present invention
R³ represents H or a C₁₋₄ aliphatic residue, unsubstituted or mono- or polysubstituted with one or more substituents each selected independently of one another from the group consisting of F, Cl, Br, I, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl and SCF₃.

Preferably,
R³ represents H or a C₁₋₄ aliphatic residue, unsubstituted or mono- or polysubstituted with one or more substituents each selected independently of one another from the group consisting of F, Cl, Br, I and OH.

More preferably,
R³ represents H or an unsubstituted C₁₋₄ aliphatic residue, preferably selected from the group consisting of methyl, ethyl, n-propyl, isopropyl, n-butyl, sec.-butyl, and tert.-butyl.

In particular,
R³ is selected from the group consisting of H, methyl and ethyl, preferably denotes H or methyl, more preferably represents H.

In a preferred embodiment of the compound of general formula (I) according to the present invention
n represents 1, 2, 3 or 4, preferably 1, 2 or 3, particularly preferably 1 or 2, most preferred 1.

In yet another preferred embodiment of the compound of general formula (I) according to the present invention
R^{3a} represents H or a C₁₋₄ aliphatic residue, unsubstituted or mono- or polysubstituted with one or more substituents each selected independently of one another from the group consisting of F, Cl, Br, I, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl and SCF₃.

Preferably,
R^{3a} represents H or a C₁₋₄ aliphatic residue, unsubstituted or mono- or polysubstituted with one or more substituents each selected independently of one another from the group consisting of F, Cl, Br, I and OH.

More preferably,
R^{3a} represents H or an unsubstituted C₁₋₄ aliphatic residue, preferably selected from the group consisting of methyl, ethyl, n-propyl, isopropyl, n-butyl, sec.-butyl, and tert.-butyl.

In particular,
R^{3a} is selected from the group consisting of H, methyl and ethyl, preferably denotes H or methyl, more preferably represents H.

In another preferred embodiment of the compound of general formula (I) according to the present invention
Y represents O or S, preferably represents O.

Preferred is also an embodiment of the compound of general formula (I) according to the present invention, wherein
R^{4a} represents H or a C₁₋₄ aliphatic residue, unsubstituted or mono- or polysubstituted with at least one substituent selected from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   or represents a C₃₋₆ cycloaliphatic residue, unsubstituted or mono- or polysubstituted with at least one substituent selected from the group consisting of F, Cl, Br, I, NO₂, CN, OH, ═O, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   or denotes an aryl, unsubstituted or mono- or polysubstituted with at least one substituent selected from the group consisting of F, Cl, Br, I, NO₂, CN, OH, O—C₁₋₄ alkyl, OCF₃, C₁₋₄ alkyl, C(═O)—OH, CF₃, CF₂H, CFH₂, CF₂Cl, CFCl₂, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl,
R^{4b} represents H or a C₁₋₄ aliphatic residue, unsubstituted or mono- or polysubstituted with at least one substituent selected from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH or
   R^{4a} and R^{4b} together with the carbon atom connecting them form a C₃₋₆ cycloaliphatic residue, unsubstituted or mono- or polysubstituted with at least one substituent selected from the group consisting of F, Cl, Br, I, NO₂, CN, OH, ═O, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ S(═O)₂OH, benzyl, phenyl, pyridyl and thienyl, wherein benzyl, phenyl, pyridyl, thienyl can be respectively unsubstituted or mono- or polysubstituted with one or more substituents selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

Preferably,
R^{4a} represents H or a C₁₋₄ aliphatic residue, unsubstituted or mono- or polysubstituted with at least one substituent selected from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   or represents a C₃₋₆ cycloaliphatic residue, unsubstituted or mono- or polysubstituted with at least one substituent selected from the group consisting of F, Cl, Br, I, NO₂, CN, OH, ═O, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
   or denotes an aryl, unsubstituted or mono- or polysubstituted with at least one substituent selected from the group consisting of F, Cl, Br, I, NO₂, CN, OH, O—C₁₋₄ alkyl, OCF₃, C₁₋₄ alkyl, C(═O)—OH, CF₃, CF₂H, CFH₂, CF₂Cl, CFCl₂, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl,
R^{4b} represents H or a C₁₋₄ aliphatic residue, unsubstituted or mono- or polysubstituted with at least one substituent selected from the group consisting of F, Cl, Br, I, NO₂, CN, OH, ═O, O—C₁₋₄ alkyl, OCF₃, C(═O)—OH, CF₃, NH₂, NH(C₁₋₄ alkyl), N(C₁₋₄ alkyl)₂, SH, S—C₁₋₄ alkyl, SCF₃ and S(═O)₂OH, or
   R^{4a} and R^{4b} together with the carbon atom connecting them form a C₃₋₆ cycloaliphatic residue, unsubstituted or mono- or polysubstituted with at least one substituent selected from the group consisting of F, Cl, Br, I, NO₂, CN, OH, ═O, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

More preferably,
R^{4a} represents H or a C₁₋₄ aliphatic residue, unsubstituted or mono- or polysubstituted with at least one substituent selected from the group consisting of F, Cl, Br, I, OH, ═O, O—C₁₋₄ alkyl, OCF₃, CF₃, and SCF₃,
   or represents a C₃₋₆ cycloaliphatic residue, unsubstituted or mono- or polysubstituted with at least one substituent selected from the group consisting of F, Cl, Br, I, OH, ═O, O—C₁₋₄ alkyl, OCF₃, C₁₋₄ alkyl, CF₃, and SCF₃,
   or denotes an aryl, preferably a phenyl, unsubstituted or mono- or polysubstituted with at least one substituent selected from the group consisting of F, Cl, Br, I, NO₂, CN, OH, O—C₁₋₄ alkyl, OCF₃, C₁₋₄ alkyl, C(═O)—OH, CF₃, CF₂H, CFH₂, CF₂Cl, CFCl₂, NH₂, NH(C₁₋₄ alkyl), N(C₁₋₄ alkyl)₂, SH, S—C₁₋₄ alkyl, SCF₃, S(═O)₂OH and NH—S(═O)₂—C₁₋₄ alkyl,
R^{4b} represents H or a C₁₋₄ aliphatic residue, unsubstituted or mono- or polysubstituted with at least one substituent selected from the group consisting of F, Cl, Br, I, OH, ═O, O—C₁₋₄ alkyl, OCF₃, CF₃, and SCF₃, or
   R^{4a} and R^{4b} together with the carbon atom connecting them form a C₃₋₆ cycloaliphatic residue, unsubstituted or mono- or polysubstituted with at least one substituent selected from the group consisting of F, Cl, Br, I, OH, ═O, O—C₁₋₄ alkyl, OCF₃, C₁₋₄ alkyl, CF₃, and SCF₃.

Even more preferably,
R^{4a} represents H or an unsubstituted C₁₋₄ aliphatic residue, preferably denotes H or is selected from the group consisting of methyl, ethyl, n-propyl, isopropyl, n-butyl, sec.-butyl, and tert.-butyl,
   or represents an unsubstituted C₃₋₆ cycloaliphatic residue, preferably selected from the group consisting of cyclopropyl, cyclobutyl, cyclopentyl and cyclohexyl,
   or denotes a phenyl, unsubstituted or mono- or polysubstituted with at least one substituent selected from the group consisting of F, Cl, Br, I, NO₂, CN, OH, O—C₁₋₄ alkyl, OCF₃, C₁ alkyl, C(═O)—OH, CF₃, CF₂H, CFH₂, CF₂Cl, CFCl₂, NH₂, NH(C₁₋₄ alkyl), N(C₁₋₄ alkyl)₂, SH, S—C₁₋₄ alkyl, SCF₃, S(═O)₂OH and NH—S(═O)₂—C₁ alkyl,
R^{4b} represents H or a C₁₋₄ aliphatic residue, unsubstituted or mono- or polysubstituted with at least one substituent selected from the group consisting of F, Cl, Br, I, OH, ═O, O—C₁₋₄ alkyl, OCF₃, CF₃, and SCF₃, or
   R^{4a} and R^{4b} together with the carbon atom connecting them form a C₃₋₆ cycloaliphatic residue, preferably selected from the group consisting of cyclopropyl, cyclobutyl, cyclopentyl and cyclohexyl, unsubstituted or mono- or polysubstituted with at least one substituent selected from the group consisting of F, Cl, Br, I, OH, ═O, O—C₁₋₄ alkyl, OCF₃, C₁ alkyl, CF₃, and SCF₃.

Still more preferably,
R^{4a} represents H; methyl, ethyl, cyclopropyl, cyclobutyl, cyclopentyl, cyclohexyl, or phenyl, wherein phenyl is unsubstituted or substituted with 1, 2, 3, 4 or 5 substituents independently selected from the group consisting of F, Cl, Br, I, NO₂, CN, CF₃, CF₂H, CFH₂, CF₂Cl, CFCl₂, OH, NH₂, NH(C₁₋₄ alkyl) and N(C₁₋₄ alkyl)(C₁₋₄ alkyl), C₁₋₄ alkyl, and O—C₁₋₄-alkyl;
   R^{4b} represents H, methyl, or ethyl,
   or R^{4a} and R^{4b} together with the carbon atom connecting them form a cyclopropyl, cyclobutyl, cyclopentyl, or cyclohexyl ring.

Particularly preferred is a compound of general formula (I) according to the present invention, wherein

R^{4a} represents H, methyl, ethyl, cyclopropyl, cyclobutyl, cyclopentyl, cyclohexyl, or phenyl, wherein phenyl is unsubstituted or substituted with 1, 2 or 3 substituents independently selected from the group consisting of F, Cl, Br, CF₃, methyl and methoxy;
R^{4b} represents H, methyl, or ethyl,
or R^{4a} and R^{4b} together with the carbon atom connecting them form cyclopropyl, cyclobutyl, cyclopentyl, or cyclohexyl ring.

Even more particularly preferred is a compound of general formula (I) according to the present invention, wherein

R^{4a} represents H, methyl, or ethyl,
R^{4b} represents H, methyl, or ethyl, preferably H or methyl, more preferably H,
or R^{4a} and R^{4b} together with the carbon atom connecting them form cyclopropyl, cyclobutyl, cyclopentyl, or cyclohexyl ring.

Most preferred is a compound of general formula (I) according to the present invention, wherein

R^{4a} represents H, methyl, or ethyl, more preferably H or methyl
R^{4b} represents H, methyl, or ethyl, preferably H or methyl, more preferably H.

In another preferred embodiment of the compound of general formula (I) according to the present invention,
R^{3a} is selected from the group consisting of H, methyl and ethyl, preferably denotes H or methyl, more preferably represents H;
R^{4a} represents H; methyl, ethyl, cyclopropyl, cyclobutyl, cyclopentyl, cyclohexyl, or phenyl, wherein phenyl is unsubstituted or substituted with 1, 2, 3, 4 or 5 substituents independently selected from the group consisting of F, Cl, Br, I, NO₂, CN, CF₃, CF₂H, CFH₂, CF₂Cl, CFCl₂, OH, NH₂, NH(C₁₋₄ alkyl) and N(C₁₋₄ alkyl)(C₁₋₄ alkyl), C₁₋₄ alkyl, and O—C₁₋₄-alkyl;
R^{4b} represents H, methyl, or ethyl,
   or R^{4a} and R^{4b} together with the carbon atom connecting them form a cyclopropyl, cyclobutyl, cyclopentyl, or cyclohexyl ring.

In yet another preferred embodiment of the compound of general formula (I) according to the present invention,

Z represents N and R^{4a} represents H; or
Z represents CR^{4b} and R^{4a} and R^{4b} each represent H; or
Z represents CR^{4b} and R^{4a} represents methyl and R^{4b} represents H.

In yet another preferred embodiment of the compound of general formula (I) according to the present invention,

Z represents N and R^{4a} represents H; or
Z represents CR^{4b} and R^{4a} and R^{4b} each represent H; or
Z represents CR^{4b} and R^{4a} represents H and R^{4b} represents methyl.

In a preferred embodiment of the compound according to the invention of general formula (I) 1 or 2 of variables T¹, U¹, V, U² and T² represent(s) a nitrogen atom, preferably only 1 of variables T¹, U¹, V, U² and T² represents a nitrogen atom, more preferably only U¹ of T¹, U¹, V, U² and T² represents a nitrogen atom, i.e. T¹ denotes C—R⁵, V denotes C—R⁷, U² denotes C—R⁸ and T² denotes C—R⁹.

In another preferred embodiment of the compound according to the invention of general formula (I), the substructure (T2) of general formula (I)

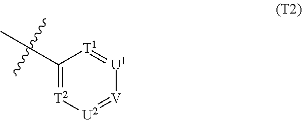

represents one or more of the substructures (T2-a), (T2-b), (T2-c), (T2-d), (T2-e), (T2-f), (T2-g), (T2-h), (T2-i), (T2-j) (T2-k), (T2-I), (T2-m), (T2-n), and/or (T2-o)

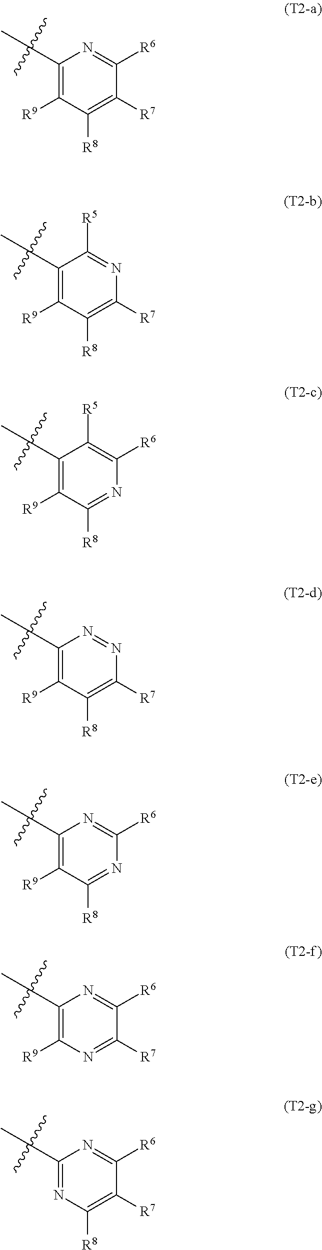

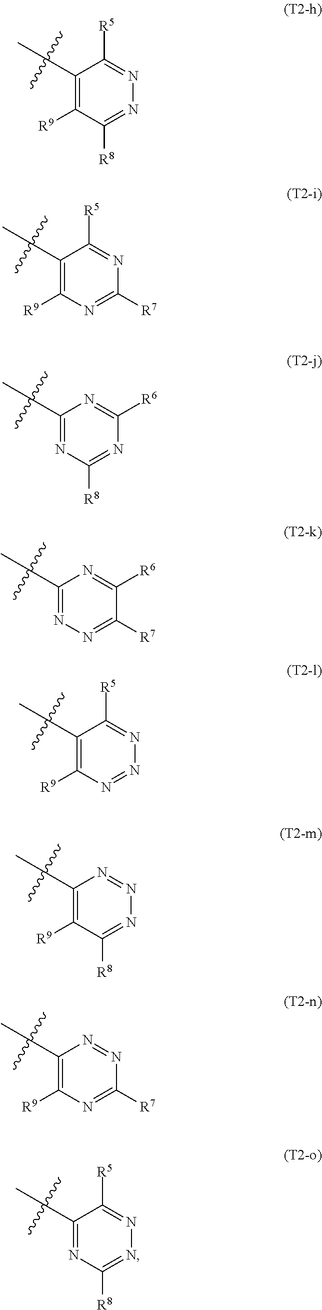

in which R⁵, R⁶, R⁷, R⁸ and R⁹ in each case independently of one another have one of the above defined meanings or have the meaning as described herein in connection with the compounds according to the invention and preferred embodiments thereof.

Preferred substructures of (T2) are (T2-a), (T2-b), (T2-c), (T2-e), (T24), (T2-h), (T2-i) and (T2-j), more preferred substructures of (T2) are (T2-a), (T2-b) and (T2-c), a particularly preferred substructure of (T2) is (T2-b).

Particularly preferred substructures of (T2-a), (T2-b) and (T2-c), respectively, are substructures (T2-a-I), (T2-b-I) and (T2-c-I)

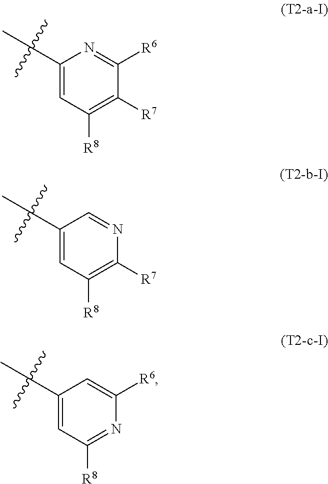

in which R⁶, R⁷, and R⁹ in each case independently of one another have one of the above defined meanings or have the meaning as described herein in connection with the compounds according to the invention and preferred embodiments thereof. Most preferred is substructure (T2-b-I).

In yet another preferred embodiment of the compound according to the invention of general formula (I),

R⁵, R⁶, R⁷, R⁸ and R⁹ are each independently of one another selected from the group consisting of
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue, a C(═O)—C₁₋₁₀ aliphatic residue, a C(═O)—NH—C₁₋₁₀ aliphatic residue,
a O—C₁₋₁₀ aliphatic residue, a O—(C₁₋₈ aliphatic group)-O—C₁₋₁₀ aliphatic residue, O—(C₁₋₈ aliphatic group)-OH,
a NH—C₁₋₁₀ aliphatic residue, a N(C₁₋₁₀ aliphatic residue)₂, a NH—[(C₁₋₈ aliphatic group)-O—C₁₋₁₀ aliphatic residue], a NH—[(C₁₋₈ aliphatic group)-OH], a N(C₁₋₁₀ aliphatic residue)[(C₁₋₈ aliphatic group)-OH], a N(C₁₋₁₀ aliphatic residue)[(C₁₋₈ aliphatic group)-O—C₁₋₁₀ aliphatic residue], a NH—C(═O)—C₁₋₁₀ aliphatic residue, a N(C₁₋₁₀ aliphatic residue)[(C(═O)—C₁₋₁₀ aliphatic residue)], a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue, a N(C₁₋₁₀ aliphatic residue)[S(═O)₂—C₁₋₁₀ aliphatic residue],
a S(═O)₂—C₁₋₁₀ aliphatic residue, a S(═O)₂—NH—C₁₋₁₀ aliphatic residue, a S(═O)₂—N(C₁₋₁₀ aliphatic residue)₂, a S—C₁₋₁₀ aliphatic residue,
   wherein each of the aforementioned C₁₋₁₀ aliphatic residues and C₁₋₈ aliphatic groups can in each case be unsubstituted or mono- or polysubstituted with one or more substituents each selected independently of one another from the group consisting of F, Cl, Br, I, NO₂, CN, OH, ═O, O—C₁₋₄ alkyl, OCF₃, CF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
a C₃₋₁₀ cycloaliphatic residue, a C(═O)—C₃₋₁₀ cycloaliphatic residue, a C(═O)NH—C₃₋₁₀ cycloaliphatic residue a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—C(═O)—C₃₋₁₀ cycloaliphatic residue, a NH—(C₁₋₈ aliphatic group)-C₃₋₁₀ cycloaliphatic residue, a N(C₁₋₁₀-aliphatic residue)(C₃₋₁₀ cycloaliphatic residue), a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a O—(C₁₋₈ aliphatic group)-(3 to 10 membered heterocycloaliphatic residue), a S-(3 to 10 membered heterocycloaliphatic residue), a S—(C₁₋₈ aliphatic group)-(3 to 10 membered heterocyclo-aliphatic residue), a NH-(3 to 10 membered heterocycloaliphatic residue), a NH—C(═O)-(3 to 10 membered heterocycloaliphatic residue), NH—(C₁₋₈ aliphatic group)-(3 to 10 membered heterocycloaliphatic residue), a N(C₁₋₁₀ aliphatic residue)(3 to 10 membered heterocycloaliphatic residue),
   wherein each of the aforementioned residues can in each case be optionally bridged via a C₁₋₈ aliphatic group,
   wherein in each case independently of one another the C₁₋₁₀ aliphatic residue, the C₁₋₈ aliphatic group, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, ═O, OCF₃, OH, SH, S—C₁₋₄ alkyl, SCF₃, SO₂—C₁₋₄ alkyl, NH₂, ═NH, ═N(OH), NH—C₁₋₄ alkyl, N(C₁₋₄ alkyl)₂, NH—SO₂—C₁₋₄ alkyl, NH—C(═O)—C₁₋₄ alkyl, phenyl and pyridyl, wherein phenyl and pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
aryl, C(═O)-aryl, C(═O)—NH-aryl, O-aryl, a O—(C₁₋₈ aliphatic group)-aryl, S-aryl, a S—(C₁₋₈ aliphatic group)-aryl, a NH-aryl, NH—C(═O)-aryl, NH—S(═O)₂-aryl a NH—(C₁₋₈ aliphatic group)-aryl, a N(C₁₋₁₀ aliphatic residue)(aryl), heteroaryl, C(═O)-heteroaryl, C(═O)—NH-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)(heteroaryl), N(C₁₋₁₀ aliphatic residue)(heteroaryl),
   wherein each of the aforementioned residues can in each case be optionally bridged via a C₁₋₈ aliphatic group,
   wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH,
   wherein in each case independently of one another the C₁₋₁₀ aliphatic residues and the C₁₋₈ aliphatic groups of the aforementioned residues, respectively, can be unsubstituted or mono- or polysubstituted with one or more substituents each selected independently of one another from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

Preferably,

R⁵, R⁶, R⁷, R⁸ and R⁹ are each independently of one another selected from the group consisting of
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue, a C(═O)—C₁₋₁₀ aliphatic residue, a C(═O)—NH—C₁₋₁₀ aliphatic residue,
a O—C₁₋₁₀ aliphatic residue, a O—(C₁₋₈ aliphatic group)-O—C₁₋₁₀ aliphatic residue, O—(C₁₋₈ aliphatic group)-OH,
a NH—C₁₋₁₀ aliphatic residue, a N(C₁₋₁₀ aliphatic residue)₂, a NH—(C₁₋₈ aliphatic group)-O—C₁₋₁₀ aliphatic residue, a NH—(C₁₋₈ aliphatic group)-OH, a N(C₁₋₁₀ aliphatic residue)[(C₁₋₈ aliphatic group)-OH], a N(C₁₋₁₀ aliphatic residue)[(C₁₋₈ aliphatic group)-O—C₁₋₁₀ aliphatic residue], a NH—C(═O)—C₁₋₁₀ aliphatic residue, a N(C₁₋₁₀ aliphatic residue)[(C(═O)—C₁₋₁₀ aliphatic residue)], a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue, a N(C₁₋₁₀ aliphatic residue)[S(═O)₂—C₁₋₁₀ aliphatic residue],
a S(═O)₂—C₁₋₁₀ aliphatic residue, a S(═O)₂—NH—C₁₋₁₀ aliphatic residue, a S(═O)₂—N(C₁₋₁₀ aliphatic residue)₂, a S—C₁₋₁₀ aliphatic residue,
   wherein each of the aforementioned C₁₋₁₀ aliphatic residues and C₁₋₈ aliphatic groups can in each case be unsubstituted or monosubstituted with OH;
a C₃₋₁₀ cycloaliphatic residue, a C(═O)—C₃₋₁₀ cycloaliphatic residue, a C(═O)NH—C₃₋₁₀ cycloaliphatic residue a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—C(═O)—C₃₋₁₀ cycloaliphatic residue, a NH—(C₁₋₈ aliphatic group)-C₃₋₁₀ cycloaliphatic residue, a N(C₁₋₁₀-aliphatic residue)(C₃₋₁₀ cycloaliphatic residue), a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a O—(C₁₋₈ aliphatic group)-(3 to 10 membered heterocycloaliphatic residue), a S-(3 to 10 membered heterocycloaliphatic residue), a S—(C₁₋₈ aliphatic group)-(3 to 10 membered heterocyclo-aliphatic residue), a NH-(3 to 10 membered heterocycloaliphatic residue), a NH—C(═O)-(3 to 10 membered heterocycloaliphatic residue), NH—(C₁₋₈ aliphatic group)-(3 to 10 membered heterocycloaliphatic residue), a N(C₁₋₁₀ aliphatic residue)(3 to 10 membered heterocycloaliphatic residue),
   wherein each of the aforementioned residues can in each case be optionally bridged via an C₁₋₈ aliphatic group,
   wherein in each case independently of one another the C₁₋₁₀ aliphatic residue and the C₁₋₈ aliphatic group can be unsubstituted or monosubstituted with OH,
   wherein in each case independently of one another, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, ═O, OCF₃, OH, SH, S—C₁₋₄ alkyl, SCF₃, SO₂—C₁₋₄ alkyl, NH₂, ═NH, ═N(OH), NH—C₁₋₄ alkyl, N(C₁₋₄ alkyl)₂, NH—SO₂—C₁₋₄ alkyl, NH—C(═O)—C₁₋₄ alkyl,
aryl, C(═O)-aryl, C(═O)—NH-aryl, O-aryl, a O—(C₁₋₈ aliphatic group)-aryl, S-aryl, a S—(C₁₋₈ aliphatic group)-aryl, a NH-aryl, NH—C(═O)-aryl, NH—S(═O)₂-aryl a NH—(C₁₋₈ aliphatic group)-aryl, a N(C₁₋₁₀ aliphatic residue)(aryl), heteroaryl, C(═O)-heteroaryl, C(═O)—NH-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)(heteroaryl), N(C₁₋₁₀ aliphatic residue)(heteroaryl),
   wherein each of the aforementioned residues can in each case be optionally bridged via a C₁₋₈ aliphatic group,
   wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH,
   wherein in each case the C₁₋₁₀ aliphatic residues and the C₁₋₈ aliphatic groups of the aforementioned residues can be unsubstituted or monosubstituted with OH.

More preferably,

R⁵, R⁶, R⁷, R⁸ and R⁹ are each independently of one another selected from the group consisting of
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue,
a O—C₁₋₁₀ aliphatic residue, a O—(C₁₋₈ aliphatic group)-O—C₁₋₁₀ aliphatic residue, O—(C₁₋₈ aliphatic group)-OH,
a NH—C₁₋₁₀ aliphatic residue, a N(C₁₋₁₀ aliphatic residue)₂, a NH—(C₁₋₈ aliphatic group)-O—C₁₋₁₀ aliphatic residue, a NH—(C₁₋₈ aliphatic group)-OH, a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue,
   wherein each of the aforementioned C₁₋₁₀ aliphatic residues and C₁₋₈ aliphatic groups can in each case be unsubstituted or monosubstituted with OH;
a C₃₋₁₀ cycloaliphatic residue, a C(═O)—C₃₋₁₀ cycloaliphatic residue, a C(═O)NH—C₃₋₁₀ cycloaliphatic residue, a O—C₃₋₁₀ cycloaliphatic residue, a NH—C₃₋₁₀ cycloaliphatic residue, a NH—C(═O)—C₃₋₁₀ cycloaliphatic residue, a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a NH-(3 to 10 membered heterocycloaliphatic residue), a NH—C(═O)-(3 to 10 membered heterocycloaliphatic residue),
wherein in each case independently of one another, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, OCF₃, OH, SH, S—C₁₋₄ alkyl, SCF₃, SO₂—C₁₋₄ alkyl, NH₂, NH—C₁₋₄ alkyl, N(C₁₋₄ alkyl)₂, NH—SO₂—C₁₋₄ alkyl, NH—C(═O)—C₁₋₄ alkyl;
aryl, C(═O)-aryl, C(═O)—NH-aryl, NH—C(═O)-aryl, heteroaryl, C(═O)-heteroaryl, C(═O)—NH-heteroaryl, NH—C(═O)-heteroaryl,
   wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH.

Even more preferably,

R⁵, R⁶, R⁷, R⁸ and R⁹ are each independently of one another selected from the group consisting of
H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
a C₁₋₄ aliphatic residue, (C₁₋₄ aliphatic group)-OH, (C₁₋₄ aliphatic group)-O—C₁₋₄ aliphatic residue, (C₁₋₄ aliphatic group)-O—(C₁₋₄ aliphatic group)-OH, (C₁₋₄ aliphatic group)-O—(C₁₋₄ aliphatic group)-O—C₁₋₄ aliphatic residue, a (C₁₋₄ aliphatic group)-NH—C₁₋₄ aliphatic residue, a (C₁₋₄ aliphatic group)-NH—(C₁₋₄ aliphatic residue)-OH, a (C₁₋₄ aliphatic group)-N(C₁₋₄ aliphatic residue)-(C₁₋₄ aliphatic residue)-OH, a (C₁₋₄ aliphatic group)-NH—S(═O)₂—C₁₋₄ aliphatic residue, a (C₁₋₄ aliphatic group)-NH—S(═O)₂—NH₂, a (C₁₋₄ aliphatic group)-S(═O)₂—C₁₋₄ aliphatic residue, a O—C₁₋₄ aliphatic residue, a O—(C₁₋₄ aliphatic group)-O—C₁₋₄ aliphatic residue, O—(C₁₋₄ aliphatic group)-OH,
a NH—C₁₋₄ aliphatic residue, a N(C₁₋₄ aliphatic residue)₂, a NH—(C₁₋₄ aliphatic group)-O—C₁₋₄ aliphatic residue, a NH—(C₁₋₄ aliphatic group)-OH, a N(C₁₋₄ aliphatic residue)[(C₁₋₄ aliphatic group)-O—C₁₋₄ aliphatic residue], a N(C₁₋₄ aliphatic residue)[(C₁₋₄ aliphatic group)-OH], a NH—S(═O)₂—C₁₋₄ aliphatic residue,
   wherein each of the aforementioned C₁₋₄ aliphatic residues and C₁₋₄ aliphatic groups can in each case be unsubstituted or monosubstituted with OH;
a C₃₋₆ cycloaliphatic residue, O—C₃₋₆ cycloaliphatic residue, a 3 to 6 membered heterocycloaliphatic residue, O-(3 to 6 membered heterocycloaliphatic residue),
   wherein in each case independently of one another, the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, OH, SH, S—C₁₋₄ alkyl, SO₂—C₁₋₄ alkyl, NH₂, NH—C₁₋₄ alkyl, N(C₁₋₄ alkyl)₂, NH—SO₂—C₁₋₄ alkyl, and NH—C(═O)—C₁₋₄ alkyl,
aryl, C(═O)—NH-aryl, NH—C(═O)-aryl, heteroaryl, C(═O)—NH-heteroaryl, NH—C(═O)-heteroaryl,
   wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, and NH—C(═O)—C₁₋₄ alkyl.

Still more preferably,

R⁵, R⁶, R⁷, R⁸ and R⁹ are each independently of one another selected from the group consisting of
H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
C₁₋₄ alkyl, C₁₋₄ alkylene-OH, C₁₋₄ alkylene-O—C₁₋₄ alkyl, C₁₋₄ alkylene-O—C₁₋₄ alkylene-OH, C₁₋₄ alkylene-O—C₁₋₄ alkylene-O—C₁₋₄ alkyl, C₁₋₄ alkylene-S(═O)₂—C₁₋₄ alkyl, C₁₋₄ alkylene-NH—S(═O)₂—C₁₋₄ alkyl, C₁₋₄ alkylene-NH—S(═O)₂—NH₂, C₁₋₄ alkylene-NH—C₁₋₄ alkylene-OH, C₁₋₄ alkylene-NH—C₁₋₄ alkylene-O—C₁₋₄ alkyl, C₁₋₄ alkylene-N(C₁₋₄ alkyl)-C₁₋₄ alkylene-OH, C₁₋₄ alkylene-N(C₁₋₄ alkyl)-C₁₋₄ alkylene-O—C₁₋₄ alkyl, O—C₁₋₄ alkyl, O—C₁₋₄ alkylene-OH, O—C₁₋₄ alkylene-O—C₁₋₄ alkyl, NH—C₁₋₄ alkyl, N(C₁₋₄ alkyl)₂, NH—C₁₋₄ alkylene-OH, NH—C₁₋₄ alkylene-O—C₁₋₄ alkyl, N(C₁₋₄ alkyl)-[C₁₋₄ alkylene-OH], N(C₁₋₄ alkyl)-[C₁₋₄ alkylene-O—C₁₋₄ alkyl], NH—S(═O)₂—C₁₋₄ alkyl,
   wherein C₁₋₄ alkylene can in each case be unsubstituted or monosubstituted with OH,
a C₃₋₆ cycloaliphatic residue, O—C₃₋₆ cycloaliphatic residue, a 3 to 6 membered heterocycloaliphatic residue,
   wherein the C₃₋₆ cycloaliphatic residue is preferably selected from the group consisting of cyclopropyl, cyclobutyl, cyclopentyl, cyclohexyl, and
   wherein the 3 to 6 membered heterocycloaliphatic residue is preferably selected from the group consisting of tetrahydropyranyl, preferably tetrahydro-2H-pyran-4-yl, azetidinyl, piperidinyl, morpholinyl and pyrrolidinyl,
   wherein the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, OH, O—C₁₋₄ alkyl, NH₂, NH(C₁₋₄ alkyl), and N(C₁₋₄ alkyl)₂, and C₁₋₄ alkyl,
phenyl, C(═O)—NH-phenyl, NH—C(═O)-phenyl, heteroaryl, C(═O)—NH-heteroaryl, NH—C(═O)-heteroaryl, preferably phenyl, C(═O)—NH-phenyl and NH—C(═O)-phenyl,
   wherein heteroaryl is preferably selected from the group consisting of pyridyl, furyl and thienyl;
   wherein in each case independently of one another phenyl and heteroaryl of the aforementioned residues, respectively, can be unsubstituted or mono- or polysubstituted with one or more substituents each selected independently of one another from the group consisting of F, Cl, Br, I, OH, O—C₁₋₄ alkyl, C₁₋₄ alkyl, and CF₃.

In yet another preferred embodiment of the compound according to the invention of general formula (I),

R⁵, R⁶, R⁸ and R⁹ are each independently of one another selected from the group consisting of
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂; a C₁₋₁₀ aliphatic residue, a NH—C₁₋₁₀ aliphatic residue, a N(C₁₋₁₀, aliphatic residue)₂ and a O—C₁₋₁₀ aliphatic residue, wherein the C₁₋₁₀ aliphatic residue can in each case be unsubstituted or mono- or disubstituted with OH;
   and R⁷ is selected from the group consisting of
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH-(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue, a C(═O)—C₁₋₁₀ aliphatic residue, a C(═O)—NH—C₁₋₁₀ aliphatic residue,
a O—C₁₋₁₀ aliphatic residue, a O—(C₁₋₈ aliphatic group)-O—C₁₋₁₀ aliphatic residue, O—(C₁₋₈ aliphatic group)-OH,
a NH—C₁₋₁₀ aliphatic residue, a N(C₁₋₁₀ aliphatic residue)₂, a NH—[(C₁₋₈ aliphatic group)-O—C₁₋₁₀ aliphatic residue], a NH—[(C₁₋₈ aliphatic group)-OH], a N(C₁₋₁₀ aliphatic residue)[(C₁₋₈ aliphatic group)-OH], a N(C₁₋₁₀ aliphatic residue)[(C₁₋₈ aliphatic group)-O—C₁₋₈ aliphatic residue], a NH—C(═O)—C₁₋₁₀ aliphatic residue, a N(C₁₋₁₀ aliphatic residue)[(C(═O)—C₁₋₁₀ aliphatic residue)], a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue, a N(C₁₋₁₀ aliphatic residue)[S(═O)₂—C₁₋₁₀ aliphatic residue],
a S(═O)₂—C₁₋₁₀ aliphatic residue, a S(═O)₂—NH—C₁₋₁₀ aliphatic residue, a S(═O)₂—N(C₁₋₁₀ aliphatic residue)₂, a S—C₁₋₁₀ aliphatic residue,
   wherein each of the aforementioned C₁₋₁₀ aliphatic residues and C₁₋₈ aliphatic groups can in each case be unsubstituted or mono- or polysubstituted with one or more substituents each selected independently of one another from the group consisting of F, Cl, Br, I, NO₂, CN, OH, ═O, O—C₁₋₄ alkyl, OCF₃, OF₃, NH₂, NH(C₁₋₄ alkyl), N(C₁₋₄ alkyl)₂, SH, S—C₁₋₄ alkyl, SCF₃, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
a C₃₋₁₀ cycloaliphatic residue, a C(═O)—C₃₋₁₀ cycloaliphatic residue, a C(═O)NH—C₃₋₁₀ cycloaliphatic residue a O—C₃₋₁₀ cycloaliphatic residue, a O—(C₁₋₈ aliphatic group)-C₃₋₁₀-cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—C(═O)—C₃₋₁₀ cycloaliphatic residue, a NH—(C₁₋₈ aliphatic group)-C₃₋₁₀ cycloaliphatic residue, a N(C₁₋₁₀-aliphatic residue)(C₃₋₁₀ cycloaliphatic residue), a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a O—(C₁₋₈ aliphatic group)-(3 to 10 membered heterocycloaliphatic residue), a S-(3 to 10 membered heterocycloaliphatic residue), a S—(C₁₋₈ aliphatic group)-(3 to 10 membered heterocyclo-aliphatic residue), a NH-(3 to 10 membered heterocycloaliphatic residue), a NH—C(═O)-(3 to 10 membered heterocycloaliphatic residue), NH—(C₁₋₈ aliphatic group)-(3 to 10 membered heterocycloaliphatic residue), a N(C₁₋₁₀ aliphatic residue)(3 to 10 membered heterocycloaliphatic residue),
   wherein each of the aforementioned residues can in each case be optionally bridged via a C₁₋₈ aliphatic group,
   wherein in each case independently of one another the C₁₋₁₀ aliphatic residue, the C₁₋₈ aliphatic group, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, ═O, OCF₃, OH, SH, S—C₁₋₄ alkyl, SCF₃, SO₂—C₁₋₄ alkyl, NH₂, ═NH, ═N(OH), NH—C₁₋₄ alkyl, N(C₁₋₄ alkyl)₂, NH—SO₂—C₁₋₄ alkyl, NH—C(═O)—C₁₋₄ alkyl, phenyl and pyridyl, wherein phenyl and pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH,
aryl, C(═O)-aryl, C(═O)—NH-aryl, O-aryl, a O—(C₁₋₈ aliphatic group)-aryl, S-aryl, a S—(C₁₋₈ aliphatic group)-aryl, a NH-aryl, NH—C(═O)-aryl, NH—S(═O)₂-aryl a NH—(C₁₋₈ aliphatic group)-aryl, a N(C₁₋₁₀ aliphatic residue)(aryl), heteroaryl, C(═O)-heteroaryl, C(═O)—NH-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)(heteroaryl), N(C₁₋₁₀ aliphatic residue)(heteroaryl),
   wherein each of the aforementioned residues can in each case be optionally bridged via a C₁₋₈ aliphatic group,
   wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH,
   wherein in each case independently of one another the C₁₋₁₀ aliphatic residues and the C₁₋₈ aliphatic groups of the aforementioned residues, respectively, can be unsubstituted or mono- or polysubstituted with one or more substituents each selected independently of one another from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, OCF₃, C₁₋₄ alkyl, C(═O)—OH, CF₃, NH₂, NH(C₁₋₄ alkyl), N(C₁₋₄ alkyl)₂, SH, S—C₁₋₄ alkyl, SCF₃ and S(═O)₂OH.

Preferably,

R⁵, R⁶, R⁸ and R⁹ are each independently of one another selected from the group consisting of
H; F; Cl; Br; I; CN; NO₂; CF₃; CF₂H; CFH₂; OH; OCF₃; OCF₂Cl; OCFCl₂; SH; SCF₃; NH₂; C(═O)—NH₂; methyl; ethyl; tert.-butyl; O-methyl; NH-methyl; N(methyl)₂;
   and R⁷ is selected from the group consisting of
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue, a C(═O)—C₁₋₁₀ aliphatic residue, a C(═O)—NH—C₁₋₁₀ aliphatic residue,
a O—C₁₋₁₀ aliphatic residue, a O—(C₁₋₈ aliphatic group)-O—C₁₋₁₀ aliphatic residue, O—(C₁₋₈ aliphatic group)-OH,
a NH—C₁₋₁₀ aliphatic residue, a N(C₁₋₁₀ aliphatic residue)₂, a NH—(C₁₋₈ aliphatic group)-—O—C₁₋₁₀ aliphatic residue, a NH—(C₁₋₈ aliphatic group)-OH, a N(C₁₋₁₀ aliphatic residue)[(C₁₋₈ aliphatic group)-OH], a N(C₁₋₁₀ aliphatic residue)[(C₁₋₈ aliphatic group)O—C₁₋₁₀ aliphatic residue], a NH—C(═O)—C₁₋₁₀ aliphatic residue, a N(C₋₁₀ aliphatic residue)[(C(═O)—C₁₋₁₀ aliphatic residue)], a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)OH], a NH—S(═O)₂—C₁₋₁₀ aliphatic residue, a N(C₁₋₁₀ aliphatic residue)[S(═O)₂—C₁₋₁₀ aliphatic residue],
a S(═O)₂—C₁₋₁₀ aliphatic residue, a S(═O)₂—NH—C₁₋₁₀ aliphatic residue, a S(═O)₂—N(C₁₋₁₀ aliphatic residue)₂, a S—C₁₋₁₀ aliphatic residue,
   wherein each of the aforementioned C₁₋₁₀ aliphatic residues and C₁₋₈ aliphatic groups can in each case be unsubstituted or monosubstituted with OH;
a C₃₋₁₀ cycloaliphatic residue, a C(═O)—C₃₋₁₀ cycloaliphatic residue, a C(═O)NH—C₃₋₁₀ cycloaliphatic residue a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—C(═O)—C₃₋₁₀ cycloaliphatic residue, a NH—(C₁₋₈ aliphatic group)-C₃₋₁₀ cycloaliphatic residue, a N(C₁₋₁₀-aliphatic residue)(C₃₋₁₀ cycloaliphatic residue), a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a O—(C₁₋₈ aliphatic group)-(3 to 10 membered heterocycloaliphatic residue), a S-(3 to 10 membered heterocycloaliphatic residue), a S—(C₁₋₈ aliphatic group)-(3 to 10 membered heterocyclo-aliphatic residue), a NH-(3 to 10 membered heterocycloaliphatic residue), a NH—C(═O)-(3 to 10 membered heterocycloaliphatic residue), NH—(C₁₋₈ aliphatic group)-(3 to 10 membered heterocycloaliphatic residue), a N(C₁₋₁₀ aliphatic residue)(3 to 10 membered heterocycloaliphatic residue),
   wherein each of the aforementioned residues can in each case be optionally bridged via an C₁₋₈ aliphatic group,
   wherein in each case independently of one another the C₁₋₁₀ aliphatic residue and the C₁₋₈ aliphatic group can be unsubstituted or monosubstituted with OH,
   wherein in each case independently of one another, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, ═O, OCF₃, OH, SH, S—C₁₋₄ alkyl, SCF₃, SO₂—C₁₋₄ alkyl, NH₂, ═NH, ═N(OH), NH—C₁₋₄ alkyl, N(C₁₋₄ alkyl)₂, NH—SO₂—C₁₋₄ alkyl, NH—C(═O)—C₁₋₄ alkyl,
aryl, C(═O)-aryl, C(═O)—NH-aryl, O-aryl, a O—(C₁₋₈ aliphatic group)-aryl, S-aryl, a S—(C₁₋₈ aliphatic group)-aryl, a NH-aryl, NH—C(═O)-aryl, NH—S(═O)₂-aryl a NH—(C₁₋₈ aliphatic group)-aryl, a N(C₁₋₁₀ aliphatic residue)(aryl), heteroaryl, C(═O)-heteroaryl, C(═O)—NH-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)(heteroaryl), N(C₁₋₁₀ aliphatic residue)(heteroaryl),
   wherein each of the aforementioned residues can in each case be optionally bridged via a C₁₋₈ aliphatic group,
   wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH,
   wherein in each case the C₁₋₁₀ aliphatic residues and the C₁₋₈ aliphatic groups of the aforementioned residues can be unsubstituted or monosubstituted with OH.

More preferably,

R⁵, R⁶, R⁸ and R⁹ are each independently of one another selected from the group consisting of
H; F; Cl; Br; I; CF₃; CF₂H; CFH₂; OH; methyl; and O-methyl;
   and R⁷ is selected from the group consisting of
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂; C(═O)—H; C(═O)—OH; S(═O)₂—OH; S(═O)₂—NH₂;
a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)-O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NH—S(═O)₂—C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue,
a O—C₁₋₁₀ aliphatic residue, a O—(C₁₋₈ aliphatic group)-O—C₁₋₁₀ aliphatic residue, O—(C₁₋₈ aliphatic group)-OH,
a NH—C₁₋₁₀ aliphatic residue, a N(C₁₋₁₀ aliphatic residue)₂, a NH—(C₁₋₈ aliphatic group)-O—C₁₋₁₀ aliphatic residue, a NH—(C₁₋₈ aliphatic group)-OH, a N(C₁₋₁₀ aliphatic residue)[(C₁₋₈ aliphatic group)-O—C₁₋₁₀ aliphatic residue], a N(C₁₋₁₀ aliphatic residue)[(C₁₋₈ aliphatic group)-OH], a NH—S(═O)₂—C₁₋₁₀ aliphatic residue,
   wherein each of the aforementioned C₁₋₁₀ aliphatic residues and C₁₋₈ aliphatic groups can in each case be unsubstituted or monosubstituted with OH;
a C₃₋₁₀ cycloaliphatic residue, a C(═O)—C₃₋₁₀ cycloaliphatic residue, a C(═O)NH—C₃₋₁₀ cycloaliphatic residue, a O—C₃₋₁₀ cycloaliphatic residue, a NH—C₃₋₁₀ cycloaliphatic residue, a NH—C(═O)—C₃₋₁₀ cycloaliphatic residue, a 3 to 10 membered heterocycloaliphatic residue, a C(═O)-(3 to 10 membered heterocycloaliphatic residue), a C(═O)—NH-(3 to 10 membered heterocycloaliphatic residue), a O-(3 to 10 membered heterocycloaliphatic residue), a NH-(3 to 10 membered heterocycloaliphatic residue), a NH—C(═O)-(3 to 10 membered heterocycloaliphatic residue),
   wherein in each case independently of one another, the C₃₋₁₀ cycloaliphatic residue and the 3 to 10 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, OCF₃, OH, SH, S—C₁₋₄ alkyl, SCF₃, SO₂—C₁₋₄ alkyl, NH₂, NH—C₁₋₄ alkyl, N(C₁₋₄ alkyl)₂, NH—SO₂—C₁₋₄ alkyl, NH—C(═O)—C₁₋₄ alkyl;
aryl, C(═O)-aryl, C(═O)—NH-aryl, NH—C(═O)-aryl, heteroaryl, C(═O)-heteroaryl, C(═O)—NH-heteroaryl, NH—C(═O)-heteroaryl,
   wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, NH—C(═O)—C₁₋₄ alkyl, phenyl and pyridyl, wherein phenyl or pyridyl are respectively unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH.

Even more preferably,

R⁵, R⁶, R⁸ and R⁹ are each independently of one another selected from the group consisting of
H; F; Cl; Br; I; CF₃; OH; methyl; and O-methyl;
   and R⁷ is selected from the group consisting of
H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
a C₁₋₄ aliphatic residue, (C₁₋₄ aliphatic group)-OH, (C₁₋₄ aliphatic group)-O—C₁₋₄ aliphatic residue, (C₁₋₄ aliphatic group)-O—(C₁₋₄ aliphatic group)-OH, (C₁₋₄ aliphatic group)-O—(C₁₋₄ aliphatic group)-O—C₁₋₄ aliphatic residue, a (C₁₋₄ aliphatic group)-NH—C₁₋₄ aliphatic residue, a (C₁₋₄ aliphatic group)-NH—(C₁₋₄ aliphatic residue)-OH, a (C₁₋₄ aliphatic group)-N(C₁₋₄ aliphatic residue)-(C₁₋₄ aliphatic residue)-OH, a (C₁₋₄ aliphatic group)-NH—S(═O)₂—C₁₋₄ aliphatic residue, a (C₁₋₄ aliphatic group)-NH—S(═O)₂—NH₂, a (C₁₋₄ aliphatic group)-S(═O)₂—C₁₋₄ aliphatic residue,
a O—C₁₋₄ aliphatic residue, a O—(C₁₋₄ aliphatic group)-O—C₁₋₄ aliphatic residue, O—(C₁₋₄ aliphatic group)-OH,
a NH—C₁₋₄ aliphatic residue, a N(C₁₋₄ aliphatic residue)₂, a NH—(C₁₋₄ aliphatic group)-O—C₁₋₄ aliphatic residue, a NH—(C₁₋₄ aliphatic group)-OH, a N(C₁₋₄ aliphatic residue)[(C₁₋₄ aliphatic group)-O—C₁₋₄ aliphatic residue], a N(C₁₋₄ aliphatic residue)[(C₁₋₄ aliphatic group)-OH], a NH—S(═O)₂—C₁₋₄ aliphatic residue,
   wherein each of the aforementioned C₁₋₄ aliphatic residues and C₁₋₄ aliphatic groups can in each case be unsubstituted or monosubstituted with OH;
a C₃₋₆ cycloaliphatic residue, O—C₃₋₆ cycloaliphatic residue, a 3 to 6 membered heterocycloaliphatic residue, O-(3 to 6 membered heterocycloaliphatic residue),
   wherein in each case independently of one another, the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, C₁₋₄ alkyl, C₁₋₄ alkylene-OH, C₁₋₄ alkylene-O—C₁₋₄ alkyl, CF₃, C(═O)—C₁₋₄ alkyl, O—C₁₋₄ alkyl, O—C₁₋₄ alkylene-OH, O—C₁₋₄ alkylene-O—C₁₋₄ alkyl, OH, SH, S—C₁₋₄ alkyl, SO₂—C₁₋₄ alkyl, NH₂, NH—C₁₋₄ alkyl, N(C₁₋₄ alkyl)₂, NH—SO₂—C₁₋₄ alkyl, and NH—C(═O)—C₁₋₄ alkyl,
aryl, C(═O)—NH-aryl, NH—C(═O)-aryl, heteroaryl, C(═O)—NH-heteroaryl, NH—C(═O)-heteroaryl,
   wherein in each case independently of one another the aryl and heteroaryl of the aforementioned residues, respectively, can be unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl, O—C₁₋₄ alkylene-OH, OCF₃, C₁₋₄ alkyl, C₁₋₄ alkylene-O—C₁₋₄-alkyl, C₁₋₄ alkylene-OH, C(═O)—C₁₋₄ alkyl, CF₃, CF₂H, CHF₂, SH, S—C₁₋₄ alkyl, SCF₃, SO₂—C₁₋₄ alkyl, NH₂, NH(C₁₋₄ alkyl), N(C₁₋₄ alkyl)₂, NH—SO₂—C₁₋₄ alkyl, and NH—C(═O)—C₁₋₄ alkyl.

Still more preferably,

R⁵, R⁶, R⁸ and R⁹ are each independently of one another selected from the group consisting of

H; F; Cl; Br; I; CF₃; OH; methyl; O-methyl;

and R⁷ is selected from the group consisting of
H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
C₁₋₄ alkyl, C₁₋₄ alkylene-OH, C₁₋₄ alkylene-O—C₁₋₄ alkyl, C₁₋₄ alkylene-O—C₁₋₄ alkylene-OH, C₁₋₄ alkylene-O—C₁₋₄ alkylene-O—C₁₋₄ alkyl, C₁₋₄ alkylene-S(═O)₂—C₁₋₄ alkyl, C₁₋₄ alkylene-NH—S(═O)₂—C₁₋₄ alkyl, C₁₋₄ alkylene-NH—S(═O)₂—NH₂, C₁₋₄ alkylene-NH—C₁₋₄ alkylene-OH, C₁₋₄ alkylene-NH—C₁₋₄ alkylene-O—C₁₋₄ alkyl, C₁₋₄ alkylene-N(C₁₋₄ alkyl)-C₁₋₄ alkylene-OH, C₁₋₄ alkylene-N(C₁₋₄ alkyl)-C₁₋₄ alkylene-O—C₁₋₄ alkyl, O—C₁₋₄ alkyl, O—C₁₋₄ alkylene-OH, O—C₁₋₄ alkylene-O—C₁₋₄ alkyl, NH—C₁₋₄ alkyl, N(C₁₋₄ alkyl)₂, NH—C₁₋₄ alkylene-OH, NH—C₁₋₄ alkylene-O—C₁₋₄ alkyl, N(C₁₋₄ alkyl)-[C₁₋₄ alkylene-OH], N(C₁₋₄ alkyl)-[C₁₋₄ alkylene-O—C₁₋₄ alkyl], NH—S(═O)₂—C₁₋₄ alkyl,
   wherein C₁₋₄ alkylene can in each case be unsubstituted or monosubstituted with OH,
a C₃₋₆ cycloaliphatic residue, O—C₃₋₆ cycloaliphatic residue, a 3 to 6 membered heterocycloaliphatic residue,
   wherein the C₃₋₆ cycloaliphatic residue is preferably selected from the group consisting of cyclopropyl, cyclobutyl, cyclopentyl, cyclohexyl, and
   wherein the 3 to 6 membered heterocycloaliphatic residue is preferably selected from the group consisting of tetrahydropyranyl, preferably tetrahydro-2H-pyran-4-yl, azetidinyl, piperidinyl, morpholinyl and pyrrolidinyl,
   wherein the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, OH, O—C₁₋₄ alkyl, NH₂, NH(C₁₋₄ alkyl), and N(C₁₋₄ alkyl)₂, and C₁₋₄ alkyl, phenyl, C(═O)—NH-phenyl, NH—C(═O)-phenyl, heteroaryl, C(═O)—NH-heteroaryl, NH—C(═O)-heteroaryl, preferably phenyl, C(═O)—NH-phenyl and NH—C(═O)-phenyl,
   wherein heteroaryl is preferably selected from the group consisting of pyridyl, furyl and thienyl;
   wherein in each case independently of one another phenyl and heteroaryl of the aforementioned residues, respectively, can be unsubstituted or mono- or polysubstituted with one or more substituents each selected independently of one another from the group consisting of F, Cl, Br, I, OH, O—C₁₋₄ alkyl, C₁₋₄ alkyl, and CF₃.

In a particularly preferred embodiment of the compound according to the invention of general formula (I),

R⁵ and R⁹ are each independently of one another selected from the group consisting of
H; F; Cl; Br; I; CF₃; OH; methyl; O-methyl; preferably both denote H,
   R⁶ and R⁸ are each independently of one another selected from the group consisting of
H; F; Cl; Br; I; CF₃; OH; methyl; O-methyl;
   and R⁷ is selected from the group consisting of
H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
C₁₋₄ alkyl, C₁₋₄ alkylene-OH, C₁₋₄ alkylene-O—C₁₋₄ alkyl, C₁₋₄ alkylene-O—C₁₋₄ alkylene-OH, C₁₋₄ alkylene-O—C₁₋₄ alkylene-O—C₁₋₄ alkyl, C₁₋₄ alkylene-S(═O)₂—C₁₋₄ alkyl, C₁₋₄ alkylene-NH—S(═O)₂—C₁₋₄ alkyl, C₁₋₄ alkylene-NH—S(═O)₂—NH₂, C₁₋₄ alkylene-NH—C₁₋₄ alkylene-OH, C₁₋₄ alkylene-NH—C₁₋₄ alkylene-O—C₁₋₄ alkyl, C₁₋₄ alkylene-N(C₁₋₄ alkyl)-C₁₋₄ alkylene-OH, C₁₋₄ alkylene-N(C₁₋₄ alkyl)-C₁₋₄ alkylene-O—C₁₋₄ alkyl, O—C₁₋₄ alkyl, O—C₁₋₄ alkylene-OH, O—C₁₋₄ alkylene-O—C₁₋₄ alkyl, NH—C₁₋₄ alkyl, N(C₁₋₄ alkyl)₂, NH—C₁₋₄ alkylene-OH, NH—C₁₋₄ alkylene-O—C₁₋₄ alkyl, N(C₁₋₄ alkyl)-[C₁₋₄ alkylene-OH], N(C₁₋₄ alkyl)-[C₁₋₄ alkylene-O—C₁₋₄ alkyl], NH—S(═O)₂—C₁₋₄ alkyl,
   wherein C₁₋₄ alkylene can in each case be unsubstituted or monosubstituted with OH,
a C₃₋₆ cycloaliphatic residue, O—C₃₋₆ cycloaliphatic residue, a 3 to 6 membered heterocycloaliphatic residue,
   wherein the C₃₋₆ cycloaliphatic residue is preferably selected from the group consisting of cyclopropyl, cyclobutyl, cyclopentyl, cyclohexyl, and
   wherein the 3 to 6 membered heterocycloaliphatic residue is preferably selected from the group consisting of tetrahydropyranyl, preferably tetrahydro-2H-pyran-4-yl, azetidinyl, piperidinyl, morpholinyl and pyrrolidinyl,
   wherein the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, OH, O—C₁₋₄ alkyl, NH₂, NH(C₁₋₄ alkyl), and N(C₁₋₄ alkyl)₂, and C₁₋₄ alkyl,
phenyl, C(═O)—NH-phenyl, NH—C(═O)-phenyl, heteroaryl, C(═O)—NH-heteroaryl, NH—C(═O)-heteroaryl, preferably phenyl, C(═O)—NH-phenyl and NH—C(═O)-phenyl,
   wherein heteroaryl is preferably selected from the group consisting of pyridyl, furyl and thienyl;
   wherein in each case independently of one another phenyl and heteroaryl of the aforementioned residues, respectively, can be unsubstituted or mono- or polysubstituted with one or more substituents each selected independently of one another from the group consisting of F, Cl, Br, I, OH, O—C₁₋₄ alkyl, C₁₋₄ alkyl, and CF₃.

In another particularly preferred embodiment of the compound according to the invention of general formula (I),

R⁵ and R⁹ both denote H,
R⁶ and R⁸ are each independently of one another selected from the group consisting of
H; F; Cl; Br; I; CF₃; OH; methyl; O-methyl;
   and R⁷ is selected from the group consisting of
H, F, Cl, Br, I, CN, CF₃, CF₂H, CFH₂, OH, OCF₃, SH, SCF₃, NH₂, C(═O)—NH₂, S(═O)₂—OH, S(═O)₂—NH₂,
CH₃, C₂H₅, CH₂—OH, C₂H₄—OH, CH₂—CH(OH)—CH₂—OH, CH₂—O—CH₃, C₂H₄—O—CH₃, CH₂—O—CH₂—OH, CH₂—O—C₂H₄—OH, CH₂—O—CH₂—O—CH₃, CH₂—O—C₂H₄—O—CH₃, CH₂—S(═O)₂—CH₃, C₂H₄—S(═O)₂—CH₃, CH₂—NH—S(═O)₂—CH₃, CH₂—NH—S(═O)₂—NH₂, CH₂—NH—CH₂—OH, CH₂—NH—C₂H₄—OH, CH₂—NH—C₂H₄—O—CH₃, CH₂—N(CH₃)—C₂H₄—OH, CH₂—N(CH₃)—C₂H₄—O—CH₃, O—CH₃, O—C₂H₄—OH, O—C₂H₄—O—CH₃, NH—CH₃, N(CH₃)₂, NH—C₂H₄—OH, NH—C₂H₄—O—CH₃, N(CH₃)—[C₂H₄—OH], N(CH₃)—[C₂H₄—O—CH₃], NH—S(═O)₂—CH₃,
cyclopropyl, cyclobutyl, cyclopentyl, cyclohexyl, O-cyclopropyl, tetrahydropyranyl, preferably tetrahydro-2H-pyran-4-yl, azetidinyl, piperidinyl, morpholinyl or pyrrolidinyl, in each case independently of one another unsubstituted or mono- or polysubstituted with one or more substituents each selected independently of one another from the group consisting of F, Cl, Br, I, OH, O—CH₃, NH₂, N(CH₃)₂, CH₃, C₂H₅ and tert.-butyl,
phenyl, C(═O)—NH-phenyl, or NH—C(═O)-phenyl, wherein in each case independently of one another phenyl can be unsubstituted or mono- or polysubstituted with one or more substituents each selected independently of one another from the group consisting of F, Cl, Br, I, OH, O—CH₃, CH₃, C₂H₅, and CF₃.

Particularly preferred residues for R⁷ are selected from the group consisting of
H, F, Cl, Br, I, CN, CF₃, CF₂H, CFH₂, OH, OCF₃, SH, SCF₃, NH₂, C(═O)—NH₂, S(═O)₂—OH, S(═O)₂—NH₂,
CH₃, C₂H₅, CH₂—OH, C₂H₄—OH, CH(OH)—CH₂OH, CH₂—CH(OH)—CH₂—OH, CH₂—O—CH₃, C₂H₄—O—CH₃, CH₂—O—CH₂—OH, CH₂—O—C₂H₄—OH, CH₂—O—CH₂—O—CH₃, CH₂—C₂H₄—CH₃, CH₂—S(═O)₂—CH₃, C₂H₄—S(═O)₂—CH₃, CH₂—NH—S(═O)₂—CH₃, CH₂—NH—S(═O)₂—NH₂, CH₂—NH—CH₂—OH, CH₂—NH—C₂H₄—OH, CH₂—NH—C₂H₄—O—CH₃, CH₂—N(CH₃)—C₂H₄—OH, CH₂—N(CH₃)—C₂H₄—O—CH₃, O—CH₃, O—C₂H₄—OH, O—C₂H₄—O—CH₃, NH—CH₃, N(CH₃)₂, NH—C₂H₄—OH, NH—C₂H₄—O—CH₃, N(CH₃)—[C₂H₄—OH], N(CH₃)—[C₂H₄—O—CH₃], NH—S(═O)₂—CH₃,
cyclopropyl, cyclobutyl, cyclopentyl, cyclohexyl, O-cyclopropyl, tetrahydropyranyl, preferably tetrahydro-2H-pyran-4-yl, azetidinyl, piperidinyl, morpholinyl or pyrrolidinyl, in each case independently of one another unsubstituted or mono- or polysubstituted with one or more substituents each selected independently of one another from the group consisting of F, Cl, Br, I, OH, O—CH₃, NH₂, N(CH₃)₂, CH₃, C₂H₅ and tert.-butyl,
phenyl, C(═O)—NH-phenyl, or NH—C(═O)-phenyl, wherein in each case independently of one another phenyl can be unsubstituted or mono- or polysubstituted with one or more substituents each selected independently of one another from the group consisting of F, Cl, Br, I, OH, O—CH₃, CH₃, C₂H₅, and CF₃.

Most preferred residues for R⁷ are selected from the group consisting of CH₂—S(═O)₂—CH₃, C₂H₄—S(═O)₂—CH₃, CH₂—O—C₂H₄—OH, CH₂—OH, CH₂—CH₂—OH, CH(OH)—CH₂OH, CH₂—NH—S(═O)₂—CH₃, CH₂—NH—S(═O)₂—NH₂, C₂H₄—OH, NH—CH₂—CH₂—OH, NH—CH₂—CH₂—OCH₃, and N(CH₃)—CH₂—CH₂—OH; particularly most preferred are C₂H₄—S(═O)₂—CH₃, CH₂—O—C₂H₄—OH, CH₂—OH, CH₂—NH—S(═O)₂—CH₃, and C₂H₄—OH.

In another preferred embodiment of the compound according to the invention of general formula (I),

at least one of R⁵ and R⁹, preferably both R⁵ and R⁹, denote(s) H.

In a further preferred embodiment of the compound according to the invention of general formula (I),

at least one, preferably one, of R⁶ and R⁸ denotes H.

In another preferred embodiment of the compound according to the invention of general formula (I),

both of R⁶ and R⁸ denote H.

In yet another preferred embodiment of the compound according to the invention of general formula (I),

at least one of R⁵ and R⁹, preferably both R⁵ and R⁹, denote(s) H and
at least one, preferably one, of R⁶ and R⁸ denotes H
or both of R⁶ and R⁸ denote H.

A particularly preferred embodiment of the present invention is the compound according to the general formula (I), wherein
R¹ represents substructure (T1)

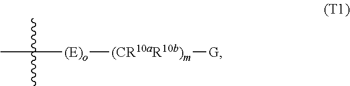

in which
E represents O or S,
o represents 0 or 1, preferably represents 0,
R^{10a} and R^{19b} are independently of one another selected from the group consisting of H, methyl and ethyl, preferably each denote H;
m represents 0, 1 or 2, more preferably 0 or 1;
G represents methyl, ethyl, n-propyl, isopropyl, n-butyl, sec.-butyl, tert.-butyl, pentyl, hexyl, or represents

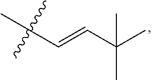 [SMILES: */C=C/C(C)(C)C]

or represents cyclopropyl, cyclobutyl, cyclopentyl and cyclohexyl, or is selected from the group consisting of piperidinyl, morpholinyl, tetrahydropyrrolyl, tetrahydroquinolinyl, tetrahydroisoquinolinyl, dihydroquinolinyl, dihydropyrrolyl, dihydropyridinyl, dihydroisoquinolinyl, tetrahydropyranyl, preferably tetrahydro-2H-pyran-4-yl, tetrahydrofuranyl and tetrahydropyridinyl, in each case independently of one another unsubstituted or mono- or polysubstituted with one or more substituents each selected independently of one another from the group consisting of F, Cl, Br, I, OH, O—C₁₋₄ alkyl, C₁₋₄ alkyl, NH₂, NH(C₁₋₄ alkyl), and N(C₁₋₄ alkyl)₂,
or represents furyl or thienyl, in each case unsubstituted, or denotes phenyl or pyridyl, in each case unsubstituted or mono- or polysubstituted with one or more substituents each selected independently of one another from the group consisting of F, Cl, Br, I, NO₂, CN, OH, O—C₁₋₄ alkyl, O—C₁₋₄ alkylene-O—C₁₋₄ alkyl,

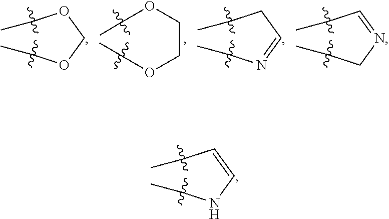

OCF₃, C₁₋₄ alkyl, C₁₋₄ alkylene-O—C₁₋₄-alkyl, CF₃, CF₂H, CFH₂, SCF₃, NH₂, NH(C₁₋₄ alkyl), and N(C₁₋₄ alkyl)₂;
R² is selected from the group consisting of CF₃, methyl, ethyl, n-propyl, isopropyl, n-butyl, sec.-butyl, and tert.-butyl, or
   is selected from the group consisting of cyclopropyl, cyclobutyl, cyclopentyl, and cyclohexyl;
R³ represents H or an unsubstituted C₁₋₄ aliphatic residue, preferably selected from the group consisting of methyl, ethyl, n-propyl, isopropyl, n-butyl, sec.-butyl, and tert.-butyl;
n represents 1, 2 or 3, preferably 1 or 2, more preferably 1,
R^{3a} represents H, methyl, or ethyl,
R^{4a} represents H, methyl, or ethyl,
Y denotes O,
Z represents N or CR^{4b},
   preferably represents N when R^{4a} denotes H or
   preferably represents CR^{4b} when R^{4a} and R^{4b} each represent H or
   preferably represents CR^{4b} when R^{4a} represents methyl and R^{4b} represents H,
R^{4b} represents H, methyl, or ethyl, preferably H or methyl, more preferably H;
T¹ represents N or C—R⁵,
U¹ represents N or C—R⁶,
V represents N or C—R⁷,
U² represents N or C—R⁸,
T² represents N or C—R⁹,
   with the proviso that 1, 2 or 3 of variables T¹, U¹, V, U² and T² represent(s) a nitrogen atom,
   R⁵ and R⁹ are each independently of one another selected from the group consisting of
   H; F; Cl; Br; I; CF₃; OH; methyl; O-methyl; preferably both denote H,
      R⁶ and R⁸ are each independently of one another selected from the group consisting of
   H; F; Cl; Br; I; CF₃; OH; methyl; O-methyl;
      and R⁷ is selected from the group consisting of
   H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
   C₁₋₄ alkyl, C₁₋₄ alkylene-OH, C₁₋₄ alkylene-O—C₁₋₄ alkyl, C₁₋₄ alkylene-O—C₁₋₄ alkylene-OH, C₁₋₄ alkylene-O—C₁₋₄ alkylene-O—C₁₋₄ alkyl, C₁₋₄ alkylene-S(═O)₂—C₁₋₄ alkyl, C₁₋₄ alkylene-NH—S(═O)₂—C₁₋₄ alkyl, C₁₋₄ alkylene-NH—S(═O)₂—NH₂, C₁₋₄ alkylene-NH—C₁₋₄ alkylene-OH, C₁₋₄ alkylene-NH—C₁₋₄ alkylene-O—C₁₋₄ alkyl, C₁₋₄ alkylene-N(C₁₋₄ alkyl)-C₁₋₄ alkylene-OH, C₁₋₄ alkylene-N(C₁₋₄ alkyl)-C₁₋₄ alkylene-O—C₁₋₄ alkyl, O—C₁₋₄ alkyl, O—C₁₋₄ alkylene-OH, O—C₁₋₄ alkylene-O—C₁₋₄ alkyl, NH—C₁₋₄ alkyl, N(C₁₋₄ alkyl)₂, NH—C₁₋₄ alkylene-OH, NH—C₁₋₄ alkylene-O—C₁₋₄ alkyl, N(C₁₋₄ alkyl)-[C₁₋₄ alkylene-OH], N(C₁₋₄ alkyl)-[C₁₋₄ alkylene-O—C₁₋₄ alkyl], NH—S(═O)₂—C₁₋₄ alkyl,
      wherein C₁₋₄ alkylene can in each case be unsubstituted or monosubstituted with OH,
   a C₃₋₆ cycloaliphatic residue, O—C₃₋₆ cycloaliphatic residue, a 3 to 6 membered heterocycloaliphatic residue,
      wherein the C₃₋₆ cycloaliphatic residue is preferably selected from the group consisting of cyclopropyl, cyclobutyl, cyclopentyl, cyclohexyl, and
      wherein the 3 to 6 membered heterocycloaliphatic residue is preferably selected from the group consisting of tetrahydropyranyl, preferably tetrahydro-2H-pyran-4-yl, azetidinyl, piperidinyl, morpholinyl and pyrrolidinyl,
      wherein the C₃₋₆ cycloaliphatic residue and the 3 to 6 membered heterocycloaliphatic residue, respectively, can be unsubstituted or mono- or polysubstituted with one or more substituents each selected independently of one another from the group consisting of F, Cl, Br, I, OH, O—C₁₋₄ alkyl, NH₂, NH(C₁₋₄ alkyl), and N(C₁₋₄ alkyl)₂, and C₁₋₄ alkyl,
   phenyl, C(═O)—NH-phenyl, NH—C(═O)-phenyl, heteroaryl, C(═O)—NH-heteroaryl, NH—C(═O)-heteroaryl, preferably phenyl, C(═O)—NH-phenyl and NH—C(═O)-phenyl,
      wherein heteroaryl is preferably selected from the group consisting of pyridyl, furyl and thienyl;
      wherein in each case independently of one another phenyl and heteroaryl of the aforementioned residues, respectively, can be unsubstituted or mono- or polysubstituted with one or more substituents each selected independently of one another from the group consisting of F, Cl, Br, I, OH, O—C₁₋₄ alkyl, C₁₋₄ alkyl, and CF₃,
         preferably R⁷ is selected from the group consisting of H, F, CH₂—OH, C₂H₄—OH, CH(OH)—CH₂OH, CH₂—O—C₂H₄—OH, CH₂—NH—S(═O)₂—CH₃, CH₂—NH—S(═O)₂—NH₂, CH₂—S(═O)₂—CH₃, C₂H₄—S(═O)₂—CH₃, O—CH₃, N(CH₃)₂, NH—C₂H₄—OH, NH—C₂H₄—O—CH₃, N(CH₃)—[C₂H₄—OH], NH—S(═O)₂—CH₃, azetidinyl, wherein azetidinyl can be unsubstituted or monosubstituted with OH, more preferably R⁷ is selected from the group consisting of H, F, CH₂—OH, C₂H₄—OH, CH₂—O—C₂H₄—OH, CH₂—NH—S(═O)₂—CH₃, C₂H₄—S(═O)₂—CH₃, O—CH₃, N(CH₃)₂, NH—C₂H₄—OH, NH—C₂H₄—O—CH₃, N(CH₃)—[C₂H₄—OH], NH—S(═O)₂—CH₃, azetidinyl, wherein azetidinyl can be unsubstituted or monosubstituted with OH,

Another preferred embodiment of the present invention is the compound according to the general formula (I), wherein
R¹ represents phenyl, unsubstituted or mono- or polysubstituted with one or more substituents each selected independently of one another from the group consisting of F, Cl, Br, I, OH, O—CH₃, CH₃, CH(CH₃)₂, tert.-butyl and CF₃, preferably mono- or disubstituted with one or more substituents each selected independently of one another from the group consisting of F, Cl, Br, I, O—CH₃, CH₃, CH(CH₃)₂, tert.-butyl and CF₃, more preferably mono with one substituent selected from the group consisting of F, Cl, Br, and I,
R² is selected from the group consisting of CF₃, tert.-butyl, and cyclopropyl,
R³ represents H or methyl, preferably represents H,
n represents 1,

R^{3a} represents H,
R^{4a} represents H, or methyl,
Y denotes O,
Z represents N or CR^{4b},
   preferably represents N when R^{4a} denotes H or
   preferably represents CR^{4b} when R^{4a} and R^{4b} each represent H or
   preferably represents CR^{4b} when R^{4a} represents methyl and R^{4b} represents H,
R^{4b} represents H or methyl,
T¹ represents C—R⁵,
U¹ represents N,

V represents C—R⁷,
U² represents N or C—R⁸, preferably C—R⁸,
T² represents C—R⁹,
R⁵ and R⁹ both denote H,
R⁸ is selected from the group consisting of
   H; F; Cl; Br; I; CF₃; OH; methyl; O-methyl;
      and R⁷ is selected from the group consisting of
   H, F, Cl, Br, I, CN, CF₃, CF₂H, CFH₂, OH, OCF₃, SH, SCF₃, NH₂, C(═O)—NH₂, S(═O)₂—OH, S(═O)₂—NH₂,
   CH₃, C₂H₅, CH₂—OH, C₂H₄—OH, CH(OH)—CH₂—OH, CH₂—O—CH₃, C₂H₄—O—CH₃, CH₂—O—CH₂—OH, CH₂—O—C₂H₄—OH, CH₂—O—CH₂—O—CH₃, CH₂—O—C₂H₄—O—CH₃, CH₂—S(═O)₂—CH₃, C₂H₄—S(═O)₂—CH₃, CH₂—NH—S(═O)₂—CH₃, CH₂—NH—S(═O)₂—NH₂, CH₂—NH—CH₂—OH, CH₂—NH—C₂H₄—OH, CH₂—NH—C₂H₄—O—CH₃, CH₂—N(CH₃)—C₂H₄—OH, CH₂—N(CH₃)—C₂H₄—O—CH₃, O—CH₃, O—C₂H₄—OH, O—C₂H₄—O—CH₃, NH—CH₃, N(CH₃)₂, NH—C₂H₄—OH, NH—C₂H₄—O—CH₃, N(CH₃)—[C₂H₄—OH], N(CH₃)—[C₂H₄—O—CH₃], NH—S(═O)₂—CH₃,
   cyclopropyl, cyclobutyl, cyclopentyl, cyclohexyl, O-cyclopropyl, tetrahydropyranyl, preferably tetrahydro-2H-pyran-4-yl, azetidinyl, piperidinyl, morpholinyl or pyrrolidinyl, in each case independently of one another unsubstituted or mono- or polysubstituted with one or more substituents each selected independently of one another from the group consisting of F, Cl, Br, I, OH, O—CH₃, NH₂, N(CH₃)₂, CH₃, C₂H₅ and tert.-butyl,
   phenyl, C(═O)—NH-phenyl, or NH—C(═O)-phenyl, wherein in each case independently of one another phenyl can be unsubstituted or mono- or polysubstituted with one or more substituents each selected independently of one another from the group consisting of F, Cl, Br, I, OH, O—CH₃, CH₃, C₂H₅, and CF₃,
      preferably R⁷ is selected from the group consisting of H, F, CH₂—OH, C₂H₄—OH, CH(OH)—CH₂OH, CH₂—O—C₂H₄—OH, CH₂—NH—S(═O)₂—CH₃, CH₂—NH—S(═O)₂—NH₂, CH₂—S(═O)₂—CH₃, C₂H₄—S(═O)₂—CH₃, O—CH₃, N(CH₃)₂, NH—C₂H₄—OH, NH—C₂H₄—O—CH₃, N(CH₃)—[C₂H₄—OH], NH—S(═O)₂—CH₃, azetidinyl, wherein azetidinyl can be unsubstituted or monosubstituted with OH, preferably H, F, CH₂—OH, C₂H₄—OH, CH₂—O—C₂H₄—OH, CH₂—NH—S(═O)₂—CH₃, C₂H₄—S(═O)₂—CH₃, O—CH₃, N(CH₃)₂, NH—C₂H₄—OH, NH—C₂H₄—O—CH₃, N(CH₃)—[C₂H₄—OH], NH—S(═O)₂—CH₃, azetidinyl, wherein azetidinyl can be unsubstituted or monosubstituted with OH,
      more preferably R⁷ is selected from the group consisting of C₂H₄—S(═O)₂—CH₃, CH₂—O—C₂H₄—OH, CH₂—OH, CH₂—NH—S(═O)₂—CH₃, and C₂H₄—OH.

Yet another preferred embodiment of the present invention is the compound according to the general formula (I), wherein
R¹ represents phenyl, monosubstituted with F, Cl or CH(CH₃)₂,
R² denotes CF₃ or tert.-butyl,
R³ represents H,
n represents 1,
R^{3a} represents H,
R^{4a} represents H, or methyl,
Y denotes O,
Z represents N or CR^{4b},
   preferably represents N when R^{4a} denotes H or
   preferably represents CR^{4b} when R^{4a} and R^{4b} each represent H or
   preferably represents CR^{4b} when R^{4a} represents methyl and R^{4b} represents H,
R^{4b} represents H,
T¹ represents C—R⁵,
U¹ represents N,
V represents C—R⁷,
U² represents N or C—R⁸,
T² represents C—R⁹,
R⁵ and R⁹ both denote H,
R⁸ is selected from the group consisting of H; F; CH₃, CF₃; OH; and O-methyl; preferably H; F; CF₃; OH; and O-methyl;
   and R⁷ is selected from the group consisting of
   H, F, CH₂—OH, C₂H₄—OH, CH(OH)—CH₂OH, CH₂—O—C₂H₄—OH, CH₂—NH—S(═O)₂—CH₃, CH₂—NH—S(═O)₂—NH₂, CH₂—S(═O)₂—CH₃, C₂H₄—S(═O)₂—CH₃, O—CH₃, N(CH₃)₂, NH—C₂H₄—OH, NH—C₂H₄—O—CH₃, N(CH₃)—[C₂H₄—OH], NH—S(═O)₂—CH₃, azetidinyl, wherein azetidinyl can be unsubstituted or monosubstituted with OH, preferably H, F, CH₂—OH, C₂H₄—OH, CH₂—O—C₂H₄—OH, CH₂—NH—S(═O)₂—CH₃, C₂H₄—S(═O)₂—CH₃, O—CH₃, N(CH₃)₂, NH—C₂H₄—OH, NH—C₂H₄—O—CH₃, N(CH₃)—[C₂H₄—OH], NH—S(═O)₂—CH₃, azetidinyl, wherein azetidinyl can be unsubstituted or monosubstituted with OH,
   C(═O)—NH-phenyl or NH—C(═O)-phenyl, wherein in each case independently of one another phenyl can be unsubstituted or mono- or polysubstituted with one or more substituents each selected independently of one another from the group consisting of F, Cl, and CF₃.

Particularly preferred are compounds according to the invention from the group
1 N-((1-(3-chlorophenyl)-3-(trifluoromethyl)-1H-pyrazol-5-yl)methyl)-2-(pyridin-2-yl)acetamide;
2 N-((3-tert-butyl-1-(3-chlorophenyl)-1H-pyrazol-5-yl)methyl)-2-(pyridin-2-yl)propanamide;
3 N-((1-(3-chlorophenyl)-3-(trifluoromethyl)-1H-pyrazol-5-yl)methyl)-2-(pyridin-2-yl)propanamide;
4 1-((3-tert-butyl-1-(3-chlorophenyl)-1H-pyrazol-5-yl)methyl)-3-(pyridin-2-yl)urea;
5 1-((1-(3-chlorophenyl)-3-(trifluoromethyl)-1H-pyrazol-5-yl)methyl)-3-(pyridin-2-yl)urea;
6 N-((3-tert-butyl-1-(3-chlorophenyl)-1H-pyrazol-5-yl)methyl)-2-(pyridin-3-yl)acetamide;
7 N-((1-(3-chlorophenyl)-3-(trifluoromethyl)-1H-pyrazol-5-yl)methyl)-2-(pyridin-3-yl)acetamide;
8 N-((1-(3-chlorophenyl)-3-(trifluoromethyl)-1H-pyrazol-5-yl)methyl)-2-(pyridin-3-yl)propanamide;
9 1-((1-(3-chlorophenyl)-3-(trifluoromethyl)-1H-pyrazol-5-yl)methyl)-3-(pyridin-3-yl)urea;
10 N-((3-tert-butyl-1-(3-chlorophenyl)-1H-pyrazol-5-yl)methyl)-2-(pyridin-4-yl)acetamide;
11 N-((1-(3-chlorophenyl)-3-(trifluoromethyl)-1H-pyrazol-5-yl)methyl)-2-(pyridin-4-yl)acetamide;
12 1-((1-(3-chlorophenyl)-3-(trifluoromethyl)-1H-pyrazol-5-yl)methyl)-3-(pyridin-4-yl)urea;
13 N-((1-(3-chlorophenyl)-3-(trifluoromethyl)-1H-pyrazol-5-yl)methyl)-2-(pyrimidin-4-yl)acetamide;
14 1N-((3-tert-butyl-1-(3-chlorophenyl)-1H-pyrazol-5-yl)methyl)-2-(pyrazin-2-yl)acetamide;
15 1-((1-(3-chlorophenyl)-3-(trifluoromethyl)-1H-pyrazol-5-yl)methyl)-3-(pyridazin-4-yl)urea;
16 N-((1-(3-chlorophenyl)-3-(trifluoromethyl)-1H-pyrazol-5-yl)methyl)-2-(pyrimidin-5-yl)acetamide;
17 N-((1-(3-chlorophenyl)-3-(trifluoromethyl)-1H-pyrazol-5-yl)methyl)-2-(6-chloropyridin-3-yl)acetamide;
18 1-((1-(3-chlorophenyl)-3-(trifluoromethyl)-1H-pyrazol-5-yl)methyl)-3-(5-fluoropyridin-3-yl)urea;
19 1-((1-(3-chlorophenyl)-3-(trifluoromethyl)-1H-pyrazol-5-yl)methyl)-3-(2-methylpyrimidin-5-yl)urea;
20 1-((1-(3-chlorophenyl)-3-(trifluoromethyl)-1H-pyrazol-5-yl)methyl)-3-(1,3,5-triazin-2-yl)urea;
21 1-((1-(3-chlorophenyl)-3-(trifluoromethyl)-1H-pyrazol-5-yl)methyl)-3-(6-(2-(methylsulfonyl)ethyl)pyridin-3-yl)urea;
22 1-((3-tert-butyl-1-(3-chlorophenyl)-1H-pyrazol-5-yl)methyl)-3-(5-fluoro-6-(2-(methylsulfonyl)ethyl)pyridin-3-yl)urea;
23 1-((1-(3-chlorophenyl)-3-(trifluoromethyl)-1H-pyrazol-5-yl)methyl)-3-(5-fluoro-6-(2-(methylsulfonyl)ethyl)pyridin-3-yl)urea;
24 1-((1-(3-chlorophenyl)-3-cyclopropyl-1H-pyrazol-5-yl)methyl)-3-(5-fluoro-6-(2-(methylsulfonyl)ethyl)pyridin-3-yl)urea;
25 5-(3-((3-tert-butyl-1-(3-chlorophenyl)-1H-pyrazol-5-yl)methyl)ureido)picolinamide;
26 5-(3-((1-(3-chlorophenyl)-3-(trifluoromethyl)-1H-pyrazol-5-yl)methyl)ureido)picolinamide;
27 N-((1-(3-chlorophenyl)-3-(trifluoromethyl)-1H-pyrazol-5-yl)methyl)-2-(6-(methylsulfonamidomethyl)pyridin-3-yl)propanamide;
28 N-((5-(3-((1-(3-chlorophenyl)-3-(trifluoromethyl)-1H-pyrazol-5-yl)methyl)ureido)pyridin-2-yl)methyl)methanesulfonamide;
29 N-((5-(3-((1-(3-chlorophenyl)-3-(trifluoromethyl)-1H-pyrazol-5-yl)methyl)ureido)pyridin-2-yl)methyl)sulfuric diamide;
30 N-((1-(3-chlorophenyl)-3-(trifluoromethyl)-1H-pyrazol-5-yl)methyl)-2-(6-(hydroxymethyl)pyridin-3-yl)propanamide;
31 (E)-1-((1-(3,3-dimethylbut-1-enyl)-3-(trifluoromethyl)-1H-pyrazol-5-yl)methyl)-3-(6-(hydroxymethyl)pyridin-3-yl)urea;
32 1-((1-(3-chlorophenyl)-3-(trifluoromethyl)-1H-pyrazol-5-yl)methyl)-3-(6-(hydroxymethyl)pyridin-3-yl)urea;
33 1-((1-(3-fluorophenyl)-3-(trifluoromethyl)-1H-pyrazol-5-yl)methyl)-3-(6-(hydroxymethyl)pyridin-3-yl)urea;
34 1-((1-(3-fluoro-4-methylphenyl)-3-(trifluoromethyl)-1H-pyrazol-5-yl)methyl)-3-(6-(hydroxymethyl)pyridin-3-yl)urea;
35 1-((1-(3-fluoro-4-methoxyphenyl)-3-(trifluoromethyl)-1H-pyrazol-5-yl)methyl)-3-(6-(hydroxymethyl)pyridin-3-yl)urea;
36 1-(6-(hydroxymethyl)pyridin-3-yl)-3-((1-m-tolyl-3-(trifluoromethyl)-1H-pyrazol-5-yl)methyl)urea;
37 1-(6-(hydroxymethyl)pyridin-3-yl)-3-((1-(4-methoxy-3-methylphenyl)-3-(trifluoromethyl)-1H-pyrazol-5-yl)methyl)urea;
38 1-(6-(hydroxymethyl)pyridin-3-yl)-3-((1-(3-isopropylphenyl)-3-(trifluoromethyl)-1H-pyrazol-5-yl)methyl)urea;
39 1-((1-(3-tert-butylphenyl)-3-(trifluoromethyl)-1H-pyrazol-5-yl)methyl)-3-(6-(hydroxymethyl)pyridin-3-yl)urea;
40 1-(6-(hydroxymethyl)pyridin-3-yl)-3-((1-(3-(methoxymethyl)phenyl)-3-(trifluoromethyl)-1H-pyrazol-5-yl)methyl)urea;
41 1-((1-(3-(difluoromethyl)phenyl)-3-(trifluoromethyl)-1H-pyrazol-5-yl)methyl)-3-(6-(hydroxymethyl)pyridin-3-yl)urea;
42 1-((1-(3-cyanophenyl)-3-(trifluoromethyl)-1H-pyrazol-5-yl)methyl)-3-(6-(hydroxymethyl)pyridin-3-yl)urea;
43 1-((1-(3-(dimethylamino)phenyl)-3-(trifluoromethyl)-1H-pyrazol-5-yl)methyl)-3-(6-(hydroxymethyl)pyridin-3-yl)urea;
44 1-((1-(5-chloropyridin-3-yl)-3-(trifluoromethyl)-1H-pyrazol-5-yl)methyl)-3-(6-(hydroxymethyl)pyridin-3-yl)urea;
45 1-(6-(hydroxymethyl)pyridin-3-yl)-3-((1-(6-methoxypyridin-3-yl)-3-(trifluoromethyl)-1H-pyrazol-5-yl)methyl)urea;
46 1-((1-(benzo[d][1,3]dioxol-5-yl)-3-(trifluoromethyl)-1H-pyrazol-5-yl)methyl)-3-(6-(hydroxymethyl)pyridin-3-yl)urea;
47 1-((1-(1H-indol-6-yl)-3-(trifluoromethyl)-1H-pyrazol-5-yl)methyl)-3-(6-(hydroxymethyl)pyridin-3-yl)urea;
48 1-((1-(furan-3-yl)-3-(trifluoromethyl)-1H-pyrazol-5-yl)methyl)-3-(6-(hydroxymethyl)pyridin-3-yl)urea;
49 1-(6-(hydroxymethyl)pyridin-3-yl)-3-((1-(thiophen-2-yl)-3-(trifluoromethyl)-1H-pyrazol-5-yl)methyl)urea;
50 1-((1-(3-chlorophenyl)-3-(trifluoromethyl)-1H-pyrazol-5-yl)methyl)-3-(5-fluoro-6-(hydroxymethyl)pyridin-3-yl)urea;
51 N-((1-(3-chlorophenyl)-3-(trifluoromethyl)-1H-pyrazol-5-yl)methyl)-2-(6-(2-hydroxyethyl)pyridin-3-yl)propanamide;
52 1-((1-(3-chlorophenyl)-3-(trifluoromethyl)-1H-pyrazol-5-yl)methyl)-3-(6-(2-hydroxyethyl)pyridin-3-yl)urea;
53 N-((1-(3-chlorophenyl)-3-(trifluoromethyl)-1H-pyrazol-5-yl)methyl)-2-(6-((2-hydroxyethoxy)methyl)pyridin-3-yl)propanamide;
54 1-((1-(3-fluorophenyl)-3-(trifluoromethyl)-1H-pyrazol-5-yl)methyl)-3-(6-(tetrahydro-2H-pyran-4-yl)pyridin-3-yl)urea;
55 5-(1-((1-(3-chlorophenyl)-3-(trifluoromethyl)-1H-pyrazol-5-yl)methylamino)-1-oxopropan-2-yl)picolinamide;
56 5-(1-((3-tert-butyl-1-(3-chlorophenyl)-1H-pyrazol-5-yl)methylamino)-1-oxopropan-2-yl)picolinamide;
57 5-(1-((1-(3-chlorophenyl)-3-(trifluoromethyl)-1H-pyrazol-5-yl)methylamino)-1-oxopropan-2-yl)-N-phenyl)picolinamide;
58 5-(1-((1-(3-chlorophenyl)-3-(trifluoromethyl)-1H-pyrazol-5-yl)methylamino)-1-oxopropan-2-yl)-N-(4-fluorophenyl)picolinamide;
59 5-(1-((1-(3-chlorophenyl)-3-(trifluoromethyl)-1H-pyrazol-5-yl)methylamino)-1-oxopropan-2-yl)-N-(4-(trifluoromethyl)phenyl)picolinamide;
60 5-(1-((3-tert-butyl-1-(3-chlorophenyl)-1H-pyrazol-5-yl)methylamino)-1-oxopropan-2-yl)-N-(4-fluorophenyl)picolinamide;
61 5-(1-((3-tert-butyl-1-(3-chlorophenyl)-1H-pyrazol-5-yl)methylamino)-1-oxopropan-2-yl)-N-(4-(trifluoromethyl)phenyl)picolinamide;
62 5-(1-((1-(3-chlorophenyl)-3-(trifluoromethyl)-1H-pyrazol-5-yl)methylamino)-1-oxopropan-2-yl)-N-phenylpyrimidine-2-carboxamide;
63 5-(1-((1-(3-chlorophenyl)-3-(trifluoromethyl)-1H-pyrazol-5-yl)methylamino)-1-oxopropan-2-yl)-N-(4-fluorophenyl)pyrimidine-2-carboxamide;
64 5-(1-((1-(3-chlorophenyl)-3-(trifluoromethyl)-1H-pyrazol-5-yl)methylamino)-1-oxopropan-2-yl)-N-(4-(trifluoromethyl)phenyl)pyrimidine-2-carboxamide;
65 5-(1-((3-tert-butyl-1-(3-chlorophenyl)-1H-pyrazol-5-yl)methylamino)-1-oxopropan-2-yl)-N-phenylpyrimidine-2-carboxamide;
66 5-(1-((3-tert-butyl-1-(3-chlorophenyl)-1H-pyrazol-5-yl)methylamino)-1-oxopropan-2-yl)-N-(4-fluorophenyl)pyrimidine-2-carboxamide;
67 5-(1-((3-tert-butyl-1-(3-chlorophenyl)-1H-pyrazol-5-yl)methylamino)-1-oxopropan-2-yl)-N-(4-(trifluoromethyl)phenyl)pyrimidine-2-carboxamide;
68 1-((3-tert-butyl-1-(3-chlorophenyl)-1H-pyrazol-5-yl)methyl)-3-(6-(2-methoxyethylamino)pyridin-3-yl)urea;
69 1-((1-(3-chlorophenyl)-3-(trifluoromethyl)-1H-pyrazol-5-yl)methyl)-3-(6-(2-methoxyethylamino)pyridin-3-yl)urea;
70 1-((3-tert-butyl-1-(3-chlorophenyl)-1H-pyrazol-5-yl)methyl)-3-(6-(2-hydroxyethylamino)pyridin-3-yl)urea;
71 1-((1-(3-chlorophenyl)-3-(trifluoromethyl)-1H-pyrazol-5-yl)methyl)-3-(6-(2-hydroxyethylamino)pyridin-3-yl)urea;
72 1-((1-(3-chlorophenyl)-3-cyclopropyl-1H-pyrazol-5-yl)methyl)-3-(6-(2-hydroxyethylamino)pyridin-3-yl)urea;
73 1-((1-(3-chlorophenyl)-4-methyl-3-(trifluoromethyl)-1H-pyrazol-5-yl)methyl)-3-(6-(2-hydroxyethylamino)pyridin-3-yl)urea;
74 1-(6-(2-hydroxyethylamino)pyridin-3-yl)-3-((1-pentyl-3-(trifluoromethyl)-1H-pyrazol-5-yl)methyl)urea;
75 1-((1-(cyclopropylmethyl)-3-(trifluoromethyl)-1H-pyrazol-5-yl)methyl)-3-(6-(2-hydroxyethylamino)pyridin-3-yl)urea;
76 1-((1-cyclohexyl-3-(trifluoromethyl)-1H-pyrazol-5-yl)methyl)-3-(6-(2-hydroxyethylamino)pyridin-3-yl)urea;
77 1-(6-(2-hydroxyethylamino)pyridin-3-yl)-3-((1-(tetrahydro-2H-pyran-4-yl)-3-(trifluoromethyl)-1H-pyrazol-5-yl)methyl)urea;
78 1-((1-(3-fluorophenyl)-3-(trifluoromethyl)-1H-pyrazol-5-yl)methyl)-3-(6-(2-hydroxyethylamino)pyridin-3-yl)urea;
79 1-((1-(3,4-difluorophenyl)-3-(trifluoromethyl)-1H-pyrazol-5-yl)methyl)-3-(6-(2-hydroxyethylamino)pyridin-3-yl)urea;
80 1-(6-(2-hydroxyethylamino)pyridin-3-yl)-3-((1-(3-methoxyphenyl)-3-(trifluoromethyl)-1H-pyrazol-5-yl)methyl)urea;
81 1-(6-(2-hydroxyethylamino)pyridin-3-yl)-3-((1-m-tolyl-3-(trifluoromethyl)-1H-pyrazol-5-yl)methyl)urea;
82 1-(6-(2-hydroxyethylamino)pyridin-3-yl)-3-((1-(3-isopropylphenyl)-3-(trifluoromethyl)-1H-pyrazol-5-yl)methyl)urea;
83 1-((1-(3-tert-butylphenyl)-3-(trifluoromethyl)-1H-pyrazol-5-yl)methyl)-3-(6-(2-hydroxyethylamino)pyridin-3-yl)urea;
84 1-((3-tert-butyl-1-(3-(trifluoromethyl)phenyl)-1H-pyrazol-5-yl)methyl)-3-(6-(2-hydroxyethylamino)pyridin-3-yl)urea;
85 1-((3-tert-butyl-1-(pyridin-2-yl)-1H-pyrazol-5-yl)methyl)-3-(6-(2-hydroxyethylamino)pyridin-3-yl)urea;
86 1-(6-(2-hydroxyethylamino)pyridin-3-yl)-3-((1-(4-methoxybenzyl)-3-(trifluoromethyl)-1H-pyrazol-5-yl)methyl)urea;
87 1-((3-tert-butyl-1-(3-chlorophenyl)-1H-pyrazol-5-yl)methyl)-3-(6-((2-hydroxyethyl)(methyl)amino)pyridin-3-yl)urea;
88 N-((1-(3-chlorophenyl)-3-(trifluoromethyl)-1H-pyrazol-5-yl)methyl)-2-(6-((2-methoxyethyl)(methyl)amino)pyridin-3-yl)propanamide;
89 1-((1-(3-chlorophenyl)-3-(trifluoromethyl)-1H-pyrazol-5-yl)methyl)-3-(6-((2-methoxyethyl)(methyl)amino)pyridin-3-yl)urea;
90 1-((1-(3-chlorophenyl)-3-(trifluoromethyl)-1H-pyrazol-5-yl)methyl)-3-(6-((2-hydroxyethyl)(methyl)amino)pyridin-3-yl)urea;
91 N-(5-(1-((3-tert-butyl-1-(3-chlorophenyl)-1H-pyrazol-5-yl)methylamino)-1-oxopropan-2-yl)pyridin-2-yl)benzamide;
92 N-(5-(1-((1-(3-chlorophenyl)-3-(trifluoromethyl)-1H-pyrazol-5-yl)methylamino)-1-oxopropan-2-yl)pyridin-2-yl)benzamide;
93 N-(5-(1-((3-tert-butyl-1-(3-chlorophenyl)-1H-pyrazol-5-yl)methylamino)-1-oxopropan-2-yl)pyridin-2-yl)-4-fluorobenzamide;
94 N-(5-(1-((1-(3-chlorophenyl)-3-(trifluoromethyl)-1H-pyrazol-5-yl)methylamino)-1-oxopropan-2-yl)pyridin-2-yl)-4-fluorobenzamide;
95 N-(5-(1-((3-tert-butyl-1-(3-chlorophenyl)-1H-pyrazol-5-yl)methylamino)-1-oxopropan-2-yl)pyridin-2-yl)-4-chlorobenzamide;
96 4-chloro-N-(5-(1-((1-(3-chlorophenyl)-3-(trifluoromethyl)-1H-pyrazol-5-yl)methylamino)-1-oxopropan-2-yl)pyridin-2-yl)benzamide;
97 4-chloro-N-(5-(1-oxo-1-((1-(pyridin-3-yl)-3-(trifluoromethyl)-1H-pyrazol-5-yl)methylamino)propan-2-yl)pyridin-2-yl)benzamide;
98 N-((3-tert-butyl-1-(3-chlorophenyl)-1H-pyrazol-5-yl)methyl)-2-(6-(methylsulfonamido)pyridin-3-yl)propanamide;
99 N-((1-(3-chlorophenyl)-3-(trifluoromethyl)-1H-pyrazol-5-yl)methyl)-2-(6-(methylsulfonamido)pyridin-3-yl)propanamide;
100 N-((1-(3-fluorophenyl)-3-(trifluoromethyl)-1H-pyrazol-5-yl)methyl)-2-(6-(methylsulfonamido)pyridin-3-yl)propanamide;
101 N-(5-(3-((1-(3-chlorophenyl)-3-(trifluoromethyl)-1H-pyrazol-5-yl)methyl)ureido)pyridin-2-yl)methanesulfonamide;
102 N-((3-tert-butyl-1-(3-chlorophenyl)-1H-pyrazol-5-yl)methyl)-2-(5-fluoro-6-(methylsulfonamido)pyridin-3-yl)propanamide;
103 N-((1-(3-chlorophenyl)-3-(trifluoromethyl)-1H-pyrazol-5-yl)methyl)-2-(5-fluoro-6-(methylsulfonamido)pyridin-3-yl)propanamide;
104 N-((1-(3-chlorophenyl)-3-(trifluoromethyl)-1H-pyrazol-5-yl)methyl)-2-(5-methoxy-6-(methylsulfonamido)pyridin-3-yl)propanamide;
105 N-((3-tert-butyl-1-(3-chlorophenyl)-1H-pyrazol-5-yl)methyl)-2-(5-methoxy-6-(methylsulfonamido)pyridin-3-yl)propanamide;
106 1-((1-(3-chlorophenyl)-3-(trifluoromethyl)-1H-pyrazol-5-yl)methyl)-3-(6-(dimethylamino)-5-(trifluoromethyl)pyridin-3-yl)urea;
107 1-((3-tert-butyl-1-(3-chlorophenyl)-1H-pyrazol-5-yl)methyl)-3-(6-(dimethylamino)-5-(trifluoromethyl)pyridin-3-yl)urea;
108 1-(6-(azetidin-1-yl)pyridin-3-yl)-3-((3-tert-butyl-1-(3-chlorophenyl)-1H-pyrazol-5-yl)methyl)urea;
109 1-(6-(azetidin-1-yl)pyridin-3-yl)-3-((1-(3-chlorophenyl)-3-(trifluoromethyl)-1H-pyrazol-5-yl)methyl)urea;
110 1-((3-tert-butyl-1-(3-fluorophenyl)-1H-pyrazol-5-yl)methyl)-3-(6-(3-hydroxyazetidin-1-yl)pyridin-3-yl)urea;
111 1-((1-(3-chlorophenyl)-3-(trifluoromethyl)-1H-pyrazol-5-yl)methyl)-3-(6-(3-hydroxyazetidin-1-yl)pyridin-3-yl)urea;
112 1-((1-(3-fluorophenyl)-3-(trifluoromethyl)-1H-pyrazol-5-yl)methyl)-3-(6-(3-hydroxyazetidin-1-yl)pyridin-3-yl)urea;
113 1-((1-(3-chloro-4-fluorophenyl)-3-(trifluoromethyl)-1H-pyrazol-5-yl)methyl)-3-(6-(3-hydroxyazetidin-1-yl)pyridin-3-yl)urea;
114 1-(6-(3-hydroxyazetidin-1-yl)pyridin-3-yl)-3-((1-m-tolyl-3-(trifluoromethyl)-1H-pyrazol-5-yl)methyl)urea;
115 1-(6-(3-hydroxyazetidin-1-yl)pyridin-3-yl)-3-((1-(3-isopropylphenyl)-3-(trifluoromethyl)-1H-pyrazol-5-yl)methyl)urea;
116 1-((1-(3-tert-butylphenyl)-3-(trifluoromethyl)-1H-pyrazol-5-yl)methyl)-3-(6-(3-hydroxyazetidin-1-yl)pyridin-3-yl)urea;
117 1-((3-tert-butyl-1-(3-chlorophenyl)-1H-pyrazol-5-yl)methyl)-3-(6-(3-hydroxyazetidin-1-yl)pyridin-3-yl)urea;
118 1-((1-(3-(dimethylamino)phenyl)-3-(trifluoromethyl)-1H-pyrazol-5-yl)methyl)-3-(6-(3-hydroxyazetidin-1-yl)pyridin-3-yl)urea;
119 1-(6-(3-hydroxyazetidin-1-yl)pyridin-3-yl)-3-((1-(3-methoxyphenyl)-3-(trifluoromethyl)-1H-pyrazol-5-yl)methyl)urea;
120 1-((3-tert-butyl-1-(3-chlorophenyl)-1H-pyrazol-5-yl)methyl)-3-(6-(pyrrolidin-1-yl)pyridin-3-yl)urea;
121 1-((1-(3-chlorophenyl)-3-(trifluoromethyl)-1H-pyrazol-5-yl)methyl)-3-(6-(pyrrolidin-1-yl)pyridin-3-yl)urea;
122 1-((1-(3-chlorophenyl)-3-(trifluoromethyl)-1H-pyrazol-5-yl)methyl)-3-(5-fluoro-6-(pyrrolidin-1-yl)pyridin-3-yl)urea;
123 1-((1-(3-chlorophenyl)-3-(trifluoromethyl)-1H-pyrazol-5-yl)methyl)-3-(5-methoxy-6-(pyrrolidin-1-yl)pyridin-3-yl)urea;
124 (R)-1-((1-(3-chlorophenyl)-3-(trifluoromethyl)-1H-pyrazol-5-yl)methyl)-3-(6-(3-hydroxypyrrolidin-1-yl)pyridin-3-yl)urea;
125 (S)-1-((1-(3-chlorophenyl)-3-(trifluoromethyl)-1H-pyrazol-5-yl)methyl)-3-(6-(3-hydroxypyrrolidin-1-yl)pyridin-3-yl)urea;
126 (R)-1-((3-tert-butyl-1-(3-chlorophenyl)-1H-pyrazol-5-yl)methyl)-3-(6-(3-hydroxypyrrolidin-1-yl)pyridin-3-yl)urea;
127 (S)-1-((3-tert-butyl-1-(3-chlorophenyl)-1H-pyrazol-5-yl)methyl)-3-(6-(3-hydroxypyrrolidin-1-yl)pyridin-3-yl)urea;
128 1-((1-(3-chlorophenyl)-3-(trifluoromethyl)-1H-pyrazol-5-yl)methyl)-3-(6-hydroxypyridin-3-yl)urea;
129 N-((1-(3-chlorophenyl)-3-(trifluoromethyl)-1H-pyrazol-5-yl)methyl)-2-(6-methoxypyridin-3-yl)propanamide;
130 1-((1-(3-chlorophenyl)-3-(trifluoromethyl)-1H-pyrazol-5-yl)methyl)-3-(2-methoxypyrimidin-5-yl)urea;
131 1-((3-tert-butyl-1-(3-chlorophenyl)-1H-pyrazol-5-yl)methyl)-3-(6-(2-methoxyethoxy)pyridin-3-yl)urea;
132 1-((1-(3-chlorophenyl)-3-(trifluoromethyl)-1H-pyrazol-5-yl)methyl)-3-(6-(2-methoxyethoxy)pyridin-3-yl)urea;
133 1-((1-(3-chlorophenyl)-3-(trifluoromethyl)-1H-pyrazol-5-yl)methyl)-3-(6-(2-hydroxyethoxy)pyridin-3-yl)urea;
134 1-((1-(3-chlorophenyl)-3-(trifluoromethyl)-1H-pyrazol-5-yl)methyl)-3-(6-((2-hydroxyethylamino)methyl)pyridin-3-yl)urea;
135 1-((1-(3-chlorophenyl)-3-(trifluoromethyl)-1H-pyrazol-5-yl)methyl)-3-(6-(((2-hydroxyethyl)(methyl)amino)methyl)pyridin-3-yl)urea;
136 1-((1-(3-chlorophenyl)-3-(trifluoromethyl)-1H-pyrazol-5-yl)methyl)-3-(6-methylpyridin-3-yl)urea;
137 1-((1-(3-chlorophenyl)-3-(trifluoromethyl)-1H-pyrazol-5-yl)methyl)-3-(5-methylpyridin-3-yl)urea;
138 1-((1-(3-chlorophenyl)-3-(trifluoromethyl)-1H-pyrazol-5-yl)methyl)-3-(4,6-dimethylpyridin-3-yl)urea;
139 1-((3-tert-butyl-1-(3-chlorophenyl)-1H-pyrazol-5-yl)methyl)-3-(5-(hydroxymethyl)pyridin-2-yl)urea;
140 1-((3-tert-butyl-1-(3-chlorophenyl)-1H-pyrazol-5-yl)methyl)-3-(5-(hydroxymethyl)pyridin-3-yl)urea;
141 1-((1-(3-chlorophenyl)-3-(trifluoromethyl)-1H-pyrazol-5-yl)methyl)-3-(6-(hydroxymethyl)-2-methylpyridin-3-yl)urea;
142 N-((1-(3-chlorophenyl)-3-(trifluoromethyl)-1H-pyrazol-5-yl)methyl)-2-(5-fluoro-6-(hydroxymethyl)pyridin-3-yl)propanamide;
143 1-((1-(3-chlorophenyl)-3-(trifluoromethyl)-1H-pyrazol-5-yl)methyl)-3-(6-(2-hydroxyethyl)-2-methylpyridin-3-yl)urea;
144 1-((3-tert-butyl-1-(3-chlorophenyl)-1H-pyrazol-5-yl)methyl)-3-(6-(1,2-dihydroxyethyl)pyridin-3-yl)urea;
145 1-((1-(3-chlorophenyl)-3-(trifluoromethyl)-1H-pyrazol-5-yl)methyl)-3-(6-(1,2-dihydroxyethyl)pyridin-3-yl)urea;
146 N-((1-(3-chlorophenyl)-3-(trifluoromethyl)-1H-pyrazol-5-yl)methyl)-2-(6-(2-hydroxyethylamino)pyridin-3-yl)propanamide;
147 N-((1-(3-chlorophenyl)-3-(trifluoromethyl)-1H-pyrazol-5-yl)methyl)-2-(6-(2-methoxyethylamino)pyridin-3-yl)propanamide;
148 N-((1-(3-chlorophenyl)-3-(trifluoromethyl)-1H-pyrazol-5-yl)methyl)-2-(6-((2-hydroxyethyl)(methyl)amino)pyridin-3-yl)propanamide;
149 N-((1-(3-chloro-4-fluorophenyl)-3-(trifluoromethyl)-1H-pyrazol-5-yl)methyl)-2-(6-(2-hydroxyethyl)pyridin-3-yl)propanamide;
150 N-((1-(3-chlorophenyl)-3-cyclopropyl-1H-pyrazol-5-yl)methyl)-2-(6-(2-hydroxyethyl)pyridin-3-yl)propanamide;
151 2-(6-(2-hydroxyethyl)pyridin-3-yl)-N-((1-m-tolyl-3-(trifluoromethyl)-1H-pyrazol-5-yl)methyl)propanamide;
152 1-((3-tert-butyl-1-(3-fluorophenyl)-1H-pyrazol-5-yl)methyl)-3-(6-(2-hydroxyethyl)pyridin-3-yl)urea;
153 1-((3-tert-butyl-1-(3-methoxyphenyl)-1H-pyrazol-5-yl)methyl)-3-(6-(2-hydroxyethyl)pyridin-3-yl)urea;
154 1-((3-tert-butyl-1-(3-fluorophenyl)-1H-pyrazol-5-yl)methyl)-3-(6-(2-hydroxyethylamino)pyridin-3-yl)urea;
155 1-((1-(3,5-difluorophenyl)-3-(trifluoromethyl)-1H-pyrazol-5-yl)methyl)-3-(6-(2-hydroxyethylamino)pyridin-3-yl)urea;
156 1-((1-(4-chloro-3-methylphenyl)-3-(trifluoromethyl)-1H-pyrazol-5-yl)methyl)-3-(6-(2-hydroxyethylamino)pyridin-3-yl)urea;
157 1-(6-(2-hydroxyethylamino)pyridin-3-yl)-3-((1-(4-methoxy-3-methylphenyl)-3-(trifluoromethyl)-1H-pyrazol-5-yl)methyl)urea;
158 1-((1-(4-fluoro-3-methylphenyl)-3-(trifluoromethyl)-1H-pyrazol-5-yl)methyl)-3-(6-(2-hydroxyethylamino)pyridin-3-yl)urea;
159 1-((3-tert-butyl-1-(3-chlorophenyl)-1H-pyrazol-5-yl)methyl)-3-(6-(methylsulfonylmethyl)pyridin-3-yl)urea;
160 1-((3-tert-butyl-1-(3-fluorophenyl)-1H-pyrazol-5-yl)methyl)-3-(6-(hydroxymethyl)pyridin-3-yl)urea;
161 N-((1-(3-chlorophenyl)-3-(trifluoromethyl)-1H-pyrazol-5-yl)methyl)-2-(6-(2-(methylsulfonyl)ethyl)pyridin-3-yl)propanamide;
162 N-((5-(3-((1-(3-chlorophenyl)-3-(trifluoromethyl)-1H-pyrazol-5-yl)methyl)ureido)pyrimidin-2-yl)methyl)methanesulfonamide; and
163 1-((3-cyclopropyl-1-(3-fluorophenyl)-1H-pyrazol-5-yl)methyl)-3-(6-(hydroxymethyl)pyridin-3-yl)urea;
   optionally in the form of a single stereoisomer or a mixture of stereoisomers, in the form of the free compound and/or a physiologically acceptable salt thereof.

Furthermore, preference may be given to compounds according to the invention that cause a 50 percent displacement of capsaicin, which is present at a concentration of 100 nM, in a FLIPR assay with CHO K1 cells which were transfected with the human VR1 gene at a concentration of less than 2,000 nM, preferably less than 1,000 nM, particularly preferably less than 300 nM, most particularly preferably less than 100 nM, even more preferably less than 75 nM, additionally preferably less than 50 nM, most preferably less than 10 nM.

In the process, the Ca²⁺ influx is quantified in the FLIPR assay with the aid of a Ca²⁺-sensitive dye (type Fluo-4, Molecular Probes Europe BV, Leiden, the Netherlands) in a fluorescent imaging plate reader (FLIPR, Molecular Devices, Sunnyvale, USA), as described hereinafter.

The substituted compounds according to the invention of the aforementioned general formula (I) and corresponding stereoisomers and also the respective corresponding acids, bases, salts and solvates are toxicologically safe and are therefore suitable as pharmaceutical active ingredients in pharmaceutical compositions.

The present invention therefore further relates to a pharmaceutical composition containing at least one compound according to the invention of the above-indicated formula (I), in each case if appropriate in the form of one of its pure stereoisomers, in particular enantiomers or diastereomers, its racemates or in the form of a mixture of stereoisomers, in particular the enantiomers and/or diastereomers, in any desired mixing ratio, or respectively in the form of a corresponding salt, or respectively in the form of a corresponding solvate, and also if appropriate one or more pharmaceutically compatible auxiliaries.

These pharmaceutical compositions according to the invention are suitable in particular for vanilloid receptor 1-(VR1/TRPV1) regulation, preferably for vanilloid receptor 1-(VR1/TRPV1) inhibition and/or for vanilloid receptor 1-(VR1/TRPV1) stimulation, i.e. they exert an agonistic or antagonistic effect.

Likewise, the pharmaceutical compositions according to the invention are preferably suitable for the inhibition and/or treatment of disorders or diseases which are mediated, at least in part, by vanilloid receptors 1.

The pharmaceutical composition according to the invention is suitable for administration to adults and children, including toddlers and babies.

The pharmaceutical composition according to the invention may be found as a liquid, semisolid or solid pharmaceutical form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, if appropriate pressed into tablets, decanted in capsules or suspended in a liquid, and also be administered as much.

In addition to at least one substituted compound of the above-indicated formula (I), if appropriate in the form of one of its pure stereoisomers, in particular enantiomers or diastereomers, its racemate or in the form of mixtures of the stereoisomers, in particular the enantiomers or diastereomers, in any desired mixing ratio, or if appropriate in the form of a corresponding salt or respectively in the form of a corresponding solvate, the pharmaceutical composition according to the invention conventionally contains further physiologically compatible pharmaceutical auxiliaries which can for example be selected from the group consisting of excipients, fillers, solvents, diluents, surface-active substances, dyes, preservatives, blasting agents, slip additives, lubricants, aromas and binders.

The selection of the physiologically compatible auxiliaries and also the amounts thereof to be used depend on whether the pharmaceutical composition is to be applied orally, subcutaneously, parenterally, intravenously, intraperitoneally, intradermally, intramuscularly, intranasally, buccally, rectally or locally, for example to infections of the skin, the mucous membranes and of the eyes. Preparations in the form of tablets, dragées, capsules, granules, pellets, drops, juices and syrups are preferably suitable for oral application; solutions, suspensions, easily reconstitutable dry preparations and also sprays are preferably suitable for parenteral, topical and inhalative application. The substituted compounds according to the invention used in the pharmaceutical composition according to the invention in a repository in dissolved form or in a plaster, agents promoting skin penetration being added if appropriate, are suitable percutaneous application preparations. Orally or percutaneously applicable preparation forms can release the respective substituted compound according to the invention also in a delayed manner.

The pharmaceutical compositions according to the invention are prepared with the aid of conventional means, devices, methods and process known in the art, such as are described for example in “Remington's Pharmaceutical Sciences”, A. R. Gennaro (Editor), 17^{th} edition, Mack Publishing Company, Easton, Pa., 1985, in particular in Part 8, Chapters 76 to 93. The corresponding description is introduced herewith by way of reference and forms part of the disclosure. The amount to be administered to the patient of the respective substituted compounds according to the invention of the above-indicated general formula I may vary and is for example dependent on the patient's weight or age and also on the type of application, the indication and the severity of the disorder. Conventionally 0.001 to 100 mg/kg, preferably 0.05 to 75 mg/kg, particularly preferably 0.05 to 50 mg of at least one such compound according to the invention are applied per kg of the patient's body weight.

The pharmaceutical composition according to the invention is preferably suitable for the treatment and/or inhibition of one or more disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Particularly preferably, the pharmaceutical composition according to the invention is suitable for the treatment and/or inhibition of one or more disorders and/or diseases selected from the group consisting of pain, preferably of pain selected from the group consisting of acute pain, chronic pain, neuropathic pain, visceral pain and joint pain; migraine; depression; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; inflammations, preferably inflammations of the intestine, the eyes, the bladder, the skin or the nasal mucous membrane; urinary incontinence; overactive bladder (OAB); medication dependency; misuse of medication; withdrawal symptoms in medication dependency; development of tolerance to medication, preferably development of tolerance to natural or synthetic opioids; drug dependency; misuse of drugs; withdrawal symptoms in drug dependency; alcohol dependency; misuse of alcohol and withdrawal symptoms in alcohol dependency.

Most particularly preferably, the pharmaceutical composition according to the invention is suitable for the treatment and/or inhibition of pain, preferably of pain selected from the group consisting of acute pain, chronic pain, neuropathic pain and visceral pain.

The present invention further relates to a substituted compound according to general formula (I) and also if appropriate to a substituted compound according to general formula (I) and one or more pharmaceutically acceptable auxiliaries for use in vanilloid receptor 1-(VR1/TRPV1) regulation, preferably for use in vanilloid receptor 1-(VR1/TRPV1) inhibition and/or vanilloid receptor 1-(VR1/TRPV1) stimulation.

The present invention therefore further relates to a substituted compound according to general formula (I) and also if appropriate to a substituted compound according to general formula (I) and one or more pharmaceutically acceptable auxiliaries for use in the inhibition and/or treatment of disorders and/or diseases which are mediated, at least in part, by vanilloid receptors 1.

In particular, the present invention therefore further relates to a substituted compound according to general formula (I) and also if appropriate to a substituted compound according to general formula (I) and one or more pharmaceutically acceptable auxiliaries for use in the inhibition and/or treatment of disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Most particularly preferred is a substituted compound according to general formula (I) and also if appropriate to a substituted compound according to general formula (I) and one or more pharmaceutically acceptable auxiliaries for use in the inhibition and/or treatment of pain, preferably of pain selected from the group consisting of acute pain, chronic pain, neuropathic pain and visceral pain.

The present invention further relates to the use of at least one compound according to general formula (I) and also if appropriate of one or more pharmaceutically acceptable auxiliaries for the preparation of a pharmaceutical composition for vanilloid receptor 1-(VR1/TRPV1) regulation, preferably for vanilloid receptor 1-(VR1/TRPV1) inhibition and/or for vanilloid receptor 1-(VR1/TRPV1) stimulation, and, further for the inhibition and/or treatment of disorders and/or diseases which are mediated, at least in part, by vanilloid receptors 1, such as e.g. disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil.

Another aspect of the present invention is a method for vanilloid receptor 1-(VR1/TRPV1) regulation, preferably for vanilloid receptor 1-(VR1/TRPV1) inhibition and/or for vanilloid receptor 1-(VR1/TRPV1) stimulation, and, further, a method of treatment and/or inhibition of disorders and/or diseases, which are mediated, at least in part, by vanilloid receptors 1, in a mammal, preferably of disorders and/or diseases selected from the group consisting of pain, preferably pain selected from the group consisting of acute pain, chronic pain, neuropathic pain, visceral pain and joint pain; hyperalgesia; allodynia; causalgia; migraine; depression; nervous affection; axonal injuries; neurodegenerative diseases, preferably selected from the group consisting of multiple sclerosis, Alzheimer's disease, Parkinson's disease and Huntington's disease; cognitive dysfunctions, preferably cognitive deficiency states, particularly preferably memory disorders; epilepsy; respiratory diseases, preferably selected from the group consisting of asthma, bronchitis and pulmonary inflammation; coughs; urinary incontinence; overactive bladder (OAB); disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations, preferably inflammations of the intestine, the eyes, the bladder, the skin or the nasal mucous membrane; diarrhoea; pruritus; osteoporosis; arthritis; osteoarthritis; rheumatic diseases; eating disorders, preferably selected from the group consisting of bulimia, cachexia, anorexia and obesity; medication dependency; misuse of medication; withdrawal symptoms in medication dependency; development of tolerance to medication, preferably to natural or synthetic opioids; drug dependency; misuse of drugs; withdrawal symptoms in drug dependency; alcohol dependency; misuse of alcohol and withdrawal symptoms in alcohol dependency; for diuresis; for antinatriuresis; for influencing the cardiovascular system; for increasing vigilance; for the treatment of wounds and/or burns; for the treatment of severed nerves; for increasing libido; for modulating movement activity; for anxiolysis; for local anaesthesia and/or for inhibiting undesirable side effects, preferably selected from the group consisting of hyperthermia, hypertension and bronchoconstriction, triggered by the administration of vanilloid receptor 1 (VR1/TRPV1 receptor) agonists, preferably selected from the group consisting of capsaicin, resiniferatoxin, olvanil, arvanil, SDZ-249665, SDZ-249482, nuvanil and capsavanil, which comprises administering an effective amount of at least one compound of general formula (I) to the mammal.

The effectiveness against pain can be shown, for example, in the Bennett or Chung model (Bennett, G. J. and Xie, Y. K., A peripheral mononeuropathy in rat that produces disorders of pain sensation like those seen in man, Pain 1988, 33(1), 87-107; Kim, S. H. and Chung, J. M., An experimental model for peripheral neuropathy produced by segmental spinal nerve ligation in the rat, Pain 1992, 50(3), 355-363), by tail flick experiments (e.g. according to D'Amour and Smith (J. Pharm. Exp. Ther. 72, 74 79 (1941)) or by the formalin test (e.g. according to D. Dubuisson et al., Pain 1977, 4, 161-174).

The present invention further relates to processes for preparing inventive compounds of the above-indicated general formula (I).

In particular, the compounds according to the present invention of general formula (I) can be prepared by a process according to which at least one compound of general formula (II),

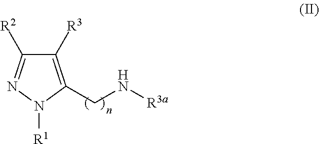

in which R¹, R², R³, R^{3a} and n have one of the foregoing meanings, is reacted in a reaction medium, if appropriate in the presence of at least one suitable coupling reagent, if appropriate in the presence of at least one base, with a compound of general formula (III) with D=OH or Hal,

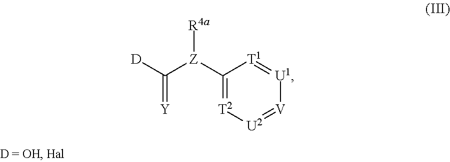

in which Hal represents a halogen, preferably Br or Cl, and R^{4a}, Y, T¹, U¹, V, T² and U² each have one of the foregoing meanings and Z denotes C—R^{4b}, wherein R^{4b} has one of the foregoing meanings, in a reaction medium, if appropriate in the presence of at least one suitable coupling reagent, if appropriate in the presence of at least one base, to form a compound of general formula (I),

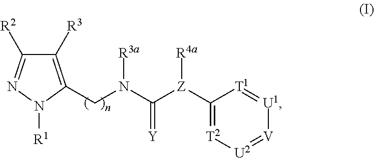

in which Z represents CR^{4b} and R¹, R², R³, R^{3a}, R^{4a}, R^{4b}, Y, T¹, U¹, V, T² and U² and n have one of the foregoing meanings;
or in that at least one compound of general formula (II),

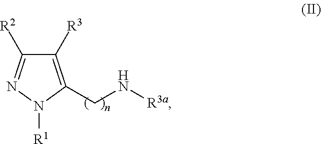

in which R¹, R², R³, R^{3a} and n have one of the foregoing meanings, is reacted to form a compound of general formula (IV)

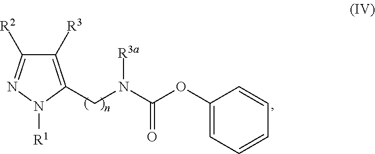

in which R¹, R², R³, R^{3a} and n have one of the foregoing meanings, in a reaction medium, in the presence of phenyl chloroformate, if appropriate in the presence of at least one base and/or at least one coupling reagent, and said compound is if appropriate purified and/or isolated, and a compound of general formula (IV) is reacted with a compound of general formula (V),

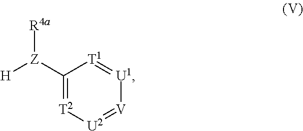

in which R^{4a}, T¹, U¹, V, T² and U² have one of the foregoing meanings, and Z denotes N, in a reaction medium, if appropriate in the presence of at least one suitable coupling reagent, if appropriate in the presence of at least one base, to form a compound of general formula (I),

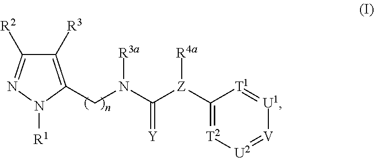

in which Z represents N and R¹, R², R³, R^{3a}, R^{4a}, Y, T¹, U¹, V, T² and U² and n have one of the foregoing meanings.

The reaction of compounds of the above-indicated general formulae (II) and (V) with carboxylic acids of the above-indicated general formula (III), particularly with D=OH, to form compounds of the above-indicated general formula (I) is carried out preferably in a reaction medium selected from the group consisting of diethyl ether, tetrahydrofuran, acetonitrile, methanol, ethanol, (1,2)-dichloroethane, dimethylformamide, dichloromethane and corresponding mixtures, if appropriate in the presence of at least one coupling reagent, preferably selected from the group consisting of 1-benzotriazolyloxy-tris-(dimethylamino)-phosphonium hexafluorophosphate (BOP), dicyclohexylcarbodiimide (DCC), N′-(3-dimethylaminopropyl)-N-ethylcarbodiimide (EDCI), diisopropylcarbodiimide, 1,1′-carbonyldiimidazole (CDI), N-[(dimethylamino)-1H-1,2,3-triazolo[4,5-b]pyridino-1-yl-methylene]-N-methylmethanaminium hexafluorophosphate N-oxide (HATU), 0-(benzotriazol-1-yl)-N,N,N′,N′-tetramethyluronium hexafluorophosphate (HBTU), 0-(benzotriazol-1-yl)-N,N,N′,N′-tetramethyluronium tetrafluoroborate (TBTU), N-hydroxybenzotriazole (HOBt) and 1-hydroxy-7-azabenzotriazole (HOAt), if appropriate in the presence of at least one organic base, preferably selected from the group consisting of triethylamine, pyridine, dimethylaminopyridine, N-methylmorpholine and diisopropylethylamine, preferably at temperatures of from −70° C. to 100° C.

Alternatively, the reaction of compounds of the above-indicated general formulae (II) and (V) with carboxylic acid halides of the above-indicated general formula (III) with D=Hal, in which Hal represents a halogen as the leaving group, preferably a chlorine or bromine atom, to form compounds of the above-indicated general formula (I) is carried out in a reaction medium preferably selected from the group consisting of diethyl ether, tetrahydrofuran, acetonitrile, methanol, ethanol, dimethylformamide, dichloromethane and corresponding mixtures, if appropriate in the presence of an organic or inorganic base, preferably selected from the group consisting of triethylamine, dimethylaminopyridine, pyridine and diisopropylamine, at temperatures of from −70° C. to 100° C.

The compounds of the above-indicated formulae (II), (III), (IV), and (V) are each commercially available and/or can be prepared using conventional processes known to the person skilled in the art. In particular, processes to prepare these compounds are e.g. disclosed in WO 2010/127855-A2, and WO 2010/127856-A2. The corresponding parts of these references are hereby deemed to be part of the disclosure.

All reactions which can be applied for synthesizing the compounds according to the present invention can each be carried out under the conventional conditions with which the person skilled in the art is familiar, for example with regard to pressure or the order in which the components are added. If appropriate, the person skilled in the art can determine the optimum procedure under the respective conditions by carrying out simple preliminary tests. The intermediate and end products obtained using the reactions described hereinbefore can each be purified and/or isolated, if desired and/or required, using conventional methods known to the person skilled in the art. Suitable purifying processes are for example extraction processes and chromatographic processes such as column chromatography or preparative chromatography. All of the process steps of the reaction sequences which can be applied for synthesizing the compounds according to the present invention as well as the respective purification and/or isolation of intermediate or end products, can be carried out partly or completely under an inert gas atmosphere, preferably under a nitrogen atmosphere.

The substituted compounds according to the invention can be isolated both in the form of their free bases, their free acids and also in the form of corresponding salts, in particular physiologically compatible salts, i.e. physiologically acceptable salts.

The free bases of the respective substituted compounds according to the invention can be converted into the corresponding salts, preferably physiologically compatible salts, for example by reaction with an inorganic or organic acid, preferably with HCl, hydrobromic acid, sulfuric acid, methanesulfonic acid, p-toluenesulfonic acid, carbonic acid, formic acid, acetic acid, oxalic acid, succinic acid, tartaric acid, mandelic acid, fumaric acid, maleic acid, lactic acid, citric acid, glutamic acid, saccharic acid, monomethylsebacic acid, 5-oxoproline, hexane-1-sulfonic acid, nicotinic acid, 2, 3 or 4-aminobenzoic acid, 2,4,6-trimethylbenzoic acid, α-lipoic acid, acetyl glycine, hippuric acid, phosphoric acid and/or aspartic acid. The free bases of the respective substituted compounds of the aforementioned general formula (I) and of corresponding stereoisomers can likewise be converted into the corresponding physiologically compatible salts using the free acid or a salt of a sugar additive, such as for example saccharin, cyclamate or acesulfame.

Accordingly, the free acids of the substituted compounds according to the invention can be converted into the corresponding physiologically compatible salts by reaction with a suitable base. Examples include the alkali metal salts, alkaline earth metals salts or ammonium salts [NHₓR₄₋ₓ]⁺, in which x=0, 1, 2, 3 or 4 and R represents a branched or unbranched C₁₋₄ aliphatic residue.

The substituted compounds according to the invention and of corresponding stereoisomers can if appropriate, like the corresponding acids, the corresponding bases or salts of these compounds, also be obtained in the form of their solvates, preferably in the form of their hydrates, using conventional methods known to the person skilled in the art.

If the substituted compounds according to the invention are obtained, after preparation thereof, in the form of a mixture of their stereoisomers, preferably in the form of their racemates or other mixtures of their various enantiomers and/or diastereomers, they can be separated and if appropriate isolated using conventional processes known to the person skilled in the art. Examples include chromatographic separating processes, in particular liquid chromatography processes under normal pressure or under elevated pressure, preferably MPLC and HPLC processes, and also fractional crystallisation processes. These processes allow individual enantiomers, for example diastereomeric salts formed by means of chiral stationary phase HPLC or by means of crystallisation with chiral acids, for example (+)-tartaric acid, (−)-tartaric acid or (+)-10-camphorsulfonic acid, to be separated from one another.

The chemicals and reaction components used in the reactions and schemes described below are available commercially or in each case can be prepared by conventional methods known to the person skilled in the art.

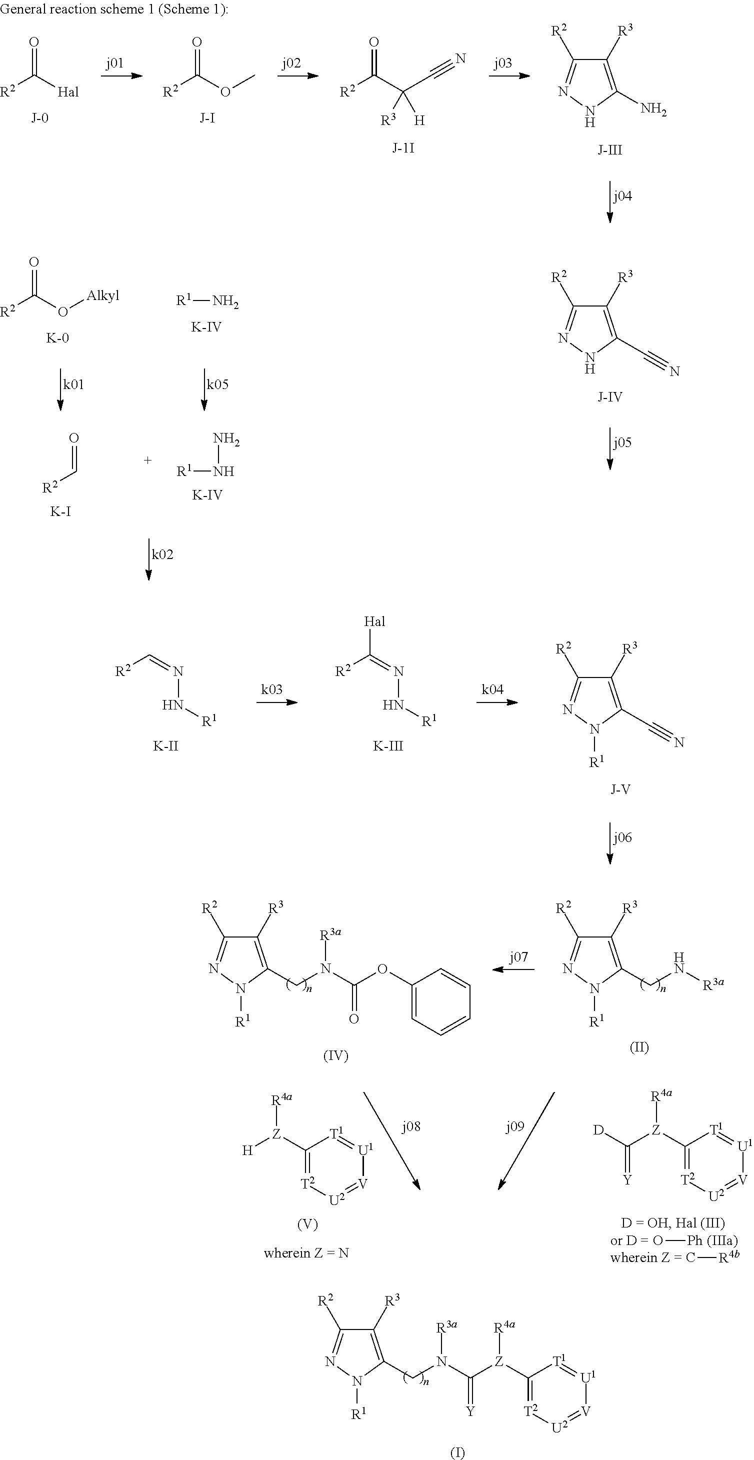

In step j01 an acid halide J-0, in which Hal preferably represents Cl or Br, can be esterified using methanol to form the compound J-I by means of methods with which the person skilled in the art is familiar.

In step j02 the methyl pivalate J-I can be converted into an oxoalkylnitrile J-II by means of methods known to the person skilled in the art, such as for example using an alkyl nitrile R³CH₂—CN, if appropriate in the presence of a base.

In step j03 the compound J-II can be converted into an amino-substituted pyrazolyl derivative J-III by means of methods known to the person skilled in the art, such as for example using hydrazine hydrate, with cyclisation.

In step j04 the amino compound J-III can first be converted into a diazonium salt by means of methods known to the person skilled in the art, such as for example using nitrite, and the diazonium salt can be converted into a cyano-substituted pyrazolyl derivative J-IV with elimination of nitrogen using a cyanide, if appropriate in the presence of a coupling reagent.

In step j05 the compound J-IV can be substituted in the N position by means of methods known to the person skilled in the art, for example using a halide R¹—Hal, if appropriate in the presence of a base and/or a coupling reagent, wherein Hal is preferably Cl, Br or I, or using a boronic acid B(OH)₂R¹ or a corresponding boronic acid ester, if appropriate in the presence of a coupling reagent and/or a base and the compound J-V can in this way be obtained. If R¹ is linked to general formula (I) via a heteroatom (if R¹ represents substructure (T−1), for example, in which o represents 1 and E can represent inter alia O, S, S(═O)₂, NH—C(═O) or NR¹¹), then the substitution can be carried out using methods known to the person skilled in the art, for example with the aid of hydroxylamine-O-sulfonic acid and subsequent conversion into secondary or tertiary amines, wherein E=NR¹¹ (e.g. as described in WO 2010/127855-A2, and WO 2010/127856-A2). In the case of E=0, the substitution can be carried out using methods known to the person skilled in the art, for example with the aid of peroxy reagents and subsequent conversion into ether. In the case of E=S(═O)₂, the substitution can be carried out by sulfonylation with sulfonyl chlorides, for example. In the case of E=S, the preparation can for example be carried out by reaction with disulfides or else with sulfenyl chlorides or sulfene amides, or else by transformation into the mercaptan by means of methods known to the person skilled in the art and subsequent conversion into the thioether.

Alternatively, a second synthesis pathway, in which in step k01 an ester K-0 is first reduced to form the aldehyde K-I by means of methods known to the person skilled in the art, for example using suitable hydrogenation reagents such as metal hydrides, is suitable for preparing the compound J-V.

In step k02 the aldehyde K-I can then be reacted with a hydrazine K-V, which can be obtained in step k05, starting from the primary amine K-IV, by means of methods known to the person skilled in the art, to form the hydrazine K-II by means of methods known to the person skilled in the art with elimination of water.

In step k03 the hydrazine K-II can be halogenated, preferably chlorinated, by means of methods known to the person skilled in the art with the double bond intact, such as for example using a chlorination reagent such as NCS, and the compound K-III can in this way be obtained.

In step k04 the hydrazonoyl halide K-III can be converted into a cyano-substituted compound J-V by means of methods known to the person skilled in the art, such as for example using a halogen-substituted nitrile, with cyclisation.

In step j06 the compound J-V can be hydrogenated by means of methods known to the person skilled in the art, for example using a suitable catalyst such as palladium/activated carbon or using suitable hydrogenation reagents, and the compound (II) can in this way be obtained, wherein R^{3a} is H. Optionally, before performing j07, a C₁₋₄ aliphatic residue, unsubstituted or mono- or polysubstituted, can be introduced into the amine (II) as R^{3a}≠H by methods known to the person skilled in the art, such as for example mono-alkylation of a primary amine.

In step j07 the compound (II) can be converted into the compound (IV) by means of methods known to the person skilled in the art, such as for example using phenyl chloroformate, if appropriate in the presence of a coupling reagent and/or a base. In addition to the methods disclosed in the present document for preparing unsymmetrical ureas using phenyl chloroformate, there are further processes with which the person skilled in the art is familiar, based on the use of activated carbonic acid derivatives or isocyanates, if appropriate.

In step j08 the amine (V) can be converted into the urea compound (I) (wherein Z=N). This can be achieved by reaction with (IV) by means of methods with which the person skilled in the art is familiar, if appropriate in the presence of a base.

In step j09 the amine (II) can be converted into the amide (I) (wherein Z═C—R^{4b}). This can for example be achieved by reaction with an acid halide, preferably a chloride of formula (III) with D=Hal by means of methods with which the person skilled in the art is familiar, if appropriate in the presence of a base or by reaction with an acid of formula (III) with D=OH, if appropriate in the presence of a suitable coupling reagent, for example HATU or CDI, if appropriate with the addition of a base. Further, the amine (II) may be converted into the amide (I) (wherein Z═C—R^{4b}) by reaction of a compound (IIIa) by means of methods with which the person skilled in the art is familiar, if appropriate in the presence of a base.

The compounds according to general formula (I), wherein Z=N, may be further prepared by a reaction sequence according to general reaction scheme 2.

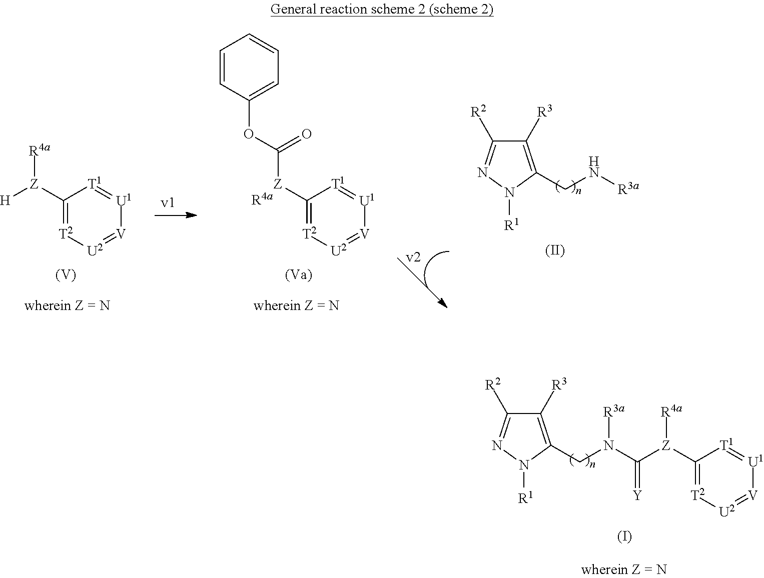

In step v1 the compound (V) can be converted into the compound (Va) by means of methods known to the person skilled in the art, such as for example using phenyl chloroformate, if appropriate in the presence of a coupling reagent and/or a base. In addition to the methods disclosed in the present document for preparing unsymmetrical ureas using phenyl chloroformate, there are further processes with which the person skilled in the art is familiar, based on the use of activated carbonic acid derivatives or isocyanates, if appropriate.

In step v2 the amine (II) can be converted into the urea compound (I) (wherein Z=N). This can be achieved by reaction with (Va) by means of methods with which the person skilled in the art is familiar, if appropriate in the presence of a base.

The methods with which the person skilled in the art is familiar for carrying out the reaction steps j01 to j09 and also k01 to k05 as well as v1 and v2 may be inferred from the standard works on organic chemistry such as, for example, J. March, Advanced Organic Chemistry, Wiley & Sons, 6th edition, 2007; F. A. Carey, R. J. Sundberg, Advanced Organic Chemistry, Parts A and B, Springer, 5th edition, 2007; team of authors, Compendium of Organic Synthetic Methods, Wiley & Sons. In addition, further methods and also literature references can be issued by the common databases such as, for example, the Reaxys® database of Elsevier, Amsterdam, NL or the SciFinder® database of the American Chemical Society, Washington, US.

The invention will be described hereinafter with the aid of a number of examples. This description is intended merely by way of example and does not limit the general idea of the invention.

### EXAMPLES

The indication “equivalents” (“eq.” or “eq”) means molar equivalents, “RT” or “rt” means room temperature (23±7° C.), “M” are indications of concentration in mol/l, “aq.” means aqueous, “sat.” means saturated, “sol.” means solution, “conc.” means concentrated.

### Further Abbreviations:

d days
BH₃.S(CH₃)₂ borane-methyl sulfide complex
BINAP 2,2″bis(diphenylphosphino)-1,1″binaphthyl brine saturated aqueous sodium chloride solution
n-BuLi n-butyllithium
CC column chromatography on silica gel
DBU 1,8-diazabicyclo[5.4.0]undec-7-en
DCM dichloromethane
DMAP 4-dimethylaminopyridine

### DMF N,N-dimethylformamide

DPPF 1,1′-bis(diphenylphosphino)ferrocene
EDC N-(3-dimethylaminopropyl)-N′-ethylcarbodiimide
EDCl N-ethyl-N′-(3-dimethylaminopropyl)carbodiimide hydrochloride ether diethyl ether
EtOAc or EE ethyl acetate
EtOH ethanol
h hour(s)
GC gas chromatography
H₂O water
m/z mass-to-charge ratio
MeOH methanol
MeCN acetonitrile
min minutes
MS mass spectrometry
NEt₃ triethylamine
NiBr₂ bipy complex of nickel(II) bromide and 2,2′-bipyridine
NMP N-methyl-2-pyrrolidon
Pd/C palladium on charcoal
Pd₂(dba)₃ tris(dibenzylideneacetone)dipalladium(0)
Pd(dppf)Cl_{2 [}1,1′-bis(diphenylphosphino)ferrocene]dichloropalladium(II)
Pd(PPh₃)₄ tetrakis(triphenylphosphine)palladium(0)
TBAF tetra-n-butylammonium fluoride
TBDMSCl tert-butyldimethylsilyl chloride
TLC thin layer chromatography
THF tetrahydrofuran
v/v volume to volume
w/w weight in weight

The yields of the compounds prepared were not optimized.

All temperatures are uncorrected.

All starting materials which are not explicitly described were either commercially available (the details of suppliers such as for example Acros, Avocado, Aldrich, Apollo, Bachem, Fluka, FluoroChem, Lancaster, Manchester Organics, MatrixScientific, Maybridge, Merck, Rovathin, Sigma, TCl, Oakwood, etc. can be found in the Symyx® Available Chemicals Database of MDL, San Ramon, US or the SciFinder® Database of the ACS, Washington D.C., US, respectively, for example) or the synthesis thereof has already been described precisely in the specialist literature (experimental guidelines can be found in the Reaxys® Database of Elsevier, Amsterdam, NL or the SciFinder® Database of the ACS, Washington D.C., US, respectively, for example) or can be prepared using the conventional methods known to the person skilled in the art.

The stationary phase used for the column chromatography was silica gel 60 (0.04-0.063 mm) from E. Merck, Darmstadt.

The mixing ratios of solvents or eluents for chromatography are specified in v/v.

All the intermediate products and example compounds were analytically characterized by means of ¹H-NMR spectroscopy. In addition, mass spectrometry tests (MS, m/z for [M+H]⁺) were carried out for all the example compounds and selected intermediate products.

### Synthesis of Intermediate Products

1. Synthesis of 3-tert-butyl-1-methyl-1H-pyrazol-5-yl-methanamine (steps j01-j06)

### Step j01:

Pivaloyl chloride (J-0) (1 eq., 60 g) was added dropwise to a solution of methanol (120 mL) within 30 min at 0° C. and the mixture was stirred for 1 h at room temperature. After the addition of water (120 mL), the separated organic phase was washed with water (120 mL), dried over sodium sulfate and codistilled with dichloromethane (150 mL). The liquid product J-I was able to be obtained at 99% purity (57 g).

### Step j02:

NaH (50% in paraffin oil) (1.2 equivalents, 4.6 g) was dissolved in 1,4-dioxane (120 mL) and the mixture was stirred for a few minutes. Acetonitrile (1.2 equivalents, 4.2 g) was added dropwise within 15 min and the mixture was stirred for a further 30 min. The methyl pivalate (J-I) (1 equivalents, 10 g) was added dropwise within 15 min and the reaction mixture was refluxed for 3 h. After complete reaction, the reaction mixture was placed in iced water (200 g), acidified to pH 4.5 and extracted with dichloromethane (12×250 mL). The combined organic phases were dried over sodium sulfate, distilled and after recrystallisation from n-hexane (100 mL) 5 g of the product (J-II) (51% yield) was able to be obtained as a solid brown substance.

### Step j03:

At room temperature 4,4-dimethyl-3-oxopentanenitrile (J-II) (1 equivalents, 5 g) was taken up in ethanol (100 mL), mixed with hydrazine hydrate (2 equivalents, 4.42 g) and refluxed for 3 h. The residue obtained after removal of the ethanol by distillation was taken up in water (100 mL) and extracted with ethyl acetate (300 mL). The combined organic phases were dried over sodium sulfate, the solvent was removed under vacuum and the product (JAI) (5 g, 89% yield) was obtained as a light red solid after recrystallisation from n-hexane (200 mL).

### Step j04:

3-Tert-butyl-1H-pyrazol-5-amine (JAI) (1 equivalents, 40 g) was dissolved in diluted HCl (120 mL of HCl in 120 mL of water) and mixed dropwise with NaNO₂ (1.03 equivalents, 25 g in 100 mL) at 0-5° C. over a period of 30 min. After stirring for 30 minutes, the reaction mixture was neutralised with Na₂CO₃. A diazonium salt obtained by reaction of KCN (2.4 equivalents, 48 g), water (120 mL) and CuCN (1.12 equivalents, 31 g) was added dropwise to the reaction mixture within 30 min and the mixture was stirred for a further 30 min at 75° C. After complete reaction, the reaction mixture was extracted with ethyl acetate (3×500 mL), the combined organic phases were dried over sodium sulfate and the solvent was removed under vacuum. The purification (silica gel: 100-200 mesh, eluent: 20% ethyl acetate/n-hexane) of the residue by column chromatography produced a white solid (J-IV) (6.5 g, 15%).

Step j05 (method 1):

3-tert.-butyl-1H-pyrazol-5-carbonitrile (J-IV) (10 mmol) was added to a suspension of NaH (60%) (12.5 mmol) in dimethylformamide (20 mL) at room temperature while stirring. After stirring for 15 minutes, methyl iodide (37.5 mmol) was added dropwise to this reaction mixture at room temperature. After stirring for 30 min at 100° C., the reaction mixture was mixed with water (150 mL) and extracted with dichloromethane (3×75 mL). The combined organic extracts were washed with water (100 mL) and sat. NaCl solution (100 mL) and dried over magnesium sulfate. After removal of the solvent under vacuum, the residue was purified by column chromatography (silica gel: 100-200 mesh, eluent: various mixtures of ethyl acetate and cyclohexane as the mobile solvent) and the product J-V was obtained.

### Step j06:

### Method 1:

J-V was dissolved together with palladium on carbon (10%, 500 mg) and concentrated HCl (3 mL) in methanol (30 mL) and exposed to a hydrogen atmosphere for 6 h at room temperature. The reaction mixture was filtered over celite and the filtrate was concentrated under vacuum. The residue was purified by means of flash chromatography (silica gel: 100-200 mesh, eluent: ethyl acetate) and the product (II) was in this way obtained.

Method 2:

J-V was dissolved in tetrahydrofuran (10 mL) and BH₃S(CH₃)₂ (2.0 M in tetrahydrofuran, 3 mL, 3 equivalents) was added thereto. The reaction mixture was heated to reflux for 8 h, aq. 2 N HCl (2 N) was added thereto and the reaction mixture was refluxed for a further 30 minutes. The reaction mixture was mixed with aq. NaOH solution (2N) and washed with ethyl acetate. The combined organic phases were washed with sat. aq. NaCl solution and dried over magnesium sulfate. The solvent is removed under vacuum and the residue is purified by column chromatography (silica gel: 100-200 mesh, eluent: various mixtures of dichloromethane and methanol as the mobile solvent) and the product (II) (3-tert-butyl-1-methyl-1H-pyrazol-5-yl)methanamine) is in this way obtained.

2. The following further intermediate products were synthesised in a similar manner using the process described hereinbefore under 1.:

(3-tert-butyl-1-(3-(trifluoromethyl)phenyl)-1H-pyrazol-5-yl)methanamine

3. Alternatively, step j05 can also be carried out as follows (method 2):

Step j05 (method 2):

A mixture of 3-tert-butyl-1H-pyrazol-5-carbonitrile (J-IV) (10 mmol), a boronic acid B(OH)₂R¹ or a corresponding boronic acid ester (20 mmol) and copper (II) acetate (15 mmol) is placed in dichloromethane (200 mL), mixed with pyridine (20 mmol) while stirring at room temperature and the mixture is stirred for 16 h. After removal of the solvent under vacuum, the residue obtained is purified by column chromatography (silica gel: 100-200 mesh, eluent: various mixtures of ethyl acetate and cyclohexane as the mobile solvent) and the product J-V is in this way obtained.

4. Synthesis of 1-(3-chlorophenyl)-3-(trifluoromethyl)-1H-pyrazol-5-yl-methanamine (steps k01-k05 and j06)

### Step k01:

LAlH (lithium aluminium hydride) (0.25 equivalents, 0.7 g) was dissolved in dry diethyl ether (30 mL) under a protective gas atmosphere and stirred for 2 h at room temperature. The suspension obtained was taken up in diethyl ether (20 mL). Ethyl-2,2,2-trifluoroacetate (K-0) (1 equivalent, 10 g) was taken up in dry diethyl ether (20 mL) and added dropwise to the suspension at −78° C. over a period of 1 h. The mixture was then the stirred for a further 2 h at −78° C. ethanol (95%) (2.5 mL) was then added dropwise, the reaction mixture was heated to room temperature and placed on iced water (30 mL) with concentrated H₂SO₄ (7.5 mL). The organic phase was separated and concentrated under vacuum and the reaction product K-I was immediately introduced into the next reaction step k02.

### Step k05:

3-chloroaniline (K-IV) (1 equivalent, 50 g) was dissolved at −5 to 0° C. in concentrated HCl (300 mL) and stirred for 10 min. A mixture of NaNO₂ (1.2 equivalents, 32.4 g), water (30 mL), SnCl₂.2H₂O (2.2 equivalents, 70.6 g) and concentrated HCl (100 mL) was added dropwise over a period of 3 h while maintaining the temperature. After stirring for a further 2 h at −5 to 0° C., the reaction mixture was set to pH 9 using NaOH solution and extracted with ethyl acetate (250 mL). The combined organic phases were dried over magnesium sulfate and the solvent was removed under vacuum. The purification by column chromatography (silica gel: 100-200 mesh, eluent: 8% ethyl acetate/n-hexane) produced 40 g (72%) of (3-chlorophenyl)hydrazine (K-IV) as a brown oil.

### Step k02:

The aldehyde (K-I) (2 equivalents, 300 mL) obtained from k01 and (3-chlorophenyl)hydrazine (K-IV) (1 equivalent, 20 g) were placed in ethanol (200 mL) and refluxed for 5 h. The solvent was removed under vacuum, the residue was purified by column chromatography (silica gel: 100-200 mesh, eluent: n-hexane) and the product (25 g, 72%) K-II was obtained as a brown oil.

### Step k03:

The hydrazine K-II (1 equivalent, 25 g) was dissolved in dimethylformamide (125 mL). N-chlorosuccinimide (1.3 equivalents, 19.5 g) was added portionwise at room temperature within 15 min and the mixture was stirred for 3 h. The dimethylformamide was removed by distillation and the residue was taken up in ethyl acetate. The ethyl acetate was removed under vacuum, the residue obtained was purified by column chromatography (silica gel: 100-200 mesh, eluent: n-hexane) and the product K-III (26.5 g, 92%) was obtained as a pink-colored oil.

### Step k04:

At room temperature the hydrazonoyl chloride K-III (1 equivalent, 10 g) was taken up in toluene (150 mL) and mixed with 2-chloroacrylonitrile (2 equivalents, 6.1 mL) and triethylamine (2 equivalents, 10.7 mL). This reaction mixture was stirred for 20 h at 80° C. The mixture was then diluted with water (200 mL) and the phases were separated. The organic phase was dried over magnesium sulfate and the solvent was removed under vacuum. The residue was purified by means of column chromatography (silica gel: 100-200 mesh, eluent: 5% ethyl acetate/n-hexane) and the product (5.5 g, 52%) was obtained as a white solid J-V.

Step j06 (method 3):

The carbonitrile J-V (1 equivalent, 1 g) was dissolved in methanolic ammonia solution (150 mL, 1:1) and hydrogenated in an H-cube (10 bar, 80° C., 1 mL/min, 0.25 mol/L). After removal of the solvent under vacuum, (1-(3-chlorophenyl)-3-(trifluoromethyl)-1H-pyrazol-5-yl)methanamine (II) was able to be obtained as a white solid (0.92 g, 91%).

5. The following further intermediate products were synthesised in a similar manner using the process described hereinbefore under 4.:

(1-(3-fluorophenyl)-3-(trifluoromethyl)-1H-pyrazol-5-yl)methanamine
(1-(3-chloro-4-fluorophenyl)-3-(trifluoromethyl)-1H-pyrazol-5-
yl)methanamine
(1-(3-methoxyphenyl)-3-(trifluoromethyl)-1H-pyrazol-5-yl)methanamine

6. Preparation of selected carbamate phenyl esters of general formula (Va) or (IV) and phenyl esters of general formula (IIIa)

6.1 Synthesis of methyl phenyl (3-tert-butyl-1-(3-chlorophenyl)-1H-pyrazol-5-yl)methylcarbamate (employed e.g. for the synthesis of example compounds no. 2, 4, 6 and 10)

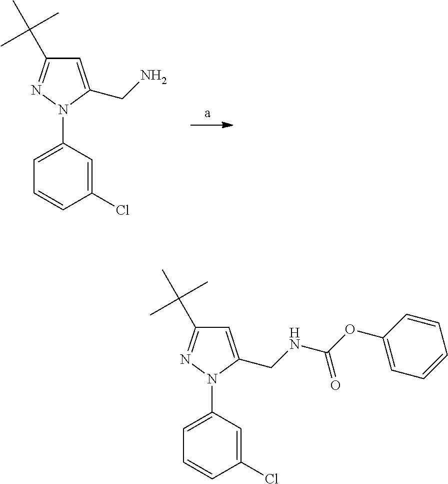 [SMILES: CC(C)(C)c1cc(CN)n(-c2cccc(Cl)c2)n1 ; CC(C)(C)c1cc(CNC(=O)Oc2ccccc2)n(-c2cccc(Cl)c2)n1]

Step a: To a solution of (3-tert-butyl-1-(3-chlorophenyl)-1H-pyrazol-5-yl)methanamine (5 g, 18 mmol) in dimethylformamide (25 mL), potassium carbonate (9.16 g, 66 mmol, 3.5 eq) was added and cooled the contents to 0° C. Then phenyl chloroformate (3.28 g (2.65 mL), 20 mmol, 1.1 equivalents) was added dropwise for 15 minutes and the overall reaction mixture was stirred for another 15 minutes at 0° C. Progress of the reaction was monitored by TLC (20% ethyl acetate-n-hexane). On completion of the reaction, reaction contents were filtered, filtrate was diluted with cold water (100 mL) and the product extracted with ethyl acetate (3×25 mL). Combined organic layer was washed with brine solution (100 mL), dried over sodium sulfate and concentrated under reduced pressure. Crude obtained was purified by column chromatography (silica gel: 100-200 mesh, eluent: 10% ethyl acetate in n-hexane) to yield the required product as a white solid (3.2 g, 45%).

7. Preparation of additional selected pyrazol derivatives according to general formula (II)

7.1 Synthesis of (1-(3-chlorophenyl)-4-methyl-3-(trifluoromethyl)-1H-pyrazol-5-yl)methanamine (employed for the synthesis of example compound no. 73)

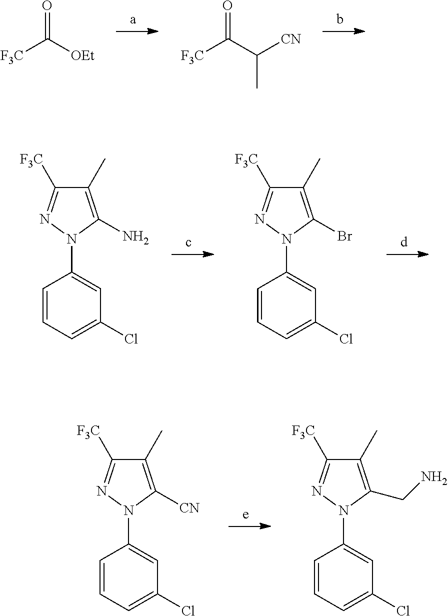

Step a: To a solution of diispropylamine (40.8 g (57 mL), 0.404 mol, 2.3 equivalents) in tetrahydrofuran (400 mL), n-BuLi (1.6 molar) (24.7 g (258.3 mL, 0.38 mol, 2.2 equivalents) was added drop wise for 2 h at 20° C. and stirred the contents for 30 45 min at 0° C. Cooled the contents to 75° C., a solution of ethyl 2,2,2-trifluoroacetate (25 g, 0.17 mol) in tetrahydrofuran (200 mL) was added drop wise for 2 h. The reaction mixture was stirred initially for 1 h at 75° C. and later for another 1 h at room temperature. Progress of the reaction was monitored by TLC (50% ethyl acetate in n-hexane). On completion of the reaction, quenched the reaction with ice water (700 mL) and the solvents were distilled off completely. Residue washed with dichloromethane (3×300 mL), acidified the contents with 30% HCl solution and the product extracted with ether (3×400 mL). Combined organic layer was dried over sodium sulfate, concentrated under reduced pressure and the crude obtained was distilled under vacuum to yield the product at 35° C./0.1 mm as a colorless liquid (17 g, 64%).

Step b: A step-a product (10 g, 0.066 mol) was taken in ethanolic HCl (300 mL) and 3-chlorophenyl hydrazine (9.43 g, 0.066 mol, 1 equivalent) was added. The reaction mixture was heated to reflux for 2 h. Progress of the reaction was monitored by TLC (20% ethyl acetate in n-hexane). On completion of the reaction, reaction contents were concentrated and the residue taken in water (200 mL). Basified the contents to a pH˜12 with 1N NaOH solution and filtered the contents. Solid obtained was taken in ethyl acetate (200 mL), dried the contents over sodium sulfate and concentrated under reduced pressure to yield the required product as a red colored solid (12 g, 65%).

Step c: Cupric bromide (11.33 g, 0.0511 mol, 1.2 equivalents) was taken in acetonitrile (176 mL) and heated to 150° C. Then n-butyl nitrite (6.59 g (7.47 mL), 0.063 mol, 1.5 eq) was added followed by a solution of step-b product (11.75 g, 0.042 mol) in acetonitrile (176 mL) was added drop wise for 30 min at 150° C. and stirred for 15 min. Progress of the reaction was monitored by TLC (5% ethyl acetate/n-hexane). On completion of the reaction, acetonitrile was distilled off, residue was taken in ice cold water (300 mL) and the product extracted with ethyl acetate (5×100 mL). Combined extract was dried over sodium sulfate, concentrated under reduced pressure and the crude obtained was subjected to column chromatography (silica gel: 100-200 mesh, eluent: pure n-hexane). Pure product was not isolated and a mixture was obtained as a red colored liquid (16 g, crude) and the same product used for the next step.

Step d: To a solution of step-c product (13 g, 0.038 mol) in NMP (130 mL), copper cyanide (6.8 g, 0.076 mol, 2 equivalents), sodium iodide (100 mg, catalytic) were added. The reaction mixture was placed in a pre-heated oil bath at 180° C. and allowed to stir for 8 h. Progress of the reaction was monitored by TLC (5% ethyl acetate in n-hexane). On completion of the reaction, diluted the reaction contents with water (200 mL) and the product extracted with ethyl acetate (5×100 mL). Combined extract was washed with cold water (5×50 mL), dried over sodium sulfate and concentrated under reduced pressure. The crude obtained was purified by column chromatography (silica gel: 100-200 mesh, eluent: 2% ethyl acetate in n-hexane) to yield the required product as a pale yellow colored solid (8 g).

Step e: To a solution of step-d product (5 g, 0.017 mol) in dry tetrahydrofuran (30 mL), Boran-tetrahydrofuran in tetrahydrofuran (70 mL) was added drop wise for 30 min at 0 5° C. Reaction mixture was slowly heated to 50° C. and allowed to stir for 12 h. Progress of the reaction was monitored by TLC (75% ethyl acetate/n-hexane). On completion of the reaction, acidified the contents to 0 5° C. with conc.HCl at 0° C. and stirred the contents for 2 h at room temperature. Then basified the contents to a pH˜12 with 10% NaOH solution and the product extracted with ethyl acetate (5×50 mL). Combined extract was dried over sodium sulfate and concentrated under reduced pressure. Solid obtained was washed with 10% ether/n-hexane and dried to yield the required product as a white colored solid (3 g, 59%).

7.2 Synthesis of (1-(3-chlorophenyl)-3-cyclopropyl-1H-pyrazol-5-yl)methanamine hydrochloride (employed e.g. for the synthesis of example compound no. 24 and 72)

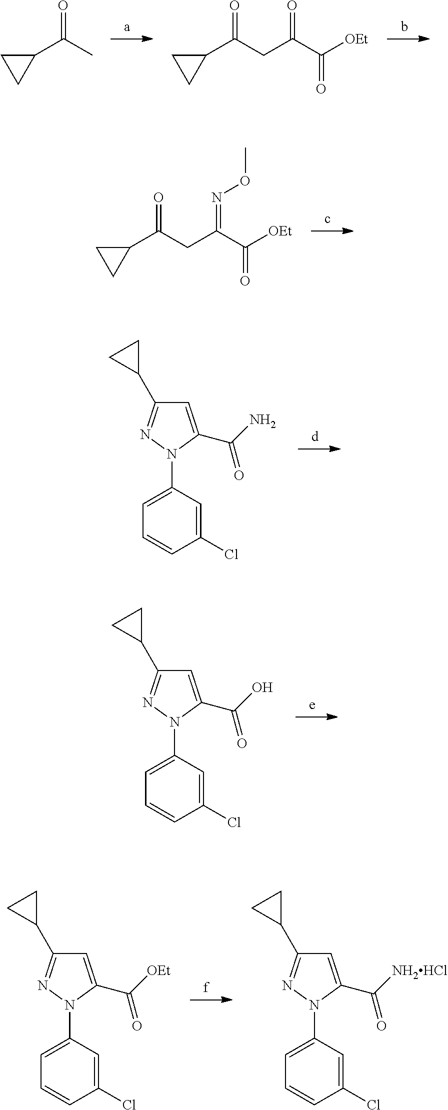

Step a: To a solution of sodium ethoxide (freshly prepared by dissolving sodium (1 g, 8.2 mmol, 1.2 equivalents) in ethanol (30 mL)), diethyl oxalate (0.92 mL, 6.85 mmol, 1 equivalent) was added at room temperature followed by addition of cyclopropyl methyl ketone (0.74 mL, 7.5 mmol, 1.1 equivalents) dropwise at 0° C. The reaction mixture was slowly warmed to room temperature and stirred for 3 h. Ice cold water (10 mL) was added and ethanol was evaporated under reduced pressure. The residual aqueous layer was diluted with 2 N aq. HCl (15 mL) and extracted with diethyl ether (2×25 mL). The organic layer was washed with brine solution and dried over sodium sulfate, filtered and concentrated to give a pale brown liquid (400 mg, 31%).

Step b: To a solution of step-a product (200 mg, 0.543 mmol, 1 equivalent) in ethanol (8 mL), methoxylamine hydrochloride (30% solution in water, 0.4 mL, 0.651 mmol, 1.2 equivalents) was added at room temperature and the reaction mixture stirred for 1 h. ethanol was evaporated under reduced pressure and the residual aqueous layer was extracted with ethyl acetate (15 mL). The organic layer was washed with water (10 mL), brine solution (10 mL), dried over sodium sulfate, filtered and concentrated under reduced pressure to give a pale yellow liquid (180 mg, 78%).

Step c: A mixture of step-b product (1.1 g, 5.164 mmol, 1 equivalent) and 3-chlorophenyl hydrazine hydrochloride (1.84 g, 10.27 mmol, 2 equivalents) was taken in acetic acid (20 mL), 2-methoxy ethanol (10 mL) and the reaction mixture was heated at 105° C. for 3 h. Solvent was evaporated and the residue was extracted with ethyl acetate (60 mL). The organic layer washed with water (10 mL), brine solution (10 mL), dried over sodium sulfate, filtered and concentrated under reduced pressure to give a residue. Purification by column chromatography (silica gel: 100-200 mesh; eluent: ethyl acetate-petroleum ether (4:96)) afforded a pale brown semi solid (1.15 g, 77%).

Step d: To a solution of step-c product (2.5 g, 8.62 mmol, 1 eq) in tetrahydrofuran (15 mL)—methanol (9 mL)-water (3 mL), lithium hydroxide (1.08 g, 25.71 mmol, 3 equivalents) was added at 0° C. and the reaction mixture was stirred for 2 h at room temperature. Solvent was evaporated and pH of the residue was adjusted to ˜3 sing 2 N aqueous HCl (1.2 mL). The acidic aqueous layer was extracted with ethyl acetate (2×60 mL); the combined organic layer washed with water (10 mL), brine solution (10 mL), dried over sodium sulfate, filtered and concentrated under reduced pressure to give an off white solid (1.4 g, 62%).

Step e: To a solution of step-d product (1.4 g, 5.34 mmol, 1 equivalent) in 1,4-dioxane (30 mL), pyridine (0.25 mL, 3.2 mmol, 0.6 equivalents) and di-tert-butyl dicarbonate (1.4 mL, 6.37 mmol, 1.2 equivalents) were added at 0° C. and the resulting mixture was stirred for 30 minutes at the same temperature. Ammonium bicarbonate (0.84 g, 10.63 mmol, 2 equivalents) was added at 0° C. and the reaction mixture was stirred at room temperature overnight. The reaction mixture was diluted with water (10 mL) and the aqueous layer was extracted with ethyl acetate (2×30 mL). The organic layer was washed with 2N HCl (20 mL), water (10 mL), brine solution (10 mL), dried over sodium sulfate, filtered and concentrated under reduced pressure to give a residue. Purification by column chromatography (silica gel: 100-200 mesh; eluent: ethyl acetate-petroleum ether (16:84)) gave a white solid (1 g, 72%).

Step f: To a solution of step-e product (2 g, 7.66 mmol, 1 equivalent) in tetrahydrofuran (25 mL), BH₃.DMS (1.44 mL, 15.32 mmol, 2 equivalents) was added at 0° C. and the reaction mixture was heated at 70° C. for 3 h. The reaction mixture was cooled to 0° C. and methanol (15 mL) was added and reaction mixture heated at reflux for 1 h. The reaction mixture was brought to room temperature and solvent was evaporated under reduced pressure. The residue was dissolved in ether (15 mL), cooled to 0° C. and a solution of HCl in 1,4-dioxane (3 mL) was added (pH of the reaction mixture 4). The precipitated solid was filtered and washed with diethyl ether (5 mL, thrice) to give the hydrochloride salt compound as a white solid (600 mg, 28%).

7.3 Synthesis of (3-tert-butyl-1-(pyridin-2-yl)-1H-pyrazol-5-yl)methanamine (employed e.g for the synthesis of example compound no. 85)

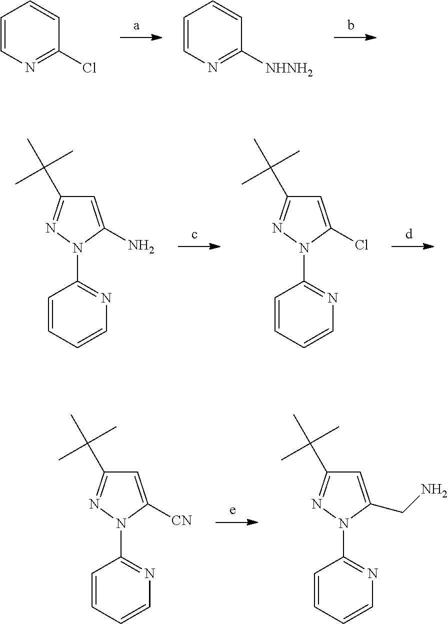 [SMILES: Clc1ccccn1 ; NNc1ccccn1 ; CC(C)(C)c1cc(N)n(-c2ccccn2)n1 ; CC(C)(C)c1cc(Cl)n(-c2ccccn2)n1 ; CC(C)(C)c1cc(C#N)n(-c2ccccn2)n1 ; CC(C)(C)c1cc(CN)n(-c2ccccn2)n1]

Step a: To a solution of 2-chloropyridine (20 g, 0.17 mol) in ethanol (100 mL), hydrazine hydrate (132 mL) was added and the reaction mixture was heated to reflux for 15 h. Progress of the reaction was monitored by TLC (40% ethyl acetate/n-hexane). As the reaction not completed, continued to reflux for another 15 h and monitored by TLC. On completion of the reaction, ethanolic hydrazine hydrochloride was distilled off completely at 100° C., residue was taken in dichloromethane (500 mL) and washed the contents with saturated sodium carbonate solution (100 mL). Combined organic layer was dried over sodium sulfate and concentrated under reduced pressure to obtain the crude product as a low melting solid (11 g, crude). The crude obtained was directly used for the next step.

Step b: To a stirred solution of step-a product (11 g, crude) in ethanol (110 mL), 4,4-dimethyl-3-oxopentanenitrile (11.3 g, 0.09 mol, 0.9 equivalents) was added portion wise followed by catalytic amount of HCl. The reaction mixture was heated to 100° C. and refluxed for 6 h. Progress of the reaction was monitored by TLC (20% ethyl acetate/n-hexane). On completion of the reaction, ethanol was distilled off, residue was taken in water (200 mL) and the product extracted with ethyl acetate (2×100 mL). Combined extract was dried over sodium sulfate, concentrated under reduced pressure and the crude obtained was purified by column chromatography (silica gel: 100-200 mesh, eluent:10% ethyl acetate in n-hexane) to yield the required product as an off white solid (18 g).

Step c: To a solution of step-b product (4 g, 0.01 mol) in acetonitrile (80 mL), cupric chloride (12.3 g, 0.09 mol, 5 equivalents) was added. A solution of tert-butyl nitrite (2.8 (3.3 mL), 0.023 mol, 1.5 equivalents) in acetonitrile (40 mL (total 120 mL)) was added drop wise for 10 min and the overall reaction mass was stirred for 5 h at room temperature. Progress of the reaction was monitored by TLC (10% ethyl acetate/n-hexane). On completion of the reaction, acetonitrile was distilled off, residue was taken in water (100 mL) and the product extracted with ethyl acetate (2×200 mL). Combined extract was dried over sodium sulfate, concentrated under reduced pressure and the crude was purified by column chromatography (silica gel: 100-200 mesh, eluent: 4% ethyl acetate in n-hexane) to yield the required product as a pale yellow colored liquid (2.1 g, 48%).

Step d: To a stirred solution of step-c product (2.1 g, 0.008 mol) in NMP (21 mL), copper cyanide (1.56 g, 0.017 mol, 2 equivalents) was added portion wise followed by a catalytic amount of sodium iodide was added. The reaction mixture was heated to 180° C. and maintained at that temperature for 4 h. Progress of the reaction was monitored by TLC (10% ethyl acetate/n-hexane). On completion of the reaction, diluted the reaction contents with ethyl acetate, filtered the contents through celite bed and the filtrate washed with cold water (50 mL). Organic layer was dried over sodium sulfate, concentrated under reduced pressure and the crude was purified by column chromatography (silica gel: 100-200 mesh, eluent: 6-8% ethyl acetate in n-hexane) to yield the required product as an off white solid (0.8 g, 40%).

Step e: To a solution of step-d product (1.5 g, 0.006 mol) in methanol (20 mL), catalytic amount of raney nickel. The reaction mixture was hydrogenated for 1 h at 60 psi. Progress of the reaction was monitored by TLC (15% ethyl acetate/n-hexane). On disappearance of the starting material, filtered the contents on celite bed and washed with methanol. To the filtrate was purified by column chromatography (silica gel: 100-200 mesh, eluent: 6% ethyl acetate in n-hexane) to yield the titled product as a cream colored oil (1.4 g, 97%).

7.4 Synthesis of (1-(pyridin-3-yl)-3-(trifluoromethyl)-1H-pyrazol-5-yl)methanamine hydrochloride (employed e.g for the synthesis of example compound no. 97)

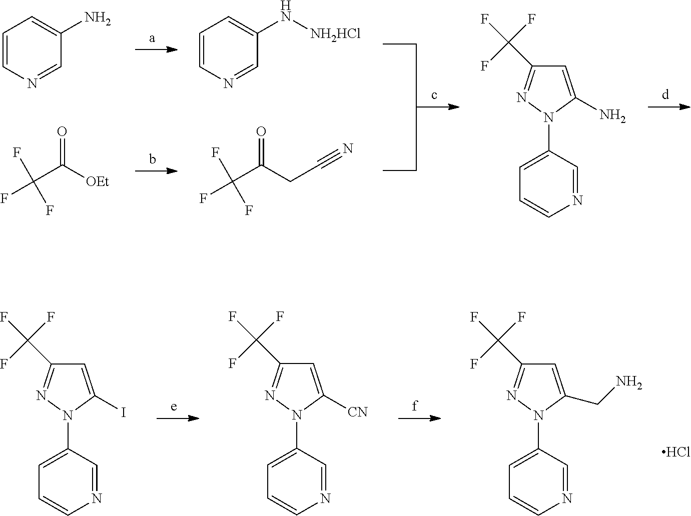

Step a: To a cold solution of pyridin-3-amine (40 g, 425.5 mmol) in conc. HCl (500 mL) at 0° C., a solution of NaNO₂ (35.23 g, 510.6 mmol) in water (40 mL) was added dropwise maintaining the temperature at 0° C. for 15 minutes. After addition the solution was stirred for 20 minutes. This solution was added to a solution of SnCl₂ (177.5 g, 936.3 mmol) in conc. HCl (100 mL) dropwise maintaining the temperature at 0° C. for 20 minutes and the resulting yellow solution was stirred at 0° C. for 30 minutes. The obtained yellow solid was filtered, washed with water (3×50 mL) and dried afford product (106.5 g, crude) as yellow solid.

Step b: To a cold suspension of NaH (60% dispersion in oil, 29.26 g, 731.7 mmol) in 1,4-dioxane (450 mL), acetonitrile (38.46 mL, 731.7 mmol) was added dropwise at 0° C. and stirred for 30 minutes. The reaction mixture was cooled to −5° C., ethyl 2,2,2-trifluoroacetate (83.12 g, 585.36 mmol) was slowly added and the reaction mixture allowed to stir at room temperature for 16 h. The reaction mixture was cooled to 0° C., quenched with methanol (150 mL), diluted with ethyl acetate (300 mL) and pH adjusted to ˜4 using diluted aqueous HCl. The organic layer was separated and the aqueous layer was extracted with ethyl acetate (2×250 mL). The combined ethyl acetate layer was washed with water (250 mL), brine solution (200 mL), dried over sodium sulfate, filtered and concentrated under reduced pressure to afford a brown liquid (57 g). The crude compound was used as such without further purification.

Step c: A solution of step-b product (57 g, crude; 416.05 mmol) and step-a product (60.5 g, 416.05 mmol) in ethanol (650 mL) was stirred at reflux for 3 h. The reaction mixture was concentrated; the obtained residue was diluted with ethyl acetate (2 L), washed with water (2×500 mL), brine solution (500 mL), dried over sodium sulfate, filtered and concentrated under reduced pressure to give a residue. Purification by column chromatography (silica gel; 100-200 mesh; eluent: 30% ethyl acetate in petroleum ether) afforded a yellow solid (31.48 g).

Step d: To a cold suspension of potassium iodide (51.3 g, 309.21 mmol) and isoamyl nitrite (41.16 mL, 309.21 mmol) in dry acetonitrile (350 mL), a solution of step-c product (23.5 g, 103.07 mmol) in acetonitrile (100 mL) was added dropwise at 0° C. and the reaction mixture was stirred at 100° C. for 20 h. The reaction mixture was concentrated; the obtained residue was diluted with ethyl acetate (1 L), washed with water (2×400 mL), brine solution (200 mL), dried over sodium sulfate, filtered and concentrated to give a residue. Purification by column chromatography (silica gel; 100-200 mesh; eluent: 30% ethyl acetate in petroleum ether) afforded a pale yellow solid (16.52 g, 37%).

Step e: To a solution of step-d product (16.5 g, 48.67 mmol) in dry NMP (150 mL), CuCN (6.53 g, 73.0 mmol) was added and the reaction mixture was stirred at 200° C. for 2 h. The reaction mixture was cooled to room temperature, quenched with ethylene diamine (50 mL) and diluted with ethyl acetate (800 mL). The obtained suspension was filtered through celite bed, washed with ethyl acetate (2×100 mL). The combine filtrate was washed with water (2×300 mL), brine solution (250 mL), dried over sodium sulfate, filtered and concentrated under reduced pressure to give a residue. Purification by column chromatography (silica gel; 100-200 mesh; eluent: 20-30% ethyl acetate in petroleum ether) to afford a yellow solid (5.12 g, 44%).

Step f: To a solution of step-e product (4.5 g, 18.9 mmol) in saturated methanolic NH₃ (50 mL), Raney-Nickel (3 g, wet, washed with methanol (4×5 mL)) was added and the mixture was hydrogenated in a Parr hydrogenator at 40 Psi pressure at room temperature for 4 h. The reaction mixture was filtered through celite and the filtrate was concentrated under reduced pressure. The obtained residue was stirred in sat. HCl in ether (50 mL) for 2 h. Ether was decanted, the obtained solid was washed with ether (3×10 mL), vacuum dried to afford product compound as light brown solid (1.2 g, 23%).

7.5 Synthesis of (1-(4-methoxybenzyl)-3-(trifluoromethyl)-1H-pyrazol-5-yl)methanamine (employed e.g for the synthesis of example compound no. 86)

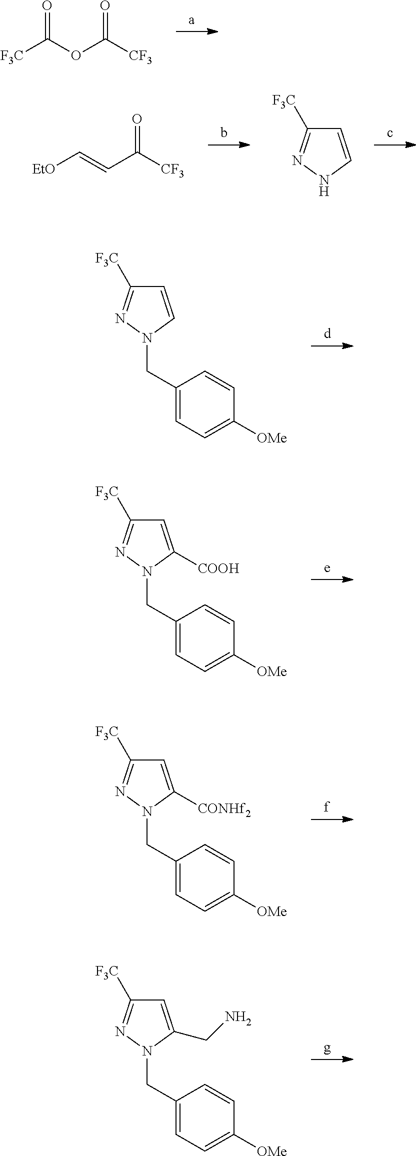

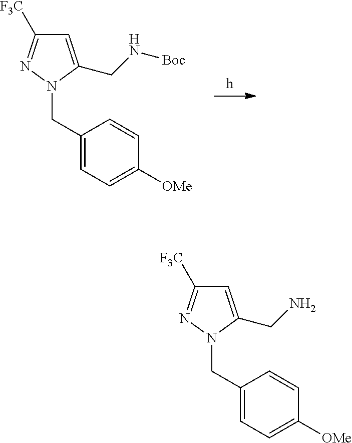 [SMILES: COc1ccc(Cn2nc(C(F)(F)F)cc2CNC(=O)OC(C)(C)C)cc1 ; COc1ccc(Cn2nc(C(F)(F)F)cc2CN)cc1]

Step a: DMAP (4.25 g, 0.034 mol, 0.01 equivalents) was added to dichloromethane (3 L) and cooled the contents to −10° C. Trifluoroacetic anhydride (765 g (510 mL), 3.2 mol, 1.05 equivalents) was added followed by ethyl vinyl ether (250 g, 3.04 mol) was added drop wise for 45 min at −10° C. Then the overall reaction mixture was initially stirred for 8 h at 0° C. and later for overnight at room temperature. Progress of the reaction was monitored by TLC (10% ethyl acetate/n-hexane). On completion of the reaction, reaction contents were quenched with saturated NaHCO₃ solution (600 mL) and organic layer was separated. Aqueous layer was extracted with dichloromethane (2×500 mL). Combined organic layer was washed with water (2×1 L), dried over sodium sulfate and concentrated under reduced pressure to obtain the crude product as a brown colored liquid (450 g, crude).

Step b: Hydrazine dihydrochloride (225 g, 2.14 mol, 1.6 equivalents) was taken in ethanol (1.4 L) and stirred well. triethylamine (135.4 g (185.4 mL), 1.34 mol, 1 equivalent) was added drop wise for 45 min at room temperature. Then step-a product (225 g, crude) was added drop wise at room temperature and the overall reaction mixture was refluxed for overnight. Progress of the reaction was monitored by TLC (20% ethyl acetate/n-hexane). On completion of the reaction, ethanol was distilled off completely, residue was taken in ice water (500 mL) and the product extracted with ethyl acetate (2×400 mL). Combined extract was washed with ice water (300 mL), dried over sodium sulfate and concentrated under reduced pressure to yield the required product as and off white solid (195 g).

Step c: NaH (33.08 g (19.85, 60%), 1.5 eq) was added to small quantity of n-hexane and stirred well for 10 min. N-hexane was decanted, dry dimethylformamide (500 mL) was added drop wise under N₂ atmosphere and stirred well. A solution of step-b product (75 g, 0.55 mol) in dimethylformamide (125 mL) was added drop wise under N₂ atmosphere. Then a solution of 4-methoxylbenzoyl chloride (86.3 g, 0.55 mol, 1 equivalent) in dimethylformamide (125 mL) was added drop wise and the overall reaction mixture was allowed to stir for 12 h at room temperature. Progress of the reaction was monitored by TLC (10% ethyl acetate in n-hexane). On completion of the reaction, reaction contents were poured into ice water (500 mL) and the product extracted with ethyl acetate (2×400 mL). Then the contents were dried over sodium sulfate and concentrated under reduced pressure to yield the required product as a brown colored liquid (125 g, 88%).

Step d: Diisopropyl amine (28.4 (39.4 mL), 1.2 equivalents) was taken in tetrahydrofuran (500 mL), stirred well and cooled the contents to 0° C. n-BuLi (234.4 mL, 1.5 eq) was added drop wise at 0° C. and cooled the contents to −78° C. A solution of step-c product (62 g, 0.24 mol) in tetrahydrofuran (200 mL) was added drop wise for 30 min and stirred the contents for another 30 min at −78° C. Then dry CO₂ gas was bubbled through the reaction mixture for 1.5 h and the progress of the reaction was monitored by TLC (10% ethyl acetate/n-hexane). On completion of the reaction, reaction contents were poured into ice water (300 mL) and the aqueous layer was extracted with ethyl acetate (2×200 mL) in basic condition. Aqueous layer was acidified with 20% HCl solution and extracted with ethyl acetate (2×200 mL). Combined organic layer was dried over sodium sulfate and concentrated under reduced pressure to yield the required product as an off white solid (42 g, 58%).

Step e: To a solution of step-d product (50 g, 0.16 mol) in dichloromethane (750 mL), catalytic amount of dimethylformamide was added and cooled to 0° C. Thionyl chloride (99.3 g (61 mL), 0.83 mol, 5 equivalents) was added drop wise for 30 min at 0° C. Overall reaction mixture was slowly heated to a reflux temperature and allowed to reflux for 2 h. Progress of the reaction was monitored by TLC (10% ethyl acetate/n-hexane). On disappearance of the starting material, dichloromethane was distilled off completely. Above prepared acid chloride was dissolved in dichloromethane (500 mL) and added drop wise to aqueous ammonia solution (600-700 mL) at 0° C. Overall reaction mixture was allowed to stir for 1 h and the progress of the reaction was monitored by TLC (10% ethyl acetate/n-hexane, Rf˜0.7). On completion of the reaction, ice cold water (200 mL) was added and the product extracted with ethyl acetate (2×200 mL). Combined organic layer was dried over sodium sulfate and concentrated under reduced pressure to yield the required product as an off white solid (37 g, crude). Crude obtained was directly used for the next step.

Step f: LAH (4.7 g, 0.12 mol, 1 equivalent) was added to small quantity of n-hexane and stirred well for 10 min. N-hexane was decanted and tetrahydrofuran (250 mL) was added to LAH under cold condition. Then a solution of step-e product (37 g, 0.12 mol) in tetrahydrofuran (120 mL) was added drop wise for 30 min at 0° C. and reaction mixture was heated to reflux for 5 h. Progress of the reaction was monitored by TLC (50% ethyl acetate/n-hexane). As the reaction moved completely, LAH (2.3 g) was added and refluxed for another 4 h. This time reaction was moved completely. Then the reaction contents were slowly added to saturated solution of sodium sulfate (1 L) and the product extracted with ethyl acetate (2×500 mL). Combined extract was dried over sodium sulfate and concentrated under reduced pressure to obtain the crude product as an off white solid (32.5 g). Crude obtained was directly used for the next step.

Step g: To a solution of step-f product ((80 g, 0.28 mol) in dichloromethane (600 mL) cooled at 0° C., triethylamine (22.7 g (30.2 mL), 0.026 mol, 0.8 equivalents) was added drop wise for 10 min. Then Boc anhydride (61.2 g (62.5 mL), 0.28 mol, 1 eq) taken in dichloromethane (200 mL) was added drop wise for 20 30 min at 0° C. Overall reaction mixture initially stirred for 30 min at 0° C. and alter for another 30 min at room temperature. Progress of the reaction was monitored by the TLC (20% ethyl acetate/n-hexane). On completion of the reaction, dichloromethane was distilled off completely, residue was taken in ice water (500 mL) and the product extracted with ethyl acetate (2×300 mL). Combined extract was dried over sodium sulfate and concentrated under reduced pressure. Crude obtained was recrystalised from n-hexane (200 mL) to yield the required product as an off white solid (80 g, 74%).

Step h: Step-g (5 g, 0.012 mol) product was taken in dichloromethane (30 mL) and cooled to 0° C. HCl gas was bubbled through the reaction mixture for 45 min at 0° C. Progress of the reaction was monitored by TLC (30% ethyl acetate/n-hexane). On completion of the reaction, dichloromethane was distilled off completely. Residue was taken in ice water (200 mL) and the product extracted with 20% ethyl acetate/n-hexane (2×100 mL). Aqueous layer was basified to a pH˜10 with 2N NaOH solution and extracted with ethyl acetate (5×100 mL). Combined organic layer was washed with water (2×200 mL), dried over sodium sulfate and concentrated under reduced pressure to yield the required product as an yellow colored liquid (2.4 g, 64%).

### Synthesis of the Example Compounds

### Preparation of amides (Z═C—R^{4b})

General directions for reacting amines of general formula (II) with carboxylic acids of general formula (III) or carboxylic acid derivatives of general formula (III) to form compounds of general formula (I), wherein Z═C—R^{4b} (amides), as in scheme 1 (step j09).

### Method A:

The acid of general formula (III) (1 equivalent), the amine of general formula (II) (1.2 equivalents) and EDCl (1.2 equivalents) are stirred in dimethylformamide (10 mmol of acid/20 mL) for 12 h at room temperature and water is subsequently added thereto. The reaction mixture is repeatedly extracted with ethyl acetate, the aqueous phase is saturated with NaCl and subsequently reextracted with ethyl acetate. The combined organic phases are washed with 1 N HCl and brine, dried over magnesium sulfate and the solvent is removed under vacuum. The residue is purified by means of flash chromatography (silica gel: 100-200 mesh, eluent: ethyl acetate/n-hexane in different ratios such as 1:2) and the product (I) is in this way obtained.

### Method B:

The acid of general formula (III) (1 equivalent) and the amine of general formulae (II) (1.1 equivalent) are dissolved in dichloromethane (1 mmol of acid in 6 mL) and mixed with EDCI (1.5 equivalents), h1-hydroxybenzotriazolhydrate (1.4 equivalents) and triethylamine (3 equivalents) at 0° C. The reaction mixture is stirred for 20 h at room temperature and the crude product is purified by means of column chromatography (silica gel: 100-200 mesh, eluent: n-hexane/ethyl acetate in different ratios such as 2:1) and (I) is in this way obtained.

### Method C:

The acid of general formula (III) with D=OH (1 equivalent) is first mixed with a chlorinating agent, preferably with thionyl chloride and the mixture obtained in this way is boiled under reflux and the acid (III) is in this way converted into the corresponding acid chloride (III) with D=Hal. The amine of general formulae (II) (1.1 equivalents) is dissolved in dichloromethane (1 mmol of acid in 6 mL) and mixed with triethylamine (3 equivalents) at 0° C. The reaction mixture is stirred for 20 h at room temperature and the crude product is purified by means of column chromatography (silica gel: 100-200 mesh, eluent:n-hexane/ethyl acetate in different ratios such as 2:1) and (I) is in this way obtained.

### Method D:

The phenyl ester (IIIa) obtained (1 equivalent) and the corresponding amine (II) (1.1 equivalents) are dissolved in tetrahydrofuran (10 mmol of the reaction mixture in 120 mL) and stirred for 16 h at room temperature after addition of DBU (1.5 equivalents). After removal of the solvent under vacuum, the residue obtained is purified by means of flash chromatography (silica gel: 100-200 mesh, eluent:ethyl acetate/n-hexane in different ratios such as 1:1) and (I) is in this way obtained.

### Preparation of Ureas (Z=N)

General directions for reacting amines of general formula (II) or (V) with phenyl chloroformate to form compounds of formula (IV) or (Va) (step j07 and step v1, respectively) and subsequent reaction of compounds of formula (V) with amines of general formula (VI) or of compounds of formula (VIa) with amines of general formula (II) to form compounds of general formula (I), wherein A=N, as in scheme 1a and 1c (step j08 and step v2, respectively):

Step j07/step v1:

The amine of general formula (II) or (V) (1 equivalent) is placed in dichloromethane (10 mmol of amine in 70 mL) and phenyl chloroformate (1.1 equivalents) is added thereto at room temperature and the mixture is stirred for 30 min. After removal of the solvent under vacuum, the residue is purified by means of flash chromatography (silica gel: 100-200 mesh, eluent: diethyl ether/n-hexane in different ratios such as 1:2) and (IV) or (Va) is in this way obtained.

Step j08/step v2:

The carbamic acid phenyl ester (IV) or (Va) obtained (1 equivalent) and the corresponding amine (V) or (II) (1.1 equivalents) are dissolved in tetrahydrofuran (10 mmol of the reaction mixture in 120 mL) and stirred for 16 h at room temperature after addition of DBU (1.5 equivalents). After removal of the solvent under vacuum, the residue obtained is purified by means of flash chromatography (silica gel: 100-200 mesh, eluent: ethyl acetate/n-hexane in different ratios such as 1:1) and (I) is in this way obtained.

The example compounds 1-19, 21-27, 30, 32, 50-51, 53-71, 79, 87-96, 98-115, 117, 120-121, 123-127 and 129-133 were obtained by one of the methods disclosed above and according to schemes 1 and 2. The example compounds 20, 28-29, 31, 33-49, 52, 72-78, 80-86, 97, 116, 118-119, 122, 128 and 134-135 can be obtained by one of the methods disclosed above. The person skilled in the art is aware which method has to be employed to obtain a particular example compound.

### Detailed Synthesis of Selected Example Compounds

### Synthesis of Example 1

### N-((1-(3-chlorophenyl)-3-(trifluoromethyl)-1H-pyrazol-5-yl)methyl)-2-(pyridin-2-yl)acetamide

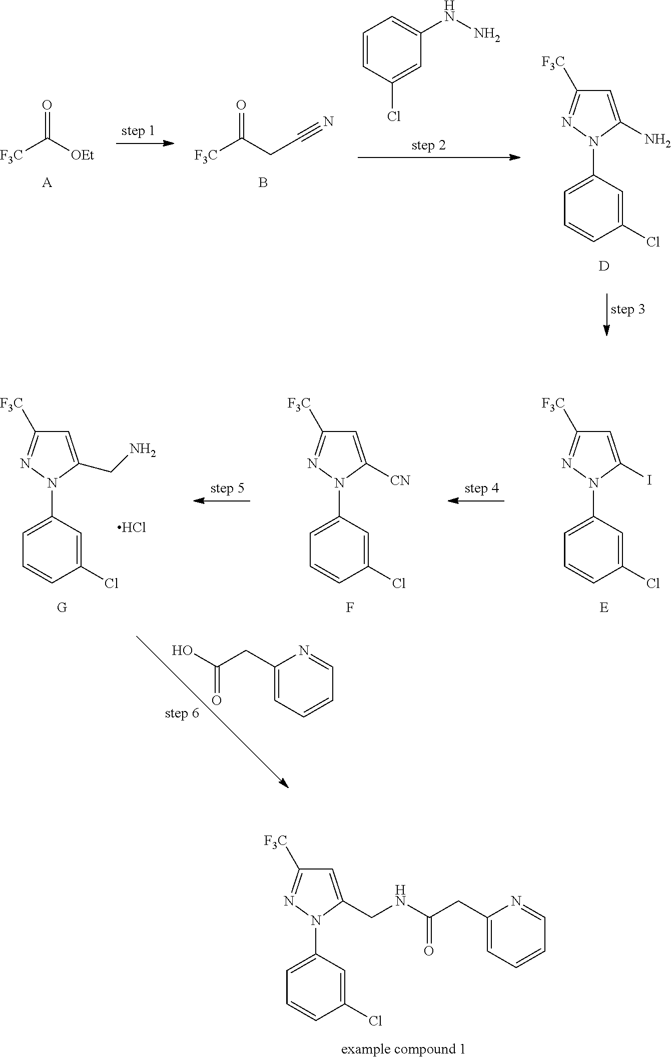

### Step 1

To a cold suspension of sodium hydride (60% dispersion in oil, 19.5 g, 487.5 mmol) in 1,4-dioxane (300 mL), acetonitrile (20 g, 487.5 mmol) was added dropwise at 0° C. and stirred for 30 min. The reaction mixture was cooled to −5° C., trifluoroethyl acetate (A) (55 g, 387.3 mmol) was slowly added and allowed to stir at room temperature for 16 h, until the complete consumption, as evidenced by GC analysis. The reaction mixture was cooled to 0° C., quenched with methanol (120 mL), diluted with ethyl acetate (200 mL) and pH adjusted to ˜4 using diluted aqueous HCl. The organic layer was separated and the aqueous layer was extracted with ethyl acetate (2×250 mL). The combined ethyl acetate layer was washed with water (250 mL), brine solution (200 mL), dried over sodium sulfate, filtered and concentrated under reduced pressure to give 4,4,4-trifluoro-3-oxobutanenitrile. The crude compound was used as such without further purification. This reaction was carried out in three batches (3×55 g) to afford crude 4,4,4-trifluoro-3-oxobutanenitrile (B) (75 g) as a brown liquid.

### Step 2

A solution of 4,4,4-trifluoro-3-oxobutanenitrile (B) (75.3 g, crude; 557 mmol (theoretical)) and 3-chloro phenyl hydrazine (C) (99.87 g, 557.7 mmol) in ethanol (1.1 L) was stirred at reflux for 3 h, until complete consumption, as evidenced by TLC analysis. The reaction mixture was concentrated; the obtained residue was diluted with ethyl acetate (1 L), washed with water (2×250 mL), brine solution (200 mL), dried over sodium sulfate, filtered and concentrated under reduced pressure to give a residue. Purification by column chromatography (silica gel: 100-200 mesh, eluent: 50-70% chloroforme in petrol ether) afforded 1-(3-chlorophenyl)-3-(trifluoromethyl)-1H-pyrazol-5-amine (D) (30.32 g, 10% over 2 steps) as a pale yellow solid.

### Step 3

To a cold suspension of potassium iodide (19.02 g, 114.55 mmol) and isoamyl nitrite (15.3 mL, 114.55 mmol) in dry acetonitrile (100 mL), a solution of 1-(3-chlorophenyl)-3-(trifluoromethyl)-1H-pyrazol-5-amine (D) (10 g, 38.16 mmol) in acetonitrile (50 mL) was added dropwise at 0° C. and the reaction mixture was stirred at 100° C. for 20 h, until the complete consumption, as evidenced by TLC analysis. The reaction mixture was concentrated; the obtained residue was diluted with ethyl acetate (1 L), washed with water (2×500 mL), brine solution (200 mL), dried over sodium sulfate, filtered and concentrated to give a residue. Purification by column chromatography (silica gel: 100-200 mesh, eluent: 5% ethyl acetate in petrol ether) afforded 1-(3-chlorophenyl)-5-iodo-3-(trifluoromethyl)-1H-pyrazole as a pale yellow solid. This reaction was carried out in three batches (3×10 g) to afford 1-(3-chlorophenyl)-5-iodo-3-(trifluoromethyl)-1H-pyrazole (E) (20.12 g, combined purification, 47%) as yellow solid.

### Step 4:

To a solution of 1-(3-chlorophenyl)-5-iodo-3-(trifluoromethyl)-1H-pyrazole (E) (20.12 g, 54.06 mmol) in dry N-methyl-2-pyrrolidone (200 mL), copper(I) cyanide (7.33 g, 81.84 mmol) was added and the reaction mixture was stirred at 200° C. for 2 h until complete consumption, as evidenced by TLC analysis. The reaction mixture was cooled to room temperature, quenched with ethylene diamine (50 mL) and diluted with ethyl acetate (200 mL). The obtained suspension was filtered through celite bed, washed with ethyl acetate (2×50 mL). The combine filtrate was washed with water (100 mL), brine solution (100 mL), dried over sodium sulfate, filtered and concentrated under reduced pressure to give a residue. Purification by column chromatography (silica gel: 100-200 mesh, eluent: petrol ether) to afford 1-(3-chlorophenyl)-3-(trifluoromethyl)-1H-pyrazole-5-carbonitrile (F) (10.12 g, 69%) as yellow solid.

### Step 5:

To a cold solution of 1-(3-chlorophenyl)-3-(trifluoromethyl)-1H-pyrazole-5-carbonitrile (F) (10.12 g, 37.13 mmol) in tetrahydrofuran (120 mL), boranedimethyl sulfide (22.6 mL, 241.35 mmol) was added at 0° C. and the reaction mixture was stirred at room temperature for 24 h, until completion, as evidenced by TLC analysis. The reaction mixture was cooled to room temperature, methanol (50 mL) was added slowly and resulting mixture heated at reflux for 30 min. The solvent was evaporated and the obtained residue was stirred in saturated HCl in diethyl ether (50 mL) for 2 h. The solid was filtered, washed with pentane (2×20 mL), 5% ethyl aceate/petrol ether (2×20 mL) and vacuum dried to afford hydrochloride of (1-(3-chlorophenyl)-3-(trifluoromethyl)-1H-pyrazol-5-yl)methanamine (G) (3.1 g, 27%) as white solid.

### Step 6:

To a stirred solution of (1-(3-chlorophenyl)-3-(trifluoromethyl)-1H-pyrazol-5-yl)methanamine (G) (75 mg, 0.275 mmol) and 2-(pyridin-2-yl)acetic acid (47 mg, 0.275 mmol) in tetrahydrofuran (2.1 mL) was added 1-hydroxybenzotriazolhydrate (0.037 mL, 0.275 mmol), O-(1H-benzotriazol-1-yl)-N,N,N′,N′-tetramethyluronium tetrafluoroborate (0.089 g, 0.275 mmol) and N-ethyldiisopropylamine (0.14 mL, 0.825 mmol) and dimethylformamide (0.1 mL). The reaction mixture was allowed to stir for 40 h. The reaction mixture was concentrated under reduced pressure and the solid obtained was purified by column chromatography (silica gel: 100-200 mesh, eluent: ethyl acetate/methanol 18:1) to afford N-((1-(3-chlorophenyl)-3-(trifluoromethyl)-1H-pyrazol-5-yl)methyl)-2-(pyridin-2-yl)acetamide (example compound 1) (102 mg, 94%) as a white solid.

Examples 6, 7, 10, 11, 13, 14, 16 and 17 were prepared in a similar manner by using commercial available substituted pyridines/pyrimidines.

### Synthesis of Example 2

### N-((3-tert-butyl-1-(3-chlorophenyl)-1H-pyrazol-5-yl)methyl)-2-(pyridin-2-yl)propanamide

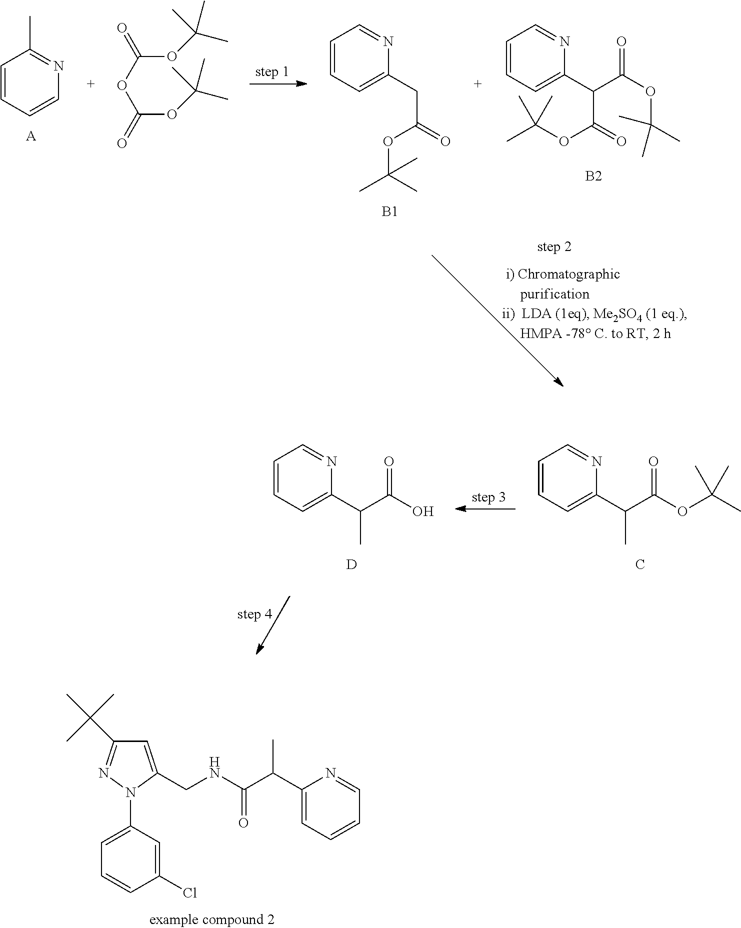

### Step 1:

To a stirred solution of diisopropylamine (10.8 g, 0.1 mol) in (20 mL) of dry tetrahydrofuran was added n-BuLi (49 mL, 2.04M, 0.10 mol) at −78° C. The reaction mixture was allowed to stir for 30 min. To this solution, 2-methylpyridine (A) (10 g, 0.107 mol) in (20 mL) of dry tetrahydrofuran was added drop wise. The reaction mixture was allowed to stir for 1 h at −78° C. To this di-tert-butyl dicarbonate (24 g, 0.11 mol) was added at −78° C. and was allowed to attain room temperature in 2 h. The reaction mixture was quenched with saturated ammonium chloride solution (50 mL), diluted with water (60 mL) and extracted with ethyl acetate (3×80 mL). The total organic layer was washed with brine (50 mL). The final organic layer was dried over magnesium sulfate and was concentrated under reduced pressure to obtain crude compound which was purified by column chromatography (silica gel: 100-200 mesh, eluent: 10% ethyl acetate in n-hexane) to afford tert-butyl 2-(pyridin-2-yl)acetate (B1) (6 g, 29%).

### Step 2:

To a stirred solution of diisopropylamine (1.56 g, 15.55 mmol) in dry tetrahydrofuran (5 mL) was added n-BuLi (7.6 mL, 2.04M, 15.55 mmol) at −78° C. The reaction mixture was allowed to stir for 30 min. To this solution, hexamethylphosphoramide (2.78 g, 15.55 mmol) and tert-butyl 2-(pyridin-2-yl)acetate (B1) (3 g, 15.55 mmol) dry tetrahydrofuran (5 mL) were added drop wise. The reaction mixture was allowed to stir for 1 h at −78° C. To this solution, dimethyl sulfate (1.95 g, 15.55 mol) in 5 mL of dry tetrahydrofuran was added at −78° C. and was allowed to attain ambient temperature in 2 h. The reaction mixture was quenched with saturated ammonium chloride solution (30 mL) and was diluted with water (50 mL) and was extracted with ethyl acetate (2×50 mL). The total organic layer was washed with brine (50 mL). The final organic layer was dried over magnesium sulfate and was concentrated under reduced pressure to obtain crude compound which was purified by using column chromatography (silica gel:100-200 mesh, eluent: 5% ethyl acetate in n-hexane) to afford tert-butyl 2-(pyridin-2-yl)propanoate (C) (1.8 g, 56%).

### Step 3:

To tert-butyl 2-(pyridin-2-yl)propanoate (C) (2.5 g, 12.07 mmol), 6N HCl (65 mL) was added and was allowed to stir for 12 h. The reaction mixture was concentrated under reduced pressure to obtain crude compound which was co-distilled with benzene (3×10 mL) to obtain 2-(pyridin-2-yl)propanoic acid (D) (1.6 g).

¹H NMR (DMSO-d₆, 400 MHz): 1.54 (d, 3H), 4.27 (d, 1H), 7.78 (t, 1H), 7.80 (d, 1H), 8.38 (t, 1H), 8.76 (d, 1H)

### Step 4:

To a stirred solution of 2-(pyridin-2-yl)propanoic acid (D) (0.097 g, 0.648 mmol) and (3-tert-butyl-1-(3-chlorophenyl)-1H-pyrazol-5-yl)methanamine (0.114 g, 0.432 mmol) in tetrahydrofuran (3.5 mL) was added 1-hydroxybenzotriazolhydrate (0.06 mL, 0.432 mmol), O-(1H-benzotriazol-1-yl)-N,N,N′,N′-tetramethyluronium tetrafluoroborate (0.139 g, 0.432 mmol) and N-ethyldiisopropylamine (0.22 mL, 1.296 mmol) to gave an suspension. After addition of dimethylformamide (1.3 mL) the reaction mixture was stirred for 36 h. The reaction mixture was concentrated under reduced pressure and the solid obtained was purified by column chromatography (silica gel: 100-200 mesh, eluent: ethyl acetate/cyclohexane 90:1) to afford N-((3-tert-butyl-1-(3-chlorophenyl)-1H-pyrazol-5-yl)methyl)-2-(pyridin-2-yl)propanamide (example compound 2) (31 mg, 18%).

Example 8 was prepared in a similar manner by using commercial available corresponding substituted pyridine.

### Synthesis of Example 3

### N-((1-(3-chlorophenyl)-3-(trifluoromethyl)-1H-pyrazol-5-yl)methyl)-2-(pyridin-2-yl)propanamide

To obtain example compound 3 reaction steps 1-3 as described for example compound 2 can be carried out followed by step 4:

### Step 4:

To a stirred solution of 2-(pyridin-2-yl)propanoic acid (0.075 g, 0.496 mmol) and ((1-(3-chlorophenyl)-3-(trifluoromethyl)-1H-pyrazol-5-yl)methanamine (0.091 g, 0.331 mmol) in tetrahydrofuran (2.5 mL) was added 1-hydroxybenzotriazolhydrate (0.045 mL, 0.331 mmol), O-(1H-benzotriazol-1-yl)-N,N,N′,N′-tetramethyluronium tetrafluoroborate (0.107 g, 0.331 mmol) and N-ethyldiisopropylamine (0.169 mL, 0.993 mmol) to gave an suspension. After addition of N,N-dimethylformamide (1 mL) the reaction mixture was stirred for 36 h. The reaction mixture was concentrated under reduced pressure and the solid obtained was purified by column chromatography (silica gel: 100-200 mesh, eluent: ethyl acetate/dichloromethane 10:1) to afford N-((1-(3-chlorophenyl)-3-(trifluoromethyl)-1H-pyrazol-5-yl)methyl)-2-(pyridin-2-yl)propanamide (example compound 3) (18 mg, 13%) as an off-white solid.

### Synthesis of Example 4

### 1-((3-tert-butyl-1-(3-chlorophenyl)-1H-pyrazol-5-yl)methyl)-3-(pyridin-2-yl)urea

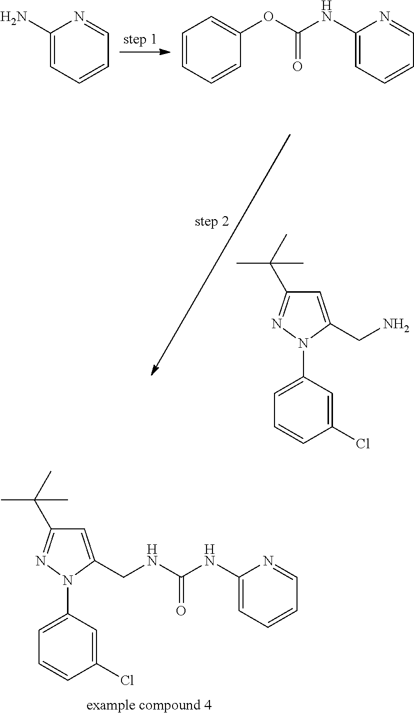 [SMILES: Nc1ccccn1 ; O=C(Nc1ccccn1)Oc1ccccc1 ; CC(C)(C)c1cc(CN)n(-c2cccc(Cl)c2)n1 ; CC(C)(C)c1cc(CNC(=O)Nc2ccccn2)n(-c2cccc(Cl)c2)n1]

### Step 1:

To a solution of 2-amino pyridine (400 mg, 4.25 mmol) in tetrahydrofuran and acetonitrile (3:4, 50 mL) was slowly added phenyl chloroformate (0.8 mL, 6.376 mmol) and pyridine (0.4 mL, 5.525 mmol) at room temperature. The reaction mixture was stirred for 3 h. TLC showed complete consumption of starting material. After adding water, the mixture was extracted with ethyl acetate. The extract was dried over magnesium sulfate and concentrated under reduced pressure. The crude residue was purified by column chromatography (silica gel: 100-200 mesh, eluent: n-hexane/ethyl acetate 4:1) to give the phenyl pyridin-2-ylcarbamate (710 mg, 78%).

### Step 2:

To a solution of phenyl pyridin-2-ylcarbamate (70 mg, 0.327 mmol) in acetonitrile (20 mL) was added DMAP (40 mg, 0.327 mmol) and (3-tert-butyl-1-(3-chlorophenyl)-1H-pyrazol-5-yl)methanamine (112 mg, 0.425 mmol) at room temperature. The reaction mixture was heated to 50° C. for 15 h. TLC showed complete consumption of starting material. The reaction mixture was diluted with water and extracted with ethyl acetate. The organic part was washed with water and brine. The organic layer was dried over magnesium sulfate and concentrated under reduced pressure. The crude was purified by column chromatography (silica gel: 100-200 mesh, eluent: n-hexane/ethyl acetate 1:1) to give 1-((3-tert-butyl-1-(3-chlorophenyl)-1H-pyrazol-5-yl)methyl)-3-(pyridin-2-yl)urea (example compound 4) (62 mg, 49%).

¹H NMR (300 MHz, CDCl₃): δ 8.11 (dd, 1H, J=5.13 Hz, Ar—H), 7.97 (s, 1H, Ar—NH), 7.59 (m, 1H, Ar—H), 7.56 (m, 1H, Ar—H), 7.36 (m, 3H, Ar—H), 6.89 (dd, 1H, J=5.1 Hz, Ar—H), 6.70 (d, 1H, J=8.25 Hz, Ar—H), 6.33 (s, 1H, Ar—H), 4.63 (d, 2H, J=5.67 Hz, Ar—CH₂), 1.33 (S, 9H, Ar—(CH₃)₃).

Examples 9, 12, 15, 18 19, 54 and 136 138 were prepared in a similar manner by using commercial available substituted pyridines/pyrimidines. Example 20 can be prepared in a similar manner.

### Synthesis of Example 5

### 1-((1-(3-chlorophenyl)-3-(trifluoromethyl)-1H-pyrazol-5-yl)methyl)-3-(pyridin-2-yl)urea

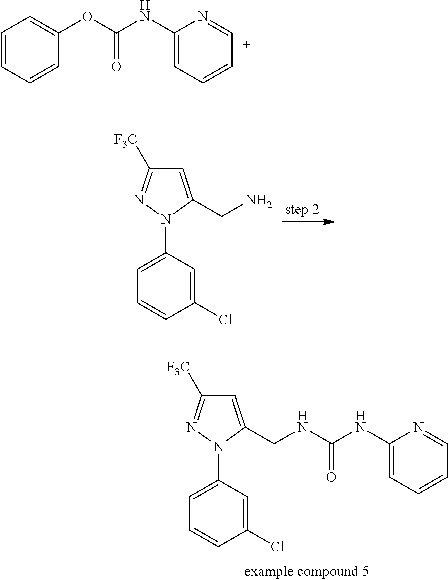 [SMILES: O=C(Nc1ccccn1)Oc1ccccc1 ; NCc1cc(C(F)(F)F)nn1-c1cccc(Cl)c1 ; O=C(NCc1cc(C(F)(F)F)nn1-c1cccc(Cl)c1)Nc1ccccn1]

To obtain example compound 5 reaction step 1 as described for example compound 4 can be carried out followed by step 2:

### Step 2:

To a solution of phenyl pyridin-2-ylcarbamate (70 mg, 0.327 mmol) in acetonitrile (20 mL) was added DMAP (40 mg, 0.327 mmol) and (1-(3-chlorophenyl)-3-(trifluoromethyl)-1H-pyrazol-5-yl)methanamine (117 mg, 0.425 mmol) at room temperature. The reaction mixture was heated to 50° C. for 15 h. TLC showed complete consumption of starting material. The reaction mixture was diluted with water and extracted with ethyl acetate. The organic part was washed with water and brine. The organic layer was dried over magnesium sulfate and concentrated under reduced pressure. The crude was purified by column chromatography (silica gel: 100-200 mesh, eluent: n-hexane/ethyl acetate 1:1) to give 1-((3-tert-butyl-1-(3-chlorophenyl)-1H-pyrazol-5-yl)methyl)-3-(pyridin-2-yl)urea (example compound 5) (61 mg, 47%).

¹H NMR (300 MHz, CDCl₃) δ 8.13 (dd, 1H, J=6.96 Hz, Ar—H), 7.62 (m, 1H, Ar—H), 7.56 (m, 1H, Ar—H), 7.45 (m, 3H, Ar—H), 6.93 (dd, 1H, J=6.6 Hz, Ar—H), 6.72 (s, 1H, Ar—NH), 6.64 (d, 1H, J=8.25 Hz, Ar—H), 4.63 (d, 2H, J=5.67 Hz, Ar—CH₂)

### Synthesis of Example 21

### 1-((1-(3-chlorophenyl)-3-(trifluoromethyl)-1H-pyrazol-5-yl)-methyl)-3-(6-(2-(methylsulfonyl)ethyl)pyridin-3-yl)urea

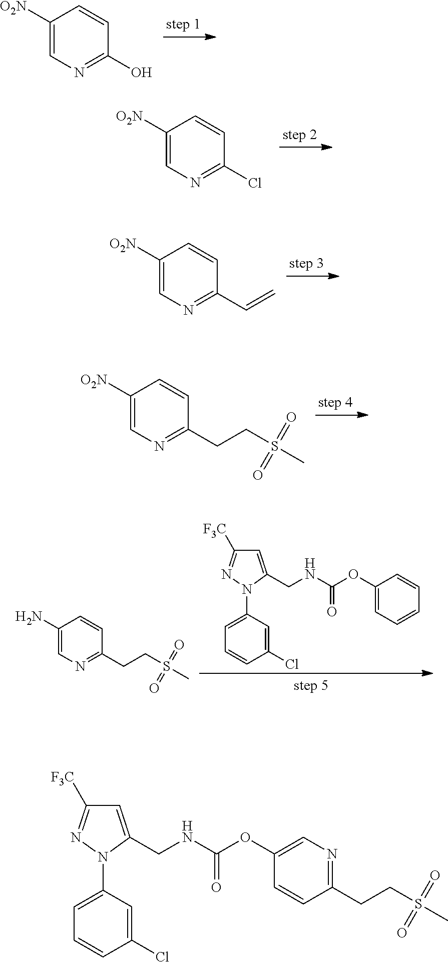

### Step 1:

To a stirred solution of 5-nitropyridin-2-ol (5 g, 35.71 mmol) in phosphorous oxychloride (50 mL), phosphorous pentachloride (11.15 g, 53.54 mmol) was added portionwise under heating at 60° C. and the reaction mass was stirred overnight at 60° C. TLC showed complete consumption of starting material after 16 h and the reaction mass was concentrated under reduced pressure to remove excess phosphorous oxychloride. The residue was poured into ice and extracted with ethyl acetate (3×100 mL). The combined organic layer was washed with brine (50 mL). The organic layer was dried over anhydrous magnesium sulfate and concentrated under reduced pressure to obtain 2-chloro-5-nitropyridine (5 g, 89%) as solid.

### Step 2:

To a stirred suspension of Pd₂(dba)₃ (144 mg, 0.15 mmol) and trifuryl phosphine (73 mg, 0.31 mmol) in tetrahydrofuran (3 mL) was added 2-chloro-5-nitropyridine (500 mg, 3.16 mmol) in tetrahydrofuran (2 mL) followed by tributylvinyl tin (1.2 g, 3.78 mmol). The reaction mixture was degasified and slowly heated to 60° C. and stirred overnight at that temperature. TLC showed complete consumption of starting material. The reaction mass was cooled to room temperature and diluted with water. It was then extracted with ethyl acetate (50 mL). The combined organic layer was washed with brine (2×50 mL) and dried over anhydrous magnesium sulfate. The solvent was concentrated under reduced pressure to afford the crude compound. The crude compound was purified by column chromatography (silica gel: 100-200 mesh, eluent: n-hexane) to 5-nitro-2-vinylpyridine (350 mg, 74%).

### Step 3:

To a stirred solution of 5-nitro-2-vinylpyridine (350 mg, 2.33 mmol) in ethanol (3.5 mL) was added sodium methane sulfinate (2.37 g, 23.21 mmol) at room temperature followed by addition of acetic acid (140 mg, 2.33 mmol). The reaction mass was refluxed at 60° C. for 14 h. The reaction mixture was cooled to room temperature and was concentrated under reduced pressure to obtain crude compound. It was washed with water (2×10 mL) and filtered through sintered funnel to afford 2-(2-(methylsulfonyl)ethyl)-5-nitropyridine (500 mg, 92%).

### Step 4:

To a stirred solution of 2-(2-(methylsulfonyl)ethyl)-5-nitropyridine (400 mg, 1.73 mmol) in ethyl acetate (8 mL) was added 10% Pd/C (40 mg). The reaction mass stirred for 6 h under hydrogen atmosphere. TLC showed complete consumption of starting material. The reaction mass was filtered and the filtrate was concentrated under reduced pressure afford solid compound which was upon washing with 20% ethyl acetate in n-hexane afforded 6-(2-(methylsulfonyl)ethyl)pyridin-3-amine (300 mg. 86%).

¹H NMR (DMSO-d₆, 400 MHz): 7.86 (s, 1H), 6.98 (d, 1H), 6.86 (dd, 1H), 5.16 (s, 2H), 3.40 (t, 2H), 2.95 (m, 5H)

### Step 5:

To a stirred solution 6-(2-(methylsulfonyl)ethyl)pyridin-3-amine (41 mg, 0.203 mmol) in tetrahydrofuran (3 mL) was added N-ethyldiisopropylamin (0.095 mL, 0.551 mmol) followed by phenyl (1-(3-chlorophenyl)-3-(trifluoromethyl)-1H-pyrazol-5-yl)methylcarbamate (75 mg, 0.19 mmol) at 150° C. and stirred for 1 h under microwave conditions (7 bar). The concentrated reaction mixture was purified by column chromatography (silica gel: 100-200 mesh, eluent: ethyl acetate/methanol 9:1) to get 1-((1-(3-chlorophenyl)-3-(trifluoromethyl)-1H-pyrazol-5-yl)methyl)-3-(6-(2-(methylsulfonyl)ethyl)pyridin-3-yl)urea (example compound 21) (44 mg, 46%) as white solid.

### Synthesis of Example 24

### 1-((1-(3-chlorophenyl)-3-cyclopropyl-1H-pyrazol-5-yl)methyl)-3-(5-fluoro-6-(2-(methylsulfonyl)ethyl)pyridin-3-yl)urea

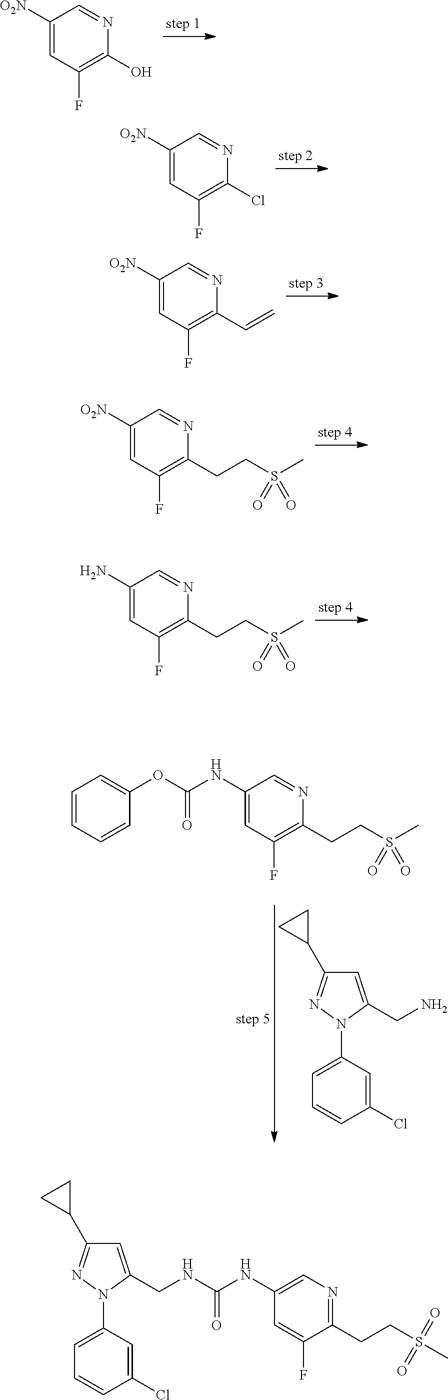

### Step 1:

To a stirred solution of 3-fluoro-5-nitropyridin-2-ol (1.5 g, 9.48 mmol) in phosphorous oxychloride (15 mL) was added phosphorous pentachloride (2.96 g, 14.22 mmol) at 60° C. The reaction mixture was allowed to stir for 10 h at the same temperature. The reaction mixture was cooled to room temperature and was poured into crushed ice and was extracted with ethyl acetate (3×20 mL). The total organic layer was washed with saturated sodium carbonate solution (25 mL). The washed organic layer was dried over anhydrous magnesium sulfate and was concentrated under reduced pressure to obtain crude compound which was purified by using column chromatography (silica gel: 100-200 mesh, eluent: 5% ethyl acetate in n-hexane) to afford 2-chloro-3-fluoro-5-nitropyridine (1.62 g, 97%).

### Step 2:

To a stirred solution of 2-chloro-3-fluoro-5-nitropyridine (1.6 g, 9.0 mmol) in tetrahydrofuran (16 mL) under nitrogen atmosphere were added tributylvinyl tin (3.42 g, 10.8 mmol), Pd₂(dba)₃ (0.42 g, 0.45 mmol) and trifuryl phosphene (0.2 g, 0.9 mmol). The reaction mixture was deoxygenated thoroughly and was heated to 60° C. for 6 h. The reaction mixture was diluted with water (20 mL) and extracted with ethyl acetate (3×25 mL). The combined organic layer was washed with brine (25 mL) and dried over anhydrous magnesium sulfate and concentrated under reduced pressure to afford the crude compound. The crude compound was purified by column chromatography (silica gel: 100-200 mesh; eluent: 5% ethyl acetate in n-hexane) to afford 3-fluoro-5-nitro-2-vinylpyridine (1.5 g, 96%).

### Step 3:

To a stirred solution of 3-fluoro-5-nitro-2-vinylpyridine (1.5 g, 8.92 mmol) in ethanol (15 mL) was added sodium methane sulfinate (9.1 g, 89.3 mmol) and acetic acid (0.53 g, 8.92 mmol) at room temperature. The reaction mixture was heated to 60° C. for 10 h. The reaction mixture was cooled to room temperature and concentrated under reduced pressure to obtain crude compound which was filtered. The obtained solid was washed with water (25 mL) to afford 3-fluoro-2-(2-(methylsulfonyl)ethyl)-5-nitropyridine (0.81 g, 36%).

### Step 4:

To 3-fluoro-2-(2-(methylsulfonyl)ethyl)-5-nitropyridine (0.8 g, 3.22 mmol) dissolved in ethyl acetate (8 mL), was added 10% Pd/C (80 mg) under argon atmosphere which was subjected to hydrogenated in Parr apparatus and the reaction was continued to stir for 2 h. The reaction mixture was filtered through celite bed, washed thoroughly with ethyl acetate and concentrated under reduced pressure to afford 5-fluoro-6-(2-(methylsulfonyl)ethyl)pyridin-3-amine (0.62 g, 88%).

¹H NMR (DMSO-d₆, 400 MHz): δ 7.73 (s, 1H), 6.72 (dd, 1H), 5.55 (s, 2H), 3.41 (t, 2H), 2.98-3.02 (m, 5H)

### Step 5

5-Fluoro-6-(2-(methylsulfonyl)ethyl)pyridin-3-amine (100 mg, 0.458 mmol) was dissolved in dichloromethane (2.5 mL). Triethylamine (0.076 mL, 0.55 mmol) and phenyl chloroformate (0.065 mL, 0.513 mmol) were added, the reaction mixture was stirred at room temperature for 12 h. The reaction mixture was extracted with saturated sodium carbonate solution (10 mL). The aqueous layer was extracted with dichloromethane (2×20 mL). The combined organic layers were dried over anhydrous magnesium sulfate and concentrated under reduced pressure to obtain crude compound which was purified by using column chromatography (silica gel: 100-200 mesh, eluent: ethyl acetate/n-cyclohexane 3:1) to afford phenyl 5-fluoro-6-(2-(methylsulfonyl)ethyl)pyridin-3-ylcarbamate (62 mg, 40%).

### Step 6:

To a stirred solution (1-(3-chlorophenyl)-3-cyclopropyl-1H-pyrazol-5-yl)methanamine (50 mg, 0.204 mmol) in tetrahydrofuran (3 mL) was added N-ethyldiisopropylamin (0.1 mL, 0.592 mmol) followed by phenyl 5-fluoro-6-(2-(methylsulfonyl)ethyl)pyridin-3-ylcarbamate (76 mg, 0.224 mmol) at 150° C. and stirred for 1 h under microwave conditions (7 bar). The concentrated reaction mixture was purified by column chromatography (silica gel: 100-200 mesh, eluent: ethyl acetate/methanol 19:1) to get 1-((1-(3-chlorophenyl)-3-cyclopropyl-1H-pyrazol-5-yl)methyl)-3-(5-fluoro-6-(2-(methylsulfonyl)ethyl)pyridin-3-yl)urea (example compound 24) (34 mg, 34%) as an orange solid.

Examples 22 and 23 were prepared in a similar manner.

### Synthesis of Example 25

### 5-(3-((3-tert-butyl-1-(3-chlorophenyl)-1H-pyrazol-5-yl)methyl)ureido)picolinamide

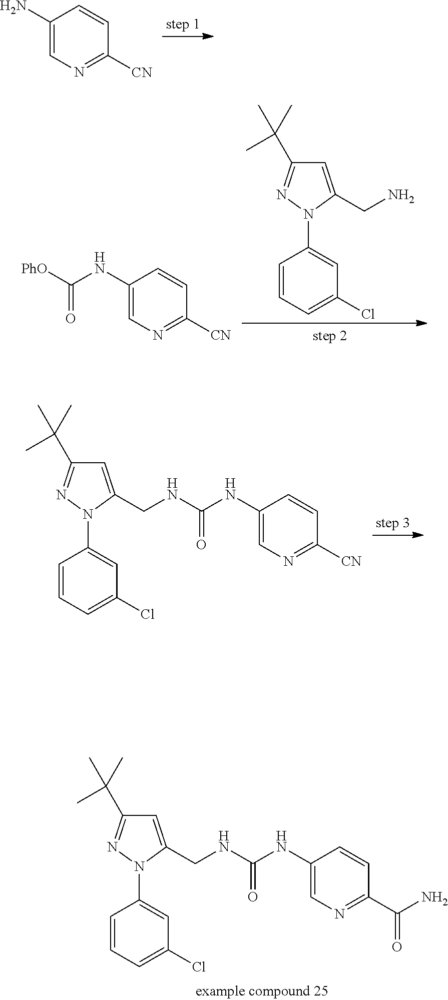 [SMILES: N#Cc1ccc(N)cn1 ; N#Cc1ccc(NC(=O)Oc2ccccc2)cn1 ; CC(C)(C)c1cc(CN)n(-c2cccc(Cl)c2)n1 ; CC(C)(C)c1cc(CNC(=O)Nc2ccc(C#N)nc2)n(-c2cccc(Cl)c2)n1 ; CC(C)(C)c1cc(CNC(=O)Nc2ccc(C(N)=O)nc2)n(-c2cccc(Cl)c2)n1]

### Step 1:

5-Amino-2-cyanopyridine (500 mg, 4.20 mmol) was dissolved in tetrahydrofuran and acetonitrile (ratio 1:1). To the reaction mixture was added pyridine (0.37 mL, 4.62 mmol, 1.1 eq) and phenyl chloroformate (0.55 mL, 4.41 mmol, 1.05 eq) and stirred at room temperature for 2 h. The reaction mixture was diluted with water and extracted with ethyl acetate. The organic part was washed with water and brine. The organic layer was dried over magnesium sulfate and concentrated under reduced pressure. The crude was purified by column chromatography to give pure phenyl 6-cyanopyridin-3-ylcarbamate (880 mg, 88%).

### Step 2:

To a solution of phenyl 6-cyanopyridin-3-ylcarbamate (150 mg, 0.63 mmol, 1.05 eq) in MeCN was added 4-dimethylaminopyridine (80 mg, 0.66 mmol, 1.1 eq) and (3-tert-butyl-1-(3-chlorophenyl)-1H-pyrazol-5-yl)methanamine (157 mg, 0.60 mmol, 1 eq) at room temperature. The reaction mixture was heated to 50° C. for overnight. The reaction mixture was diluted with water and extracted with ethyl acetate. The organic part was washed with water and brine. The organic layer was dried over magnesium sulfate and concentrated under reduced pressure. The crude was purified by column chromatography to 1-((3-tert-butyl-1-(3-chlorophenyl)-1H-pyrazol-5-yl)methyl)-3-(6-cyanopyridin-3-yl)urea (220 mg, 90%).

### Step 3:

1-((3-Tert-butyl-1-(3-chlorophenyl)-1H-pyrazol-5-yl)methyl)-3-(6-cyanopyridin-3-yl)urea (220 mg, 0.54 mmol) was dissolved in sulfuric acid (2.9 mL). The reaction mixture was stirred for 2 h at 60° C. and then cooled to room temperature. The reaction mixture was diluted with ice water and neutralized (pH=7) with 2M NaOH solution. The mixture was extracted with ethyl acetate. The organic layer was dried over magnesium sulfate and concentrated under reduced pressure. The crude was purified by column chromatography to give 5-(3-((3-tert-butyl-1-(3-chlorophenyl)-1H-pyrazol-5-yl)methyl)ureido)picolinamide (example compound 25) (90 mg, 39%).

¹H NMR (300 MHz, DMSO-d₆) δ 9.11 (br.s, NH), 8.59 (m, 1H, Ar—H), 7.94 (m, 3H, Ar—H), 7.52 (m, 5H, Ar—H), 6.92 (m, NH), 6.33 (s, 1H, pyrazole-H), 4.42 (d, 2H, J=5.67 Hz, Ar—CH₂), 1.26 (s, 9H, t-butyl-H).

### Synthesis of Example 26

### 5-(3-((1-(3-chlorophenyl)-3-(trifluoromethyl)-1H-pyrazol-5-yl)methyl)ureido)picolinamide

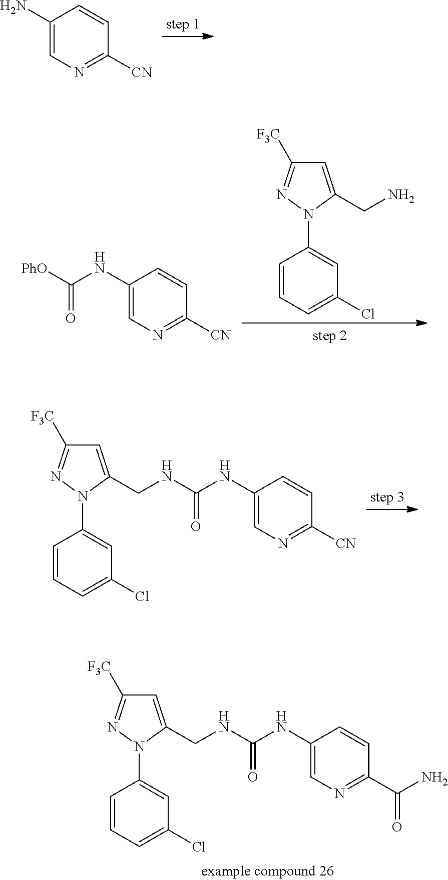 [SMILES: N#Cc1ccc(N)cn1 ; N#Cc1ccc(NC(=O)Oc2ccccc2)cn1 ; NCc1cc(C(F)(F)F)nn1-c1cccc(Cl)c1 ; N#Cc1ccc(NC(=O)NCc2cc(C(F)(F)F)nn2-c2cccc(Cl)c2)cn1 ; NC(=O)c1ccc(NC(=O)NCc2cc(C(F)(F)F)nn2-c2cccc(Cl)c2)cn1]

### Step 1:

5-Amino-2-cyanopyridine (500 mg, 4.20 mmol) was dissolved in tetrahydrofuran and acetonitrile (ratio 1:1). The reaction mixture was added pyridine (0.37 mL, 4.62 mmol, 1.1 eq) and phenyl chloroformate (0.55 mL, 4.41 mmol, 1.05 eq) and stirred at room temperature for 2 h. The reaction mixture was diluted with water and extracted with ethyl acetate. The organic part was washed with water and brine. The organic layer was dried over magnesium sulfate and concentrated under reduced pressure. The crude was purified by column chromatography to give phenyl 6-cyanopyridin-3-ylcarbamate (880 mg, 88%).

### Step 2:

To a solution of phenyl 6-cyanopyridin-3-ylcarbamate (150 mg, 0.63 mmol, 1.05 eq) in acetonitrile was added 4-dimethylaminopyridine (80 mg, 0.66 mmol, 1.1 eq) and (1-(3-chlorophenyl)-3-(trifluoromethyl)-1H-pyrazol-5-yl)methanamine (165 mg, 0.60 mmol, 1 eq) at room temperature. The reaction mixture was heated to 50° C. for overnight. The reaction mixture was diluted with water and extracted with ethyl acetate. The organic part was washed with water and brine. The organic layer was dried over magnesium sulfate and concentrated under reduced pressure. The crude was purified by column chromatography to give 1-((1-(3-chlorophenyl)-3-(trifluoromethyl)-1H-pyrazol-5-yl)methyl)-3-(6-cyanopyridin-3-yl)urea (220 mg, 88%).

### Step 3:

1-((1-(3-Chlorophenyl)-3-(trifluoromethyl)-1H-pyrazol-5-yl)methyl)-3-(6-cyanopyridin-3-yl)urea (220 mg, 0.52 mmol) was dissolved in sulfuric acid (2.8 mL). The reaction mixture was stirred for 2 h at 60° C. and then cooled to room temperature. The reaction mixture was diluted with ice water and neutralized (pH=7) with 2M NaOH solution. The mixture was extracted with ethyl acetate. The organic layer was dried over magnesium sulfate and concentrated under reduced pressure. The crude was purified by column chromatography to give 5-(3-((1-(3-chlorophenyl)-3-(trifluoromethyl)-1H-pyrazol-5-yl)methyl)ureido)picolinamide (example compound 26) (80 mg, 35%).

¹H NMR (300 MHz, Acetone-d₆) δ 8.64 (m, 2H, Ar—H, NH), 8.10 (m, 1H, Ar—H), 8.01 (m, 1H, Ar—H), 7.73 (m, 2H, Ar—H), 7.63 (m, 3H, Ar—H), 6.86 (s, 1H, pyrazole-H), 6.75 (m, NH), 6.62 (br.s, NH), 4.62 (m, 2H, Ar—CH₂).

### Synthesis of Example 27

### N-((1-(3-chlorophenyl)-3-(trifluoromethyl)-1H-pyrazol-5-yl)methyl)-2-(6-(methylsulfonamidomethyl)pyridin-3-yl)propanamide

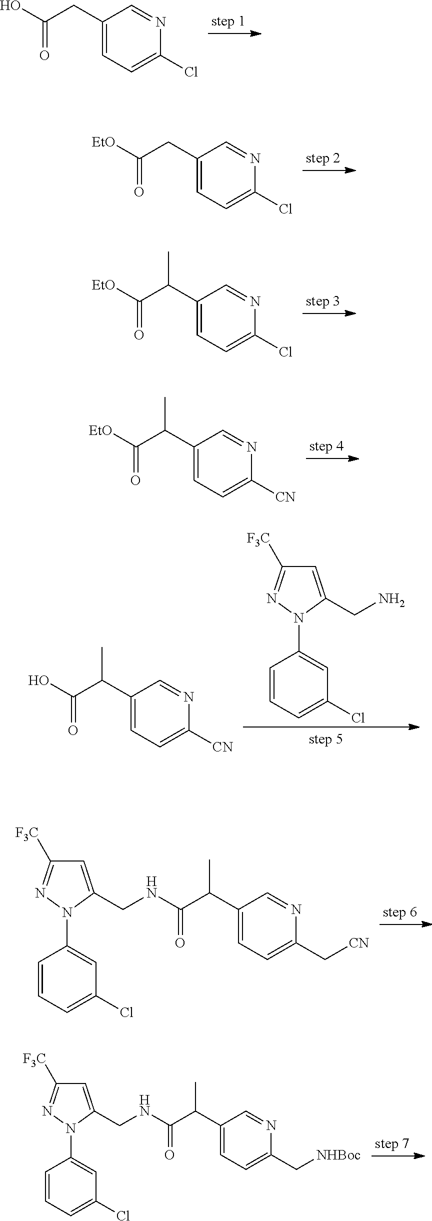

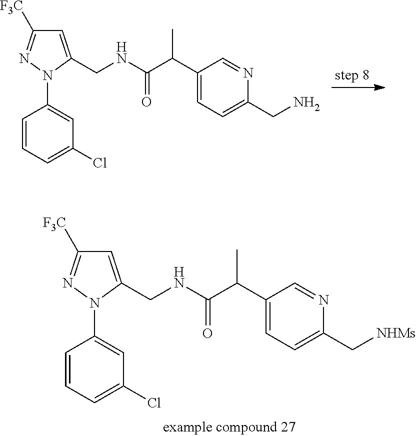

### Step 1:

To a solution of 6-chloro-3-pyridineacetic acid (3.0 g, 17.5 mmol) in ethanol was slowly added sulfuric acid (0.3 mL) at room temperature. The reaction mixture was heated to 100° C. for overnight. TLC showed complete consumption of starting material. The reaction mixture was cooled to room temperature and neutralized with NaHCO₃. The mixture was extracted with ethyl acetate and washed with water and brine. The extract was dried over magnesium sulfate and concentrated under reduced pressure to give the desired ethyl 2-(6-chloropyridin-3-yl)acetate (3.0 g, 86%).

### Step 2:

To a solution of ethyl 2-(6-chloropyridin-3-yl)acetate (3.0 g, 15.1 mmol) in anhydrous dimethylformamide was slowly added 60% sodium hydride (664 mg, 16.6 mmol, 1.1 eq) and Iodo-methane (1.0 mL, 15.9 mmol, 1.05 eq) at 0° C. The reaction mixture was heated to room temperature for 45 min under nitrogen atmosphere. TLC showed complete consumption of starting material. The reaction mixture was added water for quenching. The mixture was extracted with ethyl acetate and washed with water and brine. The extract was dried over magnesium sulfate and concentrated under reduced pressure to afford crude which was purified by column chromatography to afford ethyl 2-(6-chloropyridin-3-yl)propanoate (916 mg, 28%).

### Step 3:

To a solution of ethyl 2-(6-chloropyridin-3-yl)propanoate (3.0 g, 13.8 mmol) in anhydrous dimethylformamide was added Zn(CN)₂ (2.3 g, 19.9 mmol, 1.5 eq), Pd(PPH₃)₄ (1.5 g, 1.32 mmol, 0.1 eq). The reaction mixture was refluxed for overnight under nitrogen atmosphere. TLC showed complete consumption of starting material. The mixture was filtered through celite pad and the filtrate was concentrated under reduced pressure to afford desired compound. The mixture was extracted with ethyl acetate and washed with water and brine. The extract was dried over magnesium sulfate and concentrated under reduced pressure to afford crude which was purified by column chromatography to ethyl 2-(6-cyanopyridin-3-yl)propanoate (1.3 g, 45%).

### Step 4:

Ethyl 2-(6-cyanopyridin-3-yl)propanoate (1.3 g, 6.22 mmol) was dissolved in tetrahydrofuran and water (1:1). The reaction mixture was added NaOH (622 mg, 15.6 mmol, 2.5 eq) which is dissolved on tetrahydrofuran and water (1:1) and stirred at room temperature for 4 h under nitrogen atmosphere. TLC showed complete consumption of starting material. The mixture was diluted with water and added acetic acid until pH 3. Then the mixture is extracted with dichloromethane. The organic part was washed with water and brine. The organic layer was dried over magnesium sulfate and concentrated under reduced pressure. The crude was purified by column chromatography to give 2-(6-cyanopyridin-3-yl)propanoic acid (1.1 g, 95%).

### Step 5:

To a solution of 2-(6-cyanopyridin-3-yl)propanoic acid (364 mg, 2.07 mmol) in dimethylformamide was added N-(3-dimethylaminopropyl)-N′-ethylcarbodiimide (594 mg, 3.09 mmol, 1.5 eq), HOBt (419 mg, 3.09 mmol, 1.5 eq), triethylamine (0.72 mL, 5.17 mmol, 2.5 eq) and (1-(3-chlorophenyl)-3-(trifluoromethyl)-1H-pyrazol-5-yl)methanamine (565 mg, 2.07 mmol, 1 eq) at room temperature and stirred for overnight. TLC showed complete consumption of starting material. The reaction mixture was diluted with water and extracted with ethyl acetate. The organic part was washed with water and brine. The organic layer was dried over magnesium sulfate and concentrated under reduced pressure. The crude was purified by column chromatography to give N-((1-(3-chlorophenyl)-3-(trifluoromethyl)-1H-pyrazol-5-yl)methyl)-2-(6-(cyanomethyl)pyridin-3-yl)propanamide (378 mg, 42%).

### Step 6:

N-((1-(3-Chlorophenyl)-3-(trifluoromethyl)-1H-pyrazol-5-yl)methyl)-2-(6-(cyanomethyl)pyridin-3-yl)propanamide (372 mg, 0.86 mmol) is dissolved in Methanol. The mixture is cooled by ice bath and slowly added di-t-butyl dicarbonate (374 mg, 1.72 mmol, 2 eq), NiCl₂.6H₂O (20 mg, 0.09 mmol, 0.1 eq) and NaBH₄ (227 mg, 6.00 mmol, 7 eq) and stirred for 1 h at 0° C. After 1 h added diethylenetriamine (0.09 mL, 0.86 mmol, 1 eq) to the mixture and stirred at room temperature for 1 h. TLC showed complete consumption of starting material. The reaction mixture was diluted with water and extracted with ethyl acetate. The organic part was washed with water and brine. The organic layer was dried over magnesium sulfate and concentrated under reduced pressure. The crude was purified by column chromatography to give tert-butyl (5-(1-((1-(3-chlorophenyl)-3-(trifluoromethyl)-1H-pyrazol-5-yl)methylamino)-1-oxopropan-2-yl)pyridin-2-yl)methylcarbamate (166 mg, 41%).

### Step 7:

Tert-butyl (5-(1-((1-(3-chlorophenyl)-3-(trifluoromethyl)-1H-pyrazol-5-yl)methylamino)-1-oxopropan-2-yl)pyridin-2-yl)methylcarbamate (166 mg, 0.30 mmol) was dissolved in dichloromethane (4 mL). The reaction mixture was added trifluoro aceticacid (1 mL) and stirred at room temperature for overnight under nitrogen atmosphere. TLC showed complete consumption of starting material. The mixture was neutralized with NaHCO₃ solution and extracted with dichloromethane. The organic part was washed with water and brine. The organic layer was dried over magnesium sulfate and concentrated under reduced pressure. The crude was purified by column chromatography to give 2-(6-(aminomethyl)pyridin-3-yl)-N-((1-(3-chlorophenyl)-3-(trifluoromethyl)-1H-pyrazol-5-yl)methyl)propanamide (75 mg, 56%).

### Step 8:

To a solution of 2-(6-(aminomethyl)pyridin-3-yl)-N-((1-(3-chlorophenyl)-3-(trifluoromethyl)-1H-pyrazol-5-yl)methyl)propanamide (75 mg, 0.17 mmol) in dichloromethane was added Methane sulfonyl chloride (0.013 mL, 0.17 mmol, 1 eq) and triethylamine (0.023 mL, 0.17 mmol, 1 eq) at 0° C. The reaction mixture was stirred for 30 min. TLC showed complete consumption of starting material. The mixture was extracted with ethyl acetate and washed with water and brine. The extract was dried over magnesium sulfate and concentrated under reduced pressure. The crude was purified by column chromatography to give N-((1-(3-chlorophenyl)-3-(trifluoromethyl)-1H-pyrazol-5-yl)methyl)-2-(6-(methylsulfonamidomethyl)pyridin-3-yl)propanamide (example compound 27) (45 mg, 51%).

¹H NMR (300 MHz, CDCl₃) δ 8.42 (s, 1H, Ar—H), 7.64 (dd, 1H, J=8.07 Hz, 2.22 Hz, Ar—H), 7.43 (m, 3H, Ar—H), 7.32 (m, 2H, Ar—H), 6.46 (s, 1H, pyrazole-H), 5.79 (bs, 1H, amide-H), 5.59 (bs, 1H, amine (Ms)-H), 4.51 (d, 2H, J=5.67 Hz, pyrazole-CH₂), 4.43 (d, 2H, J=5.31 Hz, Ar—CH₂), 3.53 (m, 1H), 2.96 (s, 3H, Ms-CH₃), 1.49 (d, 3H, J=6.96 Hz)

### Synthesis of Example 50

### 1-((1-(3-chlorophenyl)-3-(trifluoromethyl)-1H-pyrazol-5-yl)methyl)-3-(5-fluoro-6-(hydroxymethyl)pyridin-3-yl)urea

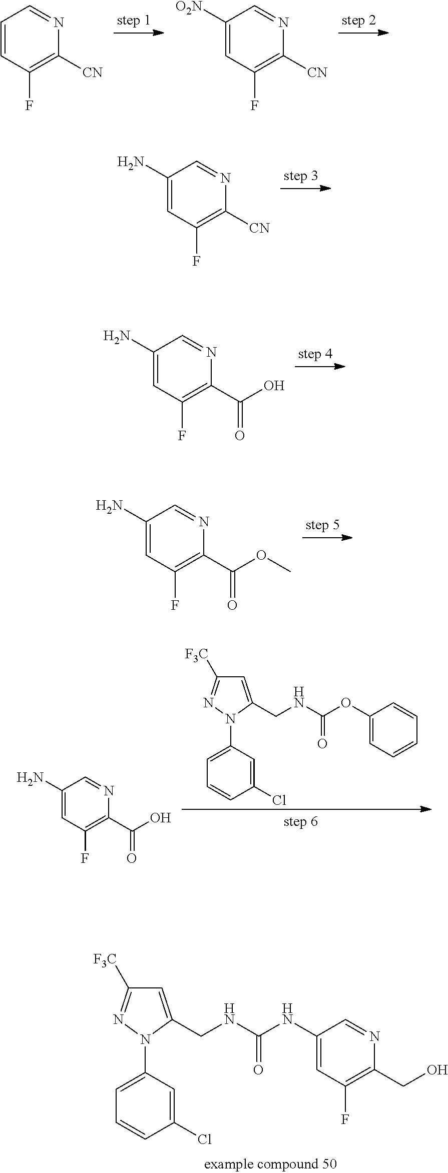

### Step 1:

TFA (12.2 mL, 164 mmol, 18.7 g, 2 eq) was added to KNO3 (16.6 g, 164 mmol, 2 eq) under nitrogen atmosphere, followed by trifluoroacetic anhydride (11.4 mL, 17.2 g, 82 mmol, 1 eq). After 15 minutes 3-fluoropicolinonitrile (10.0 g, 82 mmol) was added at once as an oil. After stirring for 48 h it was poured into saturated aq. NaHCO₃ (aq) (400 mL) and the mixture was extracted with ethyl acetate (3×300 mL). The combined organic layers were dried over sodium sulfate and concentrated to give a yellow oil. The crude product consisted of ˜20% of product and starting material according to H NMR. The oil was adsorbed on silica (100 g) using dichloromethane. The adsorbed silica was placed on top of a 10 cm pad of silica (˜1 L) and the product was eluted with 20% ethyl acetate in heptane. The product-containing fractions were pooled to give 3-fluoro-5-nitropicolinonitrile as a white solid (2.1 g, 15%).

### Step 2:

A solution of 3-fluoro-5-nitropicolinonitrile (2.1 g, 12.6 mmol) in ethyl acetate (10 mL) and acetic acid (10 mL) was heated to 65° C. and iron powder (542 mg, 9.7 mmol, 5 eq) was added. After 30 minutes a red brown suspension formed, which was filtered over celite and concentrated. The residue was added to ethyl acetate (200 mL) and saturated aq. NaHCO₃ (200 mL). The resulting dark-brown precipitate was filtered over celite. The layers were separated and the aqueous layer was extracted with ethyl acetate (3×200 mL). The combined organic layers were dried over sodium sulfate and concentrated to give 5-amino-3-fluoropicolinonitrile as a brown solid (1.52 g).

### Steps 3+4

A solution of 5-amino-3-fluoropicolinonitrile (1.52 g, 11 mmol) in conc. aq. HCl (20 mL) was heated at 80° C. overnight. The mixture was concentrated to give the crude product as a red solid. Methanol (100 mL) was added and removed by rotary evaporation at 50° C. This procedure was repeated 3 times to dry the product. Subsequently, methanol (50 mL) and sulfuric acid (1 mL) were added and the mixture was refluxed overnight. After cooling to room temperature it was poured onto NaHCO₃ (100 g) and concentrated. Water (300 mL) and ethyl acetate (200 mL) were added and the layers were separated. The aqueous layer was extracted with ethyl acetate (2×200 mL). The combined organic layers were dried over sodium sulfate and concentrated to give a brown residue which was triturated with dichloromethane (20 mL). The resulting solid was filtered off and dried to give the product (860 mg, 45%) as a light-brown solid.

### Step 5:

A solution of methyl 5-amino-3-fluoropicolinate (860 mg, 5.0 mmol) in tetrahydrofuran (50 mL) was cooled on an ice/water bath. A solution of LiAlH4 (4N in diethyl ether) (3.75 mL, 15 mmol, 3 eq) was added. After 1 h it was poured into ethyl acetate (200 mL). Water (10 mL) and saturated aq. NaHCO₃ (10 mL) were added and the mixture was stirred for 30 min. The solution was decanted from the white precipitate, washed with brine, dried over sodium sulfate and concentrated to give a brown solid. It was filtered through a short pad of silica (2 cm) to give the title compound as a yellow solid (522 mg, 3.67 mmol, 73%).

### Step 6:

To a stirred solution of (5-amino-3-fluoropyridin-2-yl)methanol (28 mg, 0.202 mmol) in tetrahydrofuran (3 mL) was added N-ethyldiisopropylamin (0.093 mL, 0.548 mmol) followed by phenyl (1-(3-chlorophenyl)-3-(trifluoromethyl)-1H-pyrazol-5-yl)methylcarbamate (74 mg, 0.189 mmol) at 150° C. and stirred for 1 h under microwave conditions (7 bar). The concentrated reaction mixture was purified by column chromatography (silica gel: 100-200 mesh, eluent: ethyl acetate/methanol 9:1) to get 1-((1-(3-chlorophenyl)-3-(trifluoromethyl)-1H-pyrazol-5-yl)methyl)-3-(5-fluoro-6-(hydroxymethyl)pyridin-3-yl)urea (example compound 50) (12 mg, 14%) as white solid.

### Synthesis of Example 52

### 1-((1-(3-chlorophenyl)-3-(trifluoromethyl)-1H-pyrazol-5-yl)methyl)-3-(6-(2-hydroxyethyl)pyridin-3-yl)urea

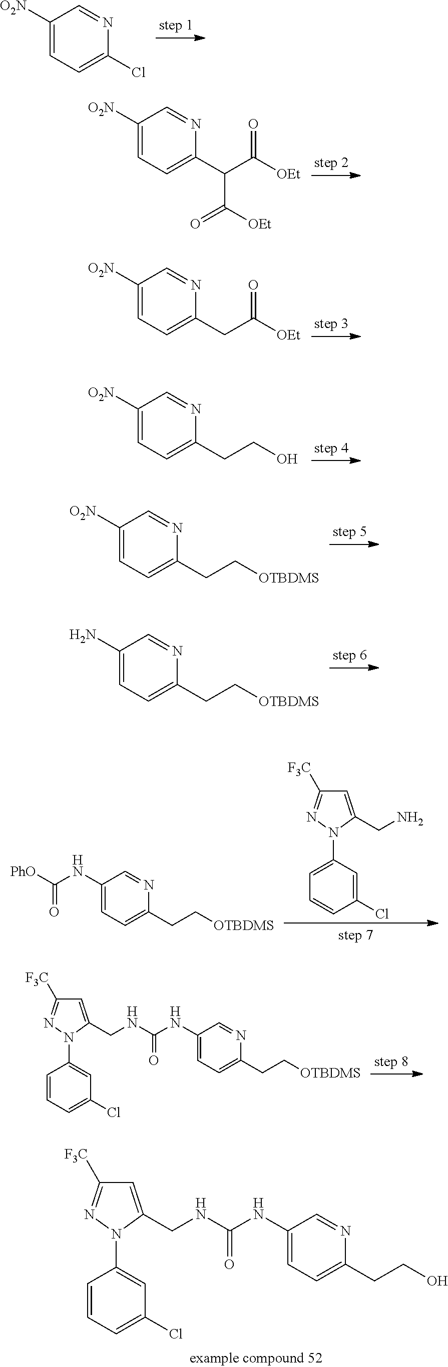

### Step 1:

To a stirred solution of diethyl malonate (9.6 mL, 63.25 mmol, 2 eq) in dimethylformamide (50 mL) at room temperature was added K₂CO₃ (12.8 g, 93.09 mmol, 3 eq) and 2-chloro-5-nitropyridine (5 g, 31.23 mmol, 1 eq) stirred for 16 h at 70° C. The reaction mixture was poured over ice cold water, and extracted with ethyl acetate (2×25 mL), dried over sodium sulfate and evaporated to get diethyl 2-(5-nitropyridin-2-yl)malonate (5.7 g, 68%).

### Step 2:

To a stirred solution of diethyl 2-(5-nitropyridin-2-yl)malonate (1.0 g, 3.5 mmol, 1.0 eq) in DMSO (15 mL) was added NaCl (0.21 g, 3.5 mmol, 1.0 eq), water (0.2 mL) and resulting reaction mixture was heated to 120° C. for 2 h. The reaction mixture was concentrated and extracted with ethyl acetate (2×20 mL), washed with brine (30 mL), dried over sodium sulfate, concentrated and crude was purified by silica gel (100-200 mesh) column chromatography using ethyl acetate/petrol ether (1:9) as eluent to get ethyl 2-(5-nitropyridin-2-yl)acetate (0.41 g, 55%).

### Step 3:

To a stirred solution of ethyl 2-(5-nitropyridin-2-yl)acetate (2.5 g, 11.91 mmol, 1.0 eq) in dry tetrahydrofuran was added DIBAL (23 mL, 23.8 mmol) dropwise at −78° C. The reaction mixture was stirred at −78° C. for 2 h then the reaction mixture was quenched with ice water and extracted with ethyl acetate (30 mL), evaporated under reduced pressure and crude was purified by silica gel (100-200 mesh) column chromatography using ethyl acetate/petrol ether (1:1) as eluent to get 2-(5-nitropyridin-2-yl)ethanol (0.5 g, 25%).

### Step 4:

To a stirred solution of 2-(5-nitropyridin-2-yl)ethanol (0.300 g, 1.78 mmol, 1.0 eq) in dichloromethane (20 mL) was added imidazole (0.182 g, 2.6 mmol, 1.5 eq), and TBDMSCl (0.390 g, 2.6 mmol) at 0° C. and allowed to stir at room temperature for 2 h. The reaction mixture was quenched with water (25 mL) and extracted with dichloromethane (2×15 mL). The organic layer was washed with brine (30 mL), dried over sodium sulfate, concentrated and crude was purified by silica gel (100-200 mesh) column chromatography using ethyl acetate/petrol ether (1:9) as eluent to get 2-(2-(tert-butyldimethylsilyloxy)ethyl)-5-nitropyridine (0.42 g, 84%).

### Step 5:

To a stirred solution of 2-(2-(tert-butyldimethylsilyloxy)ethyl)-5-nitropyridine (0.4 g, 1.4 mmol, 1.0 eq) in methanol (10 mL) was added 10% Pd/C (0.1 g) and stirred under hydrogen atmosphere at room temperature for 2 h. The reaction mixture was filtered through celite pad and filtrate was concentrated under reduced pressure. This crude was washed with diethyl ether (20 mL) to get 6-(2-(tert-butyldimethylsilyloxy)ethyl)pyridin-3-amine (0.25 g, 71%) as off-white solid.

### Step 6:

To a stirred solution of 6-(2-(tert-butyldimethylsilyloxy)ethyl)pyridin-3-amine (0.14 g, 0.5 mmol, 1.0 eq) in acetone (10 mL) were added pyridine (0.08 mL, 1.0 mmol, 2 eq), phenyl carbonochloridate (0.095 g, 0.6 mmol, 1.1 eq) at 0° C. The reaction mixture was stirred at room temperature for 2 h. The reaction mixture was concentrated and diluted with dichloromethane (10 mL), washed water (20 mL), dried over sodium sulfate and concentrated under reduced pressure to get phenyl 6-(2-(tert-butyldimethylsilyloxy)ethyl)pyridin-3-ylcarbamate (0.195 g, 94%) as off white solid.

### Step 7:

To a stirred solution of (1-(3-chlorophenyl)-3-(trifluoromethyl)-1H-pyrazol-5-yl)methanamine (150 mg, 0.48 mmol, 1.0 eq) in dichloromethane (10 mL) was added triethylamine (0.3 mL, 2.3 mmol, 5.0 eq) and stirred at room temperature for 10 min and phenyl 6-(2-(tert-butyldimethylsilyloxy)ethyl)pyridin-3-ylcarbamate (180 mg, 0.48 mmol, 1.0 eq) added and stirred at room temperature for 16 h. The reaction mixture was concentrated and the resulting crude was purified by silica gel column chromatography (100-200 mesh) and again by preparative TLC to get 1-(6-(2-(tert-butyldimethylsilyloxy)ethyl)pyridin-3-yl)-3-((1-(3-chlorophenyl)-3-(trifluoromethyl)-1H-pyrazol-5-yl)methyl)urea (198 mg, 76%) as a white solid.

### Step 8:

To a stirred solution of 1-(6-(2-(tert-butyldimethylsilyloxy)ethyl)pyridin-3-yl)-3-((1-(3-chlorophenyl)-3-(trifluoromethyl)-1H-pyrazol-5-yl)methyl)urea (198 mg, 0.35 mmol, 1.0 eq) in tetrahydrofuran (10 mL) was added 2N HCl (1.5 mL) and stirred at room temperature for 30 min. The reaction mixture basify with saturated aq. NaHCO₃ solution and extracted with ethyl acetate (10 mL), dried over sodium sulfate and evaporated to get 1-((1-(3-chlorophenyl)-3-(trifluoromethyl)-1H-pyrazol-5-yl)methyl)-3-(6-(2-hydroxyethyl)pyridin-3-yl)urea (example compound 52) (98 mg, 62%) as a solid.

### Synthesis of Example 53

### N-((1-(3-chlorophenyl)-3-(trifluoromethyl)-1H-pyrazol-5-yl)methyl)-2-(6-((2-hydroxyethoxy)methyl)pyridin-3-yl)propanamide

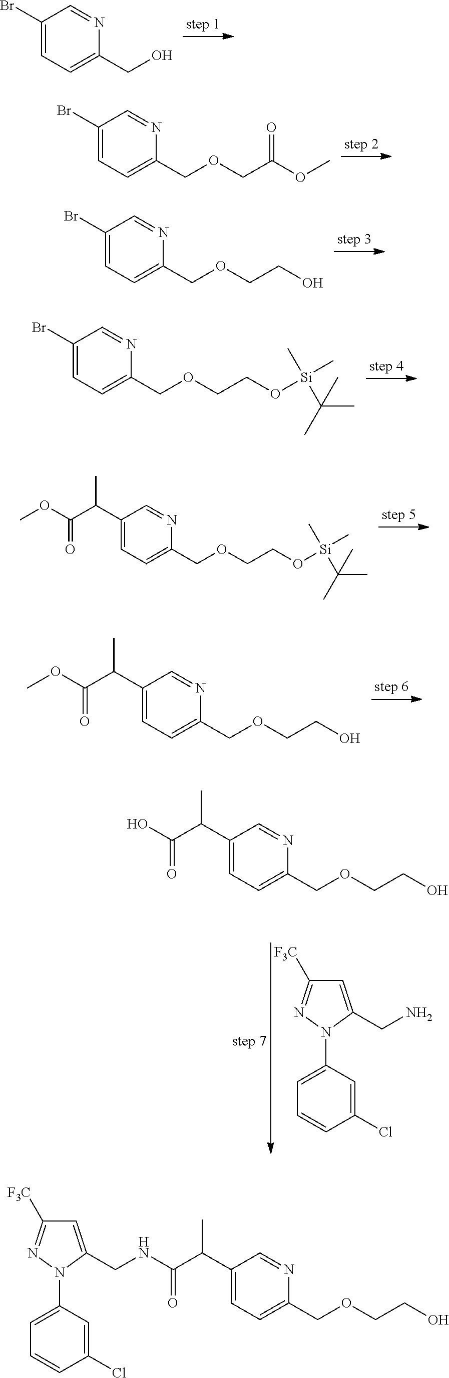

### Step 1:

To a stirred solution of (5-bromopyridin-2-yl)methanol (19 g, 101.1 mmol) in tetrahydrofuran (450 mL) was added portion wise NaH (3.636 g, 151.6 mmol). After 20 min stirring at room temperature ethyl 2-bromoacetate (20.561 g, 134.4 mmol, in 45 mL tetrahydrofuran) was added. The reaction mixture was stirred for 5 h at room temperature. After dilution with saturated sodium hydrogen carbonate solution (200 mL) the mixture was concentrated under reduced pressure and extracted with ethyl acetate (3×200 mL). The combined organic layer was washed with water (150 mL) and brine (150 mL), dried over magnesium sulfate and concentrated in vacuo to obtain crude compound which was purified by column chromatography (silica gel: 100-200 mesh, eluent: ethyl acetate/petrol ether 1:1) to afford methyl 2-((5-bromopyridin-2-yl)methoxy)acetate (19.39 g, 84%) as orange oil.

### Step 2:

Methyl 2-((5-bromopyridin-2-yl)methoxy)acetate (9 g, 34.6 mmol) was dissolved in ethanol (100 mL). After portionwise addition of sodium borohydride (3.93 g, 103.9 mmol) the reaction mixture was stirred for 3 h at room temperature, diluted with water (250 mL) and extracted with ethyl acetate (3×150 mL). The combined organic layer was dried over magnesium sulfate, concentrated under reduced pressure and purified by column chromatography (silica gel: 100-200 mesh, eluent: ethyl acetate/petrol ether 1:1) to afford 2-((5-bromopyridin-2-yl)methoxy)ethanol (5.25 g, 65%) as yellow oil.

### Step 3:

2-((5-Bromopyridin-2-yl)methoxy)ethanol (5.25 g, 22.6 mmol) was dissolved in dimethylformamide (40 mL), TBDMSCl (4.432 g, 29.4 mmol) and imidazole (3.08 g, 45.2 mmol) were added. The mixture was stirred for 2 h, diluted with water (100 mL), extracted with ethyl acetate (3×80 mL), dried over magnesium sulfate and purified by column chromatography (silica gel: 100-200 mesh, eluent: ethyl acetate/petrol ether 1:7) to afford 5-bromo-2-((2-(tert-butyldimethylsilyloxy)ethoxy)methyl)-pyridine (7.9 g, 100%) as yellow oil.

### Step 4a

Syntheses of the catalysator C: To a stirring solution of Pd(dppf)Cl₂ in absolute tetrahydrofurane (15 mL) was added DPPF (0.4 g, 0.7218 mmol) and dropwise n-BuLi (0.9 mL, 1.4 mmol) to afford C an orange suspension.

### Step 4:

Thallium(I)-acetate (7.6 g, 28.9 mmol) was dissolved in absolute tetrahydrofuran (45 mL) and C was added in nitrogen gas counterflow. The mixture was heated to 85° C. 5-Bromo-2-((2-(tert-butyldimethylsilyloxy)ethoxy)methyl)-pyridine (4.98 g, 14.4 mmol) was dissolved in absolute tetrahydrofuran (15 mL) and added dropwise to the reaction mixture and stirred for 2 h at reflux. Reaction mixture was diluted with water ethyl acetate (200 mL 1:1), filtered on celite bed, extracted with ethyl acetate (2×50 mL), dried over magnesium sulfate and concentrated in vacuo. The crude compound was purified by column chromatography (silica gel: 100-200 mesh, eluent: ethyl acetate/petrol ether 1:2) to afford methyl 2-(6-((2-(tert-butyldimethylsilyloxy)ethoxy)methyl)pyridin-3-yl)propanoate (4.05 g, 80%) as yellow oil.

### Step 5:

At 4° C. in a round bottom flask methyl 2-(6-((2-(tert-butyldimethylsilyloxy)ethoxy)-methyl)pyridin-3-yl)propanoate (4 g, 11.3 mmol) was taken under nitrogen atmosphere and TBAF (13.6 mL, 13.6 mmol) was added dropwise and stirred for 1 h at room temperature. After addition of SiO₂ (5 g) the solvent was removed under reduced pressure and the obtained crude was purified by column chromatography (silica gel: 100-200 mesh, eluent: ethyl acetate/petrol ether 2:1) to afford methyl 2-(6-((2-hydroxyethoxy)methyl)pyridin-3-yl)propanoate (2 g, 74%) as orange oil.

### Step 6:

To 2-(6-((2-hydroxyethoxy)methyl)pyridin-3-yl)propanoate ( )in tetrahydrofuran (5 mL) was added methanol (10 mL) and 1 N NaOH solution (8.3 mL). The mixture was stirred for 1 h at 75° C., concentrated in vacuo and diluted with 2 N HCl until pH 6.5. The mixture was concentrated under reduced pressure up to 2 mL volume, putted on an activated cation exchanger and purified by column chromatography (silica gel: 100-200 mesh, eluent: ethyl acetate/methanol 1:1) to afford 2-(6-((2-hydroxyethoxy)methyl)pyridin-3-yl)propanoic acid (405 mg, 44%) as yellow oil.

### Step 7:

To a solution of 2-(6-((2-hydroxyethoxy)methyl)pyridin-3-yl)propanoic acid (49 mg, 0.221 mmol) in tetrahydrofuran (1.7 mL) was added 1-hydroxybenzotriazole (31 mg, 0.221 mmol), O-(1H-benzotriazol-1-yl)-N,N,N′,N′-tetramethyluronium tetrafluoroborate (71 mg, 0.221 mmol), n-ethyldiisopropylamine (0.113 mL, 0.663 mmol) and (1-(3-chlorophenyl)-3-(trifluoromethyl)-1H-pyrazol-5-yl)methanamine (60 mg, 0.221 mmol). The reaction mixture was stirred for 48 h at room temperature. The mixture was diluted with water and extracted with ethyl acetate. The organic layer was dried over magnesium sulfate and concentrated under reduced pressure. The crude was purified by column chromatography (silica gel: 100-200 mesh, eluent: ethyl acetate/methanol 9:1) to give pure N-((1-(3-chlorophenyl)-3-(trifluoromethyl)-1H-pyrazol-5-yl)methyl)-2-(6-((2-hydroxyethoxy)methyl)pyridin-3-yl)propanamide (example compound 53) (94 mg, 88%) as white solid.

Example 161 can be prepared and examples 30, 51, 129, 142 and 149-151 were prepared in a similar manner.

### Synthesis of Example 55

### 5-(1-((1-(3-chlorophenyl)-3-(trifluoromethyl)-1H-pyrazol-5-yl)methylamino)-1-oxopropan-2-yl)picolinamide

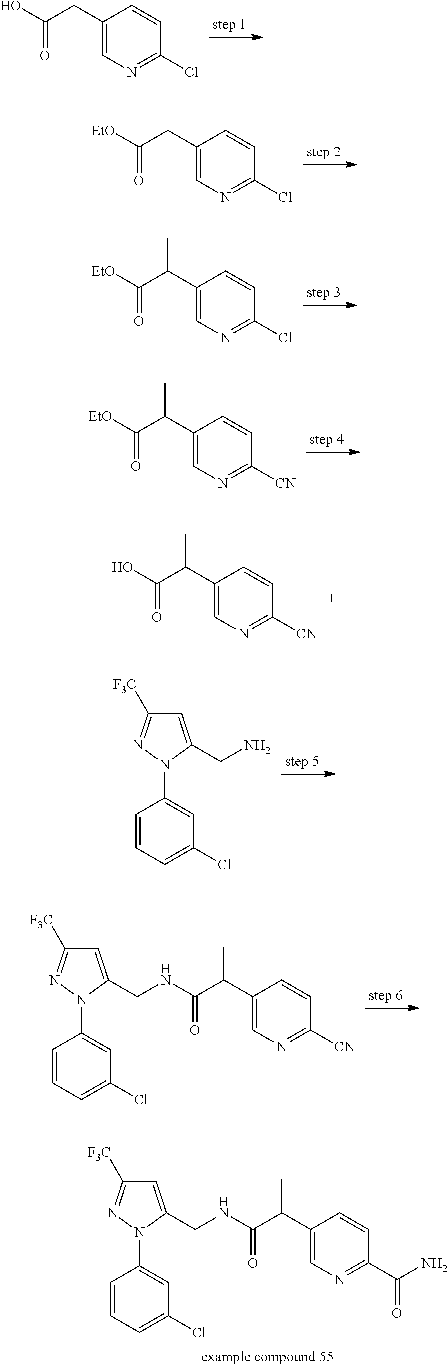

### Step 1:

To a solution of 6-chloro-3-pyridineacetic acid (1 g, 5.83 mmol) in ethanol was added sulfuric acid (1.6 mL). The mixture was refluxed for 4 h, then cooled to room temperature and concentrated. The residue was diluted with ethyl acetate and washed with a saturated sodium hydrogen carbonate solution. The resulting mixture was dried over magnesium sulfate and concentrated under reduced pressure to afford crude which was purified by column chromatography to afford ethyl 2-(6-chloropyridin-3-yl)acetate (1.1 g, 95%).

### Step 2:

To a solution of ethyl 2-(6-chloropyridin-3-yl)acetate (1.1 g, 5.51 mmol) in dimethylformamide was added slowly sodium hydride (242 mg, 6.06 mmol) at 0° C., followed by iodomethane (821 mg, 5.79 mmol). The mixture was stirred at same degree for 1 hour, and then quenched with water. The resulting mixture was diluted with ethyl acetate and washed with water. The organic layer was dried over magnesium sulfate and concentrated under reduced pressure to afford crude which was purified by column chromatography to afford ethyl 2-(6-chloropyridin-3-yl)propanoate (790 mg, 67%).

### Step 3:

To a solution of ethyl 2-(6-chloropyridin-3-yl)propanoate (790 mg, 3.7 mmol) in dimethylformamide was added zinc cyanide (434 mg, 3.7 mmol) and Pd(PPh₃)₄ (1.28 g, 1.11 mmol). The reaction mixture was stirred for 12 h at 100° C. and then cooled to room temperature. The mixture was filtered through a plug of celite and concentrated. The residue was diluted with ethyl acetate and washed with 10% HCl solution. The organic layer was dried over magnesium sulfate and concentrated under reduced pressure to afford crude which was purified by column chromatography to afford ethyl 2-(6-cyanopyridin-3-yl)propanoate (420 mg, 56%).

### Step 4:

To a solution of ethyl 2-(6-cyanopyridin-3-yl)propanoate (420 mg, 2.06 mmol) in tetrahydrofuran and water was added lithium hydroxide monohydrate (129 mg, 3.08 mmol). The reaction mixture was stirred for 2 h at 40° C. and then acidified with 10% HCl solution. The mixture was extracted with ethyl acetate. The organic layer dried over magnesium sulfate and concentrated under reduced pressure to afford the desired 2-(6-cyanopyridin-3-yl)propanoic acid (330 mg, 94%).

### Step 5:

To a solution of 2-(6-cyanopyridin-3-yl)propanoic acid (330 mg, 1.87 mmol) in acetonitrile was added 1-hydroxybenzotriazole (380 mg, 2.81 mmol), 1-ethyl-3-(3-dimethylaminopropyl) carbodiimide (537 mg, 2.81 mmol) and (1-(3-chlorophenyl)-3-(trifluoromethyl)-1H-pyrazol-5-yl)methanamine (543 mg, 1.97 mmol). The reaction mixture was stirred overnight at room temperature. The reaction mixture was added water and extracted with ethyl acetate. The organic layer was dried over magnesium sulfate and concentrated under reduced pressure. The crude was purified by column chromatography to give pure N-((1-(3-chlorophenyl)-3-(trifluoromethyl)-1H-pyrazol-5-yl)methyl)-2-(6-cyanopyridin-3-yl)propanamide (440 mg, 54%).

### Step 6:

Starting material N-((1-(3-chlorophenyl)-3-(trifluoromethyl)-1H-pyrazol-5-yl)methyl)-2-(6-cyanopyridin-3-yl)propanamide (200 mg, 0.46 mmol) was dissolved in sulfuric acid (2 mL). The reaction mixture was stirred for 2 h at 60° C. and then cooled to room temperature. The reaction mixture was diluted with ice water and neutralized (pH=7) with 2 M NaOH solution. The mixture was extracted with ethyl acetate. The organic layer was dried over magnesium sulfate and concentrated under reduced pressure. The crude was purified by column chromatography to give pure 5-(1-((1-(3-chlorophenyl)-3-(trifluoromethyl)-1H-pyrazol-5-yl)methylamino)-1-oxopropan-2-yl)picolinamide (example compound 55) (85 mg, 41%).

¹H NMR (300 MHz, CDCl₃) δ 8.44 (d, 1H, J=2.01 Hz, pyridine-H), 8.10 (d, 1H, J=7.86 Hz, pyridine-H), 7.80 (br.s, NH), 7.75 (dd, 1H, J=8.04, 2.19 Hz, pyridine-H), 7.36 (m, 4H, Ar—H), 6.52 (s, 1H, pyrazole-H), 5.88 (m, NH), 5.64 (br.s, NH), 4.52 (m, 2H, Ar—CH₂), 3.59 (quartet, 1H, J=7.14 Hz, amide-CH), 1.53 (d, 3H, J=7.14 Hz, amide-CH₃).

Example 56 was prepared in a similar manner.

### Synthesis of Example 57

### 5-(1-((1-(3-chlorophenyl)-3-(trifluoromethyl)-1H-pyrazol-5-yl)methylamino)-1-oxopropan-2-yl)-N-phenyl)picolinamide

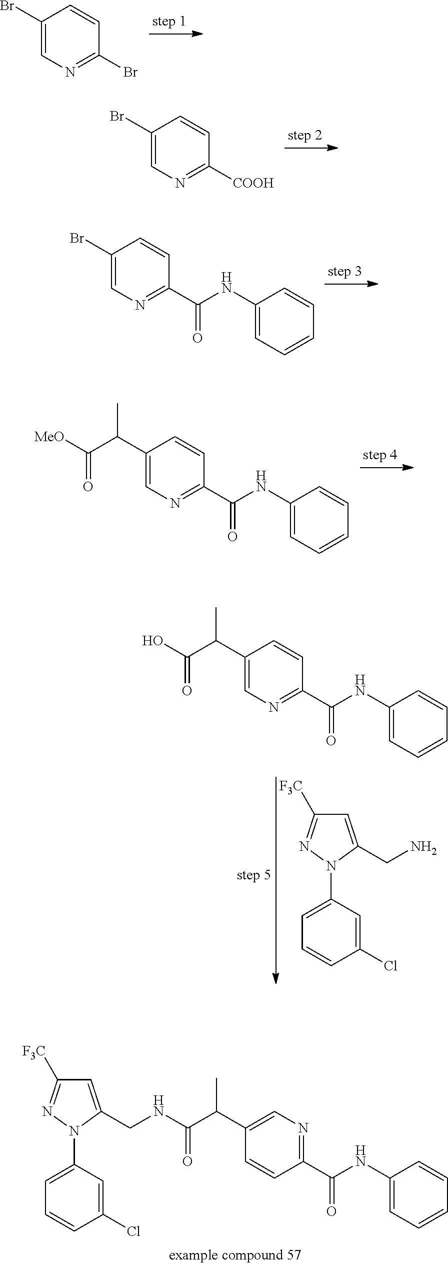

### Step 1:

Toluene (114 mL) was cooled to 78° C., n-BuLi (79.7 mL, 127 mmol) was added dropwise at the same temperature followed by 2,5-dibromopyridine (30 g, 120 mmol) in toluene (60 mL) and stirred for 2 h. The Reaction mixture was bubbled with dry carbon dioxide gas for 1 h at 78° C. Progress of the reaction was monitored by TLC. On completion of the reaction, reaction contents were warmed to room temperature and toluene was distilled under reduced pressure. Then water (200 mL) was added to the reaction mixture and filtered on celite bed. The filtrate was acidified with diluted HCl solution, solid was precipitated out which was filtered and dried over sodium sulfate to yield 5-bromopicolinic acid (12 g, 47% yield) as a brown colored solid.

### Step 2:

5-Bromopicolinic acid (8 g, 30 mmol) in tetrahydrofuran (80 mL) was charged into a 250 mL flask. Then aniline (4.44 g, 47 mmol), O-(1H-benzotriazol-1-yl)-N,N,N′,N′-tetramethyluronium tetrafluoroborate (15.33 g, 47 mmol), triethylamine (6.43 g, 63.6 mmol) were added to the reaction mixture. The overall reaction was allowed to stir for 1 h at room temperature. On completion of the reaction, tetrahydrofuran was distilled off completely. Water (100 mL) was added to the reaction mixture and basified with saturated sodium carbonate solution and extracted with ethyl acetate (2×50 mL). The organic layer was concentrated under reduced pressure to give the crude product which was purified by column chromatography (silica gel: 100 200 mesh, eluent: 2% ethyl acetate in n-hexane) to yield 5-bromo-N-phenyl)picolinamide (7.27 g, 72%) as a white solid.

### Step 3:

5-Bromo-N-phenyl)picolinamide (7.2 g, 26 mmol), methyl 2-chloropropionate (9.64 g, 79 mmol) in dimethylformamide (109 mL) were bubbled with nitrogen gas for 10 min. Manganese (2.89 g, 50 mmol) was added and the reaction mixture was bubbled with nitrogen gas for another 10 min. NiBr₂ bipy (0.97 g, 1.8 mmol) was added and furthermore bubbled with nitrogen gas for 10 min. Then catalytic amount of trifluoroacetic acid was added to reaction mixture and stirred for 30 min. On completion of the reaction, reaction contents were diluted with water and filtered on celite bed. The filtrate was extracted with ethyl acetate (3×50 mL) and the organic layer were concentrated under reduced pressure to give the crude product which was purified by column chromatography (silica gel: 100 200 mesh, eluent: 5% ethyl acetate in n-hexane) to methyl 2-(6-(phenylcarbamoyl)pyridin-3-yl)propanoate (1.5 g, 20%) as a white solid.

### Step 4:

To methyl 2-(6-(phenylcarbamoyl)pyridin-3-yl)propanoate (1.8 g, 6 mmol) in tetrahydrofuran (18 mL) and water (18 mL) was added lithium hydroxide monohydrate (0.3 g, 12 mmol) and stirred for 1 h at room temperature. On completion of the reaction, tetrahydrofuran was distilled off completely. Then ethyl acetate (100 mL) was added, followed by separation of the aqueous layer, acidification with 6N HCl (5 mL) solution and extraction with ethyl acetate (2×50 mL). The combined organic layers were dried over sodium sulfate and concentrated under reduced pressure to yield 2-(6-(phenylcarbamoyl)pyridin-3-yl)propanoic acid (1.65 g, 96%) as a white solid.

### Step 5:

In a round bottom flask 2-(6-(phenylcarbamoyl)pyridin-3-yl)propanoic acid (61 mg, 0.228 mmol) was taken under nitrogen atmosphere and dichloromethane (1.3 mL) and 1-chloro-N,N,2-trimethylprop-1-en-1-amine (47 mL, 0.355 mmol) were added and stirred for 1 h at room temperature. Followed by addition of (1-(3-chlorophenyl)-3-(trifluoromethyl)-1H-pyrazol-5-yl)methanamine (50 mg, 0.182 mmol) and triethylamine (107 mL, 0.637 mmol) the reaction mixture was allowed to stir for 48 h at room temperature. Reaction mixture was diluted with dichloromethane (10 mL), washed with saturated sodium carbonate solution (2×10 mL). The aqueous layer were extracted with dichloromethane (2×10 mL), combined, dried over sodium sulfate and filtered. The solvent was evaporated and finally purified by column chromatography (silica gel: 100 200 mesh, eluent: cyclohexane/ethyl acetate 1:2) to afford 5-(1-((1-(3-chlorophenyl)-3-(trifluoromethyl)-1H-pyrazol-5-yl)methylamino)-1-oxopropan-2-yl)-N-phenyl)picolinamide (example compound 57) (12 mg, 33%) as an off-white solid.

Examples 58, 59 and 61 were prepared in a similar manner.

### Synthesis of Example 60

### 5-(1-((3-tert-butyl-1-(3-chlorophenyl)-1H-pyrazol-5-yl)methylamino)-1-oxopropan-2-yl)-N-(4-fluorophenyl)picolinamide

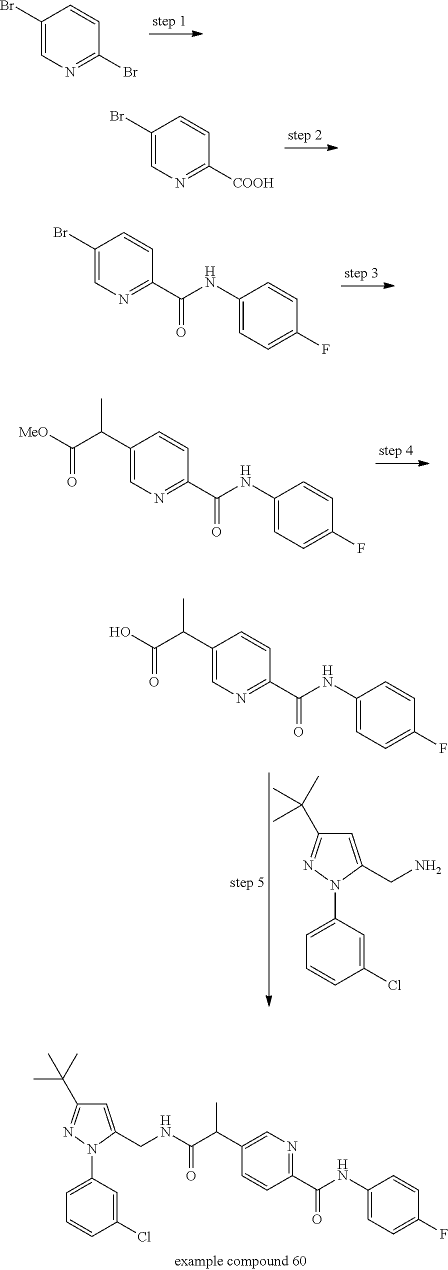

### Step 1:

as described for example 57.

### Step 2:

5-Bromopicolinic acid (7.5 g, 30 mmol) and 4-fluoro aniline (4.97 g, 40 mol) were charged in tetrahydrofuran (75 mL). Then O-(1H-benzotriazol-1-yl)-N,N,N′,N′-tetramethyluronium tetrafluoroborate (14.37 g, 40 mmol), triethylamine (6.02 g, 50 mmol) were added and the reaction mixture was stirred for 1 h at room temperature. Progress of the reaction was monitored by TLC, on completion of the reaction, tetrahydrofuran was distilled under reduced pressure and then sodium carbonate solution was added to reaction mixture and extracted with ethyl acetate (2×50 mL). The combined organic layers were dried over sodium sulfate, concentrated under reduced pressure to give the crude product which was purified by column chromatography (silica gel: 100 200 mesh, eluent: 2% ethyl acetate in n-hexane) to give 5-bromo-N-(4-fluorophenyl)picolinamide (7 g, 67%) as a yellow colored solid.

### Step 3:

5-Bromo-N-(4-fluorophenyl)picolinamide (7 g, 23.8 mmol), methyl 2-chloropropanoate (8.7 g, 71 mmol) in dimethylformamide (105 mL) were charged into a round bottom flask and bubbled with nitrogen gas for 30 min. Manganese (2.61 g, 47.6 mmol) was added to reaction mixture and bubbled with nitrogen gas for 30 min. NiBr₂ LADY (0.97 g, 1.8 mmol) was added and bubbled with nitrogen gas for another 15 min. Then catalytic amount of trifluoroacetic acid was added and stirred for 30 min. Progress of the reaction was monitored by TLC, on completion of the reaction, reaction contents were diluted with water and filtered on celite bed. The filtrate was extracted with ethyl acetate (3×50 mL) and the organic layer was concentrated under reduced pressure to give the crude product which was purified by column chromatography (silica gel: 100 200 mesh, eluent: 5% ethyl acetate in n-hexane) to yield methyl 2-(6-(4-fluorophenylcarbamoyl)pyridin-3-yl)propanoate (1.5 g, 22%) as a white solid.

### Step 4:

Methyl 2-(6-(4-fluorophenylcarbamoyl)pyridin-3-yl)propanoate (1.8 g, 6 mmol) in tetrahydrofuran (10 mL) was charged into a round bottom flask. Then water (10 mL) and lithium hydroxide (0.302 g, 12 mmol) were added. The reaction mixture was allowed to for 1 h at room temperature. Progress of the reaction was monitored by TLC, on completion of the reaction, tetrahydrofuran was distilled off. The aqueous layer was extracted with ethyl acetate (2×20 mL), acidified with diluted HCl solution (20 mL) and extracted with ethyl acetate (2×25 mL). Combined organic layers was dried over sodium sulfate and concentrated under reduced pressure to yield the required 2-(6-(4-fluorophenylcarbamoyl)pyridin-3-yl)propanoic acid (1.5 g, 88%) as a white solid.

### Step 5:

To a solution of 2 2-(6-(4-fluorophenylcarbamoyl)pyridin-3-yl)propanoic acid (59 mg, 0.208 mmol) in tetrahydrofuran (1.6 mL) was added 1-hydroxybenzotriazole (27 mg, 0.208 mmol), O-(1H-benzotriazol-1-yl)-N,N,N′,N′-tetramethyluronium tetrafluoroborate (67 mg, 0.208 mmol), triethylamine (0.07 mL, 0.416 mmol) and (3-tert-butyl-1-(3-chlorophenyl)-1H-pyrazol-5-yl)methanamine (55 mg, 0.208 mmol). The reaction mixture was stirred overnight at room temperature. The mixture was diluted with water and extracted with ethyl acetate. The organic layer was dried over magnesium sulfate and concentrated under reduced pressure. The crude was purified by column chromatography (silica gel: 100 200 mesh, eluent: cyclohexane/ethyl acetate 1:1) to give pure 5-(1-((3-tert-butyl-1-(3-chlorophenyl)-1H-pyrazol-5-yl)methylamino)-1-oxopropan-2-yl)-N-(4-fluorophenyl)picolinamide (example compound 60) (83 mg, 75%).

### Synthesis of Example 63

### 5-(1-((1-(3-chlorophenyl)-3-(trifluoromethyl)-1H-pyrazol-5-yl)methylamino)-1-oxopropan-2-yl)-N-(4-fluorophenyl)pyrimidine-2-carboxamide

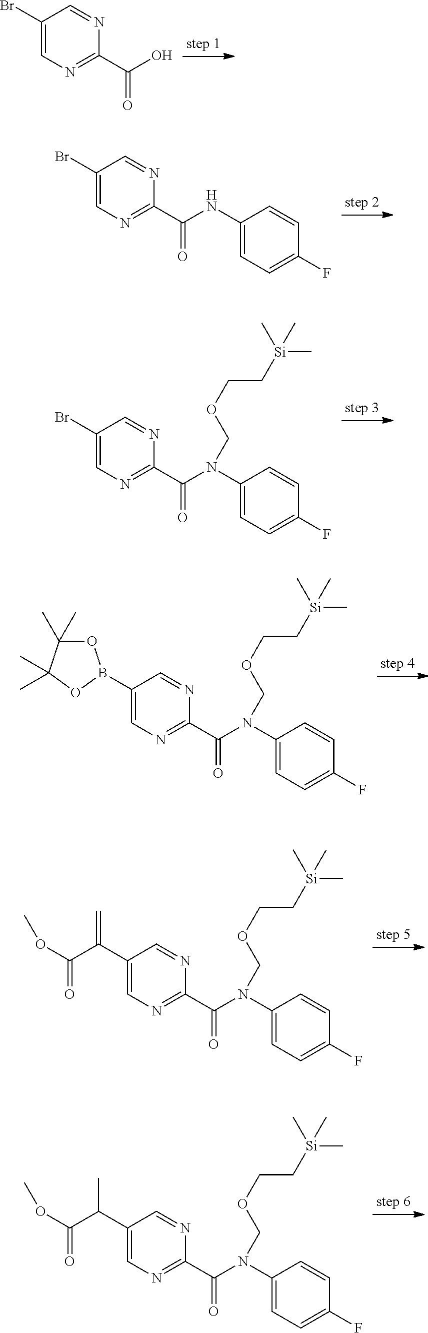

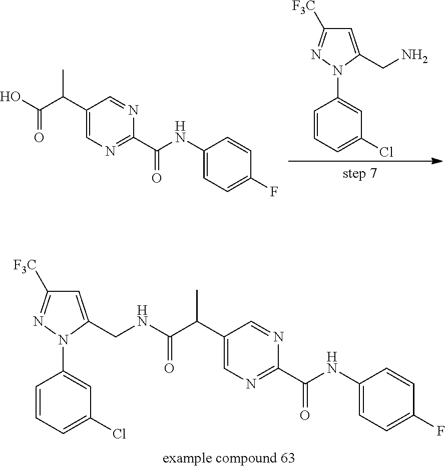 [SMILES: CC(C(=O)O)c1cnc(C(=O)Nc2ccc(F)cc2)nc1 ; NCc1cc(C(F)(F)F)nn1-c1cccc(Cl)c1 ; CC(C(=O)NCc1cc(C(F)(F)F)nn1-c1cccc(Cl)c1)c1cnc(C(=O)Nc2ccc(F)cc2)nc1]

### Step 1:

5-Bromopyrimidine-2-carboxylic acid (5 g, 24.63 mmol) was dissolved in benzene (50 mL) and thionyl chloride (5.63 mL, 73.89 mmol) was added to it in a 250 mL round bottomed flask. The reaction mixture was refluxed for 2 h at 100° C. After that thionyl chloride and benzene was removed under reduced pressure. Water was removed by making an azeotrope using benzene. The residue was dissolved in dichloromethane (100 mL) and it was added to the solution of 4-fluoroaniline (2.68 g, 24.13 mmol) in dichloromethane (100 mL) under nitrogen atmosphere. The reaction mixture was stirred for 16 h at room temperature. After total consumption of starting material, the reaction mixture was diluted with dichloromethane (50 mL) and washed with water (2×100 mL) followed by sodium bicarbonate solution (2×100 mL) and brine (100 mL). The organic layer was dried over magnesium sulfate and concentrated under reduced pressure. The crude compound was purified by column chromatography (silica gel: 100-200 mesh, eluent: 20% ethyl acetate in n-hexane) to afford 5-bromo-N-(4-fluorophenyl)pyrimidine-2-carboxamide (5.6 g, 78%).

### Step 2:

Sodium hydride (60%, 872 mg, 21.81 mmol) was taken in a 250 mL round bottomed two-necked flask and dry dimethylformamide (25 mL) was added to it under nitrogen atmosphere. To the suspension of sodium hydride in dimethylformamide solution of 5-bromo-N-(4-fluorophenyl)pyrimidine-2-carboxamide (5.4 g, 18.24 mmol) in dry dimethylformamide (30 mL) was added at −5° C. The reaction mixture was stirred at same temperature for 30 minutes. After that 2-(trimethylsilyl)ethoxymethyl chloride (4.52 g, 27.36 mmol) was added to it drop wise maintaining the temperature. The reaction mixture was stirred at ambient temperature for 2 h. After total consumption of starting material the reaction mixture was quenched with ammonium chloride solution (150 mL) and extracted with ethyl acetate (3×100 mL). The combined organic layer was dried over magnesium sulfate and concentrated under reduced pressure. The crude compound was purified by column chromatography (silica gel: 100-200 mesh, eluent: 20% ethyl acetate in n-hexane) to afford 5-bromo-N-(4-fluorophenyl)-N-((2-(trimethylsilyl)ethoxy)methyl)pyrimidine-2-carboxamide (6.5 g, 84%).

### Step 3:

5-Bromo-N-(4-fluorophenyl)-N-((2-(trimethylsilyl)ethoxy)methyl)pyrimidine-2-carboxamide (7.5 g, 17.59 mmol) was dissolved in 1,4-dioxane (86 mL) and 4,4,4′,4′,5,5,5′,5′-octamethyl-2,2′-Bi-(1,3,2-dioxaborolane) (4.7 g, 18.47 mmol) was added to it followed by potassium acetate (5.2 g, 52.77 mmol) under nitrogen atmosphere. The reaction mixture was stirred for 5 minutes and Pd(dppf)₂Cl₂ (644 mg, 0.87 mmol) was added to it. The reaction mixture was refluxed for 16 h. After total consumption of starting material the reaction mixture was diluted with water and extracted with ethyl acetate (3×100 mL). The combined organic layer was dried over magnesium sulfate and concentrated under reduced pressure. The crude N-(4-fluorophenyl)-5-(4,4,5,5-tetramethyl-1,3,2-dioxaborolan-2-yl)-N-((2-(trimethylsilyl)-ethoxy)methyl)pyrimidine-2-carboxamide was used for next step without purification (9.0 g, crude).

### Step 4:

N-(4-Fluorophenyl)-5-(4,4,5,5-tetramethyl-1,3,2-dioxaborolan-2-yl)-N-((2-(trimethylsilyl)ethoxy)methyl)pyrimidine-2-carboxamide (8.3 g, 17.59 mmol) was dissolved in toluene (83 mL) and methyl 2-(trifluoromethylsulfonyloxy)acrylate (4.94 g, 21.12 mmol) was added to it followed by 2 M sodium carbonate solution (35. mL) under nitrogen atmosphere. After that Pd(PPh₃)₄ (1.02 g, 0.87 mmol) was added to it. The reaction mixture was refluxed for 16 h. After total consumption of starting material the reaction mixture was diluted with water and extracted with ethyl acetate (3×100 mL). The combined organic layer was dried over magnesium sulfate and concentrated under reduced pressure. The crude was purified by column chromatography (100-200 mesh silica gel, eluent: 10% ethyl acetate in n-hexane) to afford methyl 2-(2-((4-fluorophenyl)((2-(trimethylsilyl)ethoxy)methyl)carbamoyl)pyrimidin-5-yl)acrylate (5 g, 67%).

### Step 5:

Methyl 2-(2-((4-fluorophenyl)((2-(trimethylsilyl)ethoxy)methyl)carbamoyl)-pyrimidin-5-yl)acrylate (5.0 g) was dissolved in ethyl acetate (50 mL) in a 500 mL Parr vessel and 10% Pd/C (500 mg) was added to it under nitrogen atmosphere. The vessel was equipped in Parr apparatus under 50 psi hydrogen pressure. After 2 h TLC showed the total consumption of starting material. The catalyst was filtered through celite bed and filtrate was concentrated under reduced pressure to afford methyl 2-(2-((4-fluorophenyl)((2-(trimethylsilyl)ethoxy)methyl)carbamoyl)pyrimidin-5-yl)propanoate (5 g, quantitative).

### Step 6:

Methyl 2-(2-((4-fluorophenyl)((2-(trimethylsilyl)ethoxy)methyl)carbamoyl)-pyrimidin-5-yl)propanoate (3.0 g, 6.92 mmol) was dissolved in ethanol (87 mL) and 6N HCl (87 mL) was added to it. The reaction mixture was refluxed for 2 h at 90° C. After complete conversion of starting material ethanol was evaporated under reduced pressure and residue was diluted with water and basified by sodium carbonate solution. The aqueous layer was washed with ethyl acetate. After that the aqueous layer was acidified with 6N HCl and extracted with ethyl acetate (3×50 mL). The combined organic layer was dried over magnesium sulfate and concentrated under reduced pressure to afford the pure 2-(2-(4-fluorophenylcarbamoyl)pyrimidin-5-yl)propanoic acid (700 mg, 35%).

¹H NMR (DMSO-d₆, 400 MHz): δ 12.82 (1H, s), 10.80 (1H, s), 8.94 (2H, s), 7.91-7.88 (2H, m), 7.20 (2H, t), 3.96 (1H, q), 1.52 (3H, d); LCMS (M+H): 290; HPLC: 97.71%

### Step 7:

To a stirred solution of (1-(3-chlorophenyl)-3-(trifluoromethyl)-1H-pyrazol-5-yl)methanamine (0.06 g, 0.218 mmol) and 2-(2-(4-fluorophenylcarbamoyl)pyrimidin-5-yl)propanoic acid (0.063 g, 0.218 mmol) in tetrahydrofuran (2 mL) was added 1-hydroxybenzotriazolhydrate (0.029 mL, 0.218 mmol), O-(1H-benzotriazol-1-yl)-N,N,N′,N′-tetramethyluronium tetrafluoroborate (0.07 g, 0.218 mmol) and N-ethyldiisopropylamine (0.074 mL, 0.436 mmol) and the reaction mixture was allowed to stir for 48 h. The reaction mixture was concentrated under reduced pressure and the solid obtained was purified by column chromatography (silica gel: 100-200 mesh, eluent: ethyl acetate/diethyl ether 2:1) to afford 5-(1-((1-(3-chlorophenyl)-3-(trifluoromethyl)-1H-pyrazol-5-yl)methylamino)-1-oxopropan-2-yl)-N-(4-fluorophenyl)pyrimidine-2-carboxamide (example compound 63) (39 mg, 33%).

Examples 62, 64, 66 and 67 were prepared in a similar manner.

### Synthesis of Example 65

### 5-(1-((3-tert-butyl-1-(3-chlorophenyl)-1H-pyrazol-5-yl)methylamino)-1-oxopropan-2-yl)-N-phenylpyrimidine-2-carboxamide

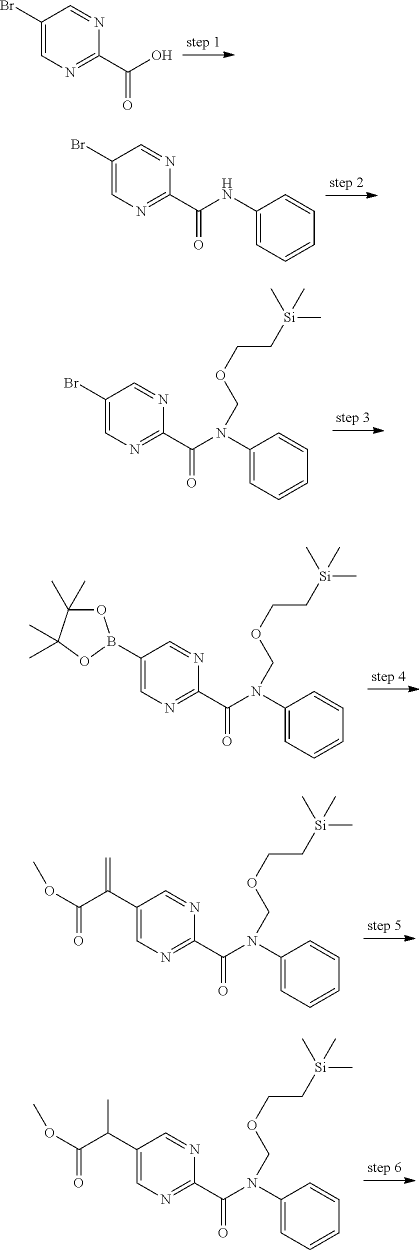

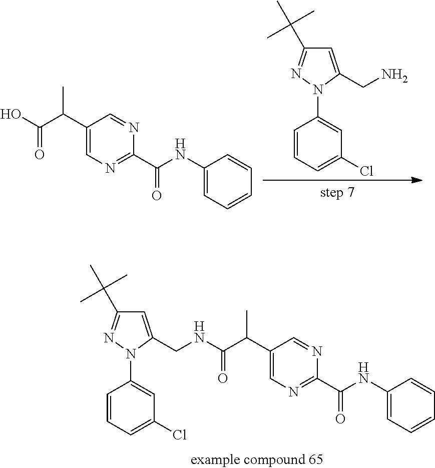 [SMILES: CC(C(=O)O)c1cnc(C(=O)Nc2ccccc2)nc1 ; CC(C)(C)c1cc(CN)n(-c2cccc(Cl)c2)n1 ; CC(C(=O)NCc1cc(C(C)(C)C)nn1-c1cccc(Cl)c1)c1cnc(C(=O)Nc2ccccc2)nc1]

### Step 1:

5-Bromopyrimidine-2-carboxylic acid (5.22 g, 24.63 mmol) was dissolved in benzene (100 mL) and thionyl chloride (5.4 mL, 73.89 mmol) was added to it in a 250 mL round bottomed flask. The reaction mixture was refluxed for 2 h at 100° C. After that thionyl chloride and benzene was removed under reduced pressure. Water was removed by making azeotrope using benzene. The residue was dissolved in dichloromethane (100 mL) and it was added to the solution of aniline (2.27 g, 24.42 mmol) in dichloromethane (100 mL) under nitrogen atmosphere. The reaction mixture was stirred for 16 h at ambient temperature. After total consumption of starting material, the reaction mixture was diluted with dichloromethane (50 mL) and washed with water (2×100 mL) followed by sodium bicarbonate solution (2×100 mL) and brine (100 mL). The organic layer was dried over magnesium sulfate and concentrated under reduced pressure. The crude compound was purified by column chromatography (silica gel: 100-200 mesh, eluent: 20% ethyl acetate in n-hexane) to get 5-bromo-N-phenylpyrimidine-2-carboxamide (5.5 g, 77%).

### Step 2:

Sodium hydride (950 mg, 23.91 mmol) was taken in a 250 mL round bottomed two-necked flask and dry dimethylformamide (20 mL) was added to it under nitrogen atmosphere. To the suspension of sodium hydride in dimethylformamide solution of 5-bromo-N-phenylpyrimidine-2-carboxamide (5.5 g, 19.92 mmol) in dry dimethylformamide (40 mL) was added at −5° C. The reaction mixture was stirred at the same temperature for 30 minutes. After that 2-(trimethylsilyl)ethoxymethyl chloride (4.98 g, 29.89 mmol) was added to it dropwise maintaining the temperature. The reaction mixture was stirred at ambient temperature for 2 h. After total consumption of starting material the reaction mixture was quenched with ammonium chloride solution (150 mL) and extracted with ethyl acetate (3×100 mL). The combined organic layer was dried over magnesium sulfate and concentrated under reduced pressure. The crude compound was purified by column chromatography (silica gel: 100-200 mesh, eluent: 20% ethyl acetate in n-hexane) to afford the pure 5-bromo-N-phenyl-N-((2-(trimethylsilyl)ethoxy)methyl)pyrimidine-2-carboxamide (7.2 g, 90%).

### Step 3:

5-Bromo-N-phenyl-N-((2-(trimethylsilyl)ethoxy)methyl)pyrimidine-2-carboxamide (6.5 g, 15.92 mmol) was dissolved in 1,4-dioxane (80 mL) and 4,4,4′,4′,5,5,5′,5′-octamethyl-2,2′-Bi-(1,3,2-dioxaborolane) (4.24 g, 16.7 mmol) was added to it followed by potassium acetate (4.68 g, 47.76 mmol) under nitrogen atmosphere. The reaction mixture was stirred for 5 minutes and Pd(dppf)Cl₂ (582 mg, 0.79 mmol) was added to it. The reaction mixture was refluxed for 16 h. After total consumption of starting material the reaction mixture was diluted with water and extracted with ethyl acetate (3×100 mL). The combined organic layer was dried over magnesium sulfate and concentrated under reduced pressure. The crude N-phenyl-5-(4,4,5,5-tetramethyl-1,3,2-dioxaborolan-2-yl)-N-((2-(trimethylsilyl)ethoxy)methyl)pyrimidine-2-carboxamide was used for next step without purification (8.0 g, crude).

### Step 4:

N-Phenyl-5-(4,4,5,5-tetramethyl-1,3,2-dioxaborolan-2-yl)-N-((2-(trimethylsilyl)ethoxy)-methyl)pyrimidine-2-carboxamide (7.3 g, 16.04 mmol) was dissolved in toluene (73 mL) and methyl 2-(trifluoromethylsulfonyloxy)acrylate (4.5 g, 19.25 mmol) was added to it followed by 2M sodium carbonate solution (32 mL) under nitrogen atmosphere. After that tetrakis (triphenylphosphine palladium (0) (927 mg, 0.80 mmol) was added to it. The reaction mixture was refluxed for 16 h. After total consumption of starting material the reaction mixture was diluted with water and extracted with ethyl acetate (3×100 mL). The combined organic layer was dried over magnesium sulfate and concentrated under reduced pressure. The crude was purified by column chromatography (silica gel: 100-200 mesh, eluent: 10% ethyl acetate in n-hexane) to afford the pure methyl 2-(2-(phenyl((2-(trimethylsilyl)ethoxy)methyl)carbamoyl)pyrimidin-5-yl)acrylate (4.3 g, 65%).

### Step 5:

Methyl 2-(2-(phenyl((2-(trimethylsilyl)ethoxy)methyl)carbamoyl)pyrimidin-5-yl)acrylate (4.3 g) was dissolved in ethyl acetate (43 mL) in a 250 mL Parr vessel and 10% Pd/C (430 mg) was added to it under nitrogen atmosphere. The vessel was equipped in Parr apparatus under 50 psi hydrogen pressure. After 2 h TLC showed the total consumption of starting material. The catalyst was filtered through celite bed and filtrate was concentrated under reduced pressure to afford methyl 2-(2-(phenyl((2-(trimethylsilyl)ethoxy)-methyl)carbamoyl)pyrimidin-5-yl)propanoate (4.0 g, 93%)

### Step 6:

Methyl 2-(2-(phenyl((2-(trimethylsilyl)ethoxy)methyl)carbamoyl)pyrimidin-5-yl)propanoate (2.5 g, 6.0 mmol) was dissolved in ethanol (75.6 mL) and 6N HCl (75.6 mL) was added to it. The reaction mixture was refluxed for 2 h at 90° C. After complete conversion of starting material ethanol was evaporated under reduced pressure and residue was diluted with water and basified by sodium carbonate solution. The aqueous layer was washed with ethyl acetate. After that the aqueous layer was acidified with 6N HCl and extracted with ethyl acetate (3×50 mL). The combined organic layer was dried over magnesium sulfate and concentrated under reduced pressure to afford the pure 2-(2-(phenylcarbamoyl)pyrimidin-5-yl)propanoic acid (750 mg, 47%).

¹H NMR (DMSO-d₆, 400 MHz): δ 12.87 (1H, s), δ 10.70 (1H, s), δ 8.97 (2H, s), δ 7.86 (2H, d), δ 7.37 (2H, t), δ 7.13 (1H, t), δ 3.97 (1H, q), δ 1.52 (3H, d); LCMS (M+H): 272.0; HPLC: 95.02%

### Step 7:

To a stirred solution of (3-tert-butyl-1-(3-chlorophenyl)-1H-pyrazol-5-yl)methanamine (0.057 g, 0.221 mmol) and 2-(2-(phenylcarbamoyl)pyrimidin-5-yl)propanoic acid (0.06 g, 0.221 mmol) in tetrahydrofuran (1.7 mL) was added 1-hydroxybenzotriazolhydrate (0.029 mL, 0.221 mmol), O-(1H-benzotriazol-1-yl)-N,N,N′,N′-tetramethyluronium tetrafluoroborate (0.071 g, 0.221 mmol) and N-ethyldiisopropylamine (0.075 mL, 0.442 mmol) and the reaction mixture was allowed to stir for 36 h. The reaction mixture was concentrated under reduced pressure and the solid obtained was purified by column chromatography (silica gel: 100-200 mesh, eluent: ethyl acetate/cyclohexane 5:1) to afford 5-(1-((3-tert-butyl-1-(3-chlorophenyl)-1H-pyrazol-5-yl)methylamino)-1-oxopropan-2-yl)-N-phenyl pyrimidine-2-carboxamide (example compound 65) (118 mg, 99%).

### Synthesis of Example 68

### 1-((3-tert-butyl-1-(3-chlorophenyl)-1H-pyrazol-5-yl)methyl)-3-(6-(2-methoxyethylamino)pyridin-3-yl)urea

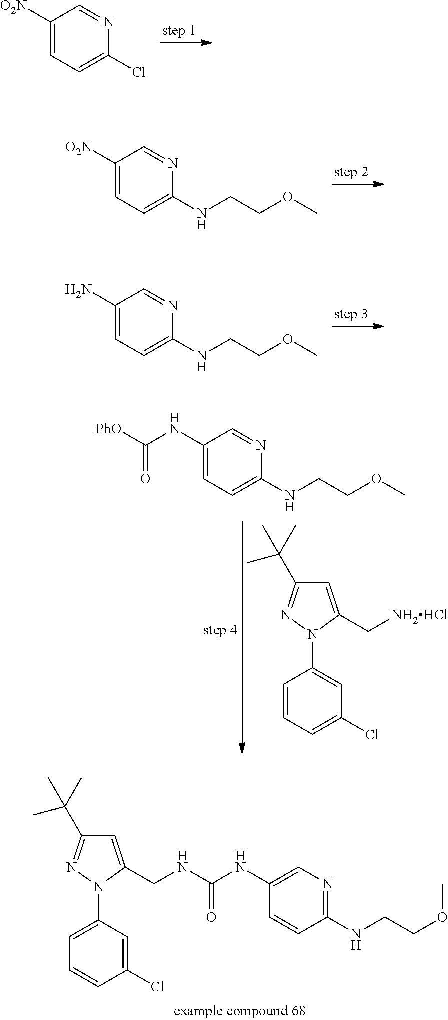

### Step 1-3

as described for example 69.

### Step 4:

To a stirred solution of (3-tert-butyl-1-(3-chlorophenyl)-1H-pyrazol-5-yl)methanamine (105 mg, 0.398 mmol) in acetonitrile (9 mL) was added triethylamine (0.22 mL, 1.592 mmol) followed by phenyl 6-(2-methoxyethylamino)pyridin-3-ylcarbamate (116 mg, 0.406 mmol) at room temperature and stirred at reflux overnight. The reaction mixture was concentrated under reduced pressure and the solid obtained was purified by column chromatography (silica gel: 100-200 mesh, eluent: ethyl acetate) to afford 1-((3-tert-butyl-1-(3-chlorophenyl)-1H-pyrazol-5-yl)methyl)-3-(6-(2-methoxyethylamino)pyridin-3-yl)urea (example compound 68) (178 mg, 98%) as an amber solid.

### Synthesis of Example 69

### 1-((1-(3-chlorophenyl)-3-(trifluoromethyl)-1H-pyrazol-5-yl)methyl)-3-(6-(2-methoxyethylamino)pyridin-3-yl)urea

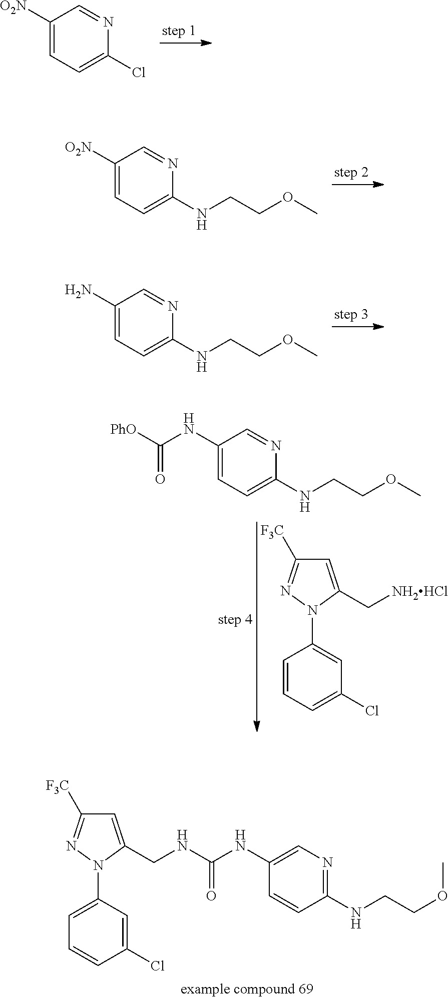

### Step 1:

2-Chloro-5-nitropyridine (4.0 g) was stirred with 2-methoxyethylamine (20 mL) at room temperature for 1 h. The reaction mixture was diluted with water (30 mL) and extracted with ethyl acetate (2×50 mL), washed with brine (20 mL), dried over sodium sulfate and evaporated under vacuum. The residue was washed with n-pentane (25 mL) to get N-(2-methoxyethyl)-5-nitropyridin-2-amine (4.8 g, 87%) as yellow solid.

### Step 2:

To a stirred solution of N-(2-methoxyethyl)-5-nitropyridin-2-amine (4.8 g, 22.84 mmol) in ethyl acetate (50 mL) was added 10% Pd/C (550 mg), then allowed to stir at room temperature for 16 h under hydrogen atmosphere. The reaction mixture was passed through celite and evaporated under reduced pressure. The residue thus obtained was washed with pentane (20 mL) to get N2-(2-methoxyethyl)pyridine-2,5-diamine (3.51 g, 87%).

### Step 3:

To a stirred solution of N2-(2-methoxyethyl)pyridine-2,5-diamine (3.8 g, 22.75 mmol) in acetone (35 mL) was added pyridine (5.5 mL, 68.25 mmol) followed by phenyl chloroformate (3.2 mL, 25.025 mmol) at 0° C. and stirred at room temperature for 1 h. The solvent was evaporated and residue obtained was dissolved in ethyl acetate (150 mL) and washed with water (50 mL), brine (50 mL), dried over sodium sulfate, evaporated and residue was purified (silica gel: 100-200 mesh, eluent: methanol/chloroforme 1:99) to get phenyl 6-(2-methoxyethylamino)pyridin-3-ylcarbamate (3.1 g, 47%) as white solid.

### Step 4:

To a stirred solution of (1-(3-chlorophenyl)-3-(trifluoromethyl)-1H-pyrazol-5-yl)methanamine hydrochloride (217 mg, 0.696 mmol) in dichloromethane (5 mL) was added triethylamine (0.3 mL, 2.088 mmol) followed by phenyl 6-(2-methoxyethylamino)pyridin-3-ylcarbamate (200 mg, 0.696 mmol) at room temperature and stirred for 16 h. The reaction mixture was diluted with dichloromethane (15 mL) and washed with water (10 mL), brine (5 mL), dried over sodium sulfate and evaporated. The residue was purified washed with ether (5 mL), dichloromethane (10 mL) to get 1-((1-(3-chlorophenyl)-3-(trifluoromethyl)-1H-pyrazol-5-yl)methyl)-3-(6-(2-methoxyethylamino)pyridin-3-yl)urea (example compound 69) (132 mg, 40%) as an off-white solid.

### Synthesis of Example 70

### 1-((3-tert-butyl-1-(3-chlorophenyl)-1H-pyrazol-5-yl)methyl)-3-(6-(2-hydroxyethylamino)pyridin-3-yl)urea

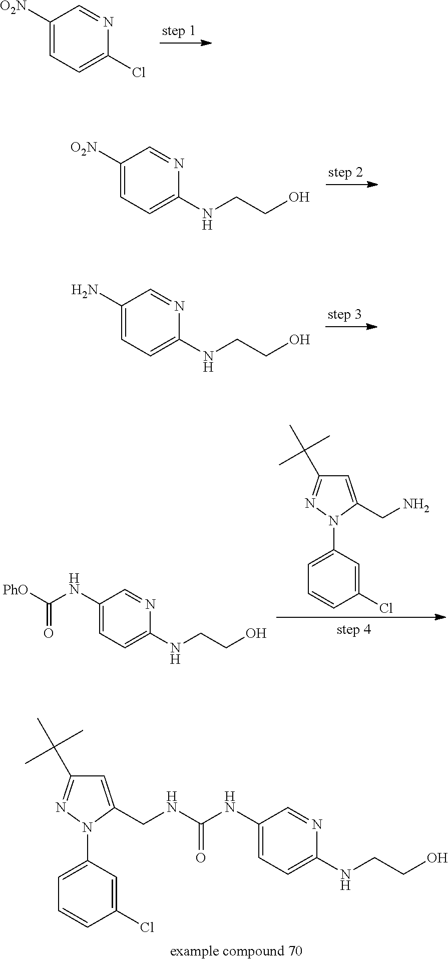 [SMILES: O=[N+]([O-])c1ccc(Cl)nc1 ; O=[N+]([O-])c1ccc(NCCO)nc1 ; Nc1ccc(NCCO)nc1 ; O=C(Nc1ccc(NCCO)nc1)Oc1ccccc1 ; CC(C)(C)c1cc(CN)n(-c2cccc(Cl)c2)n1 ; CC(C)(C)c1cc(CNC(=O)Nc2ccc(NCCO)nc2)n(-c2cccc(Cl)c2)n1]

### Step 1:

2-chloro-5-nitropyridine (4.0 g) was stirred with 2-aminoethanol (20 mL) at room temperature for 1 h. The reaction mixture was diluted with water (30 mL) and extracted with ethyl acetate (2×50 mL), washed with brine (20 mL), dried over sodium sulfate and evaporated under vacuum. The residue was washed with n-pentane (25 mL) to get 2-(5-nitropyridin-2-ylamino)ethanol (4.16 g, 91%) as yellow solid.

### Step 2:

To a stirred solution of 2-(5-nitropyridin-2-ylamino)ethanol (4.0 g, 21.85 mmol, 1 eq) in tetrahydrofuran (50 mL) was added 10% Pd/C (600 mg) and stirred at room temperature for 16 h under H₂ gas balloon pressure. The reaction mixture was passed through celite, evaporated and the residue obtained was washed with diethylether (20 mL) to get 2-(5-aminopyridin-2-ylamino)ethanol (3.02 g, 90%).

### Step 3:

To a stirred acetone (35 mL) solution of 2-(5-aminopyridin-2-ylamino)ethanol (3.0 g, 19.60 mmol, 1 eq) pyridine (4.7 mL, 58.82 mmol, 3 eq) was added followed by phenyl chloroformate (2.7 mL, 21.56 mmol, 1.1 eq) at 0° C. and stirred room temperature for 1 h. The solvent was evaporated and the residue obtained was dissolved in ethyl acetate (150 mL) and washed with water (50 mL), brine (50 mL) dried over sodium sulfate, evaporated and the residue was purified (neutral alumina, methanol/trichloromethane (1:49) as eluents) to get phenyl 6-(2-hydroxyethylamino)pyridin-3-ylcarbamate (0.80 g, 19%) as pink solid.

### Step 4:

To a stirred solution of (3-tert-butyl-1-(3-chlorophenyl)-1H-pyrazol-5-yl)methanamine (105 mg, 0.398 mmol, 1.0 eq) in acetonitrile (9 mL) was added triethylamine (0.220 mL, 1.59 mmol, 4.0 eq) followed by phenyl 6-(2-hydroxyethylamino)pyridin-3-ylcarbamate (111 mg, 0.406 mmol, 1.02 eq) and stirred for 16 h at reflux. The reaction mixture was concentrated under vacuum and the residue purified (column chromatography, silica gel, ethyl acetate/methanol (4:1) as eluent) to get 1-((3-tert-butyl-1-(3-chlorophenyl)-1H-pyrazol-5-yl)methyl)-3-(6-(2-hydroxyethylamino)pyridin-3-yl)urea (example compound 70) (125 mg, 71%).

Examples 72, 78, 80, 81 and 154-158 were prepared in a similar manner. Examples 73-77 and 82-86 can be prepared in a similar manner.

### Synthesis of Example 71

### 1-((1-(3-chlorophenyl)-3-(trifluoromethyl)-1H-pyrazol-5-yl)methyl)-3-(6-(2-hydroxyethylamino)pyridin-3-yl)urea

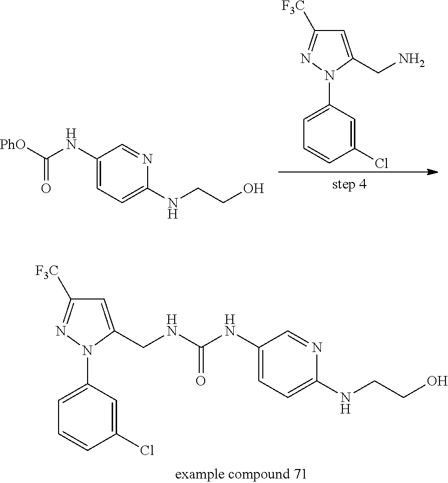 [SMILES: O=C(Nc1ccc(NCCO)nc1)Oc1ccccc1 ; NCc1cc(C(F)(F)F)nn1-c1cccc(Cl)c1 ; O=C(NCc1cc(C(F)(F)F)nn1-c1cccc(Cl)c1)Nc1ccc(NCCO)nc1]

Step 1-3: see example compound 70.

### Step 4:

To a stirred solution of (1-(3-chlorophenyl)-3-(trifluoromethyl)-1H-pyrazol-5-yl)methanamine (100 mg, 0.318 mmol, 1.0 eq) in dichloromethane (2.0 mL) was added triethylamine (0.13 mL, 0.95 mmol, 3.0 eq) followed by phenyl 6-(2-hydroxyethylamino)pyridin-3-ylcarbamate (87 mg, 0.318 mmol, 1.0 eq) at room temperature and stirred for 16 h. The reaction mixture was diluted with water (5 mL) and extracted with ethyl acetate (10 mL), washed with brine, dried over sodium sulfate and evaporated. The residue was purified by washing with ether (5 mL), dichloromethane (10 mL) to get 1-((1-(3-chlorophenyl)-3-(trifluoromethyl)-1H-pyrazol-5-yl)methyl)-3-(6-(2-hydroxyethylamino)pyridin-3-yl)urea (example compound 71) (77.7 mg, 54%) as pale pink solid.

### Synthesis of Example 79

### 1-((1-(3,4-difluorophenyl)-3-(trifluoromethyl)-1H-pyrazol-5-yl)methyl)-3-(6-(2-hydroxyethylamino)pyridin-3-yl)urea

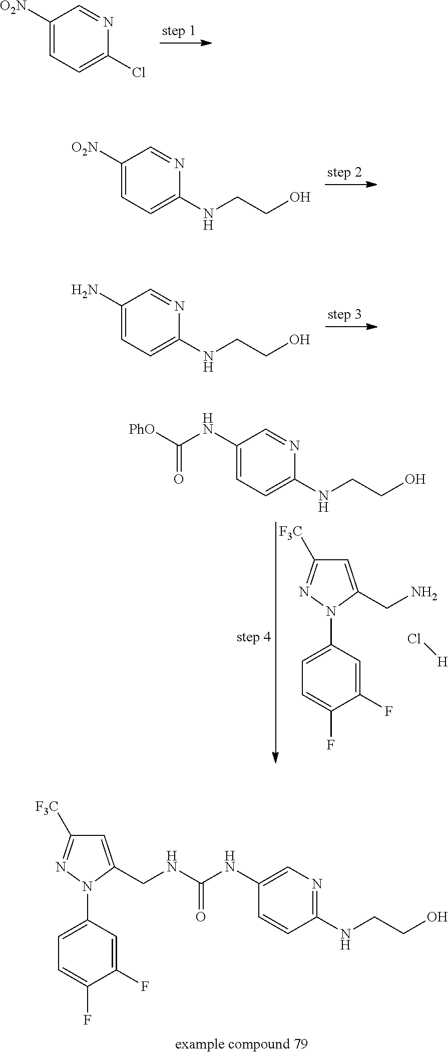

### Step 1:

2-Chloro-5-nitropyridine (4.0 g) was stirred with 2-aminoethanol (20 mL) at room temperature for 1 h. The reaction mixture was diluted with water (30 mL) and extracted with ethyl acetate (2×50 mL), washed with brine (20 mL), dried over sodium sulfate and evaporated under vacuum. The residue was washed with n-pentane (25 mL) to get 2-(5-nitropyridin-2-ylamino)ethanol (4.16 g, 91%) as yellow solid).

### Step 2:

To a stirred solution of 2-(5-nitropyridin-2-ylamino)ethanol (4.0 g, 21.85 mmol) in tetrahydrofuran (50 mL) was added 10% Pd/C (600 mg) and stirred at room temperature for 16 h under hydrogen gas balloon pressure. The reaction mixture was passed through celite, evaporated and the residue obtained was washed with diethyl ether (20 mL) to get 2-(5-aminopyridin-2-ylamino)ethanol (3.02 g, 90%).

### Step 3:

To a stirred acetone solution (35 mL) of 2-(5-aminopyridin-2-ylamino)ethanol (3.0 g, 19.60 mmol) pyridine (4.7 mL, 58.82 mmol) was added followed by phenyl chloroformate (2.7 mL, 21.56 mmol) at 0° C. and stirred at room temperature for 1 h. The solvent was evaporated and the residue obtained was dissolved in ethyl acetate (150 mL) and washed with water (50 mL), brine (50 mL), dried over sodium sulfate, evaporated and the residue was purified (neutral alumina, eluent: methanol/chloroforme 1:49) to get phenyl 6-(2-hydroxyethylamino)pyridin-3-ylcarbamate (0.8 g, 19%) as pink solid.

### Step 4:

To a stirred solution of (1-(3,4-difluorophenyl)-3-(trifluoromethyl)-1H-pyrazol-5-yl)methanamine hydrochloride (53 mg, 0.170 mmol) in tetrahydrofuran (5 mL) was added triethylamine (0.084 mL, 0.493 mmol) followed by phenyl 6-(2-hydroxyethylamino)pyridin-3-ylcarbamate (49 mg, 0.182 mmol) at 150° C. and stirred for 1.5 h under microwave conditions (7 bar). The concentrated reaction mixture was purified by column chromatography (silica gel: 100-200 mesh, eluent: ethyl acetate/methanol 10:1) to get 1-((1-(3,4-difluorophenyl)-3-(trifluoromethyl)-1H-pyrazol-5-yl)methyl)-3-(6-(2-hydroxyethylamino)pyridin-3-yl)urea (example compound 79) (46 mg, 59%) as white solid.

### Synthesis of Example 89

### 1-((1-(3-chlorophenyl)-3-(trifluoromethyl)-1H-pyrazol-5-yl)methyl)-3-(6-((2-methoxyethyl)(methyl)amino)pyridin-3-yl)urea

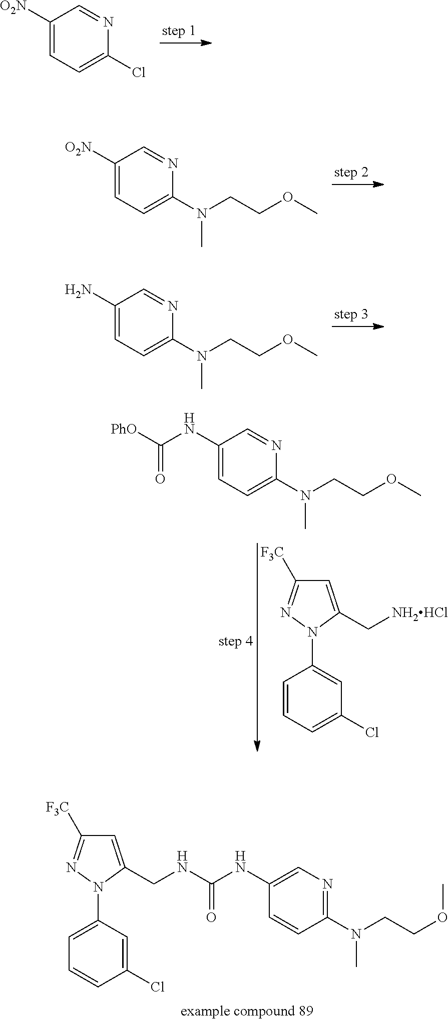

### Step 1:

2-Chloro-5-nitropyridine (3.0 g) was stirred with 2-methoxyethylmethylamine (10 mL) at room temperature for 1 h. The reaction mixture was diluted with water (50 mL), extracted with ethyl acetate (2×150 mL), washed with brine (50 mL), dried over sodium sulfate and concentrated to get N-(2-methoxyethyl)-N-methyl-5-nitropyridin-2-amine (3.3 g, 83%) as yellow solid.

### Step 2:

To a stirred solution of N-(2-methoxyethyl)-N-methyl-5-nitropyridin-2-amine (3.3 g, 15.63 mmol) in ethyl acetate (35 mL) 10% Pd/C (450 mg) was added and stirred at room temperature for 16 h under hydrogen atmosphere. The reaction mixture was then passed through celite and concentrated. The residue was washed with pentane (20 mL) to get N2-(2-methoxyethyl)-N2-methylpyridine-2,5-diamine (2.0 g, 73%).

### Step 3:

To a stirred solution of N2-(2-methoxyethyl)-N2-methylpyridine-2,5-diamine (2.0 g, 11.04 mmol) in acetone (30 mL), pyridine (4.3 mL, 33.12 mmol) was added followed by phenyl chloroformate (2.46 mL, 12.144 mmol) at 0° C. and stirred at room temperature for 1 h. The reaction mixture was and the residue was dissolved in ethyl acetate (150 mL), washed with water (50 mL), brine (50 mL), dried over sodium sulfate, evaporated and the residue was purified (silica gel: 100-200 mesh, eluent: ethyl acetate/petrol ether 2:3) to get phenyl 6-((2-methoxyethyl)(methyl)amino)pyridin-3-ylcarbamate (2.56 g, 77%) as white solid.

### Step 4:

To a stirred solution of (1-(3-chlorophenyl)-3-(trifluoromethyl)-1H-pyrazol-5-yl)methanamine hydrochloride (105 mg, 0.338 mmol) in dichloromethane (5 mL) was added triethylamine (0.15 mL, 1.014 mmol) followed by phenyl 6-((2-methoxyethyl)(methyl)amino)pyridin-3-ylcarbamate (102 mg, 0.338 mmol) at room temperature and stirred for 16 h. The reaction mixture was diluted with dichloromethane (15 mL) and washed with water (10 mL), brine (5 mL), dried over sodium sulfate and evaporated. The residue was purified (neutral alumina, eluent: methanol/chloroforme 1:49) to get 1-((1-(3-chlorophenyl)-3-(trifluoromethyl)-1H-pyrazol-5-yl)methyl)-3-(6-((2-methoxyethyl)(methyl)amino)pyridin-3-yl)urea (example compound 89) (74.8 mg, 49%) as off-white solid.

### Synthesis of Example 90

### 1-((1-(3-chlorophenyl)-3-(trifluoromethyl)-1H-pyrazol-5-yl)methyl)-3-(6-((2-hydroxyethyl)(methyl)amino)pyridin-3-yl)urea

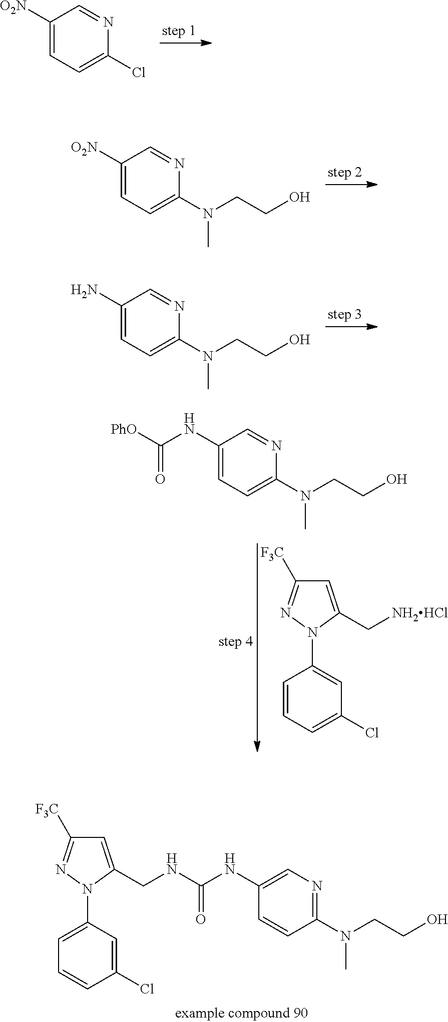

### Step 1:

2-Chloro-5-nitropyridine (4.0 g) was stirred with 2-methylaminoethanol (20 mL) at room temperature for 1 h. The reaction mixture was diluted with water (30 mL) and extracted with ethyl acetate (2×50 mL), washed with brine (20 mL), dried over sodium sulfate and evaporated under vacuum. The residue was washed with n-pentane (25 mL) to get 2-(methyl(5-nitropyridin-2-yl)amino)ethanol (4.5 g, 91%) as a yellow solid.

### Step 2:

To a stirred ethyl acetate solution (50 mL) of 2-(methyl(5-nitropyridin-2-yl)amino)ethanol (4.8 g, 24.36 mmol), 10% Pd/C (550 mg) was added and stirred at room temperature for 16 h under hydrogen atmosphere. The reaction mixture was passed through celite and evaporated under reduced pressure. The obtained residue was washed with diethyl ether (20 mL) to get 2-((5-aminopyridin-2-yl)(methyl)amino)ethanol (3.3 g, 81%).

### Step 3:

To a stirred solution of 2-((5-aminopyridin-2-yl)(methyl)amino)ethanol (3.3 g, 16.75 mmol) in acetone (40 mL), pyridine (4.0 mL, 50.25 mmol) followed by phenyl chloroformate (2.3 mL, 18.425 mmol) were added at 0° C. and stirred at room temperature for 1 h. The solvent was evaporated, the residue was dissolved in ethyl acetate (150 mL) and washed with water (50 mL), brine (50 mL), dried over sodium sulfate, evaporated and residue was purified (silica gel: 100-200 mesh, eluent: methanol/chloroforme 1:19) to get phenyl 6-((2-hydroxyethyl)(methyl)amino)pyridin-3-ylcarbamate (1.2 g, 25%) as a green solid.

### Step 4:

To a stirred solution of (1-(3-chlorophenyl)-3-(trifluoromethyl)-1H-pyrazol-5-yl)methanamine hydrochloride (108 mg, 0.348 mmol) in dichloromethane (5 mL), triethylamine (0.15 mL, 1.044 mmol) followed by phenyl 6-((2-hydroxyethyl)(methyl)amino)pyridin-3-ylcarbamate (100 mg, 0.348 mmol) at room temperature was added and stirred for 16 h. The reaction mixture was diluted with dichloromethane (15 mL) and washed with water (10 mL), brine (5 mL), dried over sodium sulfate and evaporated. The residue was washed with ether (5 mL) and dichloromethane (5 mL) to get 1-((1-(3-chlorophenyl)-3-(trifluoromethyl)-1H-pyrazol-5-yl)methyl)-3-(6-((2-hydroxyethyl)(methyl)amino)pyridin-3-yl)urea (example compound 90) (58 mg, 36%) as off-white solid.

Example 87 was prepared in a similar manner.

### Synthesis of Example 91

### N-(5-(1-((3-tert-butyl-1-(3-chlorophenyl)-1H-pyrazol-5-yl)methylamino)-1-oxopropan-2-yl)pyridin-2-yl)benzamide

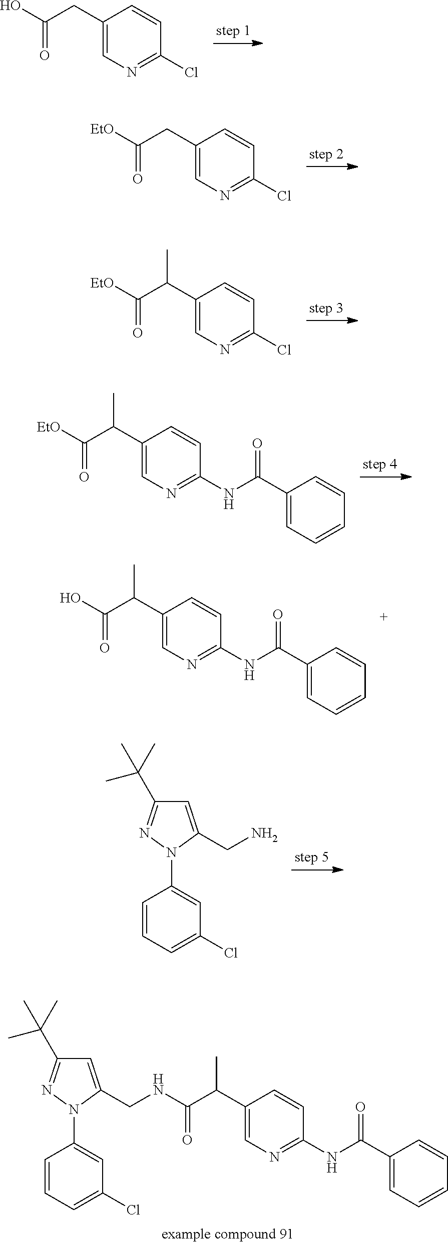

### Step 1-2

as described for example 55.

### Step 3:

The round bottom flask was charged with palladium(II) acetate (78 mg, 0.35 mmol), BINAP (218 mg, 0.35 mmol) and toluene. The mixture was stirred under nitrogen flow for 15 min and then was added ethyl 2-(6-chloropyridin-3-yl)propanoate (370 mg, 1.73 mmol), benzamide (189 mg, 1.56 mmol) and caesium carbonate (2.258 g, 6.93 mmol). The reaction mixture was refluxed overnight and then cooled to room temperature. The mixture was filtered through a plug of celite and concentrated. The residue was diluted with ethyl acetate and washed with 10% HCl solution. The organic layer was dried over magnesium sulfate and concentrated under reduced pressure to afford crude which was purified by column chromatography to afford ethyl 2-(6-benzamidopyridin-3-yl)propanoate (295 mg, 63%).

### Step 4:

To a solution of ethyl 2-(6-benzamidopyridin-3-yl)propanoate (295 mg, 0.99 mmol) in tetrahydrofuran and water was added lithium hydroxide monohydrate (62 mg, 1.48 mmol). The reaction mixture was stirred for 2 h at 40° C. and then acidified with 10% HCl solution. The mixture was extracted with ethyl acetate. The organic was dried over magnesium sulfate and concentrated under reduced pressure to afford 2-(6-benzamidopyridin-3-yl)propanoic acid (250 mg, 94%).

### Step 5:

To a solution of 2-(6-benzamidopyridin-3-yl)propanoic acid (80 mg, 0.30 mmol) in dimethylformamide was added 1-hydroxybenzotriazole (60 mg, 0.44 mmol), 1-ethyl-3-(3-dimethylaminopropyl) carbodiimide (85 mg, 0.44 mmol), triethylamine (0.08 mL, 0.59 mmol) and (3-tert-butyl-1-(3-chlorophenyl)-1H-pyrazol-5-yl)methanamine (78 mg, 0.30 mmol). The reaction mixture was stirred for overnight at room temperature. The mixture was diluted with water and extracted with ethyl acetate. The organic layer was dried over magnesium sulfate and concentrated under reduced pressure. The crude was purified by column chromatography to give pure N-(5-(1-((3-tert-butyl-1-(3-chlorophenyl)-1H-pyrazol-5-yl)methylamino)-1-oxopropan-2-yl)pyridin-2-yl)benzamide (example compound 91) (105 mg, 69%).

¹H NMR (400 MHz, CDCl₃) δ 8.53 (br.s, NH), 8.34 (d, 1H, J=8.58 Hz, pyridine-H), 8.14 (d, 1H, J=2.09 Hz, pyridine-H), 7.90 (m, 2H, Ar—H), 7.63 (dd, 1H, J=8.61, 2.29 Hz, pyridine-H), 7.54 (m, 3H, Ar—H), 7.40 (m, 1H, Ar—H), 7.27 (m, 3H, Ar—H), 6.06 (s, 1H, pyrazole-H), 5.47 (m, NH), 4.48 (m, 2H, Ar—CH₂), 3.46 (quartet, 1H, J=7.14 Hz, amide-CH), 1.48 (d, 3H, J=7.17 Hz, amide-CH₃), 1.28 (s, 9H, t-butyl-H).

### Synthesis of Example 92

### N-(5-(1-((1-(3-chlorophenyl)-3-(trifluoromethyl)-1H-pyrazol-5-yl)methylamino)-1-oxopropan-2-yl)pyridin-2-yl)benzamide

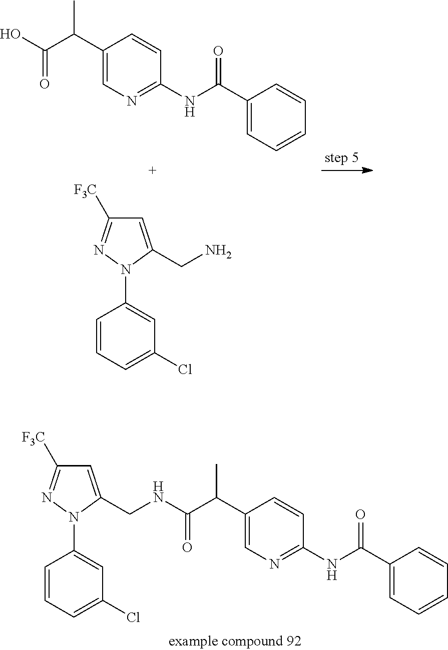 [SMILES: CC(C(=O)O)c1ccc(NC(=O)c2ccccc2)nc1 ; NCc1cc(C(F)(F)F)nn1-c1cccc(Cl)c1 ; CC(C(=O)NCc1cc(C(F)(F)F)nn1-c1cccc(Cl)c1)c1ccc(NC(=O)c2ccccc2)nc1]

### Step 1-4

as described for example 91

### Step 5:

To a solution of 2-(6-benzamidopyridin-3-yl)propanoic acid (80 mg, 0.30 mmol) in dimethylformamide was added 1-hydroxybenzotriazole (60 mg, 0.44 mmol), 1-ethyl-3-(3-dimethylaminopropyl) carbodiimide (85 mg, 0.44 mmol), triethylamine (0.08 mL, 0.59 mmol) and (1-(3-chlorophenyl)-3-(trifluoromethyl)-1H-pyrazol-5-yl)methanamine (81 mg, 0.30 mmol). The reaction mixture was stirred overnight at room temperature. The mixture was diluted with water and extracted with ethyl acetate. The organic layer was dried over magnesium sulfate and concentrated under reduced pressure. The crude was purified by column chromatography to give pure N-(5-(1-((1-(3-chlorophenyl)-3-(trifluoromethyl)-1H-pyrazol-5-yl)methylamino)-1-oxopropan-2-yl)pyridin-2-yl)benzamide (example compound 92) (125 mg, 80%).

¹H NMR (400 MHz, CDCl₃) δ 8.54 (br.s, NH), 8.34 (d, 1H, J=8.62 Hz, pyridine-H), 8.16 (d, 1H, J=2.04 Hz, pyridine-H), 7.90 (m, 2H, Ar—H), 7.64 (m, 1H, pyridine-H), 7.57 (m, 1H, Ar—H), 7.50 (m, 2H, Ar—H), 7.40 (m, 3H, Ar—H), 7.28 (m, 1H, Ar—H), 6.49 (s, 1H, pyrazole-H), 5.59 (m, NH), 4.49 (m, 2H, Ar—CH₂), 3.49 (quartet, 1H, J=7.12 Hz, amide-CH), 1.49 (d, 3H, J=7.14 Hz, amide-CH₃).

### Synthesis of Example 93

### N-(5-(1-((3-tert-butyl-1-(3-chlorophenyl)-1H-pyrazol-5-yl)methylamino)-1-oxopropan-2-yl)pyridin-2-yl)-4-fluorobenzamide

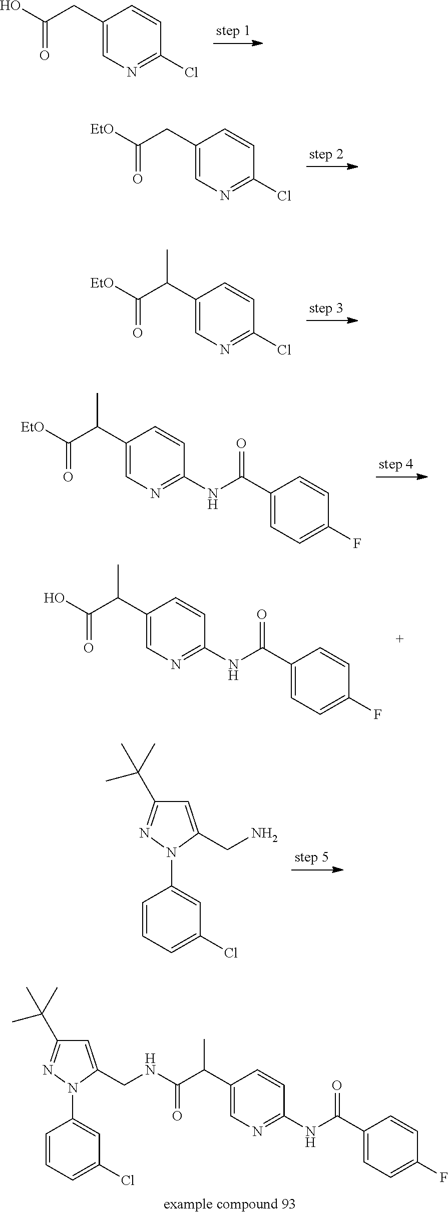

### Step 1-2

as described for example 55.

### Step 3:

The round bottom flask was charged with palladium(II) acetate (42 mg, 0.19 mmol), BINAP (118 mg, 0.19 mmol) and toluene. The mixture was stirred under nitrogen flow for 15 min and then was added ethyl 2-(6-chloropyridin-3-yl)propanoate (200 mg, 0.94 mmol), 4-fluorobenzamide (130 mg, 0.94 mmol) and caesium carbonate (1.225 g, 3.76 mmol). The reaction mixture was refluxed overnight and then cooled to room temperature. The mixture was filtered through a plug of celite and concentrated. The residue was diluted with ethyl acetate and washed with 10% HCl solution. The organic layer was dried over magnesium sulfate and concentrated under reduced pressure to afford the crude compound, which was purified by column chromatography to afford the pure ethyl 2-(6-(4-fluorobenzamido)pyridin-3-yl)propanoate (160 mg, 54%).

### Step 4:

To a solution of ethyl 2-(6-(4-fluorobenzamido)pyridin-3-yl)propanoate (160 mg, 0.51 mmol) in tetrahydrofuran and water was added lithium hydroxide monohydrate (32 mg, 0.76 mmol). The reaction mixture was stirred for 2 h at 40° C. and then acidified with 10% HCl solution. The mixture was extracted with ethyl acetate. The organic layer was dried over magnesium sulfate and concentrated under reduced pressure to 2-(6-(4-fluorobenzamido)pyridin-3-yl)propanoic acid (150 mg, 99%).

### Step 5:

To a solution of 2-(6-(4-fluorobenzamido)pyridin-3-yl)propanoic acid (50 mg, 0.17 mmol) in dimethylformamide was added 1-hydroxybenzotriazole (35 mg, 0.26 mmol), 1-ethyl-3-(3-dimethylaminopropyl) carbodiimide (50 mg, 0.26 mmol), triethylamine (0.05 mL, 0.35 mmol) and (3-tert-butyl-1-(3-chlorophenyl)-1H-pyrazol-5-yl)methanamine (46 mg, 0.17 mmol). The reaction mixture was stirred for overnight at room temperature. The mixture was diluted with water and extracted with ethyl acetate. The organic layer was dried over magnesium sulfate and concentrated under reduced pressure. The crude was purified by column chromatography to give pure N-(5-(1-((3-tert-butyl-1-(3-chlorophenyl)-1H-pyrazol-5-yl)methylamino)-1-oxopropan-2-yl)pyridin-2-yl)-4-fluorobenzamide (example compound 93) (46 mg, 50%).

¹H NMR (300 MHz, CDCl₃) δ 8.56 (br.s, NH), 8.32 (d, 1H, J=8.68 Hz, pyridine-H), 8.15 (d, 1H, J=2.41 Hz, pyridine-H), 7.94 (m, 2H, Ar—H), 7.66 (dd, 1H, J=8.43, 2.39 Hz, pyridine-H), 7.39 (m, 1H, Ar—H), 7.24 (m, 5H, Ar—H), 6.08 (s, 1H, pyrazole-H), 5.54 (t, NH, J=5.42 Hz), 4.50 (d, 2H, J=5.41 Hz, Ar—CH₂), 3.48 (quartet, 1H, J=7.21 Hz, amide-CH), 1.49 (d, 3H, J=7.19 Hz, amide-CH₃), 1.30 (s, 9H, t-butyl-H).

### Synthesis of Example 94

### N-(5-(1-((1-(3-chlorophenyl)-3-(trifluoromethyl)-1H-pyrazol-5-yl)methylamino)-1-oxopropan-2-yl)pyridin-2-yl)-4-fluorobenzamide

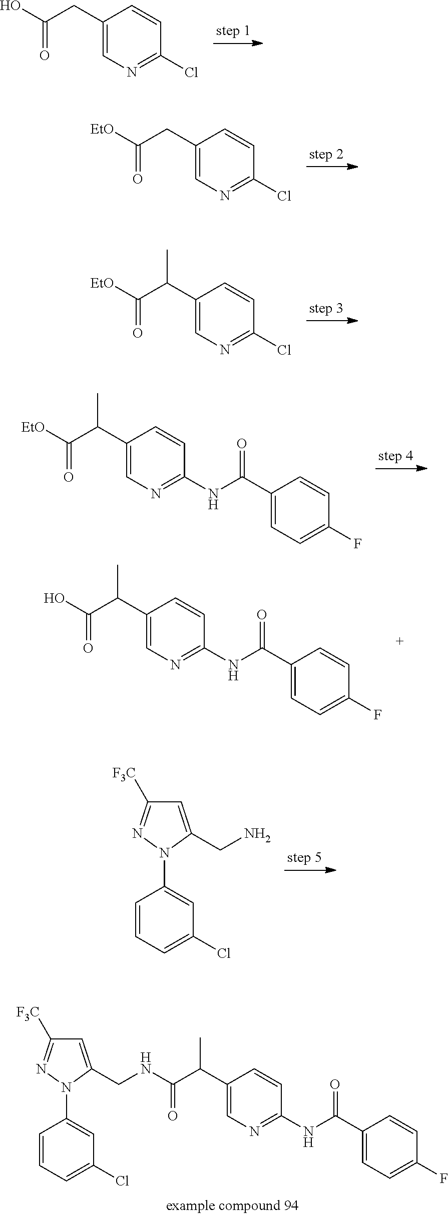

### Step 1-4

as described for example 93.

### Step 5:

To a solution of 2-(6-(4-fluorobenzamido)pyridin-3-yl)propanoic acid (50 mg, 0.17 mmol) in dimethylformamide was added 1-hydroxybenzotriazole (35 mg, 0.26 mmol), 1-ethyl-3-(3-dimethylaminopropyl) carbodiimide (50 mg, 0.26 mmol), triethylamine (0.05 mL, 0.35 mmol) and (1-(3-chlorophenyl)-3-(trifluoromethyl)-1H-pyrazol-5-yl)methanamine (48 mg, 0.17 mmol). The reaction mixture was stirred for overnight at room temperature. The mixture was diluted with water and extracted with ethyl acetate. The organic layer was dried over magnesium sulfate and concentrated under reduced pressure. The crude was purified by column chromatography to give pure N-(5-(1-((1-(3-chlorophenyl)-3-(trifluoromethyl)-1H-pyrazol-5-yl)methylamino)-1-oxopropan-2-yl)pyridin-2-yl)-4-fluorobenzamide (example compound 94) (44 mg, 46%).

¹H NMR (400 MHz, CDCl₃) δ 8.48 (br.s, NH), 8.31 (d, 1H, J=8.60 Hz, pyridine-H), 8.16 (d, 1H, J=2.10 Hz, pyridine-H), 7.92 (m, 2H, Ar—H), 7.64 (dd, 1H, J=8.58, 2.27 Hz, pyridine-H), 7.40 (m, 3H, Ar—H), 7.28 (m, 3H, Ar—H), 7.18 (m, 2H, Ar—H), 6.48 (s, 1H, pyrazole-H), 5.58 (m, NH), 4.49 (m, 2H, Ar—CH₂), 3.48 (quartet, 1H, J=7.14 Hz, amide-CH), 1.49 (d, 3H, J=7.14 Hz, amide-CH₃).

### Synthesis of Example 95

### N-(5-(1-((3-tert-butyl-1-(3-chlorophenyl)-1H-pyrazol-5-yl)methylamino)-1-oxopropan-2-yl)pyridin-2-yl)-4-chlorobenzamide

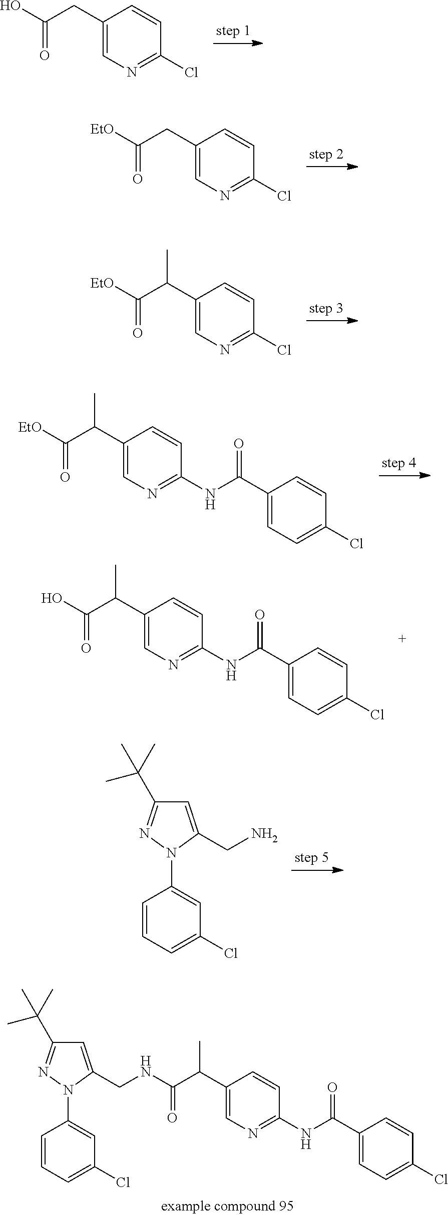

### Step 1-2

as described for example 55.

### Step 3:

The round bottom flask was charged with palladium(II) acetate (84 mg, 0.37 mmol), BINAP (233 mg, 0.37 mmol) and toluene. The mixture was stirred under nitrogen flow for 15 min and then was added ethyl 2-(6-chloropyridin-3-yl)propanoate (400 mg, 1.87 mmol), 4-chlorobenzamide (291 mg, 1.87 mmol) and caesium carbonate (2.44 g, 7.49 mmol). The reaction mixture was refluxed overnight and then cooled to room temperature. The mixture was filtered through a plug of celite and concentrated. The residue was diluted with ethyl acetate and washed with 10% HCl solution. The organic layer was dried over magnesium sulfate and concentrated under reduced pressure to afford crude which was purified by column chromatography to afford ethyl 2-(6-(4-chlorobenzamido)pyridin-3-yl)propanoate (360 mg, 58%).

### Step 4:

To a solution of ethyl 2-(6-(4-chlorobenzamido)pyridin-3-yl)propanoate (360 mg, 1.08 mmol) in tetrahydrofuran and water was added lithium hydroxide monohydrate (68 mg, 1.62 mmol). The reaction mixture was stirred for 2 h at 40° C. and then acidified with 10% HCl solution. The mixture was extracted with ethyl acetate. The organic layer was dried over magnesium sulfate and concentrated under reduced pressure to afford 2-(6-(4-chlorobenzamido)pyridin-3-yl)propanoic acid (300 mg, 91%).

### Step 5:

To a solution of 2-(6-(4-chlorobenzamido)pyridin-3-yl)propanoic acid (70 mg, 0.23 mmol) in dimethylformamide was added 1-hydroxybenzotriazole (46 mg, 0.34 mmol), 1-ethyl-3-(3-dimethylaminopropyl) carbodiimide (66 mg, 0.34 mmol), triethylamine (0.06 mL, 0.46 mmol) and (3-tert-butyl-1-(3-chlorophenyl)-1H-pyrazol-5-yl)methanamine (60 mg, 0.23 mmol). The reaction mixture was stirred for overnight at room temperature. The mixture was diluted with water and extracted with ethyl acetate. The organic layer was dried over magnesium sulfate and concentrated under reduced pressure. The crude was purified by column chromatography to give pure N-(5-(1-((3-tert-butyl-1-(3-chlorophenyl)-1H-pyrazol-5-yl)methylamino)-1-oxopropan-2-yl)pyridin-2-yl)-4-chlorobenzamide (example compound 95) (70 mg, 55%).

¹H NMR (300 MHz, CDCl₃) δ 8.68 (br.s, NH), 8.32 (d, 1H, J=8.62 Hz, pyridine-H), 8.12 (d, 1H, J=2.24 Hz, pyridine-H), 7.86 (m, 2H, Ar—H), 7.66 (dd, 1H, J=8.63, 2.38 Hz, pyridine-H), 7.49 (m, 2H, Ar—H), 7.42 (m, 1H, Ar—H), 7.28 (m, 3H, Ar—H), 6.08 (s, 1H, pyrazole-H), 5.60 (t, NH, J=5.53 Hz), 4.50 (d, 2H, J=5.52 Hz, Ar—CH₂), 3.47 (quartet, 1H, J=7.14 Hz, amide-CH), 1.48 (d, 3H, J=7.14 Hz, amide-CH₃), 1.29 (s, 9H, t-butyl-H).

### Synthesis of Example 96

### 4-chloro-N-(5-(1-((1-(3-chlorophenyl)-3-(trifluoromethyl)-1H-pyrazol-5-yl)methylamino)-1-oxopropan-2-yl)pyridin-2-yl)benzamide

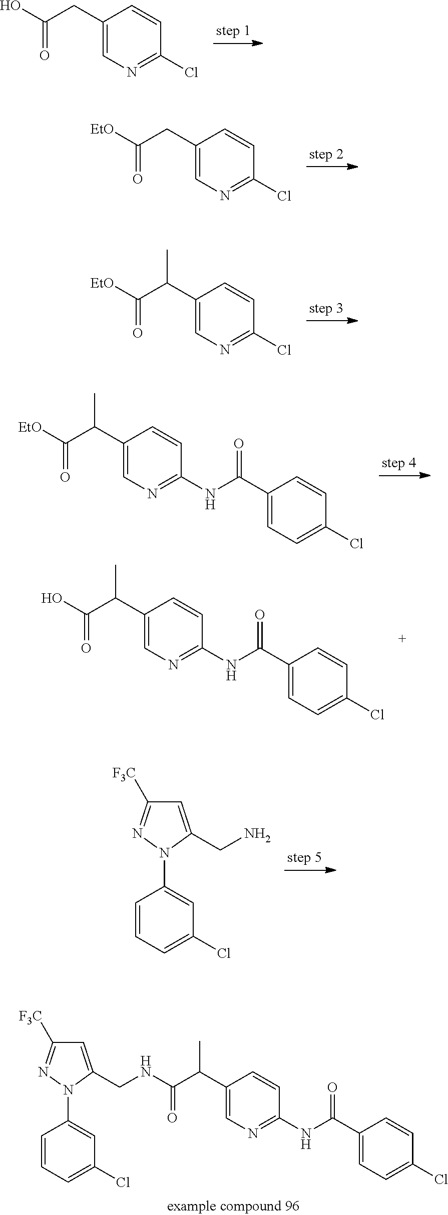

### Step 1-4

as described for example 95.

### Step 5:

To a solution of 2-(6-(4-chlorobenzamido)pyridin-3-yl)propanoic acid (70 mg, 0.23 mmol) in dimethylformamide was added 1-hydroxybenzotriazole (46 mg, 0.34 mmol), 1-ethyl-3-(3-dimethylaminopropyl) carbodiimide (66 mg, 0.34 mmol), triethylamine (0.06 mL, 0.46 mmol) and (1-(3-chlorophenyl)-3-(trifluoromethyl)-1H-pyrazol-5-yl)methanamine (63 mg, 0.23 mmol). The reaction mixture was stirred overnight at room temperature. The mixture was diluted with water and extracted with ethyl acetate. The organic layer was dried over magnesium sulfate and concentrated under reduced pressure. The crude was purified by column chromatography to give pure 4-chloro-N-(5-(1-((1-(3-chlorophenyl)-3-(trifluoromethyl)-1H-pyrazol-5-yl)methylamino)-1-oxopropan-2-yl)pyridin-2-yl)benzamide (example compound 96) (81 mg, 63%).

¹H NMR (300 MHz, CDCl₃) δ 8.56 (br.s, NH), 8.32 (d, 1H, J=8.77 Hz, pyridine-H), 8.16 (d, 1H, J=2.24 Hz, pyridine-H), 7.86 (m, 2H, Ar—H), 7.66 (dd, 1H, J=8.64, 2.38 Hz, pyridine-H), 7.45 (m, 5H, Ar—H), 7.29 (m, 1H, Ar—H), 6.50 (s, 1H, pyrazole-H), 5.67 (m, NH), 4.51 (m, 2H, Ar—CH₂), 3.50 (quartet, 1H, J=7.08 Hz, amide-CH), 1.51 (d, 3H, J=7.12 Hz, amide-CH₃).

Example 97 can be prepared in a similar manner.

### Synthesis of Example 102

### N-((3-tert-butyl-1-(3-chlorophenyl)-1H-pyrazol-5-yl)methyl)-2-(5-fluoro-6-(methylsulfonamido)pyridin-3-yl)propanamide

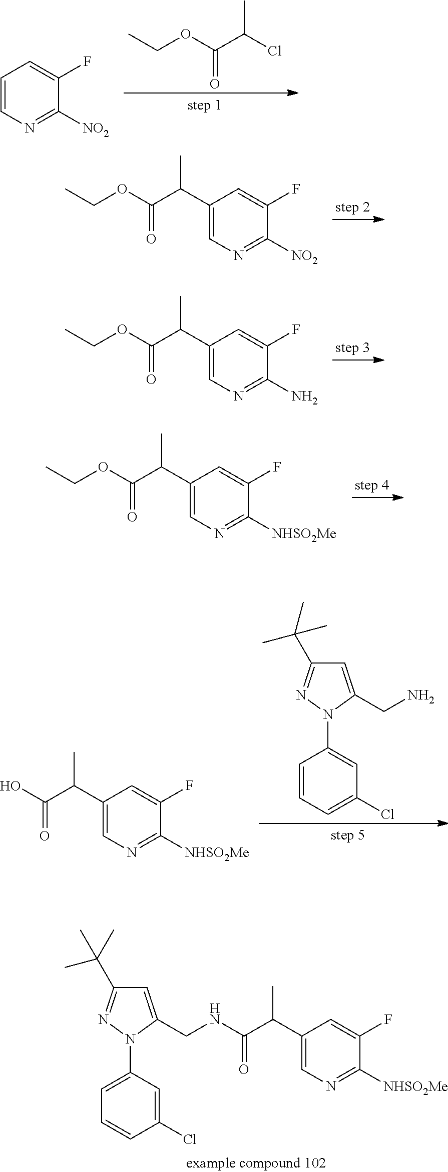

### Step 1:

In a round bottom flask potassium tertiary butoxide (0.473 g, 4221 mmol) was taken under nitrogen atmosphere, anhydrous dimethylformamide (5 mL) was added and stirred at room temperature for 10 min. Then cooled to 20° C. and 2-nitro-3-fluoropyridine (200 mg, 1.407 mmol) was added followed by dropwise addition of 2-chloropropionic acid ethyl ester (0.273 mL, 2.111 mol) and stirred for 20 min. Then dilute HCl was added and stirred at room temperature for 10 min. Extracted in ethyl acetate, washed with water dried over magnesium sulfate, filtered and solvent was evaporated and finally purified by column chromatography. to afford ethyl 2-(5-fluoro-6-nitropyridin-3-yl)propanoate (153 mg, 45%).

### Step 2:

In a round bottom ethyl 2-(5-fluoro-6-nitropyridin-3-yl)propanoate (100 mg) was taken followed by addition of ethanol and Pd/C (20 wt %) stirred at room temperature in presence of hydrogen for 2 h. Then celite filtration and solvent was evaporated to afford ethyl 2-(6-amino-5-fluoropyridin-3-yl)propanoate (69 mg, 79%)

### Step 3:

In a round bottom flask ethyl 2-(6-amino-5-fluoropyridin-3-yl)propanoate (1.525 g, 7.185 mmol) was taken under nitrogen atmosphere, Anhydrous tetrahydrofuran (14 mL) was added and stirred Then cooled to 0° C. and triethylamine (2.181 mL, 21.555 mmol) was added followed by addition methanesulfonylchloride (0.837 mL, 10.778 mmol) and stirred at room temperature for 2 h. Reaction mixture was extracted in ethyl acetate, washed with water dried over magnesium sulfate, filtered and solvent was evaporated and finally purified by column chromatography to afford ethyl 2-(5-fluoro-6-(methylsulfonamido)pyridin-3-yl)propanoate (1.39 g, 67%).

### Step 4:

In a round bottom flask ethyl 2-(5-fluoro-6-(methylsulfonamido)pyridin-3-yl)propanoate (110 mg, 0.378 mmol) ethyl ester was taken then tetrahydrofuran (5 mL) was added and cooled to 0° C. and lithiumhydroxide monohydrate (0.039 g, 0.947 mmol) solution in water (5 mL) was added dropwise and stirred at room temperature for 2 h. Then reaction mixture was extracted in ethyl acetate, washed with water and aqueous layer was acidified by using dilute HCl and extracted in ethyl acetate washed with water, dried magnesium sulfate, filtered and solvent was evaporated to 2-(5-fluoro-6-(methylsulfonamido)pyridin-3-yl)propanoic acid (59 mg, 60%).

### Step 5:

In a round bottom flask 2-(5-fluoro-6-(methylsulfonamido)pyridin-3-yl)propanoic acid (100 mg, 0.365 mmol) was taken under nitrogen atmosphere dimethylformamide (5 mL) was added Followed by addition of N-(3-dimethylaminopropyl)-N′-ethylcarbodiimide (104 mg, 0.547 mmol) and HOBt (74 mg, 0.547 mmol) stirred for 1 h. (3-Tert-butyl-1-(3-chlorophenyl)-1H-pyrazol-5-yl)methanamine (96 mg, 0.365 mmol) was added and stirred at room temperature for 4 h. Reaction mixture was extracted in ethyl acetate, washed with water dried magnesium sulfate, filtered and solvent was evaporated and finally purified by column chromatography to N-((3-tert-butyl-1-(3-chlorophenyl)-1H-pyrazol-5-yl)methyl)-2-(5-fluoro-6-(methylsulfonamido)pyridin-3-yl)propanamide as a white solid (example compound 102, 130 mg, 67%).

¹H NMR (300 MHz, CDCl₃) δ 7.97 (s, 1H, Ar—H), 7.41 (d, 3H, J=1.5 Hz, 2HAr—H) 7.36 (s, 2H, Ar—H), 6.13 (s, 1H, Ar—H), 5.64 (s, 1H,R—NH), 4.50 (d, 2H, J=4.2 Hz,Ar—CH₂), 3.46 (s, 3H, ArSO₂—CH₃), 3.4 3 (q, 1H, J=6.9 Hz, Ar—CH), 1.45 (d, 3H, J=6.9 Hz, ArCH—CH₃), 1.31 (s, 9H, R—C(CH₃)₃)

Examples 98-100 were prepared in a similar manner.

### Synthesis of Example 103

### N-((1-(3-chlorophenyl)-3-(trifluoromethyl)-1H-pyrazol-5-yl)methyl)-2-(5-fluoro-6-(methylsulfonamido)pyridin-3-yl)propanamide

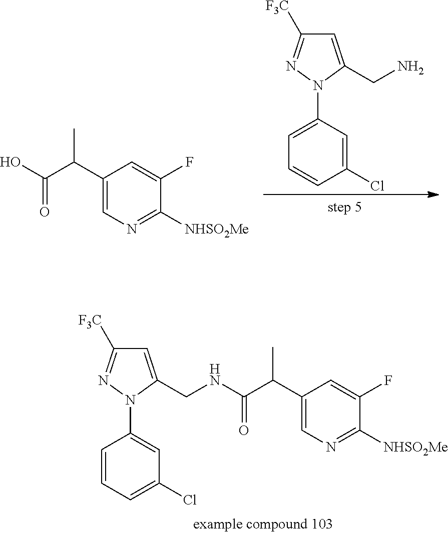

### Step 1-4

see example compound 102.

### Step 5:

In a round bottom flask 2-(5-fluoro-6-(methylsulfonamido)pyridin-3-yl)propanoic acid (100 mg, 0.365 mmol) was taken under nitrogen atmosphere dimethylformamide (5 mL) was added followed by addition of N-(3-dimethylaminopropyl)-N′-ethylcarbodiimide (104 mg, 0.547 mmol) and HOBt (74 mg, 0.547 mmol) stirred for 1 h. (1-(3-chlorophenyl)-3-(trifluoromethyl)-1H-pyrazol-5-yl)methanamine (99 mg, 0.365 mmol) was added and stirred at room temperature for 4 h. Reaction mixture was extracted in ethyl acetate, washed with water, dried over magnesium sulfate, filtered and solvent was evaporated and finally purified by column chromatography to afford N-((1-(3-chlorophenyl)-3-(trifluoromethyl)-1H-pyrazol-5-yl)methyl)-2-(5-fluoro-6-(methylsulfonamido)pyridin-3-yl)propanamide (example compound 103) as a white solid (140 mg, 71%).

¹H NMR (300 MHz, CDCl₃) δ 7.98 (s, 1H, Ar—H), 7.41 (d, 2H, J=1.5 Hz, 2H,Ar—H), 7.40 (s, 2H, Ar—H), 7.33 (m, 1H, Ar—H), 6.52 (s, 1H, Ar—H), 5.80 (s, 1H, NH), 4.50 (m, 2H, Ar—CH₂, 3.48 (s, 3H, ArSO₂—CH₃), 3.4 8 (q, 1H, J=11.91 Hz, Ar—CH), 1.46 (d, 3H, J=6.9 Hz, ArCH—CH₃).

### Synthesis of Example 104

### N-((1-(3-chlorophenyl)-3-(trifluoromethyl)-1H-pyrazol-5-yl)methyl)-2-(5-methoxy-6-(methylsulfonamido)pyridin-3-yl)propanamide

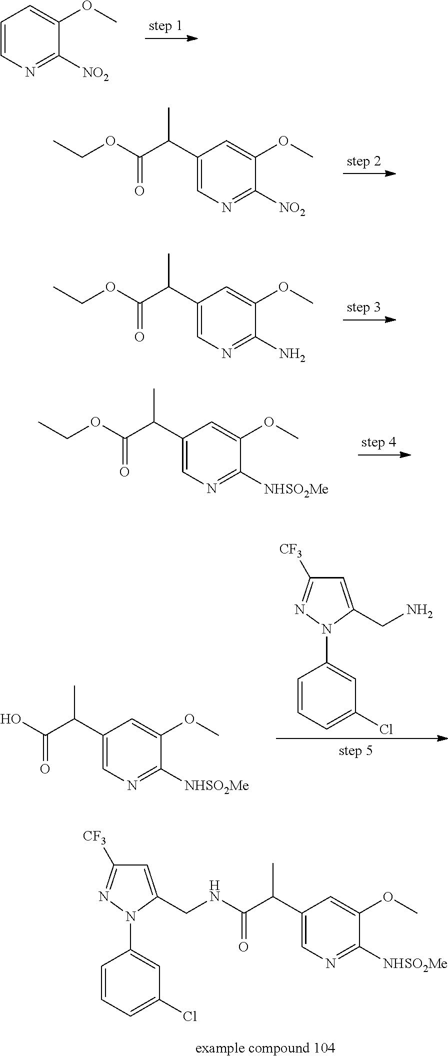

### Step 1:

In a round bottom flask potassium tertiary butoxide (146 mg, 1.297 mmol) was taken under nitrogen atmosphere, dimethylformamide (3 mL) was added, stirred at room temperature for 10 min and then cooled to −40° C. and commercially available 2-nitro-3-methoxypyridine (100 mg, 0.648 mmol) was added followed by dropwise addition of 2-chloro-propionic acid ethyl ester (0.0908 mL, 0.712 mmol) and stirred for 20 min. Then diluted HCl was added and stirred at room temperature for 10 min. Extracted in ethyl acetate, washed with water dried over magnesium sulfate, filtered and solvent was evaporated and finally purified by column chromatography to afford 2-(5-methoxy-6-nitro-pyridin-3-yl)-propionic acid ethyl ester (82 mg, 50%).

### Step 2:

In a round bottom flask 2-(5-methoxy-6-nitro-pyridin-3-yl)-propionic acid ethyl (100 mg) ester was taken followed by addition of ethanol and 20% Pd/C then stirred at room temperature in presence of hydrogen for 2 h. Then celite filtration and solvent was evaporated to afford 2-(6-Amino-5-methoxy-pyridin-3-yl)-propionic acid ethyl ester (68 mg, 78%).

### Step 3:

In a round bottom flask 2-(6-amino-5-methoxy-pyridin-3-yl)-propionic acid ethyl ester (200 mg, 0.891 mmol)was taken under nitrogen atmosphere, tetrahydrofuran was added and stirred Then cooled to 0° C. and triethylamine (0.137 mL, 0.981 mmol) was added. Followed by addition of methanesulfonylchloride (0.076 mL, 0.981 mmol) and stirred at room temperature for 2 h. Reaction mixture was extracted in ethyl acetate, washed with water, dried over magnesium sulfate, filtered and solvent was evaporated and finally purified by column chromatography to afford 2-(6-methanesulfonylamino-5-methoxy-pyridin-3-yl)-propionic acid ethyl ester (180 mg, 67%).

### Step 4:

In a round bottom flask 2-(5-methoxy-6-methanesulfonylamino-pyridin-3-yl)-propionic acid ethyl ester (1.6 g, 5.291 mmol) was taken, then tetrahydrofuran was added and cooled to 0° C. Lithiumhydroxide monohydrate (556 mg, 13.229 mmol) solution in water (10 mL) was added dropwise and stirred at room temperature for 2 h. Then reaction mixture was extracted in ethyl acetate, washed with water and aqueous layer was acidified by using diluted HCl and extracted in ethylacetate washed with water, dried over magnesium sulfate, filtered and solvent was evaporated to afford 2-(5-methoxy-6-(methylsulfonamido)pyridin-3-yl)propanoic acid (870 mg, 60%).

### Step 5:

In a round bottom flask 2-(5-methoxy-6-(methylsulfonamido)pyridin-3-yl)propanoic acid (109 mg, 0.362 mmol) was taken under nitrogen atmosphere dimethylformamide (5 mL) was added. Followed by addition of 1-ethyl-3-(3-dimethylaminopropyl) carbodiimide (104 mg, 0.543 mmol) and 1-hydroxybenzotriazole (73 mg, 0543 mmol) and stirred for 1 h. Then (1-(3-chlorophenyl)-3-(trifluoromethyl)-1H-pyrazol-5-yl)methanamine (100 mg, 0.362 mmol) was added and stirred at room temperature for 4 h. Reaction mixture was extracted in ethyl acetate, washed with water, dried over magnesium sulfate, filtered and solvent was evaporated and finally purified by column chromatography. to afford N-((1-(3-chlorophenyl)-3-(trifluoromethyl)-1H-pyrazol-5-yl)methyl)-2-(5-methoxy-6-(methylsulfonamido)pyridin-3-yl)propanamide (example compound 104) (145 mg, 69%) as a white solid.

¹H NMR (300 MHz, CDCl₃) δ 7.74 (s, 1H, Ar—H), 7.43 (m, 3H, Ar—H,), 7.30 (s, 1H, Ar—H), 7.05 (s, 1H, Ar—H), 6.46 (s, 1H, Ar—H), 5.83 (s, 1H, R—NH), 4.47 (d, 2H, J=4.02 Hz, ArCH₂), 3.86 (s, 3H, Ar—OCH₃), 3.46 (s, 3H, RSO₂—CH₃), 3.46 (q, 1H, J=6.96 Hz, Ar—CH), 1.44 (d, 3H, J=7.14 Hz, ArCH—CH₃).

### Synthesis of Example 105

### N-((3-tert-butyl-1-(3-chlorophenyl)-1H-pyrazol-5-yl)methyl)-2-(5-methoxy-6-(methylsulfonamido)pyridin-3-yl)propanamide

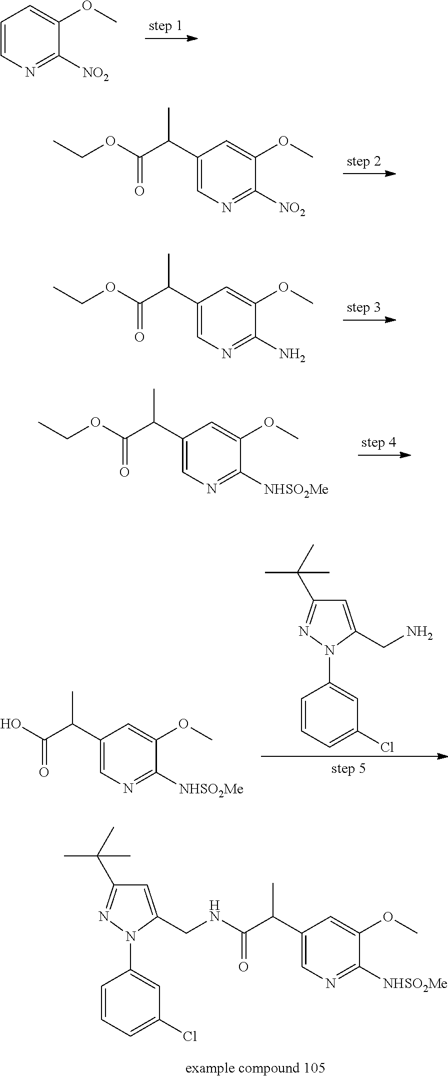

### Step 1-4

as described for example 104.

### Step 5:

In a round bottom flask 2-(5-methoxy-6-(methylsulfonamido)pyridin-3-yl)propanoic acid (80 mg, 0.292 mmol) was taken under nitrogen atmosphere dimethylformamide (5 mL) was added, followed by addition of 1-ethyl-3-(3-dimethylaminopropyl) carbodiimide (76 mg, 0.397 mmol) and 1-hydroxybenzotriazole (53 mg, 0.397 mmol) and stirred for 1 h. (3-tert-butyl-1-(3-chlorophenyl)-1H-pyrazol-5-yl)methanamine (70 mg, 0.265 mmol) was added and stirred at room temperature for 4 h. Reaction mixture was extracted in ethyl acetate, washed with water, dried over magnesium sulfate, filtered and solvent was evaporated and finally purified by column chromatography. to afford N-((3-tert-butyl-1-(3-chlorophenyl)-1H-pyrazol-5-yl)methyl)-2-(5-methoxy-6-(methylsulfonamido)pyridin-3-yl)propanamide (example compound 105) (121 mg, 80%), a white solid.

¹H NMR (300 MHz, CDCl₃): δ 7.75 (s, 1H, Ar—H), 7.33 (m, 2H, Ar—H), 7.10 (s, 1H, Ar—H), 6.08 (s, 1H, Ar—H), 4.47 (m, 2H, Ar—CH₂), 3.86 (s, 3H, Ar—OCH₃), 3.46 (s, 3H, ArSO₂—CH₃), 3.4 2 (q, 1H, J=11.01 Hz, Ar—CH), 1.4 6 (d, 3H, J=7.14 Hz, ArCH—CH₃), 1.29 (s, 9H, R—C(CH₃)₃).

Examples 28, 29 and example 162 can be prepared in a similar manner.

### Synthesis of Example 106

### 1-((1-(3-chlorophenyl)-3-(trifluoromethyl)-1H-pyrazol-5-yl)methyl)-3-(6-(dimethylamino)-5-(trifluoromethyl)pyridin-3-yl)urea

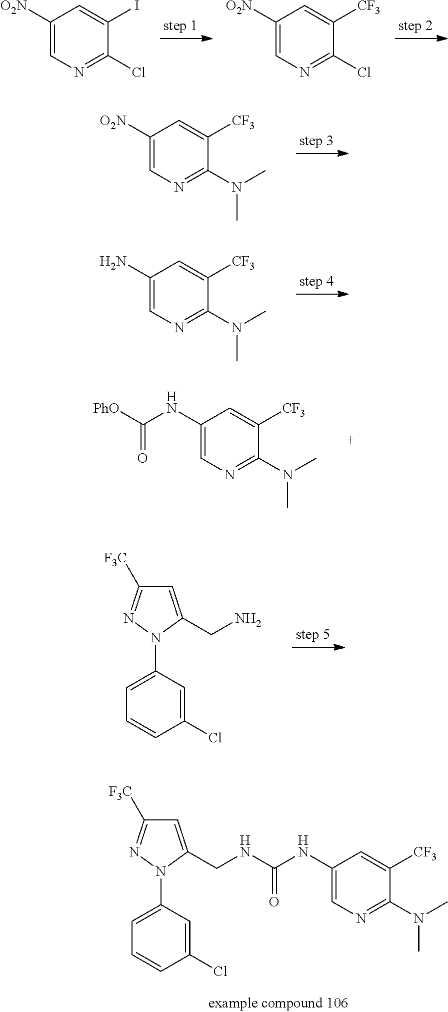

### Step 1:

In a 100 mL round bottom flask, a mixture of 2-chloro-3-iodo-5-nitropyridine (250 mg, 0.88 mmol), methyl 2,2-difluoro-2-(fluorosulfonyl)acetate (0.06 mL, 0.44 mmol) and Copper(I) iodide (25 mg, 0.13 mmol) in dimethylformamide was heated at 70° C. for 3 h under hydrogen atmosphere. Another 0.03 mL methyl 2,2-difluoro-2-(fluorosulfonyl)acetate was added and the mixture was heated at 70° C. for 16 h. The reaction mixture was cooled to room temperature, diluted with water and extracted with ethyl acetate. The organic layer was concentrated under reduced pressure to afford the crude which was purified by column chromatography to give 2-chloro-5-nitro-3-(trifluoromethyl)pyridine (41 mg, 21%).

### Step 2:

2-Chloro-5-nitro-3-(trifluoromethyl)pyridine (41 mg, 0.18 mmol), dimethylamine hydrochloride (18 mg, 0.22 mmol), potassium carbonate (88 mg, 0.63 mmol) and 1,4,7,10,13,16-hexaoxacyclooctadecane (10 mg) was dissolved in acetonitrile. The reaction mixture was refluxed for 12 h. The reaction mixture was cooled to room temperature and then was concentrated under reduced pressure. Then the mixture was extracted with ethyl acetate and washed with water. The organic layer was concentrated under reduced pressure. The crude was purified by column chromatography to give N,N-dimethyl-5-nitro-3-(trifluoromethyl)pyridin-2-amine (36 mg, 84%).

### Step 3:

N,N-dimethyl-5-nitro-3-(trifluoromethyl)pyridin-2-amine (200 mg, 0.85 mmol) was dissolved in methanol. 10% Pd/C (40 mg) was added to it. The resulting mixture was stirred at room temperature under hydrogen atmosphere for 1 h. The mixture was filtered through celite bed and the filtrate was concentrated under reduced pressure to afford the N2,N2-dimethyl-3-(trifluoromethyl)pyridine-2,5-diamine (60 mg, 34%).

### Step 4:

N2,N2-dimethyl-3-(trifluoromethyl)pyridine-2,5-diamine (60 mg, 0.29 mmol) was dissolved in acetonitrile. The reaction mixture was added pyridine (0.03 mL, 0.35 mmol) and phenyl chloroformate (0.04 mL, 0.31 mmol), respectively and stirred at room temperature for 1 h. The reaction mixture was diluted with water and extracted with ethyl acetate. The organic layer was concentrated under reduced pressure. The crude was purified by column chromatography to give phenyl 6-(dimethylamino)-5-(trifluoromethyl)pyridin-3-ylcarbamate (47 mg, 49%).

### Step 5:

Phenyl 6-(dimethylamino)-5-(trifluoromethyl)pyridin-3-ylcarbamate (47 mg, 0.14 mmol) and (1-(3-chlorophenyl)-3-(trifluoromethyl)-1H-pyrazol-5-yl)methanamine (42 mg, 0.15 mmol) was dissolved in dimethyl sulfoxide. Then triethylamine (0.04 mL, 0.29 mmol) was added to it. The mixture was stirred at room temperature overnight. The reaction mixture was diluted with water and extracted with ethyl acetate. The organic layer was concentrated under reduced pressure. The crude was purified by column chromatography to give 1-((1-(3-chlorophenyl)-3-(trifluoromethyl)-1H-pyrazol-5-yl)methyl)-3-(6-(dimethylamino)-5-(trifluoromethyl)pyridin-3-yl)urea (example compound 106) (52 mg, 71%).

¹H NMR (300 MHz, CD₃OD): 8.33 (d, 1H, J=2.58 Hz, Ar—H), 8.07 (d, 1H, J=2.55 Hz, Ar—H), 7.64 (m, 1H, Ar—H), 7.57 (m, 3H, Ar—H), 6.77 (s, 1H, Ar—H), 4.48 (s, 2H, Ar—CH₂), 2.85 (s, 6H, Ar—N(CH₃)₂).

### Synthesis of Example 107

### 1-((3-tert-butyl-1-(3-chlorophenyl)-1H-pyrazol-5-yl)methyl)-3-(6-(dimethylamino)-5-(trifluoromethyl)pyridin-3-yl)urea

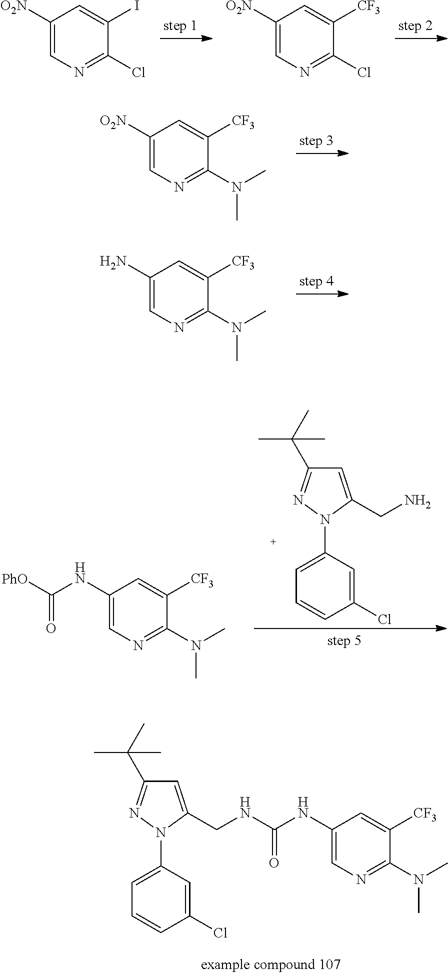

### Step 1-4

as described for example 106.

### Step 5:

Phenyl 6-(dimethylamino)-5-(trifluoromethyl)pyridin-3-ylcarbamate (39 mg, 0.12 mmol) and (3-tert-butyl-1-(3-chlorophenyl)-1H-pyrazol-5-yl)methanamine (34 mg, 0.13 mmol) was dissolved in dimethyl sulfoxide. Then triethylamine (0.03 mL, 0.24 mmol) was added to it. The mixture was stirred at room temperature overnight. The reaction mixture was diluted with water and extracted with ethyl acetate. The organic layer was concentrated under reduced pressure. The crude was purified by column chromatography to give 1-((3-tert-butyl-1-(3-chlorophenyl)-1H-pyrazol-5-yl)methyl)-3-(6-(dimethylamino)-5-(trifluoromethyl)pyridin-3-yl)urea (example compound 107) (41 mg, 69%).

¹H NMR (300 MHz, CD₃OD): δ 8.33 (d, 1H, J=2.73 Hz,Ar—H), 8.08 (d, 1H, J=2.73 Hz, Ar—H), 7.55 (m, 4H, Ar—H), 6.37 (s, 1H, Ar—H), 4.43 (s, 2H, Ar—CH₂), 2.85 (s, 6H, Ar—N(CH₃)₂), 1.32 (s, 9H, Ar—C(CH₃)₃).

### Synthesis of Example 108

### 1-(6-(azetidin-1-yl)pyridin-3-yl)-3-((3-tert-butyl-1-(3-chlorophenyl)-1H-pyrazol-5-yl)methyl)urea

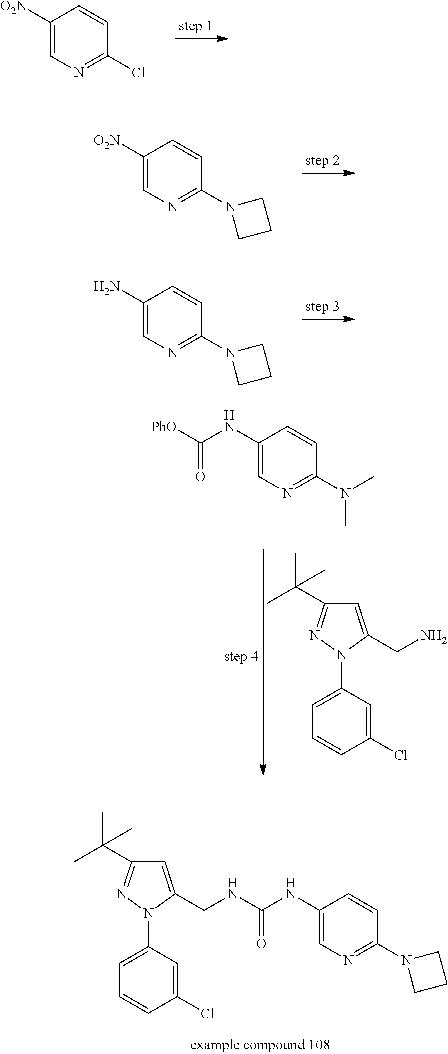

### Step 1:

2-Chloro-5-nitropyridine (300 mg, 1.89 mmol), azetidine hydrochloride (212 mg, 2.27 mmol), potassium carbonate (915 mg, 6.62 mmol) and 1,4,7,10,13,16-hexaoxacyclo-octadecane (60 mg) was dissolved in acetonitrile. The reaction mixture was refluxed overnight. The reaction mixture was cooled to room temperature and then was concentrated under reduced pressure. Then the mixture was extracted with ethyl acetate and washed with water. The organic layer was concentrated under reduced pressure. The crude was purified by column chromatography to give 2-(azetidin-1-yl)-5-nitropyridine (196 mg, 58%).

### Step 2:

2-(Azetidin-1-yl)-5-nitropyridine (185 mg, 1.03 mmol) was dissolved in methanol. 10% Pd/C (37 mg) was added to it. The resulting mixture was stirred at room temperature under hydrogen atmosphere for 1 h. The mixture was filtered through celite bed and the filtrate was concentrated under reduced pressure to afford the 6-(azetidin-1-yl)pyridin-3-amine (154 mg, 99%).

### Step 3:

6-(Azetidin-1-yl)pyridin-3-amine (154 mg, 1.03 mmol) was dissolved in acetonitrile. To the reaction mixture was added pyridine (0.1 mL, 1.24 mmol) and phenyl chloroformate (0.14 mL, 1.08 mmol), respectively and stirred at room temperature for 1 h. The reaction mixture was diluted with water and extracted with ethyl acetate. The organic layer was concentrated under reduced pressure. The crude was purified by column chromatography to give phenyl 6-(azetidin-1-yl)pyridin-3-ylcarbamate (123 mg, 44%).

### Step 4:

Phenyl 6-(azetidin-1-yl)pyridin-3-ylcarbamate (60 mg, 0.22 mmol) and (3-tert-butyl-1-(3-chlorophenyl)-1H-pyrazol-5-yl)methanamine (62 mg, 0.23 mmol) was dissolved in dimethyl sulfoxide. Then triethylamine (0.06 mL, 0.45 mmol) was added to it. The mixture was stirred at room temperature overnight. The reaction mixture was diluted with water and extracted with ethyl acetate. The organic layer was concentrated under reduced pressure. The crude was purified by column chromatography to give 1-(6-(azetidin-1-yl)pyridin-3-yl)-3-((3-tert-butyl-1-(3-chlorophenyl)-1H-pyrazol-5-yl)methyl)urea (example compound 108) (56 mg, 57%).

¹H NMR (300 MHz, CD₃OD): δ 7.94 (d, 1H, J=2.58 Hz, Ar—H), 7.55 (m, 5H, Ar—H), 6.38 (d, 2H, J=7.14 Hz, Ar—H), 4.40 (s, 2H, Ar—CH₂), 4.00 (m, 4H, azetidine-CH₂), 2.42 (m, 2H, azetidine —CH₂), 1.32 (s, 9H, Ar—C(CH₃)₃).

Example 101 was prepared in a similar manner.

### Synthesis of Example 109

### 1-(6-(azetidin-1-yl)pyridin-3-yl)-3-((1-(3-chlorophenyl)-3-(trifluoromethyl)-1H-pyrazol-5-yl)methyl)urea

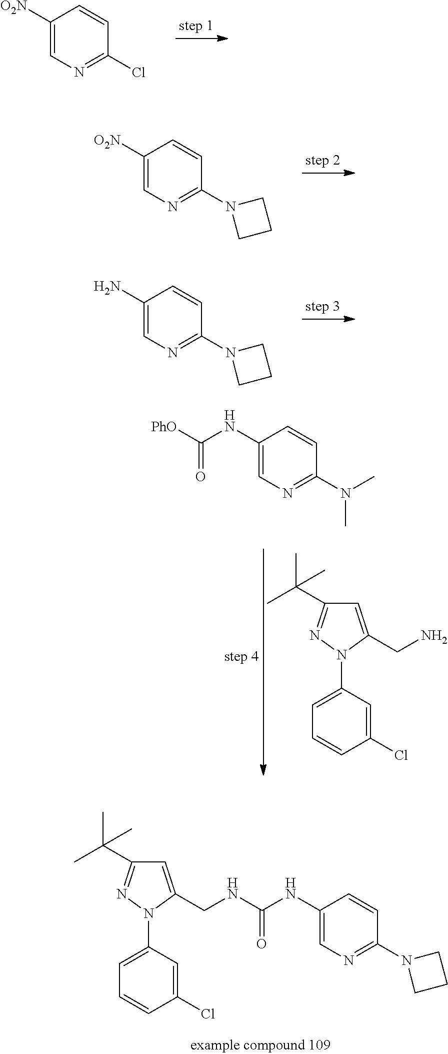

### Step 1-3

as described for example 108.

### Step 4:

Phenyl 6-(azetidin-1-yl)pyridin-3-ylcarbamate (60 mg, 0.22 mmol) and (1-(3-chlorophenyl)-3-(trifluoromethyl)-1H-pyrazol-5-yl)methanamine (65 mg, 0.23 mmol) was dissolved in dimethyl sulfoxide. Then triethylamine (0.06 mL, 0.45 mmol) was added to it. The mixture was stirred at room temperature overnight. The reaction mixture was diluted with water and extracted with ethyl acetate. The organic layer was concentrated under reduced pressure. The crude was purified by column chromatography to give 1-(6-(azetidin-1-yl)pyridin-3-yl)-3-((1-(3-chlorophenyl)-3-(trifluoromethyl)-1H-pyrazol-5-yl)methyl)urea (example compound 109) (80 mg, 80%).

¹H NMR (300 MHz, CD₃OD): δ 7.94 (d, 1H, J=2.58 Hz, Ar—H), 7.63 (m, 5H, Ar—H), 6.75 (s, 1H, Ar—H), 6.47 (d, 1H, J=8.79 Hz, Ar—H), 4.45 (s, 2H, Ar—CH₂), 4.00 (m, 4H, azetidine-CH₂), 2.42 (m, 2H, azetidine —CH₂).

### Synthesis of Example 111

### 1-((1-(3-chlorophenyl)-3-(trifluoromethyl)-1H-pyrazol-5-yl)methyl)-3-(6-(3-hydroxyazetidin-1-yl)pyridin-3-yl)urea

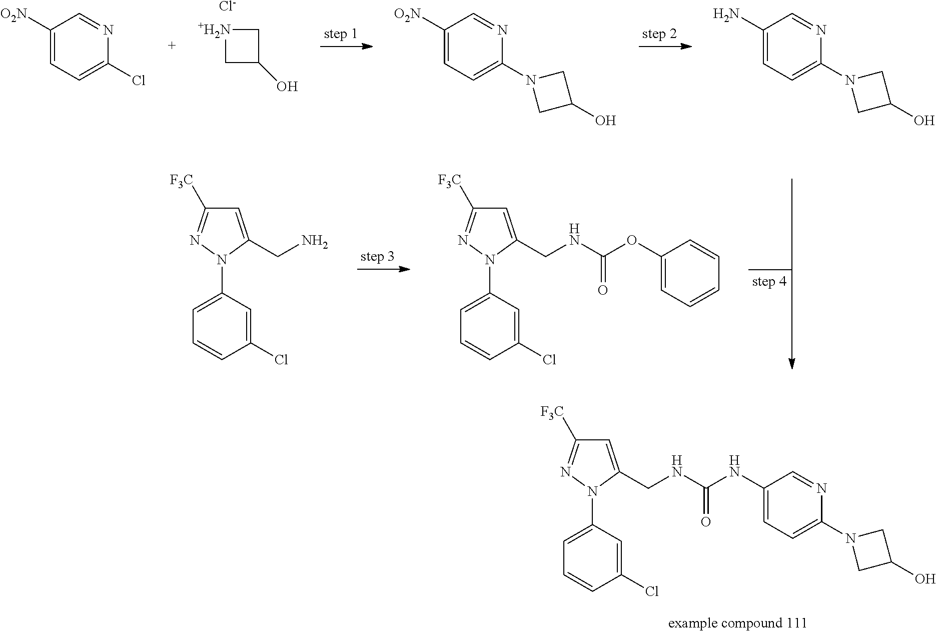

### Step 1:

To a cooled solution of 2-chloro-5-nitropyridine (554 mg, 3.50 mmol, 1 eq) and 3-hydroxyazetidinium chloride (459 mg, 4.20 mmol, 1.2 eq) in dimethylformamide (7 mL), 1.46 mL of triethylamine were added and the mixture was stirred for 30 min after which the reaction was judged complete by TLC. The solvent was evaporated, the residue was suspended in 25 mL of water and extracted with ethyl acetate (3×25 mL). The organic layers were combined, washed with water (2×25 mL) and brine (1×25 mL) and dried over magnesium sulfate. The solvent was evaporated and the residue was purified by column chromatography (silica gel, ethyl acetate/n-hexane 1/2, v/v as eluent) 1-(5-nitropyridin-2-yl)azetidin-3-ol (572 mg, 84%) as a yellow solid.

### Step 2:

1-(5-Nitropyridin-2-yl)azetidin-3-ol (570 mg, 2.92 mmol, 1 eq) was dissolved in ethanol (30 mL) and hydrogenated on an H-cube using 10% Pd/C (10 bar H₂, 1 mL/min). The mixture was evaporated a to yield 1-(5-aminopyridin-2-yl)azetidin-3-ol (480 mg) which was used without further purification.

### Step 3:

To a stirred solution of (1-(3-chlorophenyl)-3-(trifluoromethyl)-1H-pyrazol-5-yl)methanamine (551 mg, 2.00 mmol, 1 eq) in dichloromethane (14 mL) phenyl chloroformate (285 μL, 2.24 mmol, 1.12 eq) and triethylamine (333 μL, 2.4 mmol, 1.2 eq) were added and stirred room temperature overnight The reaction mixture was washed with sodium carbonate (c=1 mol/L, 1×20 mL), the aqueous phase was extracted with dichloromethane (2×10 mL) and the organic phases were dried over magnesium sulfate. The solvent was evaporated and the residue was purified by column chromatography to yield 2-(5-aminopyridin-2-yloxy)ethanol (silica gel, ethyl acetate/n-hexane 1/4, v/v as eluent) to yield phenyl (1-(3-chlorophenyl)-3-(trifluoromethyl)-1H-pyrazol-5-yl)methylcarbamate (352 mg, 44%).

### Step 4:

To a stirred solution of phenyl (1-(3-chlorophenyl)-3-(trifluoromethyl)-1H-pyrazol-5-yl)methylcarbamate (87 mg, 0.22 mmol, 1.0 eq) in acetonitrile (6 mL) was added triethylamine (90 μL, 0.66 mmol, 3.0 eq) followed by 1-(5-aminopyridin-2-yl)azetidin-3-ol (38 mg, 0.24 mmol, 1.02 eq) and stirred for 16 h at reflux. The reaction mixture was concentrated under vacuum and the residue was purified (column chromatography, silica gel, ethyl acetate/methanol, 6/1, v/v as eluent) to yield 1-((1-(3-chlorophenyl)-3-(trifluoromethyl)-1H-pyrazol-5-yl)methyl)-3-(6-(3-hydroxyazetidin-1-yl)pyridin-3-yl)urea (example compound III) 56 mg, 55%).

Examples 110, 112-115 and 117 were prepared in a similar manner. Examples 116 and 118-119 can be prepared in a similar manner.

### Synthesis of Example 120:1-((3-tert-butyl-1-(3-chlorophenyl)-1H-pyrazol-5-yl)methyl)-3-(6-(pyrrolidin-1-yl)pyridin-3-yl)urea

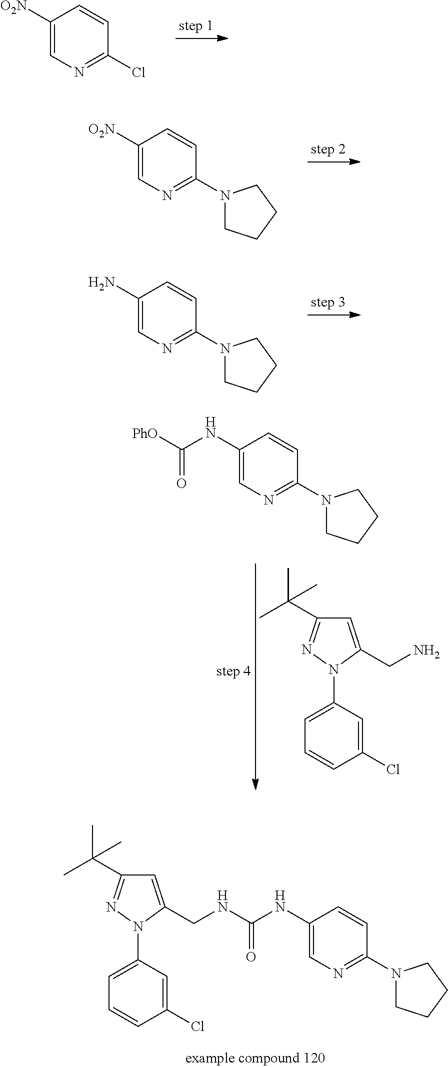

### Step 1:

2-Chloro-5-nitropyridine (300 mg, 1.89 mmol), pyrrolidine (0.19 mL, 2.27 mmol), potassium carbonate (785 mg, 5.68 mmol) and 1,4,7,10,13,16-hexaoxacyclooctadecane (60 mg) was dissolved in acetonitrile. The reaction mixture was refluxed overnight. The reaction mixture was cooled to room temperature and then was concentrated under reduced pressure. Then the mixture was extracted with ethyl acetate and washed with water. The organic layer was concentrated under reduced pressure. The crude was purified by column chromatography to give 5-nitro-2-(pyrrolidin-1-yl)pyridine (317 mg, 87%).

### Step 2:

5-Nitro-2-(pyrrolidin-1-yl)pyridine (317 mg, 1.65 mmol) was dissolved in methanol. 10% Pd/C (64 mg) was added to it. The resulting mixture was stirred at room temperature under hydrogen for 1 h. The mixture was filtered through celite bed and the filtrate was concentrated under reduced pressure to afford the 6-(pyrrolidin-1-yl)pyridin-3-amine (261 mg, 97%).

### Step 3:

6-(Pyrrolidin-1-yl)pyridin-3-amine (261 mg, 1.6 mmol) was dissolved in acetonitrile.

To the reaction mixture was added pyridine (0.16 mL, 1.92 mmol) and phenyl chloroformate (0.21 mL, 1.68 mmol), respectively and stirred at room temperature for 1 h. The reaction mixture was diluted with water and extracted with ethyl acetate. The organic layer was concentrated under reduced pressure. The crude was purified by column chromatography to give phenyl 6-(pyrrolidin-1-yl)pyridin-3-ylcarbamate (218 mg, 48%).

### Step 4:

Phenyl 6-(pyrrolidin-1-yl)pyridin-3-ylcarbamate (70 mg, 0.25 mmol) and (3-tert-butyl-1-(3-chlorophenyl)-1H-pyrazol-5-yl)methanamine (69 mg, 0.26 mmol) was dissolved in dimethyl sulfoxide. Then triethylamine (0.07 mL, 0.49 mmol) was added to it. The mixture was stirred at room temperature overnight. The reaction mixture was diluted with water and extracted with ethyl acetate. The organic layer was concentrated under reduced pressure. The crude was purified by column chromatography to give 1-((3-tert-butyl-1-(3-chlorophenyl)-1H-pyrazol-5-yl)methyl)-3-(6-(pyrrolidin-1-yl)pyridin-3-yl)urea (example compound 120) (99 mg, 88%).

¹H NMR (300 MHz, CD₃OD): δ 7.92 (d, 1H, J=2.37 Hz, Ar—H), 7.56 (m, 5H, Ar—H), 6.47 (d, 1H, J=8.97 Hz, Ar—H), 6.35 (s, 1H, Ar—H), 4.40 (s, 2H, Ar—CH₂), 3.42 (m, 4H, pyrrolidine-CH₂), 2.04 (m, 4H, pyrrolidine-CH₂), 1.32 (s, 9H, Ar—C(CH₃)₃).

### Synthesis of Example 121

### 1-((1-(3-chlorophenyl)-3-(trifluoromethyl)-1H-pyrazol-5-yl)methyl)-3-(6-(pyrrolidin-1-yl)pyridin-3-yl)urea

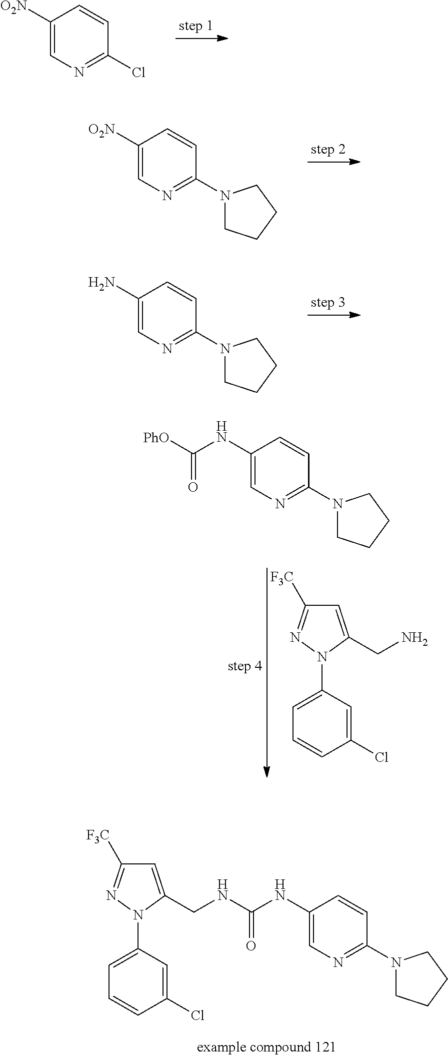 [SMILES: O=[N+]([O-])c1ccc(Cl)nc1 ; O=[N+]([O-])c1ccc(N2CCCC2)nc1 ; Nc1ccc(N2CCCC2)nc1 ; O=C(Nc1ccc(N2CCCC2)nc1)Oc1ccccc1 ; NCc1cc(C(F)(F)F)nn1-c1cccc(Cl)c1 ; O=C(NCc1cc(C(F)(F)F)nn1-c1cccc(Cl)c1)Nc1ccc(N2CCCC2)nc1]

Step 1-3: as described for example 120.

### Step 4:

Phenyl 6-(pyrrolidin-1-yl)pyridin-3-ylcarbamate (70 mg, 0.25 mmol) and (1-(3-chlorophenyl)-3-(trifluoromethyl)-1H-pyrazol-5-yl)methanamine (72 mg, 0.26 mmol) was dissolved in dimethyl sulfoxide. Then triethylamine (0.07 mL, 0.49 mmol) was added to it. The mixture was stirred at room temperature overnight. The reaction mixture was diluted with water and extracted with ethyl acetate. The organic layer was concentrated under reduced pressure. The crude was purified by column chromatography to give 1-((1-(3-chlorophenyl)-3-(trifluoromethyl)-1H-pyrazol-5-yl)methyl)-3-(6-(pyrrolidin-1-yl)pyridin-3-yl)urea (example compound 121) (114 mg, 99%).

¹H NMR (300 MHz, CD₃OD): δ 7.91 (d, 1H, J=2.19 Hz, Ar—H), 7.63 (s, 1H, Ar—H), 7.57 (m, 4H, Ar—H), 6.74 (s, 1H, Ar—H), 6.47 (d, 1H, J=8.61 Hz, Ar—H), 4.45 (s, 2H, Ar—CH₂), 3.42 (m, 4H, pyrrolidine-CH₂), 2.03 (m, 4H, pyrrolidine-CH₂).

Example 122 can be prepared in a similar manner.

### Synthesis of Example 123

### 1-((1-(3-chlorophenyl)-3-(trifluoromethyl)-1H-pyrazol-5-yl)methyl)-3-(5-methoxy-6-(pyrrolidin-1-yl)pyridin-3-yl)urea

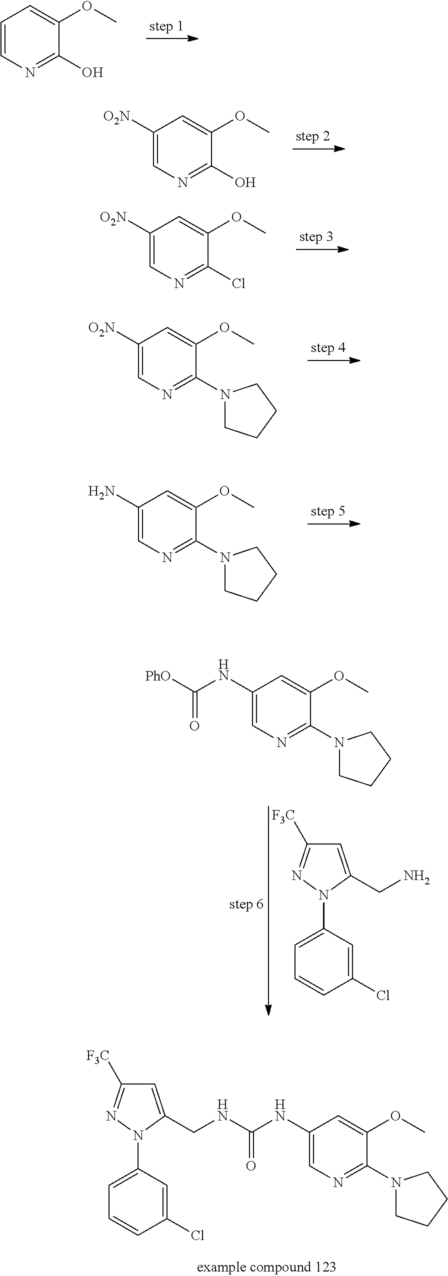

### Step 1:

To a solution of 3-methoxypyridin-2-ol (925 mg, 7.39 mmol) in sulfuric acid (7.4 mL) was slowly added nitric acid (60%) (0.64 mL, 11.09 mmol) at 0° C. After finishing adding nitric acid, the reaction mixture was heated at 40° C. for 3 h. The reaction mixture was cooled to room temperature, then crushed ice was added. After stirring for 30 min, the solution was filtered to afford 3-methoxy-5-nitropyridin-2-ol (1.043 g, 83%).

### Step 2:

3-Methoxy-5-nitropyridin-2-ol (1.043 g, 6.13 mmol) and phosphorus pentachloride (0.638 g, 3.07 mmol) was dissolved in phosphoryl chloride (1.71 mL). The reaction mixture was refluxed for 4 h. After cooling to room temperature, the reaction mixture was poured to crushed ice and stirred for 30 min. Then the mixture was basified by using Na₂CO₃ to pH 7. The solution was extracted with ethyl acetate. The organic layer was concentrated under reduced pressure to afford the crude which was purified by column chromatography to give 2-chloro-3-methoxy-5-nitropyridine (920 mg, 80%).

### Step 3:

2-Chloro-3-methoxy-5-nitropyridine (400 mg, 2.12 mmol), pyrrolidine (0.21 mL, 2.55 mmol), potassium carbonate (880 mg, 6.38 mmol) and 1,4,7,10,13,16-hexaoxacyclooctadecane (80 mg) was dissolved in acetonitrile. The reaction mixture was refluxed overnight. The reaction mixture was cooled to room temperature and then was concentrated under reduced pressure. Then the mixture was extracted with ethyl acetate and washed with water. The organic layer was concentrated under reduced pressure. The crude was purified by column chromatography to give 3-methoxy-5-nitro-2-(pyrrolidin-1-yl)pyridine (458 mg, 97%).

### Step 4:

3-Methoxy-5-nitro-2-(pyrrolidin-1-yl)pyridine (458 mg, 2.05 mmol) was dissolved in methanol. 10% Pd/C (92 mg) was added to it. The resulting mixture was stirred at room temperature under hydrogen atmosphere for 1 h. The mixture was filtered through celite bed and the filtrate was concentrated under reduced pressure to afford 5-methoxy-6-(pyrrolidin-1-yl)pyridin-3-amine (396 mg, 99%).

### Step 5:

5-Methoxy-6-(pyrrolidin-1-yl)pyridin-3-amine (396 mg, 2.05 mmol) was dissolved in acetonitrile. The reaction mixture was added pyridine (0.20 mL, 2.46 mmol) and phenyl chloroformate (0.27 mL, 2.15 mmol), respectively and stirred at room temperature for 1 h. The reaction mixture was diluted with water and extracted with ethyl acetate. The organic layer was concentrated under reduced pressure. The crude was purified by column chromatography to give phenyl 5-methoxy-6-(pyrrolidin-1-yl)pyridin-3-ylcarbamate (364 mg, 57%).

### Step 6:

Phenyl 5-methoxy-6-(pyrrolidin-1-yl)pyridin-3-ylcarbamate (186 mg, 0.59 mmol) and (1-(3-chlorophenyl)-3-(trifluoromethyl)-1H-pyrazol-5-yl)methanamine (172 mg, 0.62 mmol) was dissolved in dimethyl sulfoxide. Then triethylamine (0.17 mL, 1.19 mmol) was added to it. The mixture was stirred at room temperature overnight. The reaction mixture was diluted with water and extracted with ethyl acetate. The organic layer was concentrated under reduced pressure. The crude was purified by column chromatography to give 1-((1-(3-chlorophenyl)-3-(trifluoromethyl)-1H-pyrazol-5-yl)methyl)-3-(5-methoxy-6-(pyrrolidin-1-yl)pyridin-3-yl)urea (example compound 123) (186 mg, 63%).

¹H NMR (300 MHz, CD₃OD): δ 7.63 (s, 1H, Ar—H), 7.56 (m, 4H, Ar—H), 7.24 (s, 1H, Ar—H), 6.75 (s, 1H, Ar—H), 4.45 (s, 2H, Ar—CH₂), 3.78 (s, 3H, Ar—OCH₃), 3.53 (m, 4H, pyrrolidine-CH₂), 1.90 (m, 4H, pyrrolidine-CH₂).

### Synthesis of Example 124

### (R)-1-((1-(3-chlorophenyl)-3-(trifluoromethyl)-1H-pyrazol-5-yl)methyl)-3-(6-(3-hydroxypyrrolidin-1-yl)pyridin-3-yl)urea

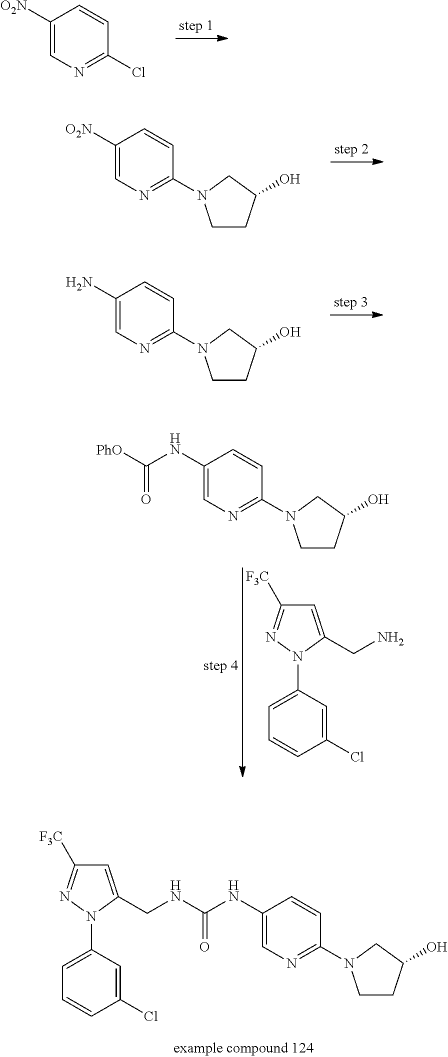

### Step 1:

2-Chloro-5-nitropyridine (365 mg, 2.30 mmol), (R)-3-pyrrolidinol (241 mg, 2.76 mmol), potassium carbonate (955 mg, 6.91 mmol) and 1,4,7,10,13,16-hexaoxacycloocta-decane (73 mg) was dissolved in acetonitrile. The reaction mixture was refluxed overnight. The reaction mixture was cooled to room temperature and then was concentrated under reduced pressure. Then the mixture was extracted with ethyl acetate and washed with water. The organic layer was concentrated under reduced pressure. The crude was purified by column chromatography to give (R)-1-(5-nitropyridin-2-yl)pyrrolidin-3-ol (452 mg, 94%).

### Step 2:

(R)-1-(5-Nitropyridin-2-yl)pyrrolidin-3-ol (452 mg, 2.16 mmol) was dissolved in methanol. 10% Pd/C (91 mg) was added to it. The resulting mixture was stirred at room temperature under hydrogen atmosphere for 1 h. The mixture was filtered through celite bed and the filtrate was concentrated under reduced pressure to afford (R)-1-(5-aminopyridin-2-yl)pyrrolidin-3-ol (386 mg, 99%).

### Step 3:

(R)-1-(5-Aminopyridin-2-yl)pyrrolidin-3-ol (386 mg, 2.16 mmol) was dissolved in acetonitrile. The reaction mixture was added pyridine (0.21 mL, 2.59 mmol) and phenyl chloroformate (0.29 mL, 2.26 mmol), respectively and stirred at room temperature for 1 h. The reaction mixture was diluted with water and extracted with ethyl acetate. The organic layer was concentrated under reduced pressure. The crude was purified by column chromatography to give (R)-phenyl 6-(3-hydroxypyrrolidin-1-yl)pyridin-3-ylcarbamate (125 mg, 19%).

### Step 4:

(R)-Phenyl 6-(3-hydroxypyrrolidin-1-yl)pyridin-3-ylcarbamate (60 mg, 0.20 mmol) and (1-(3-chlorophenyl)-3-(trifluoromethyl)-1H-pyrazol-5-yl)methanamine (58 mg, 0.21 mmol) was dissolved in dimethyl sulfoxide. Then triethylamine (0.06 mL, 0.40 mmol) was added to it. The mixture was stirred at room temperature overnight. The reaction mixture was diluted with water and extracted with ethyl acetate. The organic layer was concentrated under reduced pressure. The crude was purified by column chromatography to give (R)-1-((1-(3-chlorophenyl)-3-(trifluoromethyl)-1H-pyrazol-5-yl)methyl)-3-(6-(3-hydroxypyrrolidin-1-yl)pyridin-3-yl)urea (example compound 124) (186 mg, 64%).

¹H NMR (300 MHz, CD₃OD): δ 7.92 (d, 1H, J=1.77 Hz, Ar—H), 7.62 (s, 1H, Ar—H), 7.56 (m, 4H, Ar—H), 6.74 (s, 1H, Ar—H), 6.47 (d, 1H, J=6.75 Hz, Ar—H), 4.50 (m, 1H, pyrrolidinol-CH), 4.44 (s, 2H, Ar—CH₂), 3.56 (m, 4H, pyrrolidinol-CH₂), 2.17 (m, 2H, pyrrolidinol-CH₂).

### Synthesis of Example 125

### (S)-1-((1-(3-chlorophenyl)-3-(trifluoromethyl)-1H-pyrazol-5-yl)methyl)-3-(6-(3-hydroxypyrrolidin-1-yl)pyridin-3-yl)urea

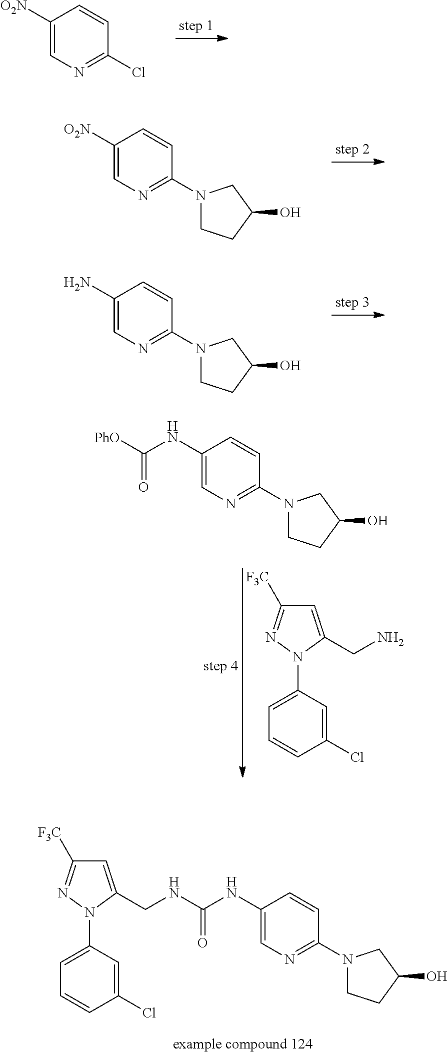

### Step 1:

2-Chloro-5-nitropyridine (459 mg, 2.90 mmol), (S)-3-pyrrolidinol (303 mg, 3.48 mmol), potassium carbonate (1.202 g, 8.70 mmol) and 1,4,7,10,13,16-hexaoxacyclo-octadecane (92 mg) was dissolved in acetonitrile. The reaction mixture was refluxed overnight. The reaction mixture was cooled to room temperature and then was concentrated under reduced pressure. Then the mixture was extracted with ethyl acetate and washed with water. The organic layer was concentrated under reduced pressure. The crude was purified by column chromatography to (S)-1-(5-nitropyridin-2-yl)pyrrolidin-3-ol (574 mg, 95%).

### Step 2:

(S)-1-(5-Nitropyridin-2-yl)pyrrolidin-3-ol (574 mg, 2.74 mmol) was dissolved in methanol. 10% Pd/C (115 mg) was added to it. The resulting mixture was stirred at room temperature under hydrogen atmosphere for 1 h. The mixture was filtered through celite bed and the filtrate was concentrated under reduced pressure to afford (S)-1-(5-aminopyridin-2-yl)pyrrolidin-3-ol (491 mg, 99%).

### Step 3:

(S)-1-(5-Aminopyridin-2-yl)pyrrolidin-3-ol (491 mg, 2.74 mmol) was dissolved in acetonitrile. The reaction mixture was added pyridine (0.27 mL, 3.29 mmol) and phenyl chloroformate (0.36 mL, 2.88 mmol), respectively and stirred at room temperature for 1 h. The reaction mixture was diluted with water and extracted with ethyl acetate. The organic layer was concentrated under reduced pressure. The crude was purified by column chromatography to give (S)-phenyl 6-(3-hydroxypyrrolidin-1-yl)pyridin-3-ylcarbamate (274 mg, 33%).

### Step 4:

(S)-Phenyl 6-(3-hydroxypyrrolidin-1-yl)pyridin-3-ylcarbamate (138 mg, 0.46 mmol) and (1-(3-chlorophenyl)-3-(trifluoromethyl)-1H-pyrazol-5-yl)methanamine (133 mg, 0.48 mmol) was dissolved in dimethyl sulfoxide. Then triethylamine (0.13 mL, 0.92 mmol) was added to it. The mixture was stirred at room temperature overnight. The reaction mixture was diluted with water and extracted with ethyl acetate. The organic layer was concentrated under reduced pressure. The crude was purified by column chromatography to give (S)-1-((1-(3-chlorophenyl)-3-(trifluoromethyl)-1H-pyrazol-5-yl)methyl)-3-(6-(3-hydroxypyrrolidin-1-yl)pyridin-3-yl)urea (example compound 125) (157 mg, 71%).

¹H NMR (300 MHz, CD₃OD): δ 7.93 (d, 1H, J=2.4 Hz, Ar—H), 7.63 (m, 1H, Ar—H), 7.57 (m, 4H, Ar—H), 6.75 (s, 1H, Ar—H), 6.49 (d, 1H, J=9.3 Hz, Ar—H), 4.51 (m, 1H, pyrrolidinol-CH), 4.45 (s, 2H, Ar—CH₂), 3.59 (m, 4H, pyrrolidinol-CH₂), 2.15 (m, 2H, pyrrolidinol-CH₂).

### Synthesis of Example 131

### 1-((3-tert-butyl-1-(3-chlorophenyl)-1H-pyrazol-5-yl)methyl)-3-(6-(2-methoxyethoxy)pyridin-3-yl)urea

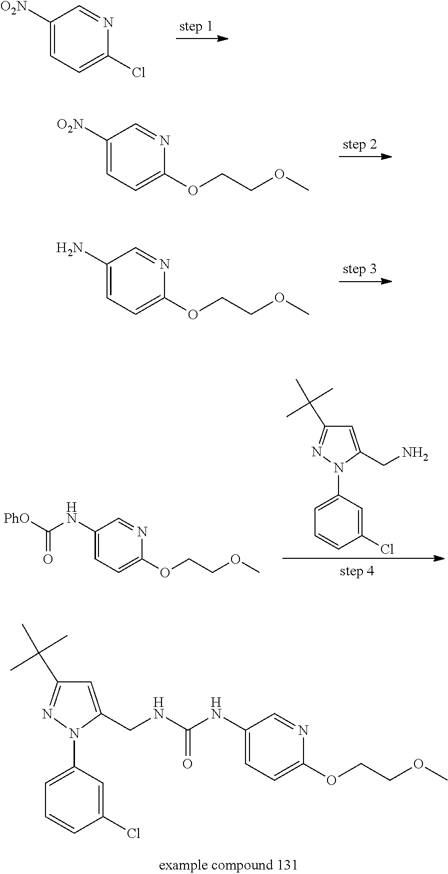 [SMILES: O=[N+]([O-])c1ccc(Cl)nc1 ; COCCOc1ccc([N+](=O)[O-])cn1 ; COCCOc1ccc(N)cn1 ; COCCOc1ccc(NC(=O)Oc2ccccc2)cn1 ; CC(C)(C)c1cc(CN)n(-c2cccc(Cl)c2)n1 ; COCCOc1ccc(NC(=O)NCc2cc(C(C)(C)C)nn2-c2cccc(Cl)c2)cn1]

### Step 1:

2-Chloro-5-nitropyridine (5.00 g, 31.6 mmol, 1 eq) and 2-methoxyethanol (2.52 g, 33.1 mmol, 1.05 eq) were dissolved in dimethylformamide (32 mL) and cooled to 0° C. Sodium hydride (60% w/w in mineral oil, 1.30 mg, 32.5 mmol, 1.03 eq) was added in portions and the mixture was allowed to warm to room temperature overnight. After the reaction was complete (TLC), acetic acid (5 mL) was added and the solvent was evaporated. The residue was suspended in diethyl ether (100 mL) and filtered. The filter cake was washed with dichloromethane (2×50 mL), the filtrate was evaporated and purified by column chromatography (silica gel, ethyl acetate/n-hexane 1/4, v/v as eluent) to yield 2-(2-methoxyethoxy)-5-nitropyridine (3.96 g, 63%) as a yellow solid.

### Step 2:

2-(2-Methoxyethoxy)-5-nitropyridine (3.95 g, 19.9 mmol, 1 eq) was dissolved in ethanol (180 mL) and hydrogenated on an H-cube using 10% Pd/C. The mixture was evaporated to yield 6-(2-methoxyethoxy)pyridin-3-amine (3.30 mg, 98%) as a colorless solid which was used without further purification.

### Step 3:

To a stirred solution of 6-(2-methoxyethoxy)pyridin-3-amine (501 mg, 2.98 mmol, 1 eq) in acetone (10 mL) pyridine (722 μL, 8.94 mmol, 3 eq) was added followed by phenyl chloroformate (489 μL, 3.87 mmol, 1.3 eq) at 0° C. and stirred at room temperature overnight. The reaction mixture was evaporated and purified by column chromatography to yield 2-(5-aminopyridin-2-yloxy)ethanol (silica gel, methyl tert-butyl ether/methanol 1/1, v/v as eluent) to yield phenyl 6-(2-methoxyethoxy)pyridin-3-ylcarbamate (686 mg, 80%) as a colorless solid.

### Step 4:

To a stirred solution of (3-tert-butyl-1-(3-chlorophenyl)-1H-pyrazol-5-yl)methanamine (102 mg, 0.387 mmol, 1.0 eq) in acetonitrile (9 mL) was added triethylamine (0.214 mL, 1.55 mmol, 4.0 eq) followed by phenyl 6-(2-methoxyethoxy)pyridin-3-ylcarbamate (113 mg, 0.395 mmol, 1.02 eq) and stirred for 16 h at reflux. The reaction mixture was concentrated under vacuum and the residue was purified (column chromatography, silica gel, ethyl acetate/n-hexane, 4/1, v/v as eluent) to yield 1-((3-tert-butyl-1-(3-chlorophenyl)-1H-pyrazol-5-yl)methyl)-3-(6-(2-methoxyethoxy)pyridin-3-yl)urea (example compound 131) (159 mg, 90%) as a colorless solid.

Examples 128 can be and example 130 was prepared in a similar manner.

### Synthesis of Example 132

### 1-((1-(3-chlorophenyl)-3-(trifluoromethyl)-1H-pyrazol-5-yl)methyl)-3-(6-(2-methoxyethoxy)pyridin-3-yl)urea

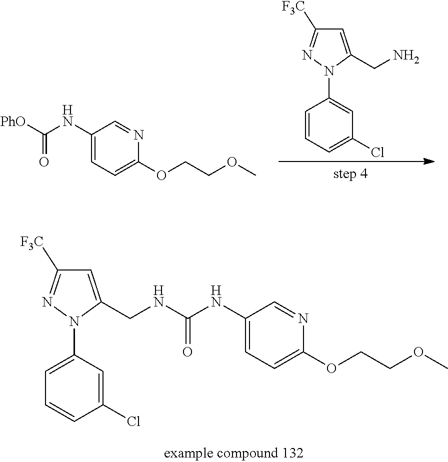 [SMILES: COCCOc1ccc(NC(=O)Oc2ccccc2)cn1 ; NCc1cc(C(F)(F)F)nn1-c1cccc(Cl)c1 ; COCCOc1ccc(NC(=O)NCc2cc(C(F)(F)F)nn2-c2cccc(Cl)c2)cn1]

Step 1-3: see example compound 131

### Step 4:

To a stirred solution of (1-(3-chlorophenyl)-3-(trifluoromethyl)-1H-pyrazol-5-yl)methanamine (108 mg, 0.392 mmol, 1.0 eq) in acetonitrile (9 mL) was added triethylamine (0.216 mL, 1.57 mmol, 4.0 eq) followed by phenyl 6-(2-methoxyethoxy)pyridin-3-ylcarbamate (114 mg, 0.400 mmol, 1.02 eq) and stirred for 16 h at reflux. The reaction mixture was concentrated under vacuum and the residue was purified (column chromatography, silica gel, ethyl acetate/n-hexane, 4/1, v/v as eluent) to yield 1-((1-(3-chlorophenyl)-3-(trifluoromethyl)-1H-pyrazol-5-yl)methyl)-3-(6-(2-methoxyethoxy)pyridin-3-yl)urea (example compound 132) (156 mg; 85%) as a colorless solid.

### Synthesis of Example 133

### 1-((1-(3-chlorophenyl)-3-(trifluoromethyl)-1H-pyrazol-5-yl)methyl)-3-(6-(2-hydroxyethoxy)pyridin-3-yl)urea

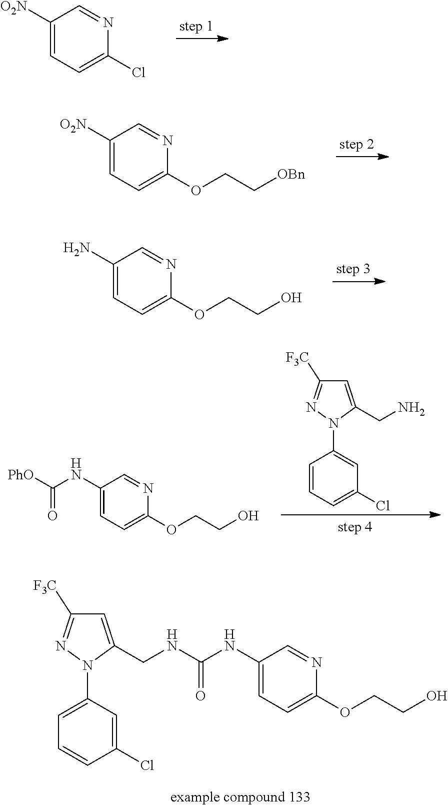 [SMILES: O=[N+]([O-])c1ccc(Cl)nc1 ; O=[N+]([O-])c1ccc(OCCOCc2ccccc2)nc1 ; Nc1ccc(OCCO)nc1 ; O=C(Nc1ccc(OCCO)nc1)Oc1ccccc1 ; NCc1cc(C(F)(F)F)nn1-c1cccc(Cl)c1 ; O=C(NCc1cc(C(F)(F)F)nn1-c1cccc(Cl)c1)Nc1ccc(OCCO)nc1]

### Step 1:

2-Chloro-5-nitropyridine (1.51 g, 9.55 mmol, 1 eq) and 2-(benzyloxy)ethanol (1.53 g, 10.0 mmol, 1.05 eq) were dissolved in dimethylformamide (9 mL) and cooled to 0° C. Sodium hydride (60% w/w in mineral oil, 392 mg, 9.84 mmol, 1.03 eq) was added in portions and the mixture was allowed to warm to room temperature overnight. After the reaction was complete (TLC), acetic acid (1 mL) was added and the solvent was evaporated. The residue was suspended in diethyl ether (20 mL) and filtered. The filter cake was washed with dichloromethane (2×2 mL), the filtrate was evaporated and purified by column chromatography (silica gel, ethyl acetate/n-hexane 1/4, v/v as eluent) to yield 2-(2-(benzyloxy)ethoxy)-5-nitropyridine (2.09 g, 80%) as a yellow solid.

### Step 2:

2-(2-(Benzyloxy)ethoxy)-5-nitropyridine (2.09 g, 7.61 mmol, 1 eq) was dissolved in ethanol (90 mL) and hydrogenated on an H-cube using 10% Pd/C. The mixture was evaporated and the residue was purified by column chromatography to yield 2-(5-aminopyridin-2-yloxy)ethanol (silica gel, methyl tert-butyl ether/MeOH 9/1, v/v as eluent) to yield (209 mg, 18%) as a colorless solid.

### Step 3:

To a stirred solution of 2-(5-aminopyridin-2-yloxy)ethanol (209 mg, 1.36 mmol, 1 eq) in acetone (5 mL) pyridine (329 μL, 4.07 mmol, 3 eq) was added followed by phenyl chloroformate (276 μL, 1.76 mmol, 1.3 eq) at 0° C. and stirred room temperature overnight The reaction mixture was evaporated and purified by column chromatography to yield 2-(5-aminopyridin-2-yloxy)ethanol (silica gel, methyl tert-butyl ether/methanol 9/1, v/v as eluent) to yield phenyl 6-(2-hydroxyethoxy)pyridin-3-ylcarbamate (138 mg, 37%) as a colorless solid.

### Step 4:

To a stirred solution of (1-(3-chlorophenyl)-3-(trifluoromethyl)-1H-pyrazol-5-yl)methanamine (62 mg, 0.23 mmol, 1.0 eq) in acetonitrile (5 mL) was added triethylamine (0.124 mL, 0.90 mmol, 4.0 eq) followed by phenyl 6-(2-hydroxyethoxy)pyridin-3-ylcarbamate (63 mg, 0.23 mmol, 1.02 eq) and stirred for 16 h at reflux. The reaction mixture was concentrated under vacuum and the residue was purified (column chromatography, silica gel, ethyl acetate/n-hexane, 4/1, v/v as eluent) to yield 1-((1-(3-chlorophenyl)-3-(trifluoromethyl)-1H-pyrazol-5-yl)methyl)-3-(6-(2-hydroxyethoxy)pyridin-3-yl)urea (example compound 133) (92 mg, 90%) as a colorless solid.

### Synthesis of Example 134

### 1-((1-(3-chlorophenyl)-3-(trifluoromethyl)-1H-pyrazol-5-yl)methyl)-3-(6-((2-hydroxyethylamino)methyl)pyridin-3-yl)urea

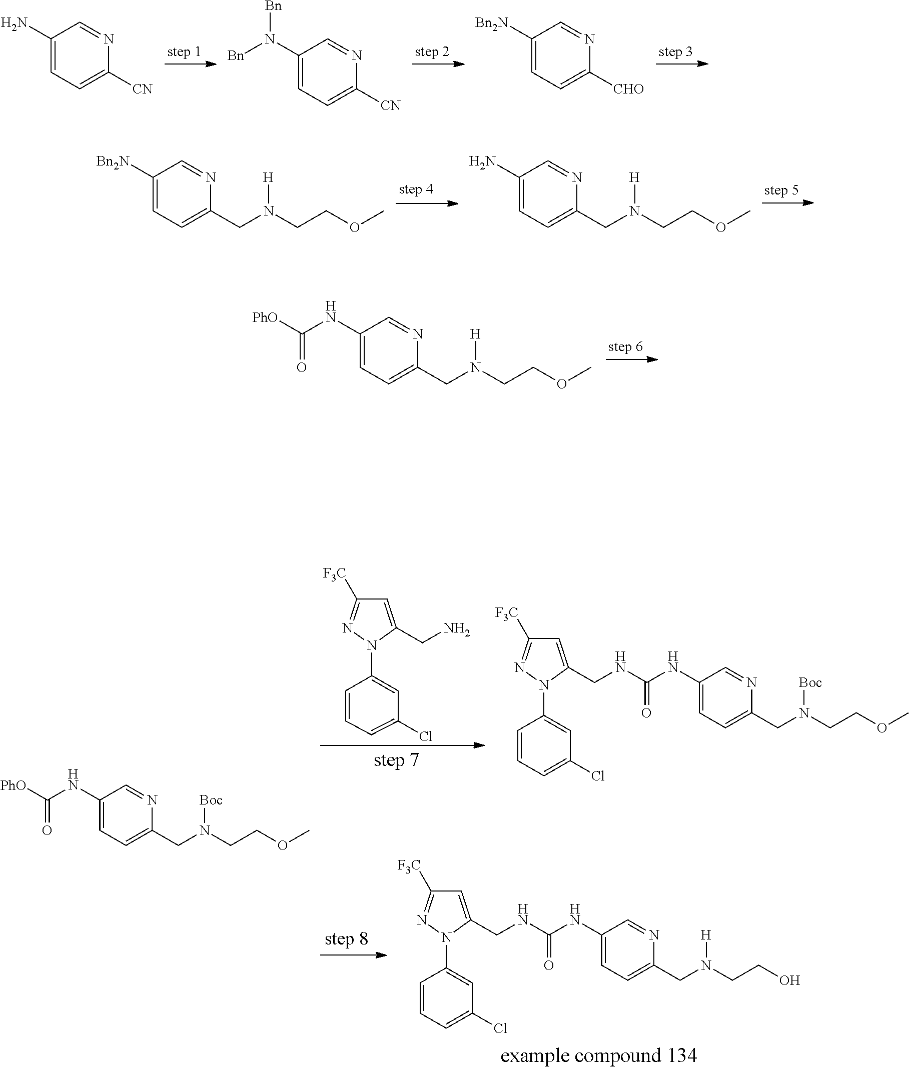

### Step 1:

To a stirred solution of 5-aminopicolinonitrile (10 g, 83.94 mmol, 1.0 eq) in dimethylformamide (100 mL) was added sodium hydride (6.0 g, 251.82 mmol, 3.0 eq) portion wise at 0° C. and then benzyl bromide was added and stirred for 3 h at room temperature. The reaction mixture was diluted with water (200 mL), extracted with ethyl acetate (2×150 mL) washed with brine (150 mL). The organic layer was dried over anhydrous sodium sulfate and evaporated under vacuum. The crude was purified by using silica gel chromatography (100-200 mesh) using ethyl acetate/petrol ether (3:7) to get 5-(dibenzylamino)picolinonitrile (17 g, 68%) as a pale yellow solid.

### Step 2:

To a stirred solution of 5-(dibenzylamino)picolinonitrile (200 mg, 0.668 mmol, 1.0 eq) in tetrahydrofuran (100 mL) cooled to −78° C. was added 1M DIBAL in toluene (1.3 mL, 1.337 mmol, 2.0 eq) slowly and stirred for 3 h at −78° C. The reaction mixture was diluted with water (150 mL), extracted with ethyl acetate (70 mL×2) and the organic layer was washed with brine (100 mL) and dried over sodium sulfate and evaporated under vacuum. The crude was purified by silica gel chromatography (100-200 mesh) using ethyl acetate/petrol ether (2:3) to get 5-(dibenzylamino)picolinaldehyde (100 mg, 50%) as solid.

### Step 3:

To a stirred solution of 5-(dibenzylamino)picolinaldehyde (50 mg, 0.615 mmol, 1.0 eq) in tetrahydrofuran (30 mL) was added 2-methoxyethanamine (18.6 mg, 0.248 mmol, 1.5 eq), catalytic amount of acetic acid (1 drop) and sodium triacetoxyborohydride (140 mg, 0.662 mmol, 2.0 eq) portion wise at 0° C. and stirred at room temperature for 3 h. The reaction mixture was neutralized by using NaHCO₃ and it was diluted with water (50 mL), extracted with ethyl acetate (2×60 mL). The organic layer was washed with brine (50 mL), dried over sodium sulfate and evaporated under vacuum. The residue obtained was purified by neutral alumina using ethyl acetate/petrol ether (3:7) as eluent to get N,N-dibenzyl-6-((2-methoxyethylamino)methyl)pyridin-3-amine (35 mg, 58%) as white solid.

### Step 4:

N,N-dibenzyl-6-((2-methoxyethylamino)methyl)pyridin-3-amine (2 g, 8.3 mmol, 1.0 eq) was dissolved in conc. sulfuric acid (5 mL) and heated to 50° C. for 3 h. The pH≈9 of the reaction mixture was adjusted with 2N NaOH solution and extracted with ethyl acetate (2×100 mL). The organic layer was separated and washed with brine (2×10 mL), dried over sodium sulfate and evaporated under vacuum to get 6-((2-methoxyethylamino)methyl)pyridin-3-amine (550 mg, 53%, brown oil).

### Step 5:

To a stirred solution of 6-((2-methoxyethylamino)methyl)pyridin-3-amine (800 mg, 4.4 mmol, 1.0 eq) in acetone (10 mL) was added pyridine (0.69 mL, 8.8 mmol, 2.0 eq) and phenyl chloro formate (55 mg, 4.4 mmol, 1.0 eq) at 0° C. and stirred at room temperature for 1 h. The reaction mixture was diluted with water (100 mL), extracted with ethyl acetate (150 mL×2) and the combined organic layer was separated and washed with brine (100 mL), dried over sodium sulfate and evaporated under vacuum. The residue was purified by silica gel column (100-200 mesh) using ethyl acetate/petether (1:4) as eluent to get phenyl 6-((2-methoxyethylamino)methyl)pyridin-3-ylcarbamate (700 mg, 47%) as off white solid.

### Step 6:

To a stirred solution of phenyl 6-((2-methoxyethylamino)methyl)pyridin-3-ylcarbamate (700 mg, 2.083 mmol, 1.0 eq) in dichloromethane (10 mL) was added triethylamine (0.57 mL, 4.166 mmol, 2.0 eq) and di-tert-butyl dicarbonate (0.54 mL, 2.499 mmol, 1.2 eq) at 0° C. and stirred at room temperature for 1 h. The reaction mixture was diluted with water (100 mL), extracted with ethyl acetate (2×150 mL) and the combined organic layer was separated and washed with brine (100 mL), dried over sodium sulfate and evaporated under vacuum. The residue was purified by silica gel column (100-200 mesh) using ethyl acetate/petether (3:2) as eluent to get 6-((2-methoxyethyl-N-tert-butoxycarbonyl-amino)methyl)pyridin-3-ylcarbamate (650 mg, 68%) as colorless viscous liquid.

### Step 7:

To a stirred solution of compound 6-((2-methoxyethyl-N-tert-butoxycarbonyl-amino)methyl)pyridin-3-ylcarbamate (150 mg, 0.37 mmol, 1.0 eq) in dichloromethane (10 mL) was added triethylamine (0.1 mL, 0.74 mmol, and 2.0 eq) followed by compound (1-(3-chlorophenyl)-3-(trifluoromethyl)-1H-pyrazol-5-yl)methanamine (117 mg, 0.37 mmol, and 1.0 eq) at room temperature and stirred for 16 h. dichloromethane was evaporated, diluted with water (30 mL), extracted with (ethyl acetate 50 mL) washed with brine (30 mL), dried over anhydrous sodium sulfate and evaporated under vacuum. Crude was purified by silica gel column (100-200 mesh) chromatography using methanol/trichloromethane (1:19) to get tert-butyl (5-(3-((1-(3-chlorophenyl)-3-(trifluoromethyl)-1H-pyrazol-5-yl)methyl)ureido)pyridin-2-yl)methyl(2-methoxyethyl)carbamate (200 mg, 55%) as white solid.

### Step 8:

To a stirred solution of tert-butyl (5-(3-((1-(3-chlorophenyl)-3-(trifluoromethyl)-1H-pyrazol-5-yl)methyl)ureido)pyridin-2-yl)methyl(2-methoxyethyl)carbamate (200 mg, 0.343 mmol, 1.0 eq) in dichloromethane (10 mL) was added boron tribromide (0.68 mL, 0.686 mmol, 2.0 eq) and stirred for 3 h at −78° C. The reaction mixture was quenched with NaHCO3 sol. (10 mL), extracted with ethyl acetate (2×30 mL), washed with brine (15 mL), dried over anhydrous sodium sulfate and evaporated under vacuum. Crude was purified by neutral alumina column chromatography using methanol/dichloromethane/ammonia (1:4:0.5) as eluent to get 1-((1-(3-chlorophenyl)-3-(trifluoromethyl)-1H-pyrazol-5-yl)methyl)-3-(6-((2-hydroxyethylamino)methyl)pyridin-3-yl)urea (example compound 134) (90 mg, 56%) as off white solid.

### Synthesis of Example 135

### 1-((1-(3-chlorophenyl)-3-(trifluoromethyl)-1H-pyrazol-5-yl)methyl)-3-(6-(((2-hydroxyethyl)(methyl)amino)methyl)pyridin-3-yl)urea

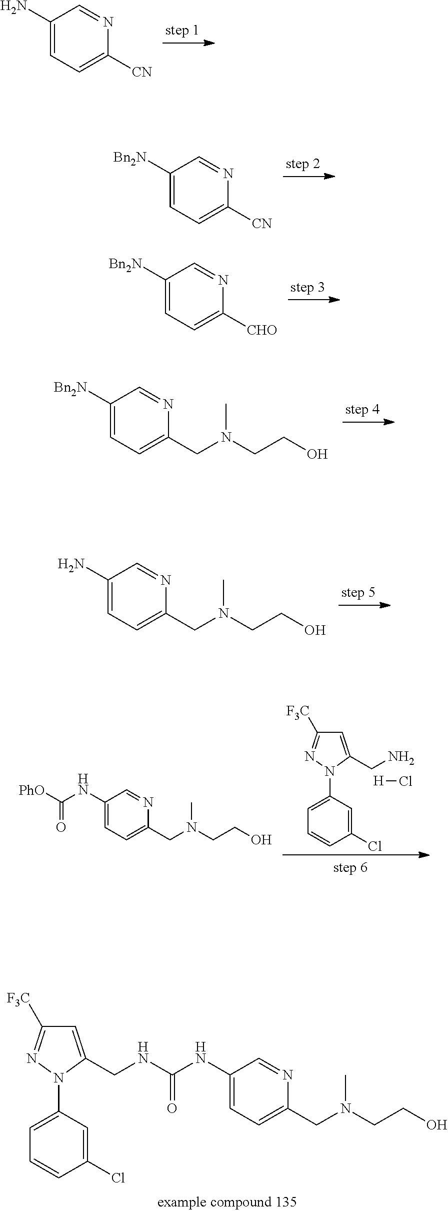

### Step 1:

To a stirred solution of 5-aminopicolinonitrile (10 g, 83.94 mmol, 1.0 eq) in dimethylformamide (100 mL) was added sodium hydride (60%) (6.0 g, 251.82 mmol, 3.0 eq) portion wise at 0° C. and then benzyl bromide was added and stirred for 3 h at room temperature. The reaction mixture was diluted with water (200 mL), extracted with ethyl acetate (2×150 mL) washed with brine (150 mL). The organic layer was dried over anhydrous sodium sulfate and evaporated under vacuum. The crude was purified by using silica gel chromatography (100-200 mesh) using ethyl acetate/petrol ether (3:7) to get 5-(dibenzylamino)picolinonitrile (17 g, 68%) as a pale yellow solid.

### Step 2:

To a stirred solution of 5-(dibenzylamino)picolinonitrile (200 mg, 0.668 mmol, 1.0 eq) in tetrahydrofuran (100 mL) cooled to −78° C. was added 1M DIBAL in toluene (1.3 mL, 1.337 mmol, 2.0 eq) slowly and stirred for 3 h at −78° C. The reaction mixture was diluted with water (150 mL), extracted with ethyl acetate (2×70 mL) and the organic layer was washed with brine (100 mL) and dried over sodium sulfate and evaporated under vacuum. The crude was purified by silica gel chromatography (100-200 mesh) using ethyl acetate/petrol ether (2:3) to get 5-(dibenzylamino)picolinaldehyde (100 mg, 50%) as solid.

### Step 3:

To a stirred solution of 5-(dibenzylamino)picolinaldehyde (100 mg, 0.33 mmol, 1.0 eq) in tetrahydrofuran (10 mL) was added 2-(methylamino)ethanol (37 mg, 0.49 mmol, 1.5 eq), catalytic amount of acetic acid and sodium triacetoxyborohydride (175 mg, 0.827 mmol, 2.5 eq) portion wise at 0° C. and stirred at room temperature for 3 h. The reaction mixture was neutralized by using NaHCO₃ and diluted with water (50 mL), extracted with ethyl acetate (2×60 mL). The organic layer was washed with brine (50 mL), dried over sodium sulfate and evaporated under vacuum. The residue obtained was purified by neutral alumina using ethyl acetate/petrol ether (1:4) as eluent to get 2-(((5-(dibenzylamino)pyridin-2-yl)methyl)(methyl)amino)ethanol (50 mg, 42%) as white solid.

### Step 4:

2-(((5-(dibenzylamino)pyridin-2-yl)methyl)(methyl)amino)ethanol (3 g, 8.31 mmol, 1.0 eq) was dissolved in conc. sulfuric acid (10 mL) and heated to 50° C. for 3 h. The reaction mixture was cooled, pH adjusted to ˜9 with 2N NaOH solution and extracted with ethyl acetate (2×100 mL). The organic layer was separated and washed with brine (2×10 mL), dried over sodium sulfate and evaporated under vacuum to get 2-(((5-aminopyridin-2-yl)methyl)(methyl)amino)ethanol (800 mg, 53%) as a brown oil. The isolated compound used directly for the next stage.

### Step 5:

To a stirred solution 2-(((5-aminopyridin-2-yl)methyl)(methyl)amino)ethanol (600 mg, 3.30 mmol, 1.0 eq) in acetone (10 mL) was added pyridine (0.78 mL, 9.90 mmol, 3.0 eq) and phenyl chloroformate (0.41 mL, 3.30 mmol, 1.0 eq) at 0° C. and stirred at room temperature for 1 h. The reaction mixture was diluted with water (50 mL), extracted with ethyl acetate (2×50 mL) and the combined organic layer was separated and washed with brine (50 mL), dried over sodium sulfate and evaporated under vacuum. The residue was purified by silica gel column (100-200 mesh) using methanol/trichloromethane (1:19) as eluent to get compound phenyl 6-(((2-hydroxyethyl)(methyl)amino)methyl)pyridin-3-ylcarbamate (300 mg, 23%) as off white solid.

### Step 6:

To a stirred solution of (1-(3-chlorophenyl)-3-(trifluoromethyl)-1H-pyrazol-5-yl)methanamine hydrochloride (103.4 mg, 0.332 mmol, 1.0 eq) in dichloromethane (5 mL) was added triethylamine (0.13 mL, 0.996 mmol, 3.0 eq) followed by phenyl 6-(((2-hydroxyethyl)(methyl)amino)methyl)pyridin-3-ylcarbamate (100 mg, 0.332 mmol, 1.0 eq) at room temperature and stirred for overnight. Dichloromethane is evaporated, then reaction mixture was diluted with water (50 mL), extracted with ethyl acetate (2×30 mL). The combined organic layer was washed with brine (5 mL), dried over anhydrous sodium sulfate and evaporated under vacuum. The crude was purified by silica gel (100-200 mesh) column chromatography using methanol/trichloromethane (3:17) to get compound 1-((1-(3-chlorophenyl)-3-(trifluoromethyl)-1H-pyrazol-5-yl)methyl)-3-(6-(((2-hydroxyethyl)(methyl)amino)methyl)pyridin-3-yl)urea (example compound 135) (70 mg, 43%) as a white solid.

### Synthesis of Example 139

### 1-((3-tert-butyl-1-(3-chlorophenyl)-1H-pyrazol-5-yl)methyl)-3-(5-(hydroxymethyl)pyridin-2-yl)urea

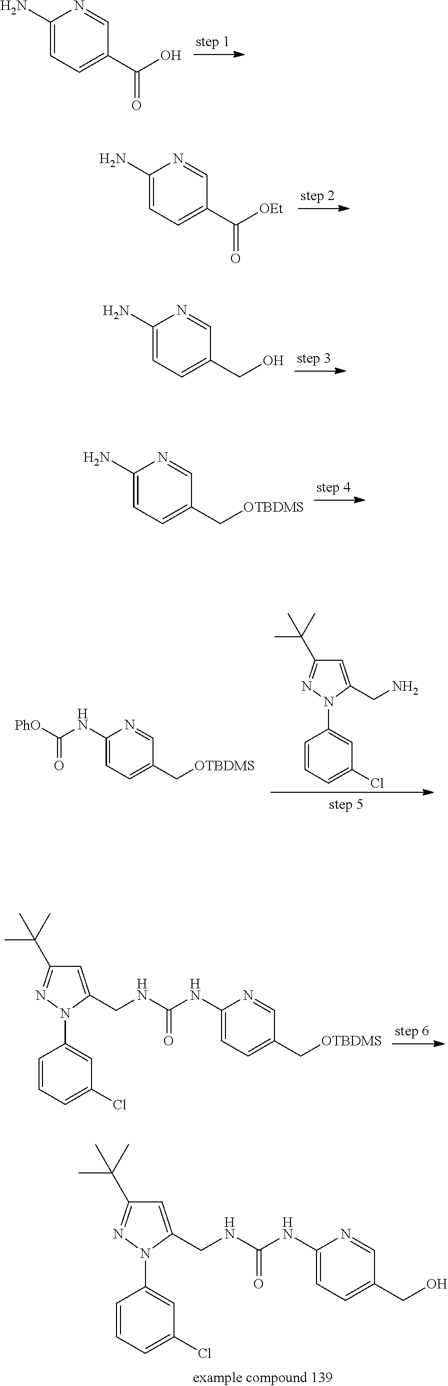

### Step 1:

To a stirred solution of 6-aminonicotinic acid (218 mg, 1.58 mmol) in ethanol was slowly added thionyl chloride (0.56 mL, 4.74 mmol) at 0° C. The reaction mixture was stirred overnight under reflux. Then the mixture was cooled to room temperature and the solvent was removed in vacuo. Then it was dissolved in ethyl acetate and washed with saturated sodium bicarbonate solution. The organic layer was dried (magnesium sulfate) and filtered. The filtrate was removed in vacuo. The crude condition of ethyl 6-aminonicotinate (200 mg, crude) was obtained in 76% yield.

### Step 2:

To a stirred solution of lithium aluminium hydride (183 mg, 4.83 mmol) in tetrahydrofuran was slowly added solution of ethyl 6-aminonicotinate (200 mg, 1.21 mmol) in tetrahydrofuran at 0° C. under nitrogen atmosphere. The reaction mixture was stirred at 0° C. for 30 minutes then at room temperature for 3 h. The mixture was quenched at 0° C. with 1N HCl until pH is 3 then basified with sodium carbonate solution until pH is 7. Then the mixture was filtered using celite to remove LAH residue and it was dissolved in ethyl acetate and washed with saturated sodium carbonate solution. The organic layer was dried (magnesium sulfate) and filtered. The filtrate was removed in vacuo. The crude condition of (6-aminopyridin-3-yl)methanol (55 mg, crude) was obtained in 75% yield.

### Step 3:

To a stirred solution of (6-aminopyridin-3-yl)methanol (55 mg, 0.44 mmol) in dimethylformamide were added imidazole (60 mg, 0.88 mmol) and tert-butyldimethylchlorosilane (66 mg, 0.44 mmol). The reaction mixture was stirred at room temperature for 5 h. The mixture dissolved in ethyl acetate and washed with water several times. The organic layer was dried over magnesium sulfate and filtered. The filtrate was removed in vacuo. The crude was purified by column chromatography. 5-((tert-butyldimethylsilyloxy)methyl)pyridin-2-amine (44 mg) was obtained in 42% yield.

### Step 4:

To a stirred solution of 5-((tert-butyldimethylsilyloxy)methyl)pyridin-2-amine (44 mg, 0.18 mmol) in tetrahydrofuran and acetonitrile as co-solvent were added phenylchloroformate (0.03 mL, 0.20 mmol) and pyridine (0.018 mL, 0.22 mmol). The reaction mixture was stirred for an hour at room temperature. The mixture dissolved in ethyl acetate and washed with water and brine. The organic layer was dried over magnesium sulfate and filtered. The filtrate removed in vacuo. The crude was purified by column chromatography. Phenyl 5-((tert-butyldimethylsilyloxy)methyl)pyridin-2-ylcarbamate (52 mg) was obtained in 79% yield.

### Step 5:

To a stirred solution of phenyl 5-((tert-butyldimethylsilyloxy)methyl)pyridin-2-ylcarbamate (50 mg, 0.15 mmol) and (3-tert-butyl-1-(3-chlorophenyl)-1H-pyrazol-5-yl)methanamine (40 mg, 0.15 mmol) in acetonitrile were added 4-dimethylaminopyridine (30 mg, 0.15 mmol). The reaction mixture was stirred overnight at 50° C. The mixture dissolved in ethyl acetate and washed with water and brine. The organic layer was dried over magnesium sulfate and filtered. The filtrate removed in vacuo. The crude was purified by column chromatography. 1-((3-Tert-butyl-1-(3-chlorophenyl)-1H-pyrazol-5-yl)methyl)-3-(5-((tert-butyldimethylsilyloxy)methyl)pyridin-2-yl)urea (68 mg) was obtained as 86% yield.

### Step 6:

To a stirred solution of 1-((3-tert-butyl-1-(3-chlorophenyl)-1H-pyrazol-5-yl)methyl)-3-(5-((tert-butyldimethylsilyloxy)methyl)pyridin-2-yl)urea (68 mg, 0.13 mmol) in tetrahydrofuran was added 1M tetra-n-butylammoniumfluoride (0.26 mL, 0.26 mmol). The reaction mixture was stirred for 18 h at room temperature. Then another portion of 1M tetra-n-butylammoniumfluoride (0.39 mL, 0.39 mmol) was added and the mixture was stirred for another 4 h. The mixture was quenched with saturated sodium bicarbonate solution then dissolved in ethyl acetate and washed with water. The organic layer was dried over magnesium sulfate and filtered. The filtrate removed in vacuo. The crude was purified by column chromatography. 1-((3-tert-butyl-1-(3-chlorophenyl)-1H-pyrazol-5-yl)methyl)-3-(5-(hydroxymethyl)pyridin-2-yl)urea (example compound 139) (31 mg) was obtained in 56% yield.

¹H NMR (300 MHz, CD₃OD) 8.08 (s, 1H, Ar), 7.66 (dd, 1H, J=8.57 Hz, Ar), 7.52 (m, 1H, Ar), 7.44 (br m, 3H, J=7.42-7.47, Ar), 6.98 (d, 1H, J=8.43 Hz, Ar), 6.35 (s, 1H, pyrazole), 4.55 (s, 2H, CH₂), 4.53 (s, 2H, CH₂), 1.31 (s, 9H, t-butyl)

### Synthesis of Example 140

### 1-((3-tert-butyl-1-(3-chlorophenyl)-1H-pyrazol-5-yl)methyl)-3-(5-(hydroxymethyl)pyridin-3-yl)urea

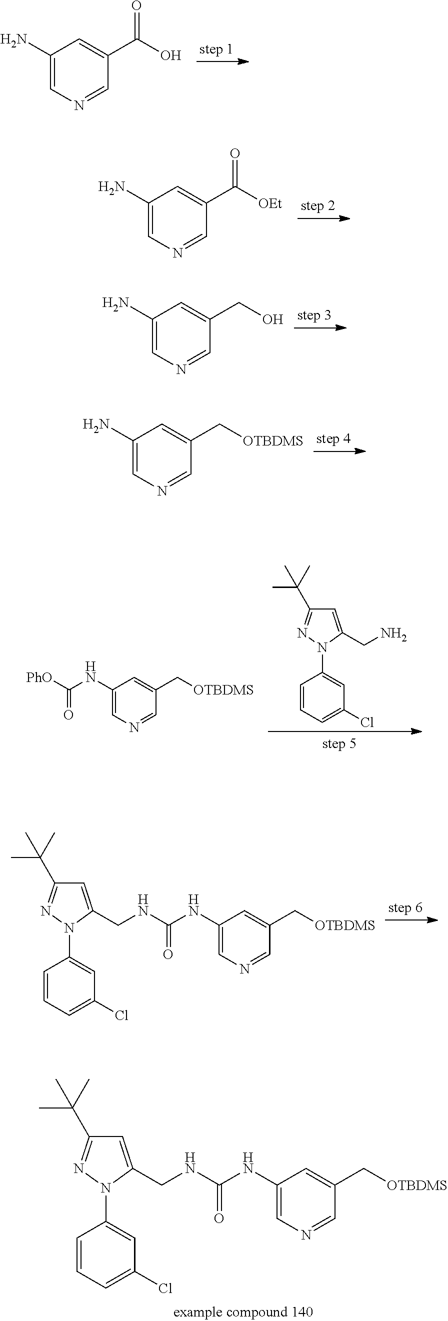

### Step 1:

To a stirred solution of 5-aminonicotinic acid (300 mg, 2.17 mmol) in ethanol was slowly added thionyl chloride (0.47 mL, 6.51 mmol) at 0° C. The reaction mixture was stirred overnight under reflux. Then the mixture was cooled to room temperature and the solvent was removed in vacuo. Then it was dissolved in ethyl acetate and washed with saturated sodium bicarbonate solution. The organic layer was dried (magnesium sulfate) and filtered. The filtrate was removed in vacuo. The crude condition of ethyl 5-aminonicotinate (315 mg, crude) was obtained in 89% yield.

### Step 2:

To a stirred solution of lithium aluminium hydride (254 mg, 5.36 mmol) in tetrahydrofuran was slowly added solution of ethyl 5-aminonicotinate (223 mg, 1.34 mmol) in tetrahydrofuran at 0° C. under nitrogen atmosphere. The reaction mixture was stirred at 0° C. for 30 minutes then at room temperature for 3 h. The mixture was quenched at 0° C. with 1N HCl until pH is 3 then basified with sodium carbonate solution until pH is 7. Then the mixture was filtered using celite to remove LAH residue and it was dissolved in ethyl acetate and washed with saturated sodium carbonate solution. The organic layer was dried over magnesium sulfate and filtered. The filtrate was removed in vacuo. The crude condition of (5-aminopyridin-3-yl)methanol (111 mg, crude) was obtained in 54% yield.

### Step 3:

To a stirred solution of (5-aminopyridin-3-yl)methanol (87 mg, 0.89 mmol) in dimethylformamide were added imidazole (12 mg, 1.77 mmol) and tert-butyldimethylchlorosilane (134 mg, 0.89 mmol). The reaction mixture was stirred at room temperature for 5 hours. The mixture dissolved in ethyl acetate and washed with water several times. The organic layer was dried over magnesium sulfate and filtered. The filtrate was removed in vacuo. The crude was purified by column chromatography. 5-((Tert-butyldimethylsilyloxy)methyl)pyridin-3-amine (132 mg) was obtained in 50% yield.

### Step 4:

To a stirred solution of 5-((tert-butyldimethylsilyloxy)methyl)pyridin-3-amine (132 mg, 0.55 mmol) in tetrahydrofuran and acetonitrile as co-solvent were added phenylchloroformate (0.073 mL, 0.58 mmol) and pyridine (0.054 mL, 0.66 mmol). The reaction mixture was stirred for one hour at room temperature. The mixture dissolved in ethyl acetate and washed with water and brine. The organic layer was dried (magnesium sulfate) and filtered. The filtrate removed in vacuo. The crude was purified by column chromatography. Phenyl 5-((tert-butyldimethylsilyloxy)methyl)pyridin-3-ylcarbamate (171 mg) was obtained in 86% yield.

### Step 5:

To a stirred solution of phenyl 5-((tert-butyldimethylsilyloxy)methyl)pyridin-3-ylcarbamate (80 mg, 0.22 mmol) and (3-tert-butyl-1-(3-chlorophenyl)-1H-pyrazol-5-yl)methanamine (59 mg, 0.22 mmol) in acetonitrile were added 4-dimethylaminopyridine (27 mg, 0.22 mmol). The reaction mixture was stirred overnight at 50° C. The mixture dissolved in ethyl acetate and washed with water and brine. The organic layer was dried (magnesium sulfate) and filtered. The filtrate removed in vacuo. The crude was purified by column chromatography. 1-((3-Tert-butyl-1-(3-chlorophenyl)-1H-pyrazol-5-yl)methyl)-3-(5-((tert-butyldimethylsilyloxy)methyl)pyridin-3-yl)urea (86 mg) was obtained as 73% yield.

### Step 6:

To a stirred solution of 1-((3-tert-butyl-1-(3-chlorophenyl)-1H-pyrazol-5-yl)methyl)-3-(5-((tert-butyldimethylsilyloxy)methyl)pyridin-3-yl)urea (86 g, 0.16 mmol) in tetrahydrofuran was added 1M tetra-n-butylammoniumfluoride (0.18 mL, 0.18 mmol). The reaction mixture was stirred for 18 h at room temperature. Then another portion of 1M tetra-n-butylammoniumfluoride (0.47 mL, 0.47 mmol) was added and the mixture was stirred for another 4 h. The mixture was quenched with saturated sodium bicarbonate solution then dissolved in ethyl acetate and washed with water. The organic layer was dried (magnesium sulfate) and filtered. The filtrate removed in vacuo. The crude was purified by column chromatography. 1-((3-Tert-butyl-1-(3-chlorophenyl)-1H-pyrazol-5-yl)methyl)-3-(5-((tert-butyldimethylsilyloxy)methyl)pyridin-3-yl)urea (example compound 140) (65 mg) was obtained in 96% yield.

¹H NMR (300 MHz, CD₃OD) 8.43 (d, 1H, J=2.37 Hz, Ar), 8.13 (s, 1H, Ar), 7.91 (s, 1H, Ar), 7.56 (t, 1H, J=2.01 Hz, Ar), 7.48 (br m, 3H, 7.43-7.54, Ar), 6.37 (s, 1H, pyrazole), 4.62 (s, 2H, CH₂), 4.44 (s, 2H, CH₂), 1.32 (m, 9H, t-butyl)-

### Synthesis of Example 141

### 1-((1-(3-chlorophenyl)-3-(trifluoromethyl)-1H-pyrazol-5-yl)methyl)-3-(6-(hydroxymethyl)-2-methylpyridin-3-yl)urea

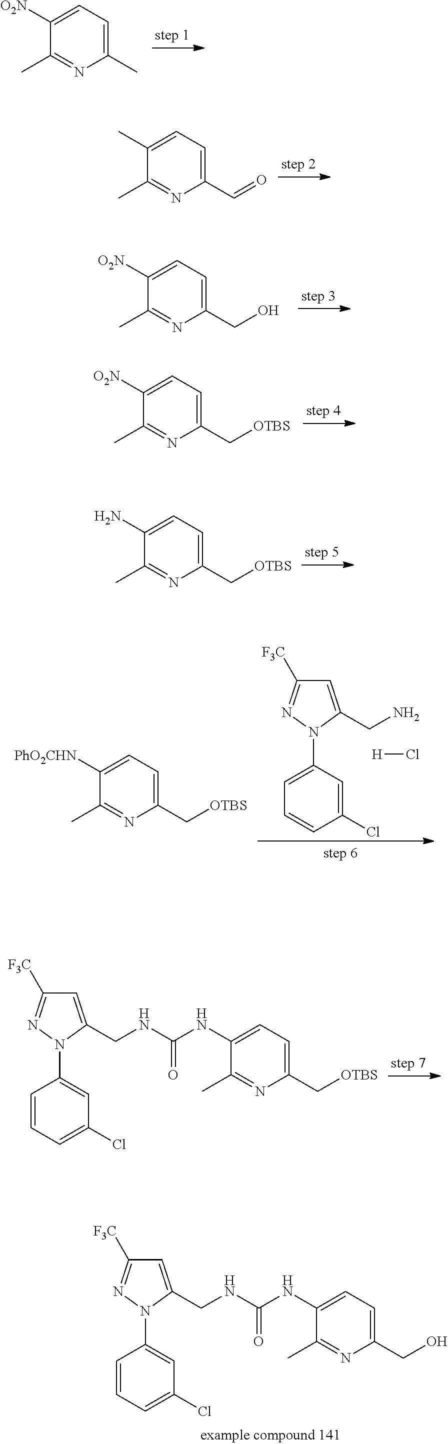

### Step 1:

To a stirred solution of 2,6-dimethyl-3-nitropyridine (3 g, 19.5 mmol, 1.0 eq) in 1,4-dioxane (20 mL) was added selenium dioxide (2.625 g, 28.80 mmol, 1.2 eq) and reaction mixture was stirred for 16 h at 100° C. The reaction mixture was filtered through celite bed and filtrate concentrated under reduced pressure to get 5,6-dimethylpicolinaldehyde (3.0 g, 95%) as brown liquid.

### Step 2:

To a stirred solution of 5,6-dimethylpicolinaldehyde (3.0 g, 18.2 mmol, 1.0 eq) in methanol (20 mL) was added NaBH₄ (720 mg, 18.2 mmol, 1 eq) at 0° C. and then stirred for 1 h at 0° C. The reaction mixture was quenched with ice water (10 mL) and concentrated under reduced pressure, extracted with dichloromethane (2×50 mL), and concentrated to get (6-methyl-5-nitropyridin-2-yl)methanol (2.4 g, 75%).

### Step 3:

To a stirred solution of (6-methyl-5-nitropyridin-2-yl)methanol (500 mg, 3.01 mmol, 1.0 eq) in dichloromethane (10 mL) was added imidazole (410 mg, 6.02 mmol, 2 eq) followed by TBDMSCl (500 mg, 3.313 mmol, and 1.1 eq) at 0° C. and stirred for 1 h at 0° C. The reaction mixture was diluted with water (50 mL), and extracted with dichloromethane (2×50 mL), concentrated to get 6-((tert-butyldimethylsilyloxy)methyl)-2-methyl-3-nitropyridine (800 mg, ˜94%).

### Step 4:

To a stirred solution 6-((tert-butyldimethylsilyloxy)methyl)-2-methyl-3-nitropyridine (800 mg, 3.54 mmol, 1.0 eq) in methanol (50 mL) was added Pd/C (400 mg) and stirred under 40 psi H₂ for 16 h. The reaction mixture was passed through celite, concentrated the filtrate under reduced pressure to get 6-((tert-butyldimethylsilyloxy)methyl)-2-methylpyridin-3-amine (700 mg, 95%).

### Step 5:

To a stirred solution of 6-((tert-butyldimethylsilyloxy)methyl)-2-methylpyridin-3-amine (700 mg, 2.7 mmol, 1.0 eq) in acetone (20 mL) was added pyridine (639 mg, 8.1 mmol, 3.0 eq) and phenyl chloroformate (422 mg, 2.7 mmol, 1.0 eq) at 0° C. and stirred at 0° C. for 4 h. Acetone was evaporated and residue was purified by silica gel (100-200 mesh) column chromatography using ethyl acetate/petrol ether (1:9) as eluent to get phenyl 6-((tert-butyldimethylsilyloxy)methyl)-2-methylpyridin-3-ylcarbamate (1.0 g, 90%) as a sticky solid.

### Step 6:

To a stirred solution of phenyl 6-((tert-butyldimethylsilyloxy)methyl)-2-methylpyridin-3-ylcarbamate (180 mg, 0.48 mmol, 1.0 eq) in dichloromethane (20 mL) were added triethylamine (154 mg, 1.44 mmol, 3 eq) and (1-(3-chlorophenyl)-3-(trifluoromethyl)-1H-pyrazol-5-yl)methanamine hydrochloride (150 mg, 0.48 mmol, 1.0 eq) at 0° C. The reaction mixture was stirred at room temperature for 12 h. The reaction mixture was diluted with dichloromethane and washed with water and extracted with dichloromethane, dried over sodium sulfate and concentrated under reduced pressure to get 1-(6-((tert-butyldimethylsilyloxy)methyl)-2-methylpyridin-3-yl)-3-((1-(3-chlorophenyl)-3-(trifluoromethyl)-1H-pyrazol-5-yl)methyl)urea (300 mg, ˜70%) as a white solid.

### Step 6:

To a stirred solution of 1-(6-((tert-butyldimethylsilyloxy)methyl)-2-methylpyridin-3-yl)-3-((1-(3-chlorophenyl)-3-(trifluoromethyl)-1H-pyrazol-5-yl)methyl)urea (300 mg, 0.541 mmol, 1.0 eq) in tetrahydrofuran (20 mL) was added 2N HCl (10 mL) at 0° C. The reaction mixture was stirred at room temperature for 3 h and tetrahydrofuran evaporated. The residue was diluted with ethyl acetate and washed with water, dried over sodium sulfate and evaporated under reduced pressure. The crude was purified by silica gel column chromatography (100-200 mesh) using methanol/dichloromethane (1:9) as eluent to get 1-((1-(3-chlorophenyl)-3-(trifluoromethyl)-1H-pyrazol-5-yl)methyl)-3-(6-(hydroxymethyl)-2-methylpyridin-3-yl)urea (example compound 141) (120 mg, ˜40%) as off white solid.

### Synthesis of Example 143

### 1-((1-(3-chlorophenyl)-3-(trifluoromethyl)-1H-pyrazol-5-yl)methyl)-3-(6-(2-hydroxyethyl)-2-methylpyridin-3-yl)urea

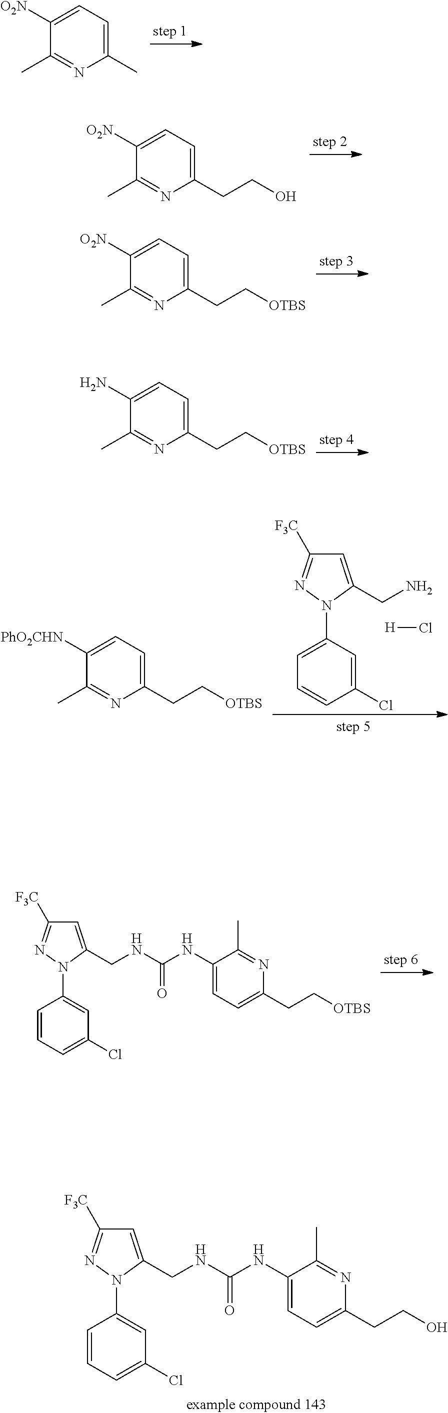

### Step 1:

To a stirred solution of 2,6-dimethyl-3-nitropyridine (200 mg, 1.3 mmol, 1.0 eq) in ethanol (10 mL) was added 40% aqs. formaldehyde (15 mL) followed by water (10 mL) room temperature and stirred for 48 h at 200° C. The aqs solvent was evaporated and crude purified by silica gel column chromatography (100-200 mesh) using ethyl acetate/petrol ether (2:8) as eluent to get 2-(6-methyl-5-nitropyridin-2-yl)ethanol (220 mg, 55%) as yellow liquid.

### Step 2:

To a stirred solution of 2-(6-methyl-5-nitropyridin-2-yl)ethanol (300 mg, 1.6 mmol, 1.0 eq) in dichloromethane (20 mL) was added imidazole (217 mg, 3.2 mmol, 2 eq) at 0° C. followed by TBDMSCl (264 mg, 1.76 mmol, 1.1 eq) and stirred for 1 h at 0° C. The reaction mixture was diluted with water (50 mL), and extracted into dichloromethane (2×50 mL), dried and concentrated to get 6-(2-(tert-butyldimethylsilyloxy)ethyl)-2-methyl-3-nitropyridine (400 mg, 82%).

### Step 3:

To a stirred solution of 6-(2-(tert-butyldimethylsilyloxy)ethyl)-2-methyl-3-nitropyridine (400 mg, 1.35 mmol, 1.0 eq) in methanol (50 mL) was added 10% Pd/C (150 mg) and stirred under H₂ atmosphere 16 h at 40 psi. The reaction mixture was passed through celite, filtrate evaporated under reduced pressure to get 6-(2-(tert-butyldimethylsilyloxy)ethyl)-2-methylpyridin-3-amine (300 mg, 83%).

### Step 4:

To a stirred solution of 6-(2-(tert-butyldimethylsilyloxy)ethyl)-2-methylpyridin-3-amine (300 mg, 1.12 mmol, 1.0 eq) in acetone (20 mL) was added pyridine (265 mg, 3.36 mmol, 3.0 eq) and phenyl chloroformate (176 mg, 1.12 mmol, 1.0 eq) at 0° C. and stirred at 0° C. for 4 h. Acetone was evaporated and residue was purified by silica gel (100-200 mesh) column chromatography using ethyl acetate/petrol ether (1:9) as eluent to get phenyl 6-(2-(tert-butyldimethylsilyloxy)ethyl)-2-methylpyridin-3-ylcarbamate (213 mg, 48%) as a sticky solid.

### Step 5:

To a stirred solution of phenyl 6-(2-(tert-butyldimethylsilyloxy)ethyl)-2-methylpyridin-3-ylcarbamate (213 mg, 0.55 mmol, 1.0 eq) in dichloromethane (5 mL) were added triethylamine (176 mg, 1.65 mmol, 3 eq), (1-(3-chlorophenyl)-3-(trifluoromethyl)-1H-pyrazol-5-yl)methanamine hydrochloride (171 mg, 0.55 mmol, 1.0 eq) at 0° C. The reaction mixture was stirred at same temperature for 12 h. Then the reaction mixture was diluted with dichloromethane and washed twice with water and extracted into dichloromethane, dried over sodium sulfate and concentrated under reduced pressure to get 1-(6-(2-(tert-butyldimethylsilyloxy)ethyl)-2-methylpyridin-3-yl)-3-((1-(3-chlorophenyl)-3-(trifluoromethyl)-1H-pyrazol-5-yl)methyl)urea (300 mg, 80%) as a yellow liquid.

### Step 6:

To a stirred solution of 1-(6-(2-(tert-butyldimethylsilyloxy)ethyl)-2-methylpyridin-3-yl)-3-((1-(3-chlorophenyl)-3-(trifluoromethyl)-1H-pyrazol-5-yl)methyl)urea (300 mg, 0.528 mmol, 1.0 eq) in tetrahydrofuran (20 mL) was added 2N HCl (10 mL), at 0° C. The reaction mixture was stirred at room temperature for 4 h and concentrated and diluted with ethyl acetate (20 mL) and washed twice with water, dried over sodium sulfate and concentrated under reduced pressure. This crude was purified by silica gel column chromatography (100-200 mesh) using methanol/dichloromethane (1:9) as eluent to get 1-((1-(3-chlorophenyl)-3-(trifluoromethyl)-1H-pyrazol-5-yl)methyl)-3-(6-(2-hydroxyethyl)-2-methylpyridin-3-yl)urea (example compound 143) (60 mg, 25%) as white solid.

### Synthesis of Example 144

### 1-((3-tert-butyl-1-(3-chlorophenyl)-1H-pyrazol-5-yl)methyl)-3-(6-(1,2-dihydroxyethyl)pyridin-3-yl)urea

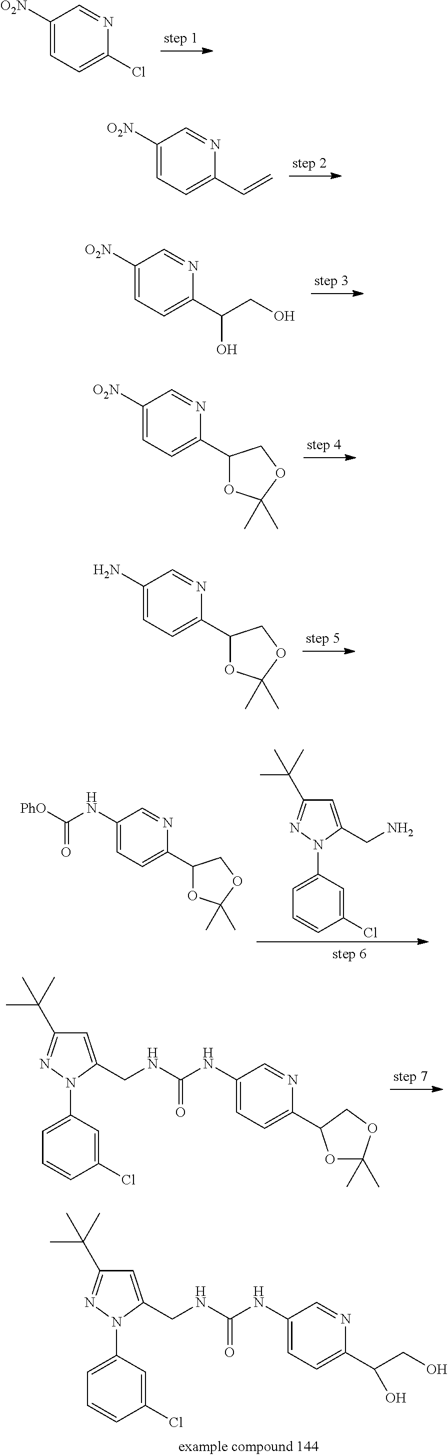

### Step 1:

To the solution 2-Chloro-4-Nitropyridine (500 mg, 3.15 mmol) in Tetrahydrofuran was added LiCl (936 mg, 22.08 mmol, 7 eq), Pd(PPh₃)₄ (547 mg, 0.47 mmol, 0.15 eq) and tributylvinyl-tin (1.84 mL, 6.31 mmol, 2 eq) at room temperature. The reaction mixture was refluxed for overnight under nitrogen atmosphere. TLC showed complete consumption of starting material. The reaction mixture was cooled to room temperature. The mixture was diluted ethyl acetate and the organic layer was washed with saturated potassium fluoride solution and then extracted with ethyl acetate. The organic part was washed with brine. The organic layer was dried over magnesium sulfate and concentrated under reduced pressure to afford crude product which was purified by column chromatography to afford 5-nitro-2-vinylpyridine (350 mg, 74%)

### Step 2:

To the solution of 5-nitro-2-vinylpyridine (350 mg, 2.33 mmol) in acetone under nitrogen atmosphere gas was added of 0.5% osmium tetroxide (in water) (2.36 mL, 0.05 mmol, 0.02 eq) and 50% 4-methylmorpholine N-oxide (in water) (1.66 mL, 6.99 mmol, 3 eq). Reaction mixture was stirred at room temperature for 4 h. TLC showed complete consumption of starting material. The reaction mixture was diluted with water and extracted with ethyl acetate. The organic part was washed with brine. The organic layer was dried over magnesium sulfate and concentrated under reduced pressure to afford crude product which was purified by column chromatography to afford 1-(5-nitropyridin-2-yl)ethane-1,2-diol (368 mg, 86%).

### Step 3:

A solution of 1-(5-nitropyridin-2-yl)ethane-1,2-diol (368 mg, 2.00 mmol) in dichloromethane was treated with ZrCl₄ (47 mg, 0.20 mmol, 0.1 eq) and 2,2-methoxypropane (0.3 mL, 2.40 mmol, 1.2 eq). The mixture was stirred for 4 h at room temperature. TLC showed complete consumption of starting material. The reaction mixture was diluted with water and extracted with ethyl acetate. The organic part was washed with brine. The organic layer was dried over magnesium sulfate and concentrated under reduced pressure to afford crude product which was purified by column chromatography to afford the 2-(2,2-dimethyl-1,3-dioxolan-4-yl)-5-nitropyridine (311 mg, 69%)

### Step 4:

2-(2,2-Dimethyl-1,3-dioxolan-4-yl)-5-nitropyridine (311 mg, 1.38 mmol) was dissolved in methanol and tetrahydrofuran(1:1, 15 mL). 10% Pd/C (31 mg, 10%) was added to it. The resulting mixture was stirred at room temperature for 3 h under H₂. TLC showed complete consumption of starting material. The mixture was filtered through celite bed and the filterate was concentrated under reduced pressure. The crude was purified by column chromatography to give 6-(2,2-dimethyl-1,3-dioxolan-4-yl)pyridin-3-amine (201 mg, 75%).

### Step 5:

6-(2,2-Dimethyl-1,3-dioxolan-4-yl)pyridin-3-amine (201 mg, 1.04 mmol) was dissolved inacetonitrile (3 mL) and tetrahydrofuran (4 mL). The reaction mixture was added pyridine (0.10 mL, 1.24 mmol, 1.2 eq) and phenyl chloroformate (0.14 mL, 1.09 mmol, 1.05 eq) and stirred at room temperature for 3 h under nitrogen atmosphere. TLC showed complete consumption of starting material. The reaction mixture was diluted with water and extracted with ethyl acetate. The organic part was washed with water and brine. The organic layer was dried over magnesium sulfate and concentrated under reduced pressure. The crude was purified by column chromatography to give phenyl 6-(2,2-dimethyl-1,3-dioxolan-4-yl)pyridin-3-ylcarbamate (321 mg, 99%).

### Step 6:

To a solution of phenyl 6-(2,2-dimethyl-1,3-dioxolan-4-yl)pyridin-3-ylcarbamate (79 mg, 0.251 mmol) in CH₃CN was added 4-dimethylaminopyridine (31 mg, 0.251 mmol) and (3-tert-butyl-1-(3-chlorophenyl)-1H-pyrazol-5-yl)methanamine (66 mg, 0.251 mmol) at room temperature. The reaction mixture was heated to 50° C. for overnight. TLC showed complete consumption of starting material. The reaction mixture was diluted with water and extracted with ethyl acetate. The organic part was washed with water and brine. The organic layer was dried over magnesium sulfate and concentrated under reduced pressure. The crude was purified by column chromatography to give 1-((3-tert-butyl-1-(3-chlorophenyl)-1H-pyrazol-5-yl)methyl)-3-(6-(2,2-dimethyl-1,3-dioxolan-4-yl)pyridin-3-yl)urea (111 mg, 91%).

### Step 7:

A solution of 1-((3-tert-butyl-1-(3-chlorophenyl)-1H-pyrazol-5-yl)methyl)-3-(6-(2,2-dimethyl-1,3-dioxolan-4-yl)pyridin-3-yl)urea (111 mg, 0.229 mmol) in methanol was added ZrCl₄ (5 mg, 0.0229 mmol) at room temperature. The reaction mixture was heated to 35° C. for overnight. TLC showed complete consumption of starting material. The reaction mixture was diluted with water and extracted with ethyl acetate. The organic part was washed with water and brine. The organic layer was dried over magnesium sulfate and concentrated under reduced pressure. The crude was purified by column chromatography to give 1-((3-tert-butyl-1-(3-chlorophenyl)-1H-pyrazol-5-yl)methyl)-3-(6-(1,2-dihydroxyethyl)pyridin-3-yl)urea (example compound 144) (21 mg, 21%).

¹H NMR (300 MHz, DMSO) δ 8.79 (br s, 1H), 8.44 (d, J=2.4 Hz, 1H), 7.82 (d, J=8.61 Hz, 1H), 7.61 (s, 1H), 7.55-7.46 (m, 3H), 7.32 (d, J=8.43 Hz, 1H), 6.84 (br t, 1H), 6.32 (s, 1H), 5.27 (d, J=4.74 Hz, 1H), 4.65 (t, J=5.7 Hz, 1H), 4.49 (m, 1H), 4.40 (d, J=5.67 Hz, 2H), 3.61 (m, 1H), 1.96 (d, J=9.87 Hz, 1H), 1.27 (s, 9H)

### Synthesis of Example 145

### 1-((1-(3-chlorophenyl)-3-(trifluoromethyl)-1H-pyrazol-5-yl)methyl)-3-(6-(1,2-dihydroxyethyl)pyridin-3-yl)urea

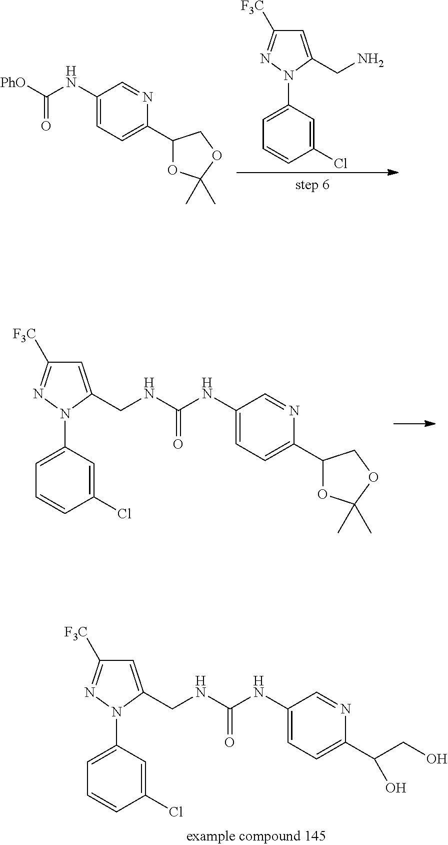

Step 1-5: see example compound 102.

### Step 6:

To a solution of phenyl 6-(2,2-dimethyl-1,3-dioxolan-4-yl)pyridin-3-ylcarbamate (200 mg, 0.64 mmol) in acetonitrile (3 mL) was added 4-dimethylaminopyridine (78 mg, 0.64 mmol, 1 eq) and (1-(3-chlorophenyl)-3-(trifluoromethyl)-1H-pyrazol-5-yl)methanamine (192 mg, 0.70 mmol, 1.1 eq) at room temperature. The reaction mixture was heated to 50° C. for overnight. TLC showed complete consumption of starting material. The reaction mixture was diluted with water and extracted with ethyl acetate. The organic part was washed with water and brine. The organic layer was dried over magnesium sulfate and concentrated under reduced pressure. The crude was purified by column chromatography to give 1-((1-(3-chlorophenyl)-3-(trifluoromethyl)-1H-pyrazol-5-yl)methyl)-3-(6-(2,2-dimethyl-1,3-dioxolan-4-yl)pyridin-3-yl)urea (303 mg, 96%)

### Step 7:

A solution of 1-((1-(3-chlorophenyl)-3-(trifluoromethyl)-1H-pyrazol-5-yl)methyl)-3-(6-(2,2-dimethyl-1,3-dioxolan-4-yl)pyridin-3-yl)urea (303 mg, 0.61 mmol) in methanol was added ZrCl₄ (28 mg, 0.12 mmol, 0.3 eq) at room temperature. The reaction mixture was heated to 35° C. for overnight. TLC showed complete consumption of starting material. The reaction mixture was diluted with water and extracted with ethyl acetate. The organic part was washed with water and brine. The organic layer was dried over magnesium sulfate and concentrated under reduced pressure. The crude was purified by column chromatography to give 1-((1-(3-chlorophenyl)-3-(trifluoromethyl)-1H-pyrazol-5-yl)methyl)-3-(6-(1,2-dihydroxyethyl)pyridin-3-yl)urea (example compound 145) (102 mg, 37%).

¹H NMR (400 MHz, CDCl₃) δ 8.45 (d, 1H, Ar), 7.86 (dd, 1H, J=2.1 Hz, 8.5 Hz, Ar), 7.64 (q, 1H, Ar), 7.51-7.56 (m, 3H, Ar), 7.45 (d, 1H, J=8.4 Hz, Ar), 6.75 (s, 1H, pyrazole), 4.69 (m, 1H, CH), 4.48 (s, 2H, CH₂), 3.60-3.80 (m, 2H, CH₂).

### Synthesis of Example 146

### N-((1-(3-chlorophenyl)-3-(trifluoromethyl)-1H-pyrazol-5-yl)methyl)-2-(6-(2-hydroxyethylamino)pyridin-3-yl)propanamide

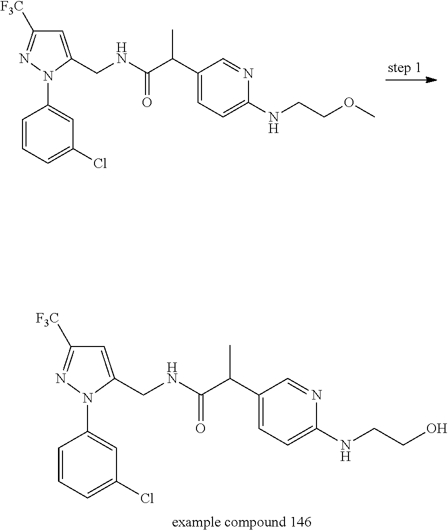 [SMILES: COCCNc1ccc(C(C)C(=O)NCc2cc(C(F)(F)F)nn2-c2cccc(Cl)c2)cn1 ; CC(C(=O)NCc1cc(C(F)(F)F)nn1-c1cccc(Cl)c1)c1ccc(NCCO)nc1]

### Step 1:

To a stirred solution N-((1-(3-chlorophenyl)-3-(trifluoromethyl)-1H-pyrazol-5-yl)methyl)-2-(6-(2-methoxyethylamino)pyridin-3-yl)propanamide (example compound 147, 300 mg, 0.623 mmol, 1.0 eq) in dichloromethane (10 mL) was added 1M boron tribromide in dichloromethane (1.87 mL, 1.871 mmol, 3.0 eq) at −78° C. and stirred at room temperature for 3 h and pH≈8 was adjusted with NaHCO₃, diluted with water (20 mL). The aqueous layer was extracted with ethyl acetate (2×50 mL) and the combined organic layer was separated and washed with brine (50 mL), dried over sodium sulfate and evaporated under vacuum. The residue was purified by silica gel column (100-200 mesh) using methanol/trichloromethane (1:9) as eluent to get N-((1-(3-chlorophenyl)-3-(trifluoromethyl)-1H-pyrazol-5-yl)methyl)-2-(6-(2-hydroxyethylamino)pyridin-3-yl)propanamide (example compound 146) (140 mg, 48%) as off white solid.

### Synthesis of Example 147

### N-((1-(3-chlorophenyl)-3-(trifluoromethyl)-1H-pyrazol-5-yl)methyl)-2-(6-(2-methoxyethylamino)pyridin-3-yl)propanamide

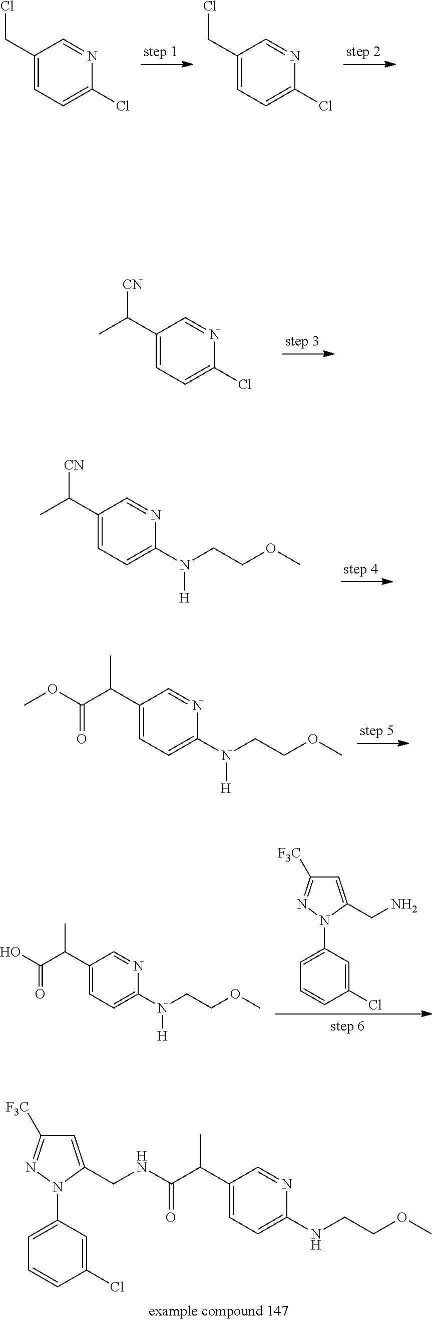

### Step 1:

To a stirred solution of 2-chloro-5-(chloromethyl)pyridine (1 g, 6.17 mmol, 1.0 eq) in ethanol (10 mL) was added the solution of NaCN (325 mg, 6.79 mmol, 1.1 eq) in water (10 mL) dropwise at 0° C. and then stirred for 3 h at 100° C. The reaction mixture was diluted with water (50 mL), extracted with ethyl acetate (2×70 mL) washed with brine (20 mL). The organic layer was dried over anhydrous sodium sulfate and evaporated under vacuum. The crude was purified by using silica gel chromatography (100-200 mesh) using ethyl acetate/petrol ether (3:7) to get 2-(6-chloropyridin-3-yl)acetonitrile (400 mg, 63%) as a yellow solid.

### Step 2:

To a stirred solution of 2-(6-chloropyridin-3-yl)acetonitrile (10 g, 65.7 mmol, 1.0 eq) in tetrahydrofuran (100 mL) was added sodium hydride (1.578 g, 65.7 mmol, 1.0 eq) as portion wise stirred for 10 min at 0° C. followed by methyl iodide (4.02 mL, 65.7 mmol, 1.0 eq). The reaction mixture was diluted slowly with water (150 mL) at 0° C., extracted with ethyl acetate (2×100 mL) and brine (100 mL) and dried over sodium sulfate and evaporated under vacuum. The crude was purified by silica gel chromatography (100-200 mesh) using ethyl acetate/petrol ether (1:4) to get 2-(6-chloropyridin-3-yl)propanenitrile (5 g, 46%) as solid.

### Step 3:

To a stirred solution of 2-(6-chloropyridin-3-yl)propanenitrile (2 g, 12.04 mmol, 1.0 eq) in DMSO (15 mL) was added triethylamine (3.34 mL, 24.09 mmol, 2.0 eq) and N-(2-methoxy ethyl)methyl amine (1.8 g, 24.09 mmol, 2.0 eq). The reaction mixture was heated to 100° C. for 16 h. The reaction mixture was diluted with water (50 mL), extracted with ethyl acetate (2×60 mL). The organic layer was washed with brine (50 mL), dried over sodium sulfate and evaporated under vacuum. The residue obtained was purified by neutral alumina using ethyl acetate/petrol ether (3:7) as eluent to get 2-(6-(2-methoxyethylamino)pyridin-3-yl)propanenitrile (500 mg, 40%) as white solid.

### Step 4:

To a stirred solution of TMSCl (4.6 mL, 20.4 mmol, 3.0 eq) in methanol (8 mL) was added 2-(6-(2-methoxyethylamino)pyridin-3-yl)propanenitrile (1.4 g, 6.8 mmol, 1.0 eq) and heated to 60° C. for 5 h. The reaction mixture was diluted with water (50 mL) and adjusted to pH≈9 with NaHCO₃ (10 mL) extracted with ethyl acetate (2×100 mL). The organic layer was separated and washed with brine (50 mL), dried over sodium sulfate and evaporated under vacuum. The residue was purified by silica gel column (100-200 mesh) using ethyl acetate/petrol ether (1:1) as eluent to get methyl 2-(6-(2-methoxyethylamino)pyridin-3-yl)propanoate (1.2 g, 73.5%) as a pale yellow liquid.

### Step 5:

To a stirred solution of methyl 2-(6-(2-methoxyethylamino)pyridin-3-yl)propanoate (200 mg, 0.840 mmol, 1.0 eq) in tetrahydrofuran/water (5 mL+5 mL) was added LiOH/water (104 mg, 2.52 mmol, 3.0 eq) at 60° C. and stirred for 2 h. The reaction mixture was diluted with water (5 mL), acidified (pH˜4) with 1N HCl, and then extracted with ethyl acetate (2×25 mL). The organic layer was washed with brine (20 mL), dried over anhydrous sodium sulfate and evaporated under vacuum to get 2-(6-(2-methoxyethylamino)pyridin-3-yl)propanoic acid (120 mg; 64%). Crude was directly used for next step without further purification.

### Step 6:

To a stirred solution of 2-(6-(2-methoxyethylamino)pyridin-3-yl)propanoic acid (224 mg, 0.446 mmol, 1.0 eq) in dichloromethane (5 mL) was added EDC.HCl (127 mg, 0.669 mmol, 1.5 eq) followed by HOBt (75 mg, 0.490 mmol, 1.1 eq), DIPEA (0.23 mL, 1.338 mmol, 3 eq) and then (1-(3-chlorophenyl)-3-(trifluoromethyl)-1H-pyrazol-5-yl)methanamine (139 mg, 0.446 mmol, 1.0 eq) at room temperature and stirred for 3 h. The reaction mixture was diluted with water (10 mL), extracted with ethyl acetate (2×25 mL). The organic layer was washed with brine (20 mL), dried over anhydrous sodium sulfate and evaporated under vacuum. Crude was purified by silica gel (100-200 mesh) column chromatography by using methanol/trichloromethane (1:19) as eluent to get N-((1-(3-chlorophenyl)-3-(trifluoromethyl)-1H-pyrazol-5-yl)methyl)-2-(6-(2-methoxyethylamino)pyridin-3-yl)propanamide (example compound 147) (80 mg, 37%) as off white solid.

Example 88 can be prepared in a similar manner.

### Synthesis of Example 148

### N-((1-(3-chlorophenyl)-3-(trifluoromethyl)-1H-pyrazol-5-yl)methyl)-2-(6-((2-hydroxyethyl)(methyl)amino)pyridin-3-yl)propanamide

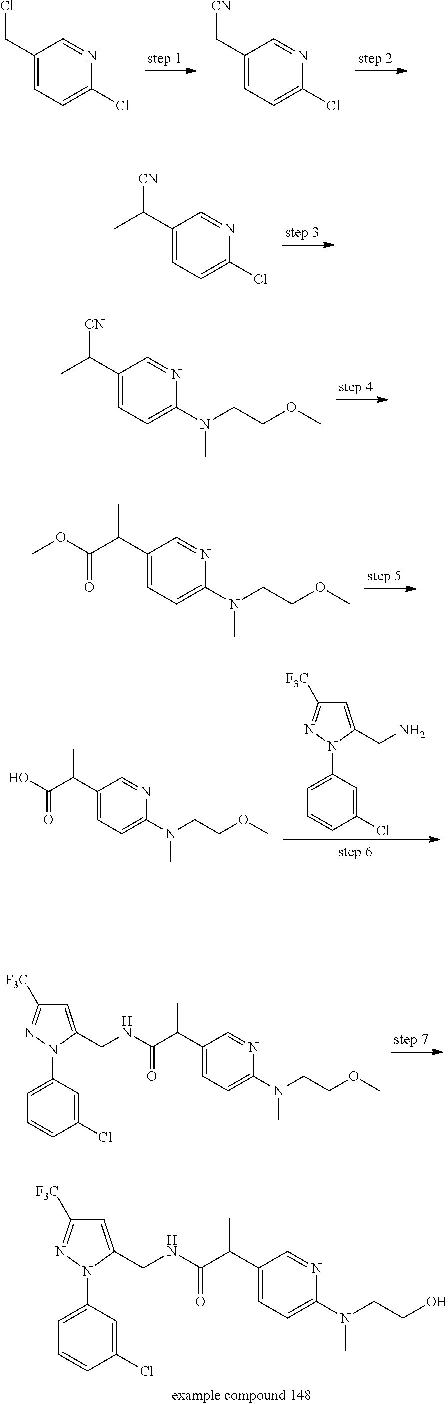

### Step 1:

To a stirred solution of 2-chloro-5-(chloromethyl)pyridine (1 g, 6.17 mmol, 1.0 eq) in ethanol (10 mL) was added the solution of NaCN (325 mg, 6.79 mmol, 1.1 eq) in water (10 mL) dropwise at 0° C. and then stirred for 3 h at 100° C. The reaction mixture was diluted with water (50 mL), extracted with ethyl acetate (2×70 mL). The organic layer was dried over sodium sulfate and evaporated under vacuum. The crude was purified by using silica gel chromatography (100-200 mesh) using ethyl acetate/petrol ether (3:7) to 2-(6-chloropyridin-3-yl)acetonitrile (400 mg, 63%) as a yellow solid.

### Step 2:

To a stirred solution of 2-(6-chloropyridin-3-yl)acetonitrile (10 g, 65.7 mmol, 1.0 eq) in tetrahydrofuran (100 mL) cooled to 0° C. was added sodium hydride (1.578 g, 65.7 mmol, 1.0 eq) in portions and stirred for 10 min. CH₃I (4.02 mL, 65.7 mmol, 1.0 eq) was added at 0° C. The reaction mixture was diluted with water (150 mL), extracted with ethyl acetate (100 mL×2) and brine (100 mL) and dried over sodium sulfate and evaporated under vacuum. The crude was purified by silica gel chromatography (100-200 mesh) using ethyl acetate/petrol ether (1:4) to get 2-(6-chloropyridin-3-yl)propanenitrile (5 g, 46%) as solid.

### Step 3:

To a stirred solution of 2-(6-chloropyridin-3-yl)propanenitrile (1 g, 6.02 mmol, 1.0 eq) in DMSO (7 mL) was added triethylamine (1.67 mL, 12.04 mmol, 2.0 eq) and N-(2-methoxy ethyl)methyl amine (1.07 g, 12.04 mmol, 2.0 eq). The reaction mixture was heated to 100° C. for 16 h. The reaction mixture was diluted with water (50 mL), extracted with ethyl acetate (2×60 mL). The organic layer was washed with brine (50 mL), dried over sodium sulfate and evaporated under vacuum. The residue obtained was purified by neutral alumina using ethyl acetate/petrol ether (1:4) as eluent to get 2-(6-((2-methoxyethyl)(methyl)amino)pyridin-3-yl)propanenitrile (600 mg, 45%) as white solid.

### Step 4:

To a stirred solution of TMSCl (3.0 mL, 13.69 mmol, 3.0 eq) and methanol (0.73 mL, 22.8 mmol, 5.0 eq) was added 2-(6-((2-methoxyethyl)(methyl)amino)pyridin-3-yl)propanenitrile (1 g, 22.8 mmol, 5.0 eq) and heated to 60° C. for 5 h. The Reaction mixture was diluted with water (50 mL) and pH≈9 adjusted with NaHCO₃ (10 mL) extracted with ethyl acetate (2×60 mL). The organic layer was separated and washed with brine (50 mL), dried over sodium sulfate and evaporated under vacuum. The residue was purified by silica gel column (100-200 mesh) using ethyl acetate/petrol ether (2:3) as eluent to get methyl 2-(6-((2-methoxyethyl)(methyl)amino)pyridin-3-yl)propanoate (700 mg, 61%) as a pale yellow oil.

### Step 5:

To a stirred solution of methyl 2-(6-((2-methoxyethyl)(methyl)amino)pyridin-3-yl)propanoate (200 mg, 0.793 mmol, 1.0 eq) in tetrahydrofuran: water (5 mL+5 mL) was added LiOH.water (99 mg, 2.380 mmol, 3.0 eq) at 60° C. and stirred for 2 h. The reaction mixture was diluted with water (5 mL), acidified with 1N HCl, and then extracted with ethyl acetate (2×25 mL). The organic layer was washed with water (20 mL), brine (20 mL), dried over anhydrous sodium sulfate and evaporated under vacuum to get 2-(6-((2-methoxyethyl)(methyl)amino)pyridin-3-yl)propanoic acid (150 mg; 79%). Crude was directly used for next step without further purification.

### Step 6:

To a stirred solution of 2-(6-((2-methoxyethyl)(methyl)amino)pyridin-3-yl)propanoic acid (150 mg, 0.630 mmol, 1.0 eq) in dichloromethane (10 mL) was added EDC.HCl (180 mg, 0.945 mmol, 1.5 eq) followed by HOBt (106 mg, 0.693 mmol, 1.1 eq), DIPEA (0.3 mL, 1.89 mmol, 3 eq) and then (1-(3-chlorophenyl)-3-(trifluoromethyl)-1H-pyrazol-5-yl)methanamine (173 mg, 0.630 mmol, 1.0 eq) at room temperature and stirred for 3 h. dichloromethane was evaporated and residue diluted with water (10 mL), extracted with ethyl acetate (2×25 mL). The organic layer was washed with brine (20 mL), dried over anhydrous sodium sulfate and evaporated under vacuum. Crude was purified by silica gel (100-200 mesh) column chromatography by using methanol/trichloromethane (1:19) as eluent to get N-((1-(3-chlorophenyl)-3-(trifluoromethyl)-1H-pyrazol-5-yl)methyl)-2-(6-((2-methoxyethyl)(methyl)amino)pyridin-3-yl)propanamide (140 mg; 45%, pale yellow viscous liquid).

### Step 7:

To a stirred solution of N-((1-(3-chlorophenyl)-3-(trifluoromethyl)-1H-pyrazol-5-yl)methyl)-2-(6-((2-methoxyethyl)(methyl)amino)pyridin-3-yl)propanamide (300 mg, 0.606 mmol, 1.0 eq) in dichloromethane (20 mL) was added 1M boron tribromide in dichloromethane (0.9 mL, 0.909 mmol, 1.5 eq) at −78° C. and stirred at room temperature for 3 h. The pH of the reaction was adjusted to ˜8 NaHCO₃ and diluted with water (20 mL). The aqueous layer was extracted with ethyl acetate (2×50 mL) and the combined organic layer was separated and washed with brine (50 mL), dried over sodium sulfate and evaporated under vacuum. The residue was purified by silica gel column (100-200 mesh) using methanol/trichloromethane (1:9) as eluent to get N-((1-(3-chlorophenyl)-3-(trifluoromethyl)-1H-pyrazol-5-yl)methyl)-2-(6-((2-hydroxyethyl)(methyl)amino)pyridin-3-yl)propanamide (example compound 148) (200 mg, 68%) as yellow solid.

### Synthesis of Example 152

### 1-((3-tert-butyl-1-(3-fluorophenyl)-1H-pyrazol-5-yl)methyl)-3-(6-(2-hydroxyethyl)pyridin-3-yl)urea

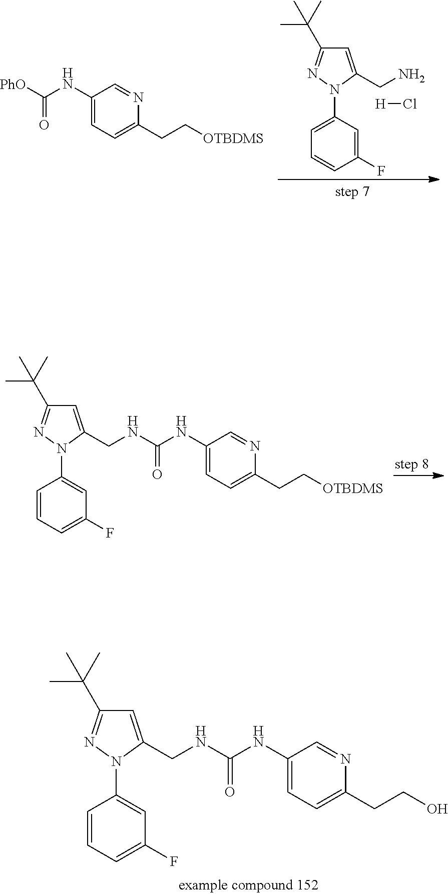

### Step 1-6:

see example compound 52.

### Step 7:

To a stirred solution of (3-tert-butyl-1-(3-fluorophenyl)-1H-pyrazol-5-yl)methanamine hydrochloride (150 mg, 0.606 mmol, 1.0 eq) in dichloromethane (10 mL) was added triethylamine (184 mg, 2.326 mmol, 3.0 eq) and stirred at room temperature for 10 min and phenyl 6-(2-(tert-butyldimethylsilyloxy)ethyl)pyridin-3-ylcarbamate (226 mg, 0.605 mmol, 1.0 eq) added and stirred at room temperature for 16 h. The reaction mixture was evaporated and the resulting crude was purified by silica gel column chromatography (60-120 mesh) to get 1-((3-tert-butyl-1-(3-fluorophenyl)-1H-pyrazol-5-yl)methyl)-3-(6-(2-(tert-butyldimethylsilyloxy)ethyl)pyridin-3-yl)urea (280 mg, 88%) as a solid.

### Step 8:

To a stirred solution of 1-((3-tert-butyl-1-(3-fluorophenyl)-1H-pyrazol-5-yl)methyl)-3-(6-(2-(tert-butyldimethylsilyloxy)ethyl)pyridin-3-yl)urea (280 mg, 0.5706 mmol, 1.0 eq) in tetrahydrofuran (3 mL) was added 2N HCl (1.5 mL) and stirred at room temperature for 2 h. The reaction mixture was neutralized with aq NaHCO₃ solution and extracted with ethyl acetate, dried over sodium sulfate and evaporated to get 1-((3-tert-butyl-1-(3-fluorophenyl)-1H-pyrazol-5-yl)methyl)-3-(6-(2-hydroxyethyl)pyridin-3-yl)urea (example compound 152) (84 mg, 35%) as a solid.

### Synthesis of Example 153

### 1-((3-tert-butyl-1-(3-methoxyphenyl)-1H-pyrazol-5-yl)methyl)-3-(6-(2-hydroxyethyl)pyridin-3-yl)urea

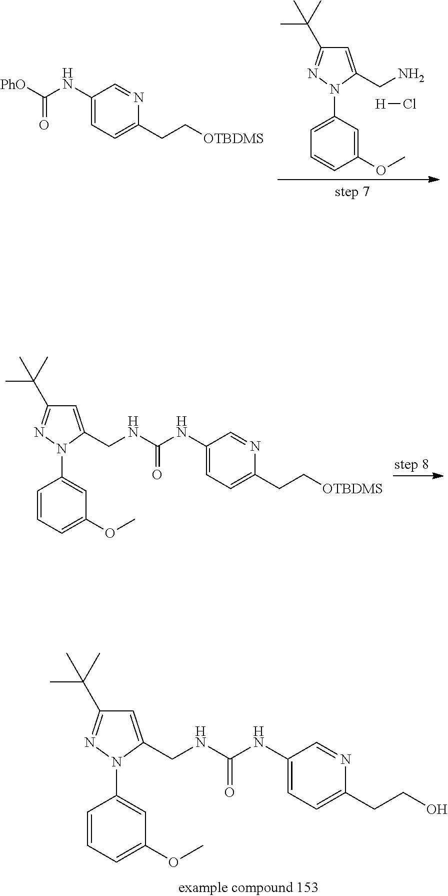

### Step 1-6:

see example compound 152.

### Step 7:

To a stirred solution of (3-tert-butyl-1-(3-methoxyphenyl)-1H-pyrazol-5-yl)methanamine hydrochloride (100 mg, 0.3855 mmol, 1.0 eq) in dichloromethane (10 mL) was added triethylamine (116 mg, 1.1485 mmol, 3.0 eq) and stirred at room temperature for 10 min and phenyl 6-(2-(tert-butyldimethylsilyloxy)ethyl)pyridin-3-ylcarbamate (144 mg, 0.386 mmol, 1.0 eq) added and stirred at room temperature for 16 h. The reaction mixture was evaporated and the resulting crude was purified by silica gel column chromatography (60-120 mesh) to get 1-((3-tert-butyl-1-(3-methoxyphenyl)-1H-pyrazol-5-yl)methyl)-3-(6-(2-(tert-butyldimethylsilyloxy)ethyl)pyridin-3-yl)urea (180 mg, 86%) as a solid.

### Step 8:

To a stirred solution of 1-((3-tert-butyl-1-(3-methoxyphenyl)-1H-pyrazol-5-yl)methyl)-3-(6-(2-(tert-butyldimethylsilyloxy)ethyl)pyridin-3-yl)urea (180 mg, 0.3347 mmol, 1.0 eq) in tetrahydrofuran (3 mL) was added 2N HCl (0.9 mL) and stirred at room temperature for 2 h. The reaction mixture was neutralized with aq NaHCO₃ solution and extracted with ethyl acetate, dried over sodium sulfate and evaporated to get 1-((3-tert-butyl-1-(3-methoxyphenyl)-1H-pyrazol-5-yl)methyl)-3-(6-(2-hydroxyethyl)pyridin-3-yl)urea (example compound 153) (64 mg, 45%) as a solid.

### Synthesis of Example 159

### 1-((3-tert-butyl-1-(3-chlorophenyl)-1H-pyrazol-5-yl)methyl)-3-(6-(methylsulfonylmethyl)pyridin-3-yl)urea

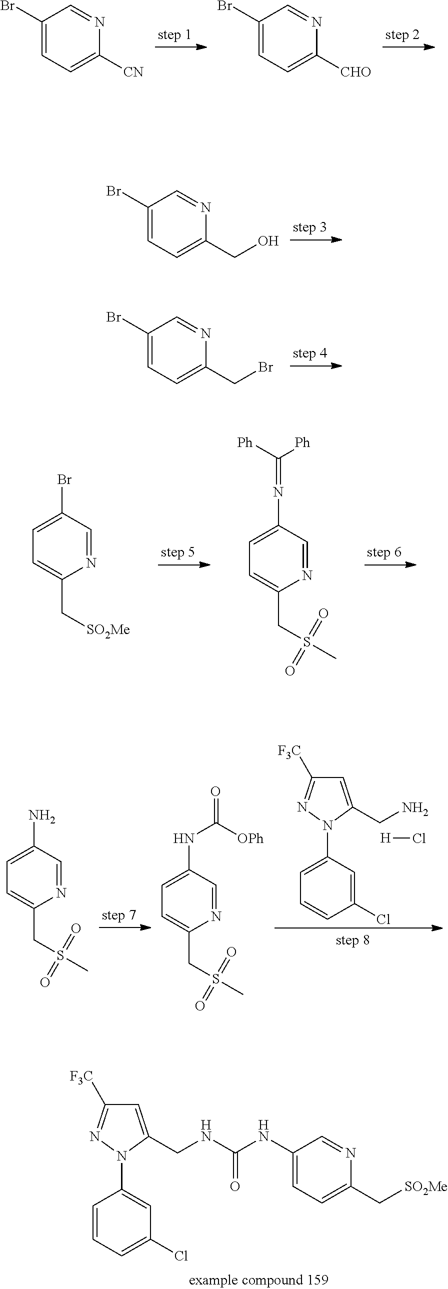

### Step 1:

To a stirred solution of 5-bromopicolinonitrile (0.5 g, 2.732 mmol, 1.0 eq) in tetrahydrofuran (10 mL) at −78° C. DIBAL (4 mL, 4.98 mmol, 1.5 eq) was added and reaction mixture was stirred for 4 h at −78° C. The reaction mixture was monitored by TLC, and quenched with 2N HCl (2 mL) and extracted with dichloromethane (10 mL), dried over sodium sulfate and evaporated to provide fairly pure 5-bromopicolinaldehyde (0.3 g, 60%) which was used to the next stage without further purification.

### Step 2:

To a stirred solution of 5-bromopicolinaldehyde (0.5 g, 2.68 mmol, 1.0 eq) in methanol (10 mL) was added NaBH₄ (0.18 g, 5.37 mmol, 2 eq) and stirred at room temperature for 4 h. methanol was evaporated and diluted with ethyl acetate (10 mL), washed with water (15 mL), dried over sodium sulfate and evaporated under reduced pressure to get crude compound. This crude was purified by column chromatography using 100-200 silica gel 20% ethyl acetate-petrol ether as eluent system to get (5-bromopyridin-2-yl)methanol (0.2 g, 50%).

### Step 3:

To a stirred solution of (5-bromopyridin-2-yl)methanol (0.2 g, 1.06 mmol, 1.0 eq) in dichloromethane (5 mL) was added triphenyl phosphine (0.4 g, 1.59 mmol, 1.5 eq), and N-bromosuccinimide (0.3 g, 1.59 mmol, 1.5 eq) at 0° C. and allowed to stir at room temperature for 1 h. The reaction mixture was quenched with water, and extracted with dichloromethane (2×10 mL). The organic layer was washed with brine (30 mL), dried over sodium sulfate, concentrated and crude was purified by silica gel (100-200 mesh) column chromatography using ethyl acetate/petrol ether (1:9) as eluent to get 5-bromo-2-(bromomethyl)pyridine (0.2 g, ˜77%).

### Step 4:

To a stirred solution of 5-bromo-2-(bromomethyl)pyridine (1.0 g, 4.29 mmol, 1.0 eq) in isopropyl alcohol (15 mL), was added sodium methanesulfinate (2.1 g, 21.45 mmol, 5.0 eq) and stirred at 70° C. for 4 h. The reaction mixture was concentrated and diluted with ethyl acetate (30 mL) and washed with water (20 mL), dried over sodium sulfate and evaporated under reduced pressure. The crude obtained was washed with diethyl ether (30 mL) to get 5-bromo-2-(methylsulfonylmethyl)pyridine (0.8 g, 80%).

### Step 5:

To a stirred solution of 5-bromo-2-(methylsulfonylmethyl)pyridine (0.1 g, 0.4 mmol, 1.0 eq) in toluene (10 mL) were added benzophenoneimine (0.086 mL, 0.48 mmol, 1.2 eq) under nitrogen atmosphere, Pd₂ dba₃ (36 mg, 0.4 mmol, 0.1 eq) Caesium carbonate (0.2 g, 0.6 mmol, 1.5 eq). The reaction mixture was refluxed for 5 h and diluted with water (5 mL) and compound, extracted with ethyl acetate (10 mL), dried over sodium sulfate to get N-(diphenylmethylene)-6-(methylsulfonylmethyl)pyridin-3-amine (90 mg, crude).

### Step 6:

To a solution of N-(diphenylmethylene)-6-(methylsulfonylmethyl)pyridin-3-amine (90 mg) in methanol was added conc HCl (2 mL) and stirred at room temperature for 30 min. The reaction mixture was diluted with water (5 mL), extracted with ethyl acetate (10 mL), evaporated under reduced pressure. The crude obtained was washed with diethyl ether (10 mL) to get 6-(methylsulfonylmethyl)pyridin-3-amine (30 mg, −52%).

### Step 7:

To a stirred solution of 6-(methylsulfonylmethyl)pyridin-3-amine (0.8 g, 4.301 mmol, 1.0 eq) in acetone (10 mL) were added phenyl carbonochloridate (0.5 mL, 4.731 mmol, 1.1 eq), and pyridine (0.96 mL, 12.90 mmol, 3 eq) at 0° C. The reaction mixture was stirred at room temperature for 1 h. Acetone was evaporated and residue, diluted with dichloromethane (15 mL), washed water (10 mL), dried over sodium sulfate and concentrated under reduced pressure. The crude was washed with diethyl ether (10 mL) to get phenyl 6-(methylsulfonylmethyl)pyridin-3-ylcarbamate (0.8 g, 50%) as off white solid.

### Step 8:

To a stirred solution of phenyl 6-(methylsulfonylmethyl)pyridin-3-ylcarbamate (100 mg, 0.31 mmol, 1.0 eq) in dichloromethane (10 mL) was added triethylamine (0.2 mL, 1.2 mmol, 3.0 eq) and stirred at room temperature for 10 min. (1-(3-chlorophenyl)-3-(trifluoromethyl)-1H-pyrazol-5-yl)methanamine hydrochloride (100 mg, 0.3 mmol) was added and stirred at room temperature for 16 h. The reaction mixture was concentrated and the resulting crude was purified by silica gel column chromatography (100-200 mesh) followed by preparative TLC to get 1-((1-(3-chlorophenyl)-3-(trifluoromethyl)-1H-pyrazol-5-yl)methyl)-3-(6-(methylsulfonylmethyl)pyridin-3-yl)urea (example compound 159) (110 mg, 40%) as off-white solid.

### Synthesis of Example 160

### 1-((3-tert-butyl-1-(3-fluorophenyl)-1H-pyrazol-5-yl)methyl)-3-(6-(hydroxymethyl)pyridin-3-yl)urea

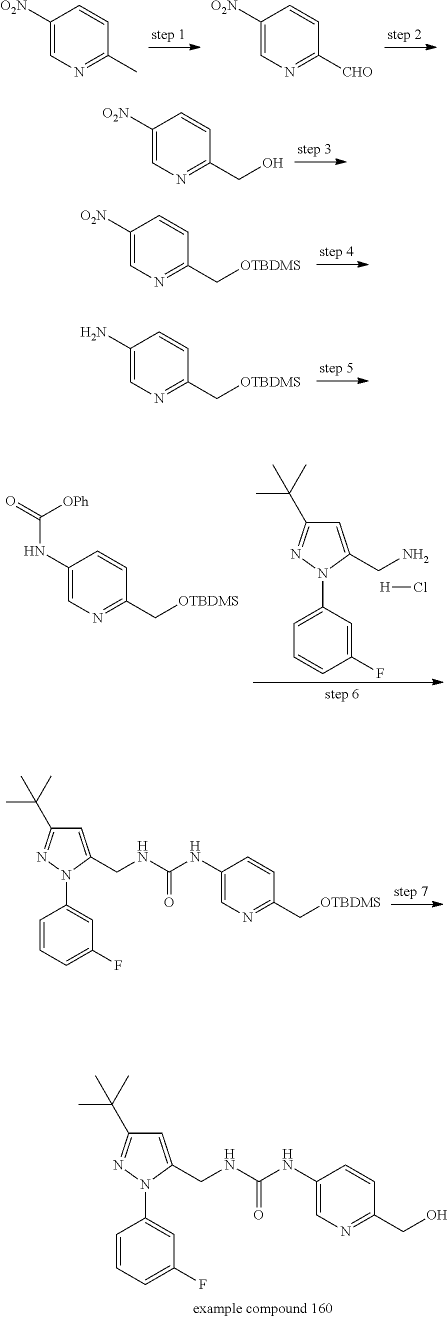

### Step 1:

To a stirred solution of 2-methyl-5-nitropyridine (3.0 g, 0.021 mol, 1 eq) in 1,4-dioxane (30 mL) at room temperature was added selenium dioxide (2.9 g, 0.026 mol, 1.2 eq) and stirred at reflux for 16 h. The reaction mixture was filtered, evaporated, diluted with ethyl acetate (50 mL) and washed with water (50 mL), dried over sodium sulfate and evaporated to get 5-nitropicolinaldehyde (3.12 g, 94%).

### Step 2:

To a stirred solution of 5-nitropicolinaldehyde (350 g, 2.3 mmol, 1.0 eq) in methanol (10 mL) was added NaBH4 (82 mg, 2.3 mmol, 1.0 eq) at 0° C. and resulting reaction mixture was stirred for 2 h. The reaction mixture was evaporated and residue dissolved in ethyl acetate (20 mL), washed with brine (30 mL), dried over sodium sulfate, evaporated to get (5-nitropyridin-2-yl)methanol (0.210 g, 60%).

### Step 3:

To a stirred solution of (5-nitropyridin-2-yl)methanol (570 mg, 3.7 mmol, 1.0 eq) in dichloromethane (10 mL) was added imidazole (377 mg, 5.5 mmol, 1.5 eq), and TBDMSCl (832 mg, 5.5 mmol, 1.5 eq) at 0° C. and allowed to stir at room temperature for 2 h. The reaction mixture was washed with water (20 mL), dried over sodium sulfate, evaporated and purified by silica gel (100-200 mesh) column chromatography using ethyl acetate/petrol ether (1:9) as eluent to get 2-((tert-butyldimethylsilyloxy)methyl)-5-nitropyridine (753 mg, 76%).

### Step 4:

To a stirred solution of 2-((tert-butyldimethylsilyloxy)methyl)-5-nitropyridine (400 mg, 1.492 mmol, 1.0 eq) in methanol (10 mL) was added 10% Pd/C (100 mg) and stirred under hydrogen atmosphere at room temperature for 1 h. The reaction mixture was filtered through celite pad and filtrate was concentrated under reduced pressure. This crude was washed with diethyl ether (20 mL) to get 6-((tert-butyldimethylsilyloxy)methyl)pyridin-3-amine (269 mg, 76%) as off-white solid.

### Step 5:

To a stirred solution of 6-((tert-butyldimethylsilyloxy)methyl)pyridin-3-amine (400 mg, 1.680 mmol, 1.0 eq) in acetone (10 mL) were added pyridine (0.27 mL, 3.20 mmol, 2 eq), phenyl carbonochloridate (0.2 mL, 1.8 mmol, 1.1 eq) at 0° C. and stirred at room temperature for 2 h. The reaction mixture was concentrated and diluted with dichloromethane (20 mL) and washed water (30 mL), dried over sodium sulfate and evaporated under reduced pressure. The crude obtained was washed twice with diethyl ether (5 mL) to get phenyl 6-((tert-butyldimethylsilyloxy)methyl)pyridin-3-ylcarbamate (517 mg, 86%) as off white solid.

### Step 6:

To a stirred solution of (3-tert-butyl-1-(3-fluorophenyl)-1H-pyrazol-5-yl)methanamine hydrochloride (82 mg, 0.331 mmol, 1.0 eq) in dichloromethane (10 mL) was added triethylamine (1.7 mL, 1.33 mmol, 4.0 eq) and stirred at room temperature for 10 min and phenyl 6-((tert-butyldimethylsilyloxy)methyl)pyridin-3-ylcarbamate (120 mg, 0.335 mmol, 1.0 eq) added and stirred at room temperature for 16 h. The reaction mixture was concentrated and the resulting crude was purified by silica gel column chromatography (100-200 mesh) and again by preparative TLC to get 1-((3-tert-butyl-1-(3-fluorophenyl)-1H-pyrazol-5-yl)methyl)-3-(6-((tert-butyldimethylsilyloxy)methyl)pyridin-3-yl)urea (119 mg, 70%) as a white solid.

### Step 7:

To a stirred solution of 1-((3-tert-butyl-1-(3-fluorophenyl)-1H-pyrazol-5-yl)methyl)-3-(6-((tert-butyldimethylsilyloxy)methyl)pyridin-3-yl)urea (119 mg, 0.0.232 mmol, 1.0 eq) in tetrahydrofuran (10 mL) was added 2N HCl (1.5 mL) and stirred room temperature for 2 h. The reaction mixture basify with aq NaHCO₃ solution and extracted with ethyl acetate (20 mL), dried over sodium sulfate and evaporated to get compound 1-((3-tert-butyl-1-(3-fluorophenyl)-1H-pyrazol-5-yl)methyl)-3-(6-(hydroxymethyl)pyridin-3-yl)urea (example compound 160) (43 mg, 47%) as a solid.

Examples 32, 33, and 35-37 were prepared in a similar manner. Examples 31, 34, 36 and 38-49 can be prepared in a similar manner.

Mass spectrometric data are cited hereinafter by way of example for the following example compounds (Tables 1a and 1b):

TABLE 1a

| Example | |
|---|---|
| compound | [M + H] |
| 1 | 395.0 |
| 2 | 397.2 |
| 3 | 409.0 |
| 4 | 383.9 |
| 5 | 395.8 |
| 6 | 383.3 |
| 7 | 395.2 |
| 8 | 409.1 |
| 9 | 396.1 |
| 10 | 383.2 |
| 11 | 395.1 |
| 12 | 396.1 |
| 13 | 396.1 |
| 14 | 384.3 |
| 15 | 397.1 |
| 16 | 396.1 |
| 17 | 428.9 |
| 18 | 414.1 |
| 19 | 411.0 |
| 21 | 501.9 |
| 22 | 508.0 |
| 23 | 520.1 |
| 24 | 492.0 |
| 25 | 427.5 |
| 26 | 439.1 |
| 27 | 516.4 |
| 30 | 439.0 |
| 32 | 426.1 |
| 50 | 443.9 |
| 51 | 452.9 |
| 53 | 483.0 |
| 54 | 464.1 |
| 55 | 452.0 |
| 56 | 440.0 |
| 57 | 528.0 |
| 58 | 546.0 |
| 59 | 595.8 |
| 60 | 534.0 |
| 61 | 583.9 |
| 62 | 529.3 |
| 63 | 547.3 |
| 64 | 597.3 |
| 65 | 517.4 |
| 66 | 535.4 |
| 67 | 585.4 |
| 68 | 457.1 |
| 69 | 469.1 |
| 70 | 443.1 |
| 71 | 455.1 |
| 79 | 457.1 |
| 87 | 457.1 |
| 88 | 496.2 |
| 89 | 483.1 |
| 90 | 469.1 |
| 91 | 516.0 |
| 92 | 528.0 |
| 93 | 534.0 |
| 94 | 546.0 |
| 95 | 550.0 |
| 96 | 562.0 |
| 98 | 490.5 |
| 99 | 502.1 |
| 100 | 486.0 |
| 101 | 489.1 |
| 102 | 508.5 |
| 103 | 520.1 |
| 104 | 531.9 |
| 105 | 520.0 |
| 106 | 507.0 |
| 107 | 495.0 |
| 108 | 439.0 |
| 109 | 451.0 |
| 110 | 439.1 |
| 111 | 466.9 |
| 112 | 451.1 |
| 113 | 484.9 |
| 114 | 447.1 |
| 115 | 475.0 |
| 117 | 455.0 |
| 120 | 453.0 |
| 121 | 465.0 |
| 123 | 495.1 |
| 124 | 481.1 |
| 125 | 481.1 |
| 126 | 469.1 |
| 127 | 469.1 |
| 129 | 439.0 |
| 130 | 427.0 |
| 131 | 458.0 |
| 132 | 470.0 |
| 133 | 456.0 |

TABLE 1b

| Example | |
|---|---|
| compound | [M + H] |
| 134 | 469.1 |
| 135 | 483.2 |
| 139 | 414.6 |
| 140 | 414.6 |
| 141 | 439.8 |
| 142 | 457.3 |
| 143 | 454.3 |
| 144 | 443.9 |
| 145 | 455.8 |
| 146 | 468.1 |
| 147 | 482.2 |
| 148 | 482.2 |
| 149 | 471.1 |
| 150 | 425.6 |
| 151 | 433.1 |
| 152 | 412.1 |
| 153 | 424.2 |
| 154 | 426.9 |
| 155 | 457.1 |
| 156 | 469.4 |
| 157 | 465.2 |
| 158 | 453.4 |
| 159 | 476.6 |
| 160 | 398.1 |

### Pharmacological Methods

### I. Functional Testing Carried Out on the Vanilloid Receptor 1 (VR1/TRPV1 Receptor)

The agonistic or antagonistic effect of the substances to be tested on the rat-species vanilloid receptor 1 (VR1/TRPV1) can be determined using the following assay. In this assay, the influx of Ca²⁺ through the receptor channel is quantified with the aid of a Ca²⁺-sensitive dye (type Fluo-4, Molecular Probes Europe BV, Leiden, the Netherlands) in a fluorescent imaging plate reader (FLIPR, Molecular Devices, Sunnyvale, USA).

### Method:

Complete medium: 50 mL HAMS F12 nutrient mixture (Gibco Invitrogen GmbH, Karlsruhe, Germany) with 10% by volume of FCS (foetal calf serum, Gibco Invitrogen GmbH, Karlsruhe, Germany, heat-inactivated); 2 mM L-glutamine (Sigma, Munich, Germany); 1% by weight of AA solution (antibiotic/antimyotic solution, PAA, Pasching, Austria) and 25 ng/mL NGF medium (2.5 S, Gibco Invitrogen GmbH, Karlsruhe, Germany)

Cell culture plate: Poly-D-lysine-coated, black 96-well plates having a clear base (96-well black/clear plate, BD Biosciences, Heidelberg, Germany) are additionally coated with laminin (Gibco Invitrogen GmbH, Karlsruhe, Germany), the laminin being diluted with PBS (Ca—Mg-free PBS, Gibco Invitrogen GmbH, Karlsruhe, Germany) to a concentration of 100 μg/mL. Aliquots having a laminin concentration of 100 μg/mL are removed and stored at −20° C. The aliquots are diluted with PBS in a ratio of 1:10 to 10 μg/mL of laminin and respectively 50 μL of the solution are pipetted into a recess in the cell culture plate. The cell culture plates are incubated for at least two hours at 37° C., the excess solution is removed by suction and the recesses are each washed twice with PBS. The coated cell culture plates are stored with excess PBS which is not removed until just before the feeding of the cells.

### Preparation of the Cells:

The vertebral column is removed from decapitated rats and placed immediately into cold HBSS buffer (Hank's buffered saline solution, Gibco Invitrogen GmbH, Karlsruhe, Germany), i.e. buffer located in an ice bath, mixed with 1% by volume (percent by volume) of an AA solution (antibiotic/antimyotic solution, PAA, Pasching, Austria). The vertebral column is cut longitudinally and removed together with fasciae from the vertebral canal. Subsequently, the dorsal root ganglia (DRG) are removed and again stored in cold HBSS buffer mixed with 1% by volume of an AA solution. The DRG, from which all blood remnants and spinal nerves have been removed, are transferred in each case to 500 μL of cold type 2 collagenase (PAA, Pasching, Austria) and incubated for 35 minutes at 37° C. After the addition of 2.5% by volume of trypsin (PAA, Pasching, Austria), incubation is continued for 10 minutes at 37° C.

After complete incubation, the enzyme solution is carefully pipetted off and 500 μL of complete medium are added to each of the remaining DRG. The DRG are respectively suspended several times, drawn through cannulae No. 1, No. 12 and No. 16 using a syringe and transferred to a 50 mL Falcon tube which is filled up to 15 mL with complete medium. The contents of each Falcon tube are respectively filtered through a 70 μm Falcon filter element and centrifuged for 10 minutes at 1,200 rpm and room temperature. The resulting pellet is respectively taken up in 250 μL of complete medium and the cell count is determined.

The number of cells in the suspension is set to 3×10⁵ per mL and 150 μL of this suspension are in each case introduced into a recess in the cell culture plates coated as described hereinbefore. In the incubator the plates are left for two to three days at 37° C., 5% by volume of CO₂ and 95% relative humidity. Subsequently, the cells are loaded with 2 μM of Fluo-4 and 0.01% by volume of Pluronic F127 (Molecular Probes Europe BV, Leiden, the Netherlands) in HBSS buffer (Hank's buffered saline solution, Gibco Invitrogen GmbH, Karlsruhe, Germany) for 30 min at 37° C., washed 3 times with HBSS buffer and after further incubation for 15 minutes at room temperature used for Ca²⁺ measurement in a FLIPR assay. The Ca²⁺-dependent fluorescence is in this case measured before and after the addition of substances (λex=488 nm, λem=540 nm). Quantification is carried out by measuring the highest fluorescence intensity (FC, fluorescence counts) over time.

### FLIPR Assay:

The FLIPR protocol consists of 2 substance additions. First the compounds to be tested (10 μM) are pipetted onto the cells and the Ca²⁺ influx is compared with the control (capsaicin 10 μM). This provides the result in % activation based on the Ca²⁺ signal after the addition of 10 μM of capsaicin (CP). After 5 minutes' incubation, 100 nM of capsaicin are applied and the Ca²⁺ influx is also determined. Desensitising agonists and antagonists lead to suppression of the Ca²⁺ influx. The % inhibition is calculated compared to the maximum achievable inhibition with 10 μM of capsazepine. Triple analyses (n=3) are carried out and repeated in at least 3 independent experiments (N=4).

Starting from the percentage displacement caused by different concentrations of the compounds to be tested of general formula I, IC₅₀ inhibitory concentrations which cause a 50-percent displacement of capsaicin were calculated. Kᵢ values for the test substances were obtained by conversion by means of the Cheng-Prusoff equation (Cheng, Prusoff; Biochem. Pharmacol. 22, 3099-3108, 1973).

### Pharmacological Data

The affinity of the compounds according to the invention for the vanilloid receptor 1 (VR1/TRPV1 receptor) was determined as described above (pharmacological method I).

The compounds according to the invention display outstanding affinity to the VR1/TRPV1 receptor (Tables 2a and 2b).

In Tables 2a and 2b the abbreviations below have the following meanings:

Cap=capsaicin
AG=agonist
NE=no effect
pAG=partial agonist

The value after the “©” symbol indicates the concentration at which the inhibition (as a percentage) was respectively determined.

TABLE 2a

| Compound according | (f) Ki (human being) |
|---|---|
| to Example | [nM] Cap |
| 1 | 31%@5 μM |
| 2 | 89%@5 μM, AG |
| 3 | 35%@5 μM |
| 4 | 47 |
| 5 | 29%@5 μM |
| 6 | 89%@5 μM, AG |
| 7 | 51%@5 μM |
| 8 | 13 |
| 9 | 72%@5 μM, AG |
| 10 | 90%@5 μM, AG |
| 11 | 79%@5 μM, AG |
| 12 | 85%@5 μM, AG |
| 13 | 30%@5 μM |
| 14 | pAG |
| 15 | AG |
| 16 | 77 |
| 17 | 29 |
| 18 | 39 |
| 19 | 45%@5 μM |
| 21 | 43 |
| 22 | 28 |
| 23 | 29 |
| 24 | 46 |
| 25 | 31 |
| 26 | 44%@5 μM |
| 27 | 61 |
| 30 | 1 |
| 32 | 32 |
| 50 | 24 |
| 51 | 8 |
| 53 | 44 |
| 54 | NE |
| 55 | 23 |
| 56 | 77%@5 μM, AG |
| 57 | 60%@5 μM |
| 58 | 5 |
| 59 | 24%@5 μM |
| 60 | 4 |
| 61 | 45 |
| 62 | 53%@5 μM |
| 63 | 6 |
| 64 | 61 |
| 65 | 70 |
| 66 | 3 |
| 67 | 13 |
| 68 | 73%@5 μM, AG |
| 69 | 51 |
| 70 | 3 |
| 71 | 8 |
| 79 | 26 |
| 87 | 17 |
| 89 | 31%@5 μM |
| 90 | 44 |
| 91 | 98%@5 μM, AG |
| 92 | 26 |
| 93 | 2 |
| 94 | 4 |
| 95 | 2 |
| 96 | 3 |
| 98 | 8 |
| 99 | 11 |
| 100 | 37 |
| 101 | 30%@5 μM |
| 102 | 8 |
| 103 | 12 |
| 104 | 3 |
| 105 | 2 |
| 106 | 25 |
| 107 | 5 |
| 108 | 43 |
| 109 | 46 |
| 110 | 37 |
| 111 | 38 |
| 112 | 48 |
| 113 | 80 |
| 114 | 35 |
| 115 | 2 |
| 117 | 18 |
| 120 | 23 |
| 121 | 47 |
| 123 | 83 |
| 124 | 64 |
| 125 | 64 |
| 126 | 61 |
| 127 | 61 |
| 129 | 13 |
| 130 | 47%@5 μM |
| 131 | 83%@5 μM, AG |
| 132 | 45%@5 μM |
| 133 | 88 |
| 123 | 83 |
| 124 | 64 |
| 125 | 64 |
| 126 | 61 |
| 127 | 61 |

TABLE 2b

| Compound according | (f) Ki (human being) |
|---|---|
| to Example | [nM] Cap |
| 33 | 41%@5 μM |
| 35 | NE |
| 36 | 101 |
| 37 | 41%@5 μM |
| 52 | 45%@5 μM |
| 72 | 17 |
| 81 | 12 |
| 88 | 55%@5 μM |
| 134 | NE |
| 135 | 22%@5 μM |
| 136 | 47 |
| 137 | 55 |
| 138 | 90 |
| 139 | 109 |
| 140 | 50 |
| 141 | 51%@5 μM |
| 142 | 1.5 |
| 143 | 84 |
| 144 | 24 |
| 145 | 121 |
| 146 | 0.6 |
| 147 | 18 |
| 148 | 15 |
| 149 | 28 |
| 150 | 59 |
| 151 | 49 |
| 152 | 85 |
| 153 | 50 |
| 154 | 38 |
| 155 | 35 |
| 156 | 29 |
| 157 | 112 |
| 158 | 77 |
| 159 | 29@1 μM |
| 160 | 75 |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A compound of formula (I): 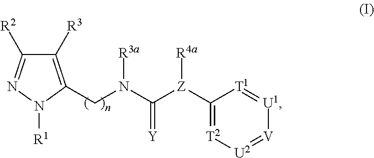
wherein
R⁰ represents a C₁₋₁₀ aliphatic residue, unsubstituted or mono- or polysubstituted; a C₃₋₁₀ cycloaliphatic residue or a 3 to 10 membered heterocycloaliphatic residue, in each case unsubstituted or mono- or polysubstituted and in each case optionally bridged via a C₁₋₈ aliphatic group, which in turn may be unsubstituted or mono- or polysubstituted; aryl or heteroaryl, in each case unsubstituted or mono- or polysubstituted and in each case optionally bridged via a C₁₋₈ aliphatic group, which in turn may be unsubstituted or mono- or polysubstituted;
R¹ represents H; R⁰; C(═O)—R⁰; C(═O)—OH; C(═O)—OR⁰; C(═O)—NHR⁰; C(═O)—N(R⁰)₂; OH; O—R⁰; SH; S—R⁰; S(═O)₂—R⁰; S(═O)₂—OR⁰; S(═O)₂—NHR⁰; S(═O)₂—N(R⁰)₂; NH₂; NHR⁰; N(R⁰)₂; NH—S(═O)₂—R⁰; N(R⁰) (S(═O)₂—R⁰; or SCl₃;
R² represents H; R⁰; F; Cl; Br; I; CN; NO₂; OH; SH; CF₃; CF₂H; CFH₂; CF₂Cl; CFCl₂; CH₂CF₃; OCF₃; OCF₂H; OCFH₂; OCF₂Cl; OCFCl₂; SCF₃; SCF₂H; SCFH₂; SCF₂Cl; SCFCl₂; S(═O)₂—CF₃; S(═O)₂—CF₂H; S(═O)₂—CFH₂; or SF₅;
R³ represents H or a C₁₋₁₀ aliphatic residue, unsubstituted or mono- or polysubstituted;
n represents 1, 2, 3 or 4;
R^{3a} represents H or a C₁₋₄ aliphatic residue, unsubstituted or mono- or polysubstituted;
R^{4a} represents H or a C₁₋₄ aliphatic residue, unsubstituted or mono- or polysubstituted, a C₃₋₆ cycloaliphatic residue, unsubstituted or mono- or polysubstituted, or an aryl, unsubstituted or mono- or polysubstituted;
Y represents O, S, or N—CN;
Z represents N or C—R^{4b};
R^{4b} represents H or a C₁₋₄ aliphatic residue, unsubstituted or mono- or polysubstituted; or
R^{4a} and R^{4b} together with the carbon atom connecting them form a C₃₋₆ cycloaliphatic residue, unsubstituted or mono- or polysubstituted;
T¹ represents N or C—R⁵;
U¹ represents N or C—R⁶;
V represents N or C—R⁷;
U² represents N or C—R⁸;
T² represents N or C—R⁹;
with the proviso that 1, 2 or 3 of variables T¹, U¹, V, U² and T² represent(s) a nitrogen atom;
R⁵, R⁶, R⁷, R⁸ and R⁹ each independently represent H; F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁹; O—C(═O)—O—R⁹; O—(C═O)—NH—R⁹; O—C(═O)—N(R⁹)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁹)₂; NH—C(═O)—R⁰; NH—C(═O)—O—R⁹; NH—C(═O)—NH₂; NH—C(═O)—NH—R⁰; NH—C(═O)—N(R⁹)₂; NR⁰—C(═O)—R⁰; NR⁹—C(═O)—O—R⁹; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R⁰)₂;
and wherein:
an “aliphatic group” or “aliphatic residue” can each independently be branched or unbranched, saturated or unsaturated;
a “cycloaliphatic residue” or a “heterocycloaliphatic residue” can each, independently be saturated or unsaturated;
“mono- or polysubstituted” with respect to an “aliphatic group”, an “aliphatic residue”, a “cycloaliphatic residue” or a “heterocycloaliphatic residue” relates in each case independently of one another, with respect to the corresponding residues or groups, to the replacement of one or more hydrogen atoms each independently of one another by at least one substituent selected from the group consisting of F; Cl; Br; I; NO₂; CN; ═O; ═NH; ═N(OH); ═C(NH₂)₂; CF₃; CF₂H; CFH₂; CF₂Cl; CFCl₂; R⁰; C(═O)—H; C(═O)—R⁰; C(═O)—OH; C(═O)—OR⁹; CO—NH₂; C(═O)—NHR⁰; C(═O)—N(R⁹)₂; OH; OCF₃; OCF₂H; OCFH₂; OCF₂Cl; OCFCl₂; OR⁰; O—C(═O)—R⁹; O—C(═O)—O—R⁹; O—(C═O)—NH—R⁹; O—C(═O)—N(R⁹)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NH—R⁰; N(R⁹)₂; NH—C(═O)—R⁰; NH—C(═O)—O—R⁹; NH—C(═O)—NH₂; NH—C(═O)—NHR⁰; NH—C(═O)—N(R⁹)₂; NR⁰—C(═O)—R⁰; NR⁰—C(═O)—O—R⁹; NR⁰—C(═O)—NH₂; NR⁰—C(═O)—NHR⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂—R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and S(═O)₂—N(R⁰)₂; and
“mono- or polysubstituted” with respect to “aryl” or a “heteroaryl” relates, with respect to the corresponding residues, in each case independently of one another, to the replacement of one or more hydrogen atoms each independently of one another by at least one substituent selected from the group consisting of F; Cl; Br; I; NO₂; CN; CF₃; CF₂H; CFH₂; CF₂Cl; CFCl₂; R⁰; C(═O)—H; C(═O)—R⁰; C(═O)—OH; C(═O)—OR⁰; CO—NH₂; C(═O)—NHR⁰; C(═O)—N(R⁰)₂; OH; 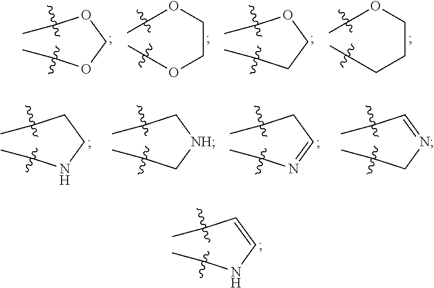
OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂—OH; O—S(═O)₂—OR⁰; O—S(═O)₂—NH₂; O—S(═O)₂—NHR⁰; O—S(═O)₂—N(R⁰)₂; NH₂; NHR⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂—OH; NH—S(═O)₂—R⁰; NH—S(═O)₂—OR⁰; NH—S(═O)₂—NH₂; NH—S(═O)₂—NHR⁰; NH—S(═O)₂—N(R⁰)₂; NR⁰—S(═O)₂—OH; NR⁰—S(═O)₂R⁰; NR⁰—S(═O)₂—OR⁰; NR⁰—S(═O)₂—NH₂; NR⁰—S(═O)₂—NHR⁰; NR⁰—S(═O)₂—N(R⁰)₂; SH; SCF₃; SCF₂H; SCFH₂; SCF₂Cl; SCFCl₂; SR⁰; S(═O)—R⁰; S(═O)₂—R⁰; S(═O)₂—OH; S(═O)₂—OR⁰; S(═O)₂—NH₂; S(═O)₂—NHR⁰; and)S(═O)₂—N(R⁰)₂;
optionally in the form of a single stereoisomer or a mixture of stereoisomers, in the form of the free compound and/or a physiologically acceptable salt or a solvate, in particular hydrate, thereof.

2. A compound according to claim 1, wherein n represents 1.

3. A compound according to claim 1, wherein Y represents O.

4. A compound according to claim 1, wherein
R¹ represents a substructure (T1) 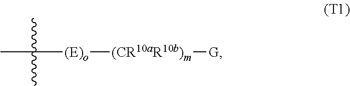
wherein
E represents O, S or NR¹¹, wherein
R¹¹ represents H or an unsubstituted C₁₋₄ aliphatic residue;
o represents 0 or 1;
R^{10a} and R^{10b} each independently represent H, F, Cl, Br, I or an unsubstituted C₁₋₄ aliphatic residue,
m represents 0, 1 or 2; and
G represents:
a C₁₋₈ aliphatic residue, unsubstituted or mono- or polysubstituted with one or more substituents each selected independently from the group consisting of F, Cl, Br, I, OH, O—C₁₋₄ alkyl, O—C₁₋₄ alkylene-OH, O—C₁₋₄ alkylene-O—C₁₋₄ alkyl, OCF₃, CF₃, NH₂, NH(C₁₋₄ alkyl), N(C₁₋₄ alkyl)₂, SH, S—C₁₋₄ alkyl, and SCF₃; or
a C₃₋₁₀ cycloaliphatic residue or a 3 to 10 membered heterocyclo-aliphatic residue, in each case unsubstituted or mono- or polysubstituted with one or more substituents selected independently from the group consisting of F, Cl, Br, I, NO₂, CN, OH, O—C₁₋₄ alkyl, OCF₃, C₁₋₄ alkyl, CF₃, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents selected independently from the group consisting of F, Cl, Br, I, NO₂, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl, and SCF₃; or
an aryl or heteroaryl, in each case unsubstituted or mono- or polysubstituted with one or more substituents selected independently from the group consisting of F, Cl, Br, I, NO₂, CN, OH, O—C₁₋₄ alkyl, 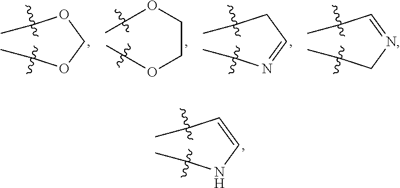
O—C₁₋₄ alkylene-O—C₁₋₄ alkyl, OCF₃, C₁₋₄ alkyl, C₁₋₄ alkylene-O—C₁₋₄-alkyl, CF₃, CF₂H, CFH₂, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents selected independently from the group consisting of F, Cl, Br, I, NO₂, CN, OH, O—C₁₋₄ alkyl, OCF₃, C₁₋₄ alkyl, CF₃, NH₂, NH(C₁₋₄ alkyl), N(C₁₋₄ alkyl)₂, SH, S—C₁₋₄ alkyl, and SCF₃.

5. A compound according to claim 1, wherein R² represents:
a C₁₋₄ aliphatic residue, unsubstituted or mono- or polysubstituted with one or more substituents independently selected from the group consisting of F, Cl, Br and I; or
an unsubstituted C₃₋₆ cycloaliphatic residue.

6. A compound according to claim 1, wherein R³ is selected from the group consisting of H, methyl and ethyl.

7. A compound according to claim 1, wherein
R^{3a} is selected from the group consisting of H, methyl and ethyl;
R^{4a} represents H, methyl, ethyl, cyclopropyl, cyclobutyl, cyclopentyl, cyclohexyl, or phenyl, wherein phenyl is unsubstituted or substituted with 1, 2, 3, 4 or 5 substituents independently selected from the group consisting of F, Cl, Br, I, NO₂, CN, CF₃, CF₂H, CFH₂, CF₂Cl, CFCl₂, OH, NH₂, NH(C₁ alkyl) and N(C₁ alkyl)(C₁₋₄ alkyl), C₁ alkyl, and O—C₁₋₄-alkyl; and
R^{4b} represents H, methyl, or ethyl, or
R^{4a} and R^{4b} together with the carbon atom connecting them form a cyclopropyl, cyclobutyl, cyclopentyl, or cyclohexyl ring.

8. A compound according to claim 1, wherein:
Z represents N, and R^{4a} represents H; or
Z represents CR^{4b}, and R^{4a} and R^{4b} each represent H; or
Z represents CR^{4b}, R^{4a} represents methyl, and R^{4b} represents H.

9. A compound according to claim 1, wherein the substructure (T2) of formula (I) 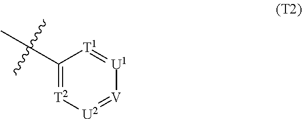
represents a substructure selected from the group consisting of:

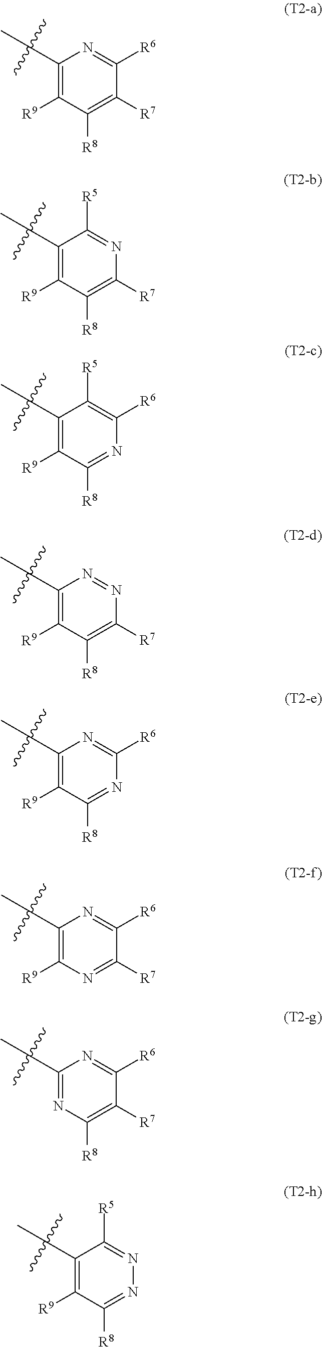

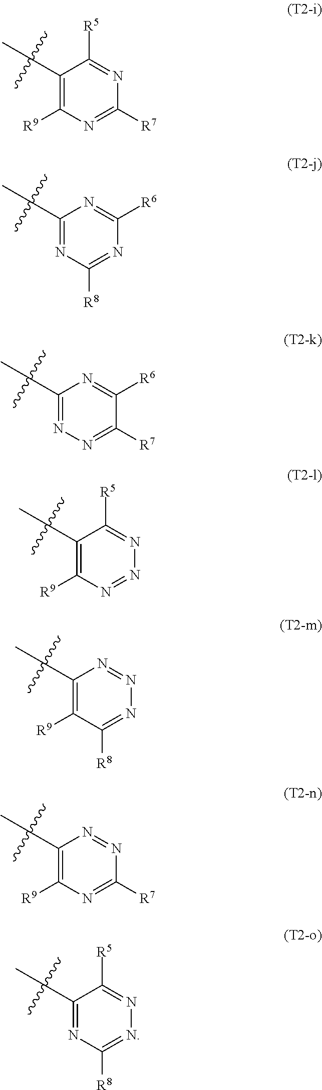

10. A compound according to claim 1, wherein:
R⁵ and R⁹ are each independently selected from the group consisting of H; F; Cl; Br; I; CN; NO₂; CF₃; CF₂H; CFH₂; OH; OCF₃; OCF₂Cl; OCFCl₂; SH; SCF₃; NH₂; C(═O)—NH₂; methyl; ethyl; tert.-butyl; O-methyl; NH-methyl; and N(methyl)₂;
R⁶ and R⁸ are each independently selected from the group consisting of H; F; Cl; Br; I; CN; NO₂; CF₃; CF₂H; CFH₂; OH; OCF₃; OCF₂Cl; OCFCl₂; SH; SCF₃; NH₂; C(═O)—NH₂; methyl, ethyl, tert.-butyl, O-methyl, NH-methyl, and N(methyl)₂; and
R⁷ is selected from the group consisting of:
H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; NH₂; C(═O)—NH₂
a C₁₋₁₀ aliphatic residue, (C₁₋₈ aliphatic group)-OH, (C₁₋₈ aliphatic group)-O—C₁₋₁₀ aliphatic residue, (C₁₋₈ aliphatic group)-O—(C₁₋₈ aliphatic group)-OH, (C₁₋₈ aliphatic group)O—(C₁₋₈ aliphatic group)-O—C₁₋₁₀ aliphatic residue, a (C₁₋₈ aliphatic group)-NH—C₁₋₁₀ aliphatic residue, a (C₁₋₈ aliphatic group)-NH—(C₁₋₈ aliphatic residue)-OH, a (C₁₋₈ aliphatic group)-N(C₁₋₁₀ aliphatic residue)-(C₁₋₈ aliphatic residue)-OH, a (C₁₋₈ aliphatic group)-NHS(═O) 2-C₁₋₁₀ aliphatic residue, a (C₁₋₈ aliphatic group)-NH—S(═O)₂—NH₂, a (C₁₋₈ aliphatic group)-S(═O)₂—C₁₋₁₀ aliphatic residue, a C(═O)—C₁₋₁₀ aliphatic residue, a C(═O)—NH—C₁₋₁₀ aliphatic residue,
an O—C₁₋₁₀ aliphatic residue, an O—(C₁₋₈ aliphatic group)-O—C₁₋₁₀ aliphatic residue, O—(C₁₋₈ aliphatic group)-OH,
an NH—C₁₋₁₀ aliphatic residue, a N(C₁₋₁₀ aliphatic residue)₂, an NH—(C₁₋₈ aliphatic group)-O—C₁₋₁₀ aliphatic residue, an NH—(C₁₋₈ aliphatic group)-OH, an NH—C(═O)—C₁₋₁₀ aliphatic residue, an N(C₁₋₁₀ aliphatic residue)(C(═O)—C₁₋₁₀ aliphatic residue), an N(C₁₋₁₀ aliphatic residue)[(C₁₋₈ aliphatic group)-O—C₁₋₁₀ aliphatic residue], an N(C₁₋₁₀ aliphatic residue)[(C₁₋₈ aliphatic group)-OH], an NH—S(═O)₂—C₁₋₁₀ aliphatic residue, an N(C₁₋₁₀ aliphatic residue)[S(═O)₂—C₁₋₁₀ aliphatic residue],
an S(═O)₂—C₁₋₁₀ aliphatic residue, an S(═O)₂—NH—C₁₋₁₀ aliphatic residue, an S(═O)₂—N(C₁₋₁₀ aliphatic residue)₂, an S—C₁₋₁₀ aliphatic residue,
wherein each of the aforementioned C₁₋₁₀ aliphatic residues and C₁₋₈ aliphatic groups can in each case be unsubstituted or monosubstituted with OH;
a C₃₋₁₀ cycloaliphatic residue, a C(═O)—C₃₋₁₀ cycloaliphatic residue, a C(═O)NH—C₃₋₁₀ cycloaliphatic residue a O—C₃₋₁₀ cycloaliphatic residue, a O—(C₁₋₈ aliphatic group)-C₃₋₁₀ cycloaliphatic residue, a S—C₃₋₁₀ cycloaliphatic residue, a S—(C₁₋₈ aliphatic group)-C₃₋₁₀ cycloaliphatic residue, a NH—C₃₋₁₀ cycloaliphatic residue, a NH—C(═O)—C₃₋₁₀ cycloaliphatic residue, a NH—(C₁₋₈ aliphatic group)-C₃₋₁₀ cycloaliphatic residue, a N(C₁₋₁₀ aliphatic residue)(C₃₋₁₀ cycloaliphatic residue), a 3 to 10 membered heterocycloaliphatic residue, a C(═O)-(3 to 10 membered heterocycloaliphatic residue), a C(═O)—NH-(3 to 10 membered heterocycloaliphatic residue), an O-(3 to 10 membered heterocycloaliphatic residue), an O—(C₁₋₈ aliphatic group)-(3 to 10 membered heterocycloaliphatic residue), an S-(3 to 10 membered heterocycloaliphatic residue), an S—(C₁₋₈ aliphatic group)-(3 to 10 membered heterocyclo-aliphatic residue), an NH-(3 to 10 membered heterocycloaliphatic residue), an NH—C(═O)-(3 to 10 membered heterocycloaliphatic residue), an NH—(C₁₋₈ aliphatic group)-(3 to 10 membered heterocycloaliphatic residue), an N(C₁₋₁₀ aliphatic residue)(3 to 10 membered heterocycloaliphatic residue), wherein:
each of the aforementioned residues can be optionally bridged via an C₁₋₈ aliphatic group;
the C₁₋₁₀ aliphatic residues and the C₁₋₈ aliphatic groups can each independently be unsubstituted or monosubstituted with OH; and
the C₃₋₁₀ cycloaliphatic residue and the 3 to 10 membered heterocycloaliphatic residue, respectively, can each independently be unsubstituted or mono- or polysubstituted with one or more substituents each independently selected from the group consisting of F, Cl, Br, I, NO₂, CN, OH, ═O, O—C₁₋₄ alkyl, OCF₃, C₁₋₄ alkyl, CF₃, SH, S—C₁₋₄ alkyl, SCF₃, NH₂, NH(C₁₋₄ alkyl), and N(C₁₋₄ alkyl)₂;
aryl, C(═O)-aryl, C(═O)—NH-aryl, O-aryl, a O—(C₁₋₈ aliphatic group)-aryl, S-aryl, an S—(C₁₋₈ aliphatic group)-aryl, a NH-aryl, NH—C(═O)-aryl, NH—S(═O)₂-aryl, an NH—(C₁₋₈ aliphatic group)-aryl, an N(C₁₋₁₀, aliphatic residue)(aryl), heteroaryl, C(═O)-heteroaryl, C(═O)—NH-heteroaryl, O-heteroaryl, O—(C₁₋₈ aliphatic group)-heteroaryl, S-(heteroaryl), S—(C₁₋₈ aliphatic group)-(heteroaryl), NH-(heteroaryl), NH—C(═O)-heteroaryl, NH—S(═O)₂-heteroaryl, NH—(C₁₋₈ aliphatic group)(heteroaryl), N(C₁₋₁₀, aliphatic residue)(heteroaryl), wherein:
each of the aforementioned residues can optionally be bridged via a C₁₋₈ aliphatic group;
the C₁₋₁₀ aliphatic residues and the C₁₋₈ aliphatic groups of the aforementioned residues can each independently be unsubstituted or monosubstituted with OH; and
the aryl and heteroaryl of the aforementioned residues, respectively, can each independently be unsubstituted or mono- or polysubstituted with one or more substituents each independently selected from the group consisting of F, Cl, Br, I, NO₂, CN, OH, O—C₁₋₄ alkyl, O—C₁₋₄ alkylene-O—C₁₋₄ alkyl, OCF₃, C₁₋₄ alkyl, C₁₋₄ alkylene-O—C₁₋₄-alkyl, CF₃, CF₂H, CHF₂, SH, S—C₁₋₄ alkyl, SCF₃, NH₂, NH(C₁₋₄ alkyl), N(C₁₋₄ alkyl)₂, phenyl and pyridyl, wherein phenyl or pyridyl are respectively unsubstituted or mono- or polysubstituted with one or more substituents each independently selected from the group consisting of F, Cl, Br, I, NO₂, CN, OH, O—C₁₋₄ alkyl, O—C₁₋₄ alkylene-O—C₁₋₄ alkyl OCF₃, C₁₋₄ alkyl, C₁₋₄ alkylene-O—C₁₋₄-alkyl, C(═O)—OH, CF₃, CF₂H, CHF₂, NH₂, NH(C₁₋₄ alkyl), N(C₁₋₄ alkyl)₂, SH, S—C₁₋₄ alkyl, SCF₃ and S(═O)₂OH.

11. A compound according to claim 1, wherein
R¹ represents substructure (T1) 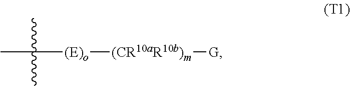
wherein
E represents O or S;
o represents 0 or 1;
R^{10a} and R^{10b} are each independently selected from the group consisting of H, methyl and ethyl;
m represents 0, 1 or 2; and
G represents:
methyl, ethyl, n-propyl, isopropyl, n-butyl, sec.-butyl, tert.-butyl, pentyl, hexyl, or 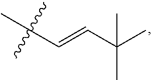 [SMILES: */C=C/C(C)(C)C]
or
cyclopropyl, cyclobutyl, cyclopentyl and cyclohexyl, or is selected from the group consisting of piperidinyl, morpholinyl, tetrahydropyrrolyl, tetrahydroquinolinyl, tetrahydroisoquinolinyl, dihydroquinolinyl, dihydropyrrolyl, dihydropyridinyl, dihydroisoquinolinyl, tetrahydropyranyl, tetrahydrofuranyl and tetrahydropyridinyl, each independently being unsubstituted or mono- or polysubstituted with one or more substituents selected independently from the group consisting of F, Cl, Br, I, OH, O—C₁₋₄ alkyl, C₁₋₄ alkyl, NH₂, NH(C₁₋₄ alkyl), and N(C₁₋₄ alkyl)₂, or furyl or thienyl, in each case unsubstituted, or
phenyl or pyridyl, in each case unsubstituted or mono- or polysubstituted with one or more substituents selected independently from the group consisting of F, Cl, Br, I, NO₂, CN, OH, O—C₁₋₄ alkyl, 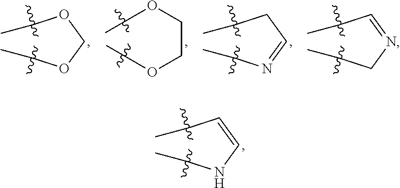
O—C₁₋₄ alkylene-O—C₁₋₄ alkyl, OCF₃, C₁₋₄ alkyl, C₁₋₄ alkylene-O—C₁₋₄-alkyl, CF₃, CF₂H, CFH₂, SCF₃, NH₂, NH(C₁₋₄ alkyl), and N(C₁₋₄ alkyl)₂;
R² is selected from:
the group consisting of CF₃, methyl, ethyl, n-propyl, isopropyl, n-butyl, sec.-butyl, and tert.-butyl, or
the group consisting of cyclopropyl, cyclobutyl, cyclopentyl, and cyclohexyl;
R³ represents H or an unsubstituted C₁₋₄ aliphatic residue;
n represents 1, 2 or 3;
R^{3a} represents H, methyl, or ethyl;
R^{4a} represents H, methyl, or ethyl;
Y represents O;
Z represents N or CR^{4b};
R^{4b} represents H, methyl, or ethyl;
T¹ represents N or C—R⁵;
U¹ represents N or C—R⁶;
V represents N or C—R⁷;
U² represents N or C—R⁸;
T² represents N or C—R⁹;
with the proviso that 1, 2 or 3 of variables T¹, U¹, V, U² and T² represent(s) a nitrogen atom;
R⁵ and R⁹ are each independently selected from the group consisting of H; F; Cl; Br; I; CF₃; OH; methyl; and O-methyl;
R⁶ and R⁸ are each independently selected from the group consisting of H; F; Cl; Br; I; CF₃; OH; methyl; and O-methyl; and
R⁷ is selected from the group consisting of:
H; F; Cl; Br; I; CN; CF₃; CF₂H; CFH₂; OH; OCF₃; SH; SCF₃; NH₂; C(═O)—NH₂; S(═O)₂—OH; S(═O)₂—NH₂;
C₁₋₄ alkyl, C₁₋₄ alkylene-OH, C₁₋₄ alkylene-O—C₁₋₄ alkyl, C₁₋₄ alkylene-O—C₁₋₄ alkylene-OH, C₁₋₄ alkylene-O—C₁₋₄ alkylene-O—C₁₋₄ alkyl, C₁₋₄ alkylene-S(═O)₂—C₁₋₄ alkyl, C₁₋₄ alkylene-NH—S(═O)₂—C₁₋₄ alkyl, C₁₋₄ alkylene-NH—S(═O)₂—NH₂, C₁₋₄ alkylene-NH—C₁₋₄ alkylene-OH, C₁₋₄ alkylene-NH—C₁₋₄ alkylene-O—C₁₋₄ alkyl, C₁₋₄ alkylene-N(C₁₋₄ alkyl)-C₁₋₄ alkylene-OH, C₁₋₄ alkylene-N(C₁₋₄ alkyl)-C₁₋₄ alkylene-O—C₁₋₄ alkyl, O—C₁₋₄ alkyl, O—C₁₋₄ alkylene-OH, O—C₁₋₄ alkylene-O—C₁₋₄ alkyl, NH—C₁₋₄ alkyl, N(C₁₋₄ alkyl)₂, NH—C₁₋₄ alkylene-OH, NH—C₁₋₄ alkylene-O—C₁₋₄ alkyl, N(C₁₋₄ alkyl)-[C₁₋₄ alkylene-OH], N(C₁₋₄ alkyl)-[C₁₋₄ alkylene-O—C₁₋₄ alkyl], NH—S(═O)₂—C₁₋₄ alkyl,
wherein C₁₋₄ alkylene can in each case be unsubstituted or monosubstituted with OH,
a C₃₋₆ cycloaliphatic residue, O—C₃₋₆ cycloaliphatic residue, a 3 to 6 membered heterocycloaliphatic residue,
wherein the C₃₋₆ cycloaliphatic residue and the 3 to 6 membered heterocycloaliphatic residue, respectively, can be unsubstituted or mono- or polysubstituted with one or more substituents selected independently from the group consisting of F, Cl, Br, I, OH, O—C₁₋₄ alkyl, NH₂, NH(C₁₋₄ alkyl), and N(C₁₋₄ alkyl)₂, and C₁₋₄ alkyl; and
phenyl, C(═O)—NH-phenyl, NH—C(═O)-phenyl, heteroaryl, C(═O)—NH-heteroaryl, NH—C(═O)-heteroaryl,
wherein phenyl and heteroaryl of the aforementioned residues, respectively, can each independently be unsubstituted or mono- or polysubstituted with one or more substituents selected independently from the group consisting of F, Cl, Br, I, OH, O—C₁₋₄ alkyl, C₁₋₄ alkyl, and CF₃.

12. A compound according to claim 11, wherein n represents 1 or 2.

13. A compound according to claim 1, selected from the group consisting of:
1 N-((1-(3-chlorophenyl)-3-(trifluoromethyl)-1H-pyrazol-5-yl)methyl)-2-(pyridin-2-yl)acetamide;
2 N-((3-tert-butyl-1-(3-chlorophenyl)-1H-pyrazol-5-yl)methyl)-2-(pyridin-2-yl)propanamide;
3 N-((1-(3-chlorophenyl)-3-(trifluoromethyl)-1H-pyrazol-5-yl)methyl)-2-(pyridin-2-yl)propanamide;
4 1-((3-tert-butyl-1-(3-chlorophenyl)-1H-pyrazol-5-yl)methyl)-3-(pyridin-2-yl)urea;
5 1-((1-(3-chlorophenyl)-3-(trifluoromethyl)-1H-pyrazol-5-yl)methyl)-3-(pyridin-2-yl)urea;
6 N-((3-tert-butyl-1-(3-chlorophenyl)-1H-pyrazol-5-yl)methyl)-2-(pyridin-3-yl)acetamide;
7 N-((1-(3-chlorophenyl)-3-(trifluoromethyl)-1H-pyrazol-5-yl)methyl)-2-(pyridin-3-yl)acetamide;
8 N-((1-(3-chlorophenyl)-3-(trifluoromethyl)-1H-pyrazol-5-yl)methyl)-2-(pyridin-3-yl)propanamide;
9 1-((1-(3-chlorophenyl)-3-(trifluoromethyl)-1H-pyrazol-5-yl)methyl)-3-(pyridin-3-yl)urea;
10 N-((3-tert-butyl-1-(3-chlorophenyl)-1H-pyrazol-5-yl)methyl)-2-(pyridin-4-yl)acetamide;
11 N-((1-(3-chlorophenyl)-3-(trifluoromethyl)-1H-pyrazol-5-yl)methyl)-2-(pyridin-4-yl)acetamide;
12 1-((1-(3-chlorophenyl)-3-(trifluoromethyl)-1H-pyrazol-5-yl)methyl)-3-(pyridin-4-yl)urea;
13 N-((1-(3-chlorophenyl)-3-(trifluoromethyl)-1H-pyrazol-5-yl)methyl)-2-(pyrimidin-4-yl)acetamide;
14 1N-((3-tert-butyl-1-(3-chlorophenyl)-1H-pyrazol-5-yl)methyl)-2-(pyrazin-2-yl)acetamide;
15 1-((1-(3-chlorophenyl)-3-(trifluoromethyl)-1H-pyrazol-5-yl)methyl)-3-(pyridazin-4-yl)urea;
16 N-((1-(3-chlorophenyl)-3-(trifluoromethyl)-1H-pyrazol-5-yl)methyl)-2-(pyrimidin-5-yl)acetamide;
17 N-((1-(3-chlorophenyl)-3-(trifluoromethyl)-1H-pyrazol-5-yl)methyl)-2-(6-chloropyridin-3-yl)acetamide;
18 1-((1-(3-chlorophenyl)-3-(trifluoromethyl)-1H-pyrazol-5-yl)methyl)-3-(5-fluoropyridin-3-yl)urea;
19 1-((1-(3-chlorophenyl)-3-(trifluoromethyl)-1H-pyrazol-5-yl)methyl)-3-(2-methylpyrimidin-5-yl)urea;
20 1-((1-(3-chlorophenyl)-3-(trifluoromethyl)-1H-pyrazol-5-yl)methyl)-3-(1,3,5-triazin-2-yl)urea;
21 1-((1-(3-chlorophenyl)-3-(trifluoromethyl)-1H-pyrazol-5-yl)methyl)-3-(6-(2-(methylsulfonyl)ethyl)pyridin-3-yl)urea;
22 1-((3-tert-butyl-1-(3-chlorophenyl)-1H-pyrazol-5-yl)methyl)-3-(5-fluoro-6-(2-(methylsulfonyl)ethyl)pyridin-3-yl)urea;
23 1-((1-(3-chlorophenyl)-3-(trifluoromethyl)-1H-pyrazol-5-yl)methyl)-3-(5-fluoro-6-(2-(methylsulfonyl)ethyl)pyridin-3-yl)urea;
24 1-((1-(3-chlorophenyl)-3-cyclopropyl-1H-pyrazol-5-yl)methyl)-3-(5-fluoro-6-(2-(methylsulfonyl)ethyl)pyridin-3-yl)urea;
25 5-(3-((3-tert-butyl-1-(3-chlorophenyl)-1H-pyrazol-5-yl)methyl)ureido)picolinamide;
26 5-(3-((1-(3-chlorophenyl)-3-(trifluoromethyl)-1H-pyrazol-5-yl)methyl)ureido)picolinamide;
27 N-((1-(3-chlorophenyl)-3-(trifluoromethyl)-1H-pyrazol-5-yl)methyl)-2-(6-(methylsulfonamidomethyl)pyridin-3-yl)propanamide;
28 N-((5-(3-((1-(3-chlorophenyl)-3-(trifluoromethyl)-1H-pyrazol-5-yl)methyl)ureido)pyridin-2-yl)methyl)methanesulfonamide;
29 N-((5-(3-((1-(3-chlorophenyl)-3-(trifluoromethyl)-1H-pyrazol-5-yl)methyl)ureido)pyridin-2-yl)methyl)sulfuric diamide;
30 N-((1-(3-chlorophenyl)-3-(trifluoromethyl)-1H-pyrazol-5-yl)methyl)-2-(6-(hydroxymethyl)pyridin-3-yl)propanamide;
31 (E)-1-((1-(3,3-dimethylbut-1-enyl)-3-(trifluoromethyl)-1H-pyrazol-5-yl)methyl)-3-(6-(hydroxymethyl)pyridin-3-yl)urea;
32 1-((1-(3-chlorophenyl)-3-(trifluoromethyl)-1H-pyrazol-5-yl)methyl)-3-(6-(hydroxymethyl)pyridin-3-yl)urea;
33 1-((1-(3-fluorophenyl)-3-(trifluoromethyl)-1H-pyrazol-5-yl)methyl)-3-(6-(hydroxymethyl)pyridin-3-yl)urea;
34 1-((1-(3-fluoro-4-methylphenyl)-3-(trifluoromethyl)-1H-pyrazol-5-yl)methyl)-3-(6-(hydroxymethyl)pyridin-3-yl)urea;
35 1-((1-(3-fluoro-4-methoxyphenyl)-3-(trifluoromethyl)-1H-pyrazol-5-yl)methyl)-3-(6-(hydroxymethyl)pyridin-3-yl)urea;
36 1-(6-(hydroxymethyl)pyridin-3-yl)-3-((1-m-tolyl-3-(trifluoromethyl)-1H-pyrazol-5-yl)methyl)urea;
37 1-(6-(hydroxymethyl)pyridin-3-yl)-3-((1-(4-methoxy-3-methylphenyl)-3-(trifluoromethyl)-1H-pyrazol-5-yl)methyl)urea;
38 1-(6-(hydroxymethyl)pyridin-3-yl)-3-((1-(3-isopropylphenyl)-3-(trifluoromethyl)-1H-pyrazol-5-yl)methyl)urea;
39 1-((1-(3-tert-butylphenyl)-3-(trifluoromethyl)-1H-pyrazol-5-yl)methyl)-3-(6-(hydroxymethyl)pyridin-3-yl)urea;
40 1-(6-(hydroxymethyl)pyridin-3-yl)-3-((1-(3-(methoxymethyl)phenyl)-3-(trifluoromethyl)-1H-pyrazol-5-yl)methyl)urea;
41 1-((1-(3-(difluoromethyl)phenyl)-3-(trifluoromethyl)-1H-pyrazol-5-yl)methyl)-3-(6-(hydroxymethyl)pyridin-3-yl)urea;
42 1-((1-(3-cyanophenyl)-3-(trifluoromethyl)-1H-pyrazol-5-yl)methyl)-3-(6-(hydroxymethyl)pyridin-3-yl)urea;
43 1-((1-(3-(dimethylamino)phenyl)-3-(trifluoromethyl)-1H-pyrazol-5-yl)methyl)-3-(6-(hydroxymethyl)pyridin-3-yl)urea;
44 1-((1-(5-chloropyridin-3-yl)-3-(trifluoromethyl)-1H-pyrazol-5-yl)methyl)-3-(6-(hydroxymethyl)pyridin-3-yl)urea;
45 1-(6-(hydroxymethyl)pyridin-3-yl)-3-((1-(6-methoxypyridin-3-yl)-3-(trifluoromethyl)-1H-pyrazol-5-yl)methyl)urea;
46 1-((1-(benzo[d][1,3]dioxol-5-yl)-3-(trifluoromethyl)-1H-pyrazol-5-yl)methyl)-3-(6-(hydroxymethyl)pyridin-3-yl)urea;
47 1-((1-(1H-indol-6-yl)-3-(trifluoromethyl)-1H-pyrazol-5-yl)methyl)-3-(6-(hydroxymethyl)pyridin-3-yl)urea;
48 1-((1-(furan-3-yl)-3-(trifluoromethyl)-1H-pyrazol-5-yl)methyl)-3-(6-(hydroxymethyl)pyridin-3-yl)urea;
49 1-(6-(hydroxymethyl)pyridin-3-yl)-3-((1-(thiophen-2-yl)-3-(trifluoromethyl)-1H-pyrazol-5-yl)methyl)urea;
50 1-((1-(3-chlorophenyl)-3-(trifluoromethyl)-1H-pyrazol-5-yl)methyl)-3-(5-fluoro-6-(hydroxymethyl)pyridin-3-yl)urea;
51 N-((1-(3-chlorophenyl)-3-(trifluoromethyl)-1H-pyrazol-5-yl)methyl)-2-(6-(2-hydroxyethyl)pyridin-3-yl)propanamide;
52 1-((1-(3-chlorophenyl)-3-(trifluoromethyl)-1H-pyrazol-5-yl)methyl)-3-(6-(2-hydroxyethyl)pyridin-3-yl)urea;
53 N-((1-(3-chlorophenyl)-3-(trifluoromethyl)-1H-pyrazol-5-yl)methyl)-2-(6-((2-hydroxyethoxy)methyl)pyridin-3-yl)propanamide;
54 1-((1-(3-fluorophenyl)-3-(trifluoromethyl)-1H-pyrazol-5-yl)methyl)-3-(6-(tetrahydro-2H-pyran-4-yl)pyridin-3-yl)urea;
55 5-(1-((1-(3-chlorophenyl)-3-(trifluoromethyl)-1H-pyrazol-5-yl)methylamino)-1-oxopropan-2-yl)picolinamide;
56 5-(1-((3-tert-butyl-1-(3-chlorophenyl)-1H-pyrazol-5-yl)methylamino)-1-oxopropan-2-yl)picolinamide;
57 5-(1-((1-(3-chlorophenyl)-3-(trifluoromethyl)-1H-pyrazol-5-yl)methylamino)-1-oxopropan-2-yl)-N-phenyl)picolinamide;
58 5-(1-((1-(3-chlorophenyl)-3-(trifluoromethyl)-1H-pyrazol-5-yl)methylamino)-1-oxopropan-2-yl)-N-(4-fluorophenyl)picolinamide;
59 5-(1-((1-(3-chlorophenyl)-3-(trifluoromethyl)-1H-pyrazol-5-yl)methylamino)-1-oxopropan-2-yl)-N-(4-(trifluoromethyl)phenyl)picolinamide;
60 5-(1-((3-tert-butyl-1-(3-chlorophenyl)-1H-pyrazol-5-yl)methylamino)-1-oxopropan-2-yl)-N-(4-fluorophenyl)picolinamide;
61 5-(1-((3-tert-butyl-1-(3-chlorophenyl)-1H-pyrazol-5-yl)methylamino)-1-oxopropan-2-yl)-N-(4-(trifluoromethyl)phenyl)picolinamide;
62 5-(1-((1-(3-chlorophenyl)-3-(trifluoromethyl)-1H-pyrazol-5-yl)methylamino)-1-oxopropan-2-yl)-N-phenylpyrimidine-2-carboxamide;
63 5-(1-((1-(3-chlorophenyl)-3-(trifluoromethyl)-1H-pyrazol-5-yl)methylamino)-1-oxopropan-2-yl)-N-(4-fluorophenyl)pyrimidine-2-carboxamide;
64 5-(1-((1-(3-chlorophenyl)-3-(trifluoromethyl)-1H-pyrazol-5-yl)methylamino)-1-oxopropan-2-yl)-N-(4-(trifluoromethyl)phenyl)pyrimidine-2-carboxamide;
65 5-(1-((3-tert-butyl-1-(3-chlorophenyl)-1H-pyrazol-5-yl)methylamino)-1-oxopropan-2-yl)-N-phenylpyrimidine-2-carboxamide;
66 5-(1-((3-tert-butyl-1-(3-chlorophenyl)-1H-pyrazol-5-yl)methylamino)-1-oxopropan-2-yl)-N-(4-fluorophenyl)pyrimidine-2-carboxamide;
67 5-(1-((3-tert-butyl-1-(3-chlorophenyl)-1H-pyrazol-5-yl)methylamino)-1-oxopropan-2-yl)-N-(4-(trifluoromethyl)phenyl)pyrimidine-2-carboxamide;
68 1-((3-tert-butyl-1-(3-chlorophenyl)-1H-pyrazol-5-yl)methyl)-3-(6-(2-methoxyethylamino)pyridin-3-yl)urea;
69 1-((1-(3-chlorophenyl)-3-(trifluoromethyl)-1H-pyrazol-5-yl)methyl)-3-(6-(2-methoxyethylamino)pyridin-3-yl)urea;
70 1-((3-tert-butyl-1-(3-chlorophenyl)-1H-pyrazol-5-yl)methyl)-3-(6-(2-hydroxyethylamino)pyridin-3-yl)urea;
71 1-((1-(3-chlorophenyl)-3-(trifluoromethyl)-1H-pyrazol-5-yl)methyl)-3-(6-(2-hydroxyethylamino)pyridin-3-yl)urea;
72 1-((1-(3-chlorophenyl)-3-cyclopropyl-1H-pyrazol-5-yl)methyl)-3-(6-(2-hydroxyethylamino)pyridin-3-yl)urea;
73 1-((1-(3-chlorophenyl)-4-methyl-3-(trifluoromethyl)-1H-pyrazol-5-yl)methyl)-3-(6-(2-hydroxyethylamino)pyridin-3-yl)urea;
74 1-(6-(2-hydroxyethylamino)pyridin-3-yl)-3-((1-pentyl-3-(trifluoromethyl)-1H-pyrazol-5-yl)methyl)urea;
75 1-((1-(cyclopropylmethyl)-3-(trifluoromethyl)-1H-pyrazol-5-yl)methyl)-3-(6-(2-hydroxyethylamino)pyridin-3-yl)urea;
76 1-((1-cyclohexyl-3-(trifluoromethyl)-1H-pyrazol-5-yl)methyl)-3-(6-(2-hydroxyethylamino)pyridin-3-yl)urea;
77 1-(6-(2-hydroxyethylamino)pyridin-3-yl)-3-((1-(tetrahydro-2H-pyran-4-yl)-3-(trifluoromethyl)-1H-pyrazol-5-yl)methyl)urea;
78 1-((1-(3-fluorophenyl)-3-(trifluoromethyl)-1H-pyrazol-5-yl)methyl)-3-(6-(2-hydroxyethylamino)pyridin-3-yl)urea;
79 1-((1-(3,4-difluorophenyl)-3-(trifluoromethyl)-1H-pyrazol-5-yl)methyl)-3-(6-(2-hydroxyethylamino)pyridin-3-yl)urea;
80 1-(6-(2-hydroxyethylamino)pyridin-3-yl)-3-((1-(3-methoxyphenyl)-3-(trifluoromethyl)-1H-pyrazol-5-yl)methyl)urea;
81 1-(6-(2-hydroxyethylamino)pyridin-3-yl)-3-((1-m-tolyl-3-(trifluoromethyl)-1H-pyrazol-5-yl)methyl)urea;
82 1-(6-(2-hydroxyethylamino)pyridin-3-yl)-3-((1-(3-isopropylphenyl)-3-(trifluoromethyl)-1H-pyrazol-5-yl)methyl)urea;
83 1-((1-(3-tert-butylphenyl)-3-(trifluoromethyl)-1H-pyrazol-5-yl)methyl)-3-(6-(2-hydroxyethylamino)pyridin-3-yl)urea;
84 1-((3-tert-butyl-1-(3-(trifluoromethyl)phenyl)-1H-pyrazol-5-yl)methyl)-3-(6-(2-hydroxyethylamino)pyridin-3-yl)urea;
85 1-((3-tert-butyl-1-(pyridin-2-yl)-1H-pyrazol-5-yl)methyl)-3-(6-(2-hydroxyethylamino)pyridin-3-yl)urea;
86 1-(6-(2-hydroxyethylamino)pyridin-3-yl)-3-((1-(4-methoxybenzyl)-3-(trifluoromethyl)-1H-pyrazol-5-yl)methyl)urea;
87 1-((3-tert-butyl-1-(3-chlorophenyl)-1H-pyrazol-5-yl)methyl)-3-(6-((2-hydroxyethyl)(methyl)amino)pyridin-3-yl)urea;
88 N-((1-(3-chlorophenyl)-3-(trifluoromethyl)-1H-pyrazol-5-yl)methyl)-2-(6-((2-methoxyethyl)(methyl)amino)pyridin-3-yl)propanamide;
89 1-((1-(3-chlorophenyl)-3-(trifluoromethyl)-1H-pyrazol-5-yl)methyl)-3-(6-((2-methoxyethyl)(methyl)amino)pyridin-3-yl)urea;
90 1-((1-(3-chlorophenyl)-3-(trifluoromethyl)-1H-pyrazol-5-yl)methyl)-3-(6-((2-hydroxyethyl)(methyl)amino)pyridin-3-yl)urea;
91 N-(5-(1-((3-tert-butyl-1-(3-chlorophenyl)-1H-pyrazol-5-yl)methylamino)-1-oxopropan-2-yl)pyridin-2-yl)benzamide;
92 N-(5-(1-((1-(3-chlorophenyl)-3-(trifluoromethyl)-1H-pyrazol-5-yl)methylamino)-1-oxopropan-2-yl)pyridin-2-yl)benzamide;
93 N-(5-(1-((3-tert-butyl-1-(3-chlorophenyl)-1H-pyrazol-5-yl)methylamino)-1-oxopropan-2-yl)pyridin-2-yl)-4-fluorobenzamide;
94 N-(5-(1-((1-(3-chlorophenyl)-3-(trifluoromethyl)-1H-pyrazol-5-yl)methylamino)-1-oxopropan-2-yl)pyridin-2-yl)-4-fluorobenzamide;
95 N-(5-(1-((3-tert-butyl-1-(3-chlorophenyl)-1H-pyrazol-5-yl)methylamino)-1-oxopropan-2-yl)pyridin-2-yl)-4-chlorobenzamide;
96 4-chloro-N-(5-(1-((1-(3-chlorophenyl)-3-(trifluoromethyl)-1H-pyrazol-5-yl)methylamino)-1-oxopropan-2-yl)pyridin-2-yl)benzamide;
97 4-chloro-N-(5-(1-oxo-1-((1-(pyridin-3-yl)-3-(trifluoromethyl)-1H-pyrazol-5-yl)methylamino)propan-2-yl)pyridin-2-yl)benzamide;
98 N-((3-tert-butyl-1-(3-chlorophenyl)-1H-pyrazol-5-yl)methyl)-2-(6-(methylsulfonamido)pyridin-3-yl)propanamide;
99 N-((1-(3-chlorophenyl)-3-(trifluoromethyl)-1H-pyrazol-5-yl)methyl)-2-(6-(methylsulfonamido)pyridin-3-yl)propanamide;
100 N-((1-(3-fluorophenyl)-3-(trifluoromethyl)-1H-pyrazol-5-yl)methyl)-2-(6-(methylsulfonamido)pyridin-3-yl)propanamide;
101 N-(5-(3-((1-(3-chlorophenyl)-3-(trifluoromethyl)-1H-pyrazol-5-yl)methyl)ureido)pyridin-2-yl)methanesulfonamide;
102 N-((3-tert-butyl-1-(3-chlorophenyl)-1H-pyrazol-5-yl)methyl)-2-(5-fluoro-6-(methylsulfonamido)pyridin-3-yl)propanamide;
103 N-((1-(3-chlorophenyl)-3-(trifluoromethyl)-1H-pyrazol-5-yl)methyl)-2-(5-fluoro-6-(methylsulfonamido)pyridin-3-yl)propanamide;
104 N-((1-(3-chlorophenyl)-3-(trifluoromethyl)-1H-pyrazol-5-yl)methyl)-2-(5-methoxy-6-(methylsulfonamido)pyridin-3-yl)propanamide;
105 N-((3-tert-butyl-1-(3-chlorophenyl)-1H-pyrazol-5-yl)methyl)-2-(5-methoxy-6-(methylsulfonamido)pyridin-3-yl)propanamide;
106 1-((1-(3-chlorophenyl)-3-(trifluoromethyl)-1H-pyrazol-5-yl)methyl)-3-(6-(dimethylamino)-5-(trifluoromethyl)pyridin-3-yl)urea;
107 1-((3-tert-butyl-1-(3-chlorophenyl)-1H-pyrazol-5-yl)methyl)-3-(6-(dimethylamino)-5-(trifluoromethyl)pyridin-3-yl)urea;
108 1-(6-(azetidin-1-yl)pyridin-3-yl)-3-((3-tert-butyl-1-(3-chlorophenyl)-1H-pyrazol-5-yl)methyl)urea;
109 1-(6-(azetidin-1-yl)pyridin-3-yl)-3-((1-(3-chlorophenyl)-3-(trifluoromethyl)-1H-pyrazol-5-yl)methyl)urea;
110 1-((3-tert-butyl-1-(3-fluorophenyl)-1H-pyrazol-5-yl)methyl)-3-(6-(3-hydroxyazetidin-1-yl)pyridin-3-yl)urea;
111 1-((1-(3-chlorophenyl)-3-(trifluoromethyl)-1H-pyrazol-5-yl)methyl)-3-(6-(3-hydroxyazetidin-1-yl)pyridin-3-yl)urea;
112 1-((1-(3-fluorophenyl)-3-(trifluoromethyl)-1H-pyrazol-5-yl)methyl)-3-(6-(3-hydroxyazetidin-1-yl)pyridin-3-yl)urea;
113 1-((1-(3-chloro-4-fluorophenyl)-3-(trifluoromethyl)-1H-pyrazol-5-yl)methyl)-3-(6-(3-hydroxyazetidin-1-yl)pyridin-3-yl)urea;
114 1-(6-(3-hydroxyazetidin-1-yl)pyridin-3-yl)-3-((1-m-tolyl-3-(trifluoromethyl)-1H-pyrazol-5-yl)methyl)urea;
115 1-(6-(3-hydroxyazetidin-1-yl)pyridin-3-yl)-3-((1-(3-isopropyl phenyl)-3-(trifluoromethyl)-1H-pyrazol-5-yl)methyl)urea;
116 1-((1-(3-tert-butylphenyl)-3-(trifluoromethyl)-1H-pyrazol-5-yl)methyl)-3-(6-(3-hydroxyazetidin-1-yl)pyridin-3-yl)urea;
117 1-((3-tert-butyl-1-(3-chlorophenyl)-1H-pyrazol-5-yl)methyl)-3-(6-(3-hydroxyazetidin-1-yl)pyridin-3-yl)urea;
118 1-((1-(3-(dimethylamino)phenyl)-3-(trifluoromethyl)-1H-pyrazol-5-yl)methyl)-3-(6-(3-hydroxyazetidin-1-yl)pyridin-3-yl)urea;
119 1-(6-(3-hydroxyazetidin-1-yl)pyridin-3-yl)-3-((1-(3-methoxyphenyl)-3-(trifluoromethyl)-1H-pyrazol-5-yl)methyl)urea;
120 1-((3-tert-butyl-1-(3-chlorophenyl)-1H-pyrazol-5-yl)methyl)-3-(6-(pyrrolidin-1-yl)pyridin-3-yl)urea;
121 1-((1-(3-chlorophenyl)-3-(trifluoromethyl)-1H-pyrazol-5-yl)methyl)-3-(6-(pyrrolidin-1-yl)pyridin-3-yl)urea;
122 1-((1-(3-chlorophenyl)-3-(trifluoromethyl)-1H-pyrazol-5-yl)methyl)-3-(5-fluoro-6-(pyrrolidin-1-yl)pyridin-3-yl)urea;
123 1-((1-(3-chlorophenyl)-3-(trifluoromethyl)-1H-pyrazol-5-yl)methyl)-3-(5-methoxy-6-(pyrrolidin-1-yl)pyridin-3-yl)urea;
124 (R)-1-((1-(3-chlorophenyl)-3-(trifluoromethyl)-1H-pyrazol-5-yl)methyl)-3-(6-(3-hydroxypyrrolidin-1-yl)pyridin-3-yl)urea;
125 (S)-1-((1-(3-chlorophenyl)-3-(trifluoromethyl)-1H-pyrazol-5-yl)methyl)-3-(6-(3-hydroxypyrrolidin-1-yl)pyridin-3-yl)urea;
126 (R)-1-((3-tert-butyl-1-(3-chlorophenyl)-1H-pyrazol-5-yl)methyl)-3-(6-(3-hydroxypyrrolidin-1-yl)pyridin-3-yl)urea;
127 (S)-1-((3-tert-butyl-1-(3-chlorophenyl)-1H-pyrazol-5-yl)methyl)-3-(6-(3-hydroxypyrrolidin-1-yl)pyridin-3-yl)urea;
128 1-((1-(3-chlorophenyl)-3-(trifluoromethyl)-1H-pyrazol-5-yl)methyl)-3-(6-hydroxypyridin-3-yl)urea;
129 N-((1-(3-chlorophenyl)-3-(trifluoromethyl)-1H-pyrazol-5-yl)methyl)-2-(6-methoxypyridin-3-yl)propanamide;
130 1-((1-(3-chlorophenyl)-3-(trifluoromethyl)-1H-pyrazol-5-yl)methyl)-3-(2-methoxypyrimidin-5-yl)urea;
131 1-((3-tert-butyl-1-(3-chlorophenyl)-1H-pyrazol-5-yl)methyl)-3-(6-(2-methoxyethoxy)pyridin-3-yl)urea;
132 1-((1-(3-chlorophenyl)-3-(trifluoromethyl)-1H-pyrazol-5-yl)methyl)-3-(6-(2-methoxyethoxy)pyridin-3-yl)urea;
133 1-((1-(3-chlorophenyl)-3-(trifluoromethyl)-1H-pyrazol-5-yl)methyl)-3-(6-(2-hydroxyethoxy)pyridin-3-yl)urea;
134 1-((1-(3-chlorophenyl)-3-(trifluoromethyl)-1H-pyrazol-5-yl)methyl)-3-(6-((2-hydroxyethylamino)methyl)pyridin-3-yl)urea;
135 1-((1-(3-chlorophenyl)-3-(trifluoromethyl)-1H-pyrazol-5-yl)methyl)-3-(6-(((2-hydroxyethyl)(methyl)amino)methyl)pyridin-3-yl)urea;
136 1-((1-(3-chlorophenyl)-3-(trifluoromethyl)-1H-pyrazol-5-yl)methyl)-3-(6-methylpyridin-3-yl)urea;
137 1-((1-(3-chlorophenyl)-3-(trifluoromethyl)-1H-pyrazol-5-yl)methyl)-3-(5-methylpyridin-3-yl)urea;
138 1-((1-(3-chlorophenyl)-3-(trifluoromethyl)-1H-pyrazol-5-yl)methyl)-3-(4,6-dimethylpyridin-3-yl)urea;
139 1-((3-tert-butyl-1-(3-chlorophenyl)-1H-pyrazol-5-yl)methyl)-3-(5-(hydroxymethyl)pyridin-2-yl)urea;
140 1-((3-tert-butyl-1-(3-chlorophenyl)-1H-pyrazol-5-yl)methyl)-3-(5-(hydroxymethyl)pyridin-3-yl)urea;
141 1-((1-(3-chlorophenyl)-3-(trifluoromethyl)-1H-pyrazol-5-yl)methyl)-3-(6-(hydroxymethyl)-2-methylpyridin-3-yl)urea;
142 N-((1-(3-chlorophenyl)-3-(trifluoromethyl)-1H-pyrazol-5-yl)methyl)-2-(5-fluoro-6-(hydroxymethyl)pyridin-3-yl)propanamide;
143 1-((1-(3-chlorophenyl)-3-(trifluoromethyl)-1H-pyrazol-5-yl)methyl)-3-(6-(2-hydroxyethyl)-2-methylpyridin-3-yl)urea;
144 1-((3-tert-butyl-1-(3-chlorophenyl)-1H-pyrazol-5-yl)methyl)-3-(6-(1,2-dihydroxyethyl)pyridin-3-yl)urea;
145 1-((1-(3-chlorophenyl)-3-(trifluoromethyl)-1H-pyrazol-5-yl)methyl)-3-(6-(1,2-dihydroxyethyl)pyridin-3-yl)urea;
146 N-((1-(3-chlorophenyl)-3-(trifluoromethyl)-1H-pyrazol-5-yl)methyl)-2-(6-(2-hydroxyethylamino)pyridin-3-yl)propanamide;
147 N-((1-(3-chlorophenyl)-3-(trifluoromethyl)-1H-pyrazol-5-yl)methyl)-2-(6-(2-methoxyethylamino)pyridin-3-yl)propanamide;
148 N-((1-(3-chlorophenyl)-3-(trifluoromethyl)-1H-pyrazol-5-yl)methyl)-2-(6-((2-hydroxyethyl)(methyl)amino)pyridin-3-yl)propanamide;
149 N-((1-(3-chloro-4-fluorophenyl)-3-(trifluoromethyl)-1H-pyrazol-5-yl)methyl)-2-(6-(2-hydroxyethyl)pyridin-3-yl)propanamide;
150 N-((1-(3-chlorophenyl)-3-cyclopropyl-1H-pyrazol-5-yl)methyl)-2-(6-(2-hydroxyethyl)pyridin-3-yl)propanamide;
151 2-(6-(2-hydroxyethyl)pyridin-3-yl)-N-((1-m-tolyl-3-(trifluoromethyl)-1H-pyrazol-5-yl)methyl)propanamide;
152 1-((3-tert-butyl-1-(3-fluorophenyl)-1H-pyrazol-5-yl)methyl)-3-(6-(2-hydroxyethyl)pyridin-3-yl)urea;
153 1-((3-tert-butyl-1-(3-methoxyphenyl)-1H-pyrazol-5-yl)methyl)-3-(6-(2-hydroxyethyl)pyridin-3-yl)urea;
154 1-((3-tert-butyl-1-(3-fluorophenyl)-1H-pyrazol-5-yl)methyl)-3-(6-(2-hydroxyethylamino)pyridin-3-yl)urea;
155 1-((1-(3,5-difluorophenyl)-3-(trifluoromethyl)-1H-pyrazol-5-yl)methyl)-3-(6-(2-hydroxyethylamino)pyridin-3-yl)urea;
156 1-((1-(4-chloro-3-methylphenyl)-3-(trifluoromethyl)-1H-pyrazol-5-yl)methyl)-3-(6-(2-hydroxyethylamino)pyridin-3-yl)urea;
157 1-(6-(2-hydroxyethylamino)pyridin-3-yl)-3-((1-(4-methoxy-3-methylphenyl)-3-(trifluoromethyl)-1H-pyrazol-5-yl)methyl)urea;
158 1-((1-(4-fluoro-3-methylphenyl)-3-(trifluoromethyl)-1H-pyrazol-5-yl)methyl)-3-(6-(2-hydroxyethylamino)pyridin-3-yl)urea;
159 1-((3-tert-butyl-1-(3-chlorophenyl)-1H-pyrazol-5-yl)methyl)-3-(6-(methylsulfonylmethyl)pyridin-3-yl)urea;
160 1-((3-tert-butyl-1-(3-fluorophenyl)-1H-pyrazol-5-yl)methyl)-3-(6-(hydroxymethyl)pyridin-3-yl)urea;
161 N-((1-(3-chlorophenyl)-3-(trifluoromethyl)-1H-pyrazol-5-yl)methyl)-2-(6-(2-(methylsulfonyl)ethyl)pyridin-3-yl)propanamide;
162 N-((5-(3-((1-(3-chlorophenyl)-3-(trifluoromethyl)-1H-pyrazol-5-yl)methyl)ureido)pyrimidin-2-yl)methyl)methanesulfonamide; and
163 1-((3-cyclopropyl-1-(3-fluorophenyl)-1H-pyrazol-5-yl)methyl)-3-(6-(hydroxymethyl)pyridin-3-yl)urea;
optionally in the form of a single stereoisomer or a mixture of stereoisomers, in the form of the free compound and/or a physiologically acceptable salt thereof.

14. A pharmaceutical composition comprising a compound according to claim 1 and at least one pharmaceutically acceptable carrier or auxiliary substance.

15. A method of treating or inhibiting a disorder or disease selected from the group consisting of pain; hyperalgesia; allodynia; causalgia; migraine; depression; nervous affection; axonal injuries; neurodegenerative diseases; cognitive dysfunctions; epilepsy; respiratory diseases; coughs; urinary incontinence; overactive bladder; disorders and/or injuries of the gastrointestinal tract; duodenal ulcers; gastric ulcers; irritable bowel syndrome; strokes; eye irritations; skin irritations; neurotic skin diseases; allergic skin diseases; psoriasis; vitiligo; herpes simplex; inflammations; diarrhoea; pruritus; osteoporosis; arthritis; osteoarthritis; rheumatic diseases; eating disorders; medication dependency; misuse of medication; withdrawal symptoms in medication dependency; development of tolerance to medication; drug dependency; misuse of drugs; withdrawal symptoms in drug dependency; alcohol dependency; misuse of alcohol and withdrawal symptoms in alcohol dependency; or of effecting diuresis; antinatriuresis; influencing the cardiovascular system; increasing vigilance; treating wounds and/or burns; treating severed nerves; increasing libido; modulating movement activity; effecting anxiolysis; local anaesthesia and/or inhibiting undesirable side effects triggered by the administration of a vanilloid receptor 1 agonist in a mammal, said method comprising administering a pharmacologically effective amount of a compound according to claim 1 to said mammal.

16. A method according to claim 15, wherein said disorder or disease is pain selected from the group consisting of acute pain, chronic pain, neuropathic pain, visceral pain and joint pain; a neurodegenerative disease selected from the group consisting of multiple sclerosis, Alzheimer's disease, Parkinson's disease and Huntington's disease; a memory disorder; a respiratory disease selected from the group consisting of asthma, bronchitis and pulmonary inflammation; an inflammation of the intestine, the eyes, the bladder, the skin or the nasal mucous membrane; an eating disorder selected from the group consisting of bulimia, cachexia, anorexia and obesity; development of tolerance to natural or synthetic opioids; or of inhibiting undesirable side effects selected from the group consisting of hyperthermia, hypertension and bronchoconstriction, triggered by administration of a vanilloid receptor 1 agonsit selected from the group consisting of capsaicin, resiniferatoxin, olvanil, arvanil, SDZ-249665, SDZ-249482, nuvanil and capsavanil.
